SEQUENCE LISTING (72 sequences)

SEQ ID NO: 1 - DNA, 21527 bp, synthetic construct
  misc_feature 1..21527: note = pJP3416-GA7 nucleotide sequence.
  source 1..21527: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgcggccgcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacgtgcttaacgtaattcaacagaaattatatgataatcatcgcaagaccggcaacaggattcaatcttaagaaactttattgccaaatgtttgaacgatcggcgcgcctcattagtgagccttctcagcctttccgttaacgtagtagtgctgtcccaccttatcaaggttagagaaagtagccttccaagcaccgtagtaagagagcaccttgtagttgagtccccacttcttagcgaaaggaacgaatcttctgctaacctcaggctgtctgaattgaggcatatcagggaagaggtggtggataacctgacagttaaggtatcccataagccagttcacgtatcctctagaaggatcgatatcaacggtgtgatcaacagcgtagttaacccaagaaaggtgcttatcagatggaacaacagggaggtgagtatgagaagtagagaagtgagcgaaaaggtacatgtaagcgatccagtttccgaaagtgaaccaccagtaagcaacaggccaagagtatccagtagcaagcttgataacagcggttctaacaacatgagaaacgagcatccaagaagcctcttcgtagttcttcttacggagaacttgtctagggtggagaacgtagatccagaaagcttgaacaagaagtccagaggtaacaggaacgaaagtccaagcttgaagtctagcccaagctctagagaatcctctaggtctgttatcctcaacagcagtgttgaagaaagccacagcaggagtggtatcaagatccatatcgtgtctaaccttttgaggggtagcatggtgcttgttatgcatctggttccacatctcaccagaagtagaaagtccgaatccacaagtcatagcctgaagtctcttgtccacgtaaacagatccggtaagagagttatgtccaccctcatgttgaacccatccacatctagctccgaagaaagcaccgtaaacaacagaagcaatgatagggtatccagcgtacataagagcagttccaagagcgaatgtagcaagaagctcgagaagtctgtaagccacatgggtgatagaaggcttgaagaatccatctctctcaagctcagcacgccatctagcgaaatcctcaagcataggagcatcctcagactcagatctcttgatctcagcaggtctagaaggcaaagctctaagcatcttccaagccttgagagaacgcatgtggaattctttgaaagcctcagtagcatcagcaccagtgttagcaagcatgtagaagatcacagatccaccagggtgcttgaagttagtcacatcgtactcaacgtcctcaactctaacccatctagtctcgaaagtagcagcaagctcatgaggctcaagagtcttaagatcaacaggagcagtagaagcatccttagcatcaagagcctcagcagaagatttagacctggtaagtggagatctaggagaagatcttccatcagtcttaggagggcacatggtatggtaattgtaaatgtaattgtaatgttgtttgttgtttgttgttgttggtaattgttgtaaaagatcctcgtgtatgtttttaatcttgtttgtatcgatgagttttggtttgagtaaagagtgaagcggatgagttaatttataggctataaaggagatttgcatggcgatcacgtgtaataatgcatgcacgcatgtgattgtatgtgtgtgctgtgagagagaagctcttaggtgtttgaagggagtgacaagtggcgaagaaaaacaattctccgcggctgcatgctatgtgtaacgtgtagctaatgttctggcatggcatcttatgaacgattctttttaaaaacaaggtaaaaacttaacttcataaaattaaaaaaaaaaacgtttactaagttggtttaaaaggggatgagactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggaattcgcccagcctcttgttgctatggctcaagagcaatacgctgctatcgatgctgttgttgctcctgctatcttctctgctactgattctatcggatggggacttaagcctatctcttctgctactaaggacttgcctcttgttgagtctcctacacctctcatcctttctttgcttgcttacttcgctatcgttggatctggactcgtttacagaaaggttttccctagaaccgtgaagggacaagatccattccttttgaaggctcttatgcttgctcacaacgtgttccttatcggactttctctttacatgtgcctcaagcttgtgtacgaggcttacgttaacaagtactctttctggggaaacgcttacaaccctgctcaaactgagatggctaaggttatctggatcttctacgtgagcaagatctacgagttcatggataccttcatcatgctcctcaagggaaatgttaaccaggttagcttccttcacgtttaccatcacggatctatctctggaatctggtggatgattacttacgctgctcctggtggtgatgcttacttctctgctgctcttaactcttgggttcacgtgtgtatgtacacctactattttatggctgccgtgcttcctaaggacgagaaaactaagagaaagtacctctggtggggaagataccttactcaaatgcagatgttccagttcttcatgaaccttctccaggctgtttaccttctctactcttcatctccttaccctaagtttatcgctcagctcctcgtggtgtacatggttactcttctcatgcttttcggaaacttctactacatgaagcaccacgctagcaagtgatgaggcgcgccgggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagtctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcgatacaaacaagattaaaaacatacacgaggatcttttacaacaattaccaacaacaacaaacaacaaacaacattacaattacatttacaattaccataccatgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatgagatcgttcaaacatttggcaataaagtttcttaagattgaatcctgttgccggtcttgcgatgattatcatataatttctgttgaattacgttaagcacgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgatagaacccacacgagcatgtgctttccatttaattttaaaaaccaagaaacatacatacataacattccatcagcctctctctctttttattacggttaatgacttaaaacacatcttattatcccatccttaacacctagcagtgtctttatacgatctcatcgatcaccacttcaaaaccatgcagactgctgctgcccctggagctggcatcggctaggctgggtgccgcactgtcccggaaggtccctagcgacttgtttagattgatgggaccacctctcaacttcctgctgctgtccctgctgctggatgtcctgcctcatctggccgattgcacgctccagtcccctgcatgtgcactcgctcctcaattgcttaagatcatcgcagcagctatcgaagtgctggctctgttgccctcctccacggccttggttgtagtagtagctgccgccgcccttctggactttttcccacaggaaccgccgaataattcgatagaaccacacgagcatgtgctttcatttattttaaaaaccaagaaacatacataacatttcatcagcctctctctctctctctctctctctctctctctctctctctctctctctctctttattacagctgttacactaacttaaaacacattcatctcattattattattattatccatccttaacacctagcagtgtctttgtacgatctcataatcgatcaccccttcatcaggtatccttaggcttcactccaacgttgttgcagttacggaacatgtacacaccatcatggttctcaacgaactggcaagatctccaagttttccaaaggctaacccacatgttctcatcggtgtgtctgtagtgctctcccataactttcttgatgcactcggtagcttctctagcatggtagaatgggatccttgaaacgtagtgatggagcacatgagtctcgatgatgtcatggaagatgattccgaggattccgaactctctatcgatagtagcagcagcacccttagcgaaagtccactcttgagcatcgtaatgaggcatagaagaatcggtgtgctgaaggaaggtaacgaaaacaagccagtggttaacaaggatccaaggacagaaccatgtgatgaaagtaggccagaatccgaaaaccttgtaagcggtgtaaacagaagtgagggtagcaaggattccaagatcagaaagaacgatgtaccagtagtccttcttatcgaaaacagggctagaaggccagtagtgagacttgaagaacttagaaacaccagggtaaggttgtccagtagcgttagtagcaaggtaaagagaaagtcctccaagctgttggaacaagagagcgaaaacagagtagataggagtttcctcagcgatatcgtgaaggctggtaacttggtgcttctctttgaattcctcggcggtgtaaggaacgaaaaccatatctctggtcatgtgtccagtagccttatggtgcttagcatgagagaacttccagctgaagtaaggaaccataacaagagagtggagaacccatccaacggtatcgttaacccatccgtagttagagaaagcagaatgtccacactcatgtccaaggatccagattccgaatccgaaacaagagatagagaacacgtaagcagaccaagcagcgaatctaaggaattcgttagggagaagagggatgtaggtaagtccaacgtaagcgatagcagagatagccacgatatctctcaccacgtaagacatagacttcacgagagatctctcgtaacagtgcttagggatagcgtcaaggatatccttgatggtgtaatctggcaccttgaaaacgtttccgaaggtatcgatagcggtcttttgctgcttgaaagatgcaacgtttccagaacgcctaacggtcttagtagatccctcaaggatctcagatccagacacggtaaccttagacatggtatggtaattgtaaatgtaattgtaatgttgtttgttgtttgttgttgttggtaattgttgtaaaatttttggtggtgattggttctttaaggtgtgagagtgagttgtgagttgtgtggtgggtttggtgagattggggatggtgggtttatatagtggagactgaggaatggggtcgtgagtgttaactttgcatgggctacacgtgggttcttttgggcttacacgtagtattattcatgcaaatgcagccaatacatatacggtattttaataatgtgtgggaatacaatatgccgagtattttactaattttggcaatgacaagtgtacatttggattatcttacttggcctctcttgctttaatttggattatttttattctcttaccttggccgttcatattcacatccctaaaggcaagacagaattgaatggtggccaaaaattaaaacgatggatatgacctacatagtgtaggatcaattaacgtcgaaggaaaatactgattctctcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaatactcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacttttacaacaattaccaacaacaacaaacaacaaacaacattacaattacatttacaattaccataccatgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatgacaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctgagtcgaacgggcagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattttacaacaattaccaacaacaacaaacaacaaacaacattacaattacatttacaattaccataccatgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatgaaggggtgatcgattatgagatcgtacaaagacactgctaggtgttaaggatggataataataataataatgagatgaatgtgttttaagttagtgtaacagctgtaataaagagagagagagagagagagagagagagagagagagagagagagagagagaggctgatgaaatgttatgtatgtttcttggtttttaaaataaatgaaagcacatgctcgtgtggttctatcgaattattcggcggttcctgtgggaaaaagtccagaagggccgccgcagctactactacaaccaaggccgtggaggagggcaacagagccagcacttcgatagctgctgcgatgatcttaagcaattgaggagcgagtgcacatgcaggggactggagcgtgcaatcggccagatgaggcaggacatccagcagcagggacagcagcaggaagttgagaggtggtcccatcaatctaaacaagtcgctagggaccttccgggacagtgcggcacccagcctagccgatgccagctccaggggcagcagcagtctgcatggttttgaagtggtgatcgatgagatcgtataaagacactgctaggtgttaaggatgggataataagatgtgttttaagtcattaaccgtaataaaaagagagagaggctgatggaatgttatgtatgtatgtttcttggtttttaaaattaaatggaaagcacatgctcgtgtgggttctatctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtcctgcaggctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggcctctatcgctatccctgctgctcttgctggaactcttggatacgttacctacaatgtggctaaccctgatatcccagcttctgagaaagttcctgcttacttcatgcaggttgagtactggggacctactatcggaactattggatacctcctcttcatctacttcggaaagcgtatcatgcagaacagatctcaacctttcggactcaagaacgctatgctcgtttacaacttctaccagaccttcttcaacagctactgcatctaccttttcgttacttctcatagggctcagggacttaaggtttggggaaacatccctgatatgactgctaactcttggggaatctctcaggttatctggcttcactacaacaacaagtacgttgagcttctcgacaccttcttcatggtgatgaggaagaagttcgaccagctttctttccttcacatctaccaccacactcttctcatctggtcatggttcgttgttatgaagcttgagcctgttggagattgctacttcggatcttctgttaacaccttcgtgcacgtgatcatgtactcttactacggacttgctgctcttggagttaactgtttctggaagaagtacatcacccagatccagatgcttcagttctgtatctgtgcttctcactctatctacaccgcttacgttcagaataccgctttctggcttccttaccttcaactctgggttatggtgaacatgttcgttctcttcgccaacttctaccgtaagaggtacaagtctaagggtgctaagaagcagtgataagggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagcctgcagggcggccgcggatcccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgaattaaatatgagccctgagaggcgtcctgttgaaatcagacctgctactgctgctgatatggctgctgtttgtgatatcgtgaaccactacatcgagacttctaccgttaacttcagaactgagcctcaaactcctcaagagtggatcgatgatcttgagagactccaagatagatacccttggcttgttgctgaggttgagggtgttgttgctggaatcgcttacgctggaccttggaaggctagaaacgcttacgattggactgttgagtctaccgtttacgtttcacacagacatcagagacttggacttggatctaccctttacactcaccttctcaagtctatggaagctcagggattcaagtctgttgttgctgttatcggactccctaacgatccttctgttagacttcatgaggctcttggatacactgctagaggaactcttagagctgctggatacaagcacggtggatggcatgatgttggattctggcaaagagatttcgagcttcctgctcctcctagacctgttagaccagttactcagatctgaatttgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 2 - DNA, 23512 bp, synthetic construct
  misc_feature 1..23512: note = pGA7- mod_B nucleotide sequence
  source 1..23512: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgcggccgcggtaccgcccgttcgactcagatcttccaaggcctcgtctccgagtccgctgcttctcgccgcgccgatcacttctccgccgccaacaaggcttgtagttaataggaatcattcagggattgtgattccgggcagtagtaattaataatatagtattagtatagataatatgtttcgtttgggatctttggaacgttgctctgttccttgttgttcattttaaagcttttgagggatagttgcagaactgttcggtgatgcttcatcctctcaagaactagatttgggtaaagaaacatccatgcatggatatggaatgttgttcttccgattggagattattttataaaatttaaaattcatgatttaaaaaaacacataaaaaccacaaaattcatgatttattgacaatacgatacaaaattagcaccaccggctactggctcattacacatttccccttcccctcattctcactttgtggctttattattattattattacatatattttaccgttattatttcacgtcacataagcttgttaattaatcattagtgagccttctcagcctttccgttaacgtagtagtgctgtcccaccttatcaaggttagagaaagtagccttccaagcaccgtagtaagagagcaccttgtagttgagtccccacttcttagcgaaaggaacgaatcttctgctaacctcaggctgtctgaattgaggcatatcagggaagaggtggtggataacctgacagttaaggtatcccataagccagttcacgtatcctctagaaggatcgatatcaacggtgtgatcaacagcgtagttaacccaagaaaggtgcttatcagatggaacaacagggaggtgagtatgagaagtagagaagtgagcgaaaaggtacatgtaagcgatccagtttccgaaagtgaaccaccagtaagcaacaggccaagagtatccagtagcaagcttgataacagcggttctaacaacatgagaaacgagcatccaagaagcctcttcgtagttcttcttacggagaacttgtctagggtggagaacgtagatccagaaagcttgaacaagaagtccagaggtaacaggaacgaaagtccaagcttgaagtctagcccaagctctagagaatcctctaggtctgttatcctcaacagcagtgttgaagaaagccacagcaggagtggtatcaagatccatatcgtgtctaaccttttgaggggtagcatggtgcttgttatgcatctggttccacatctcaccagaagtagaaagtccgaatccacaagtcatagcctgaagtctcttgtccacgtaaacagatccggtaagagagttatgtccaccctcatgttgaacccatccacatctagctccgaagaaagcaccgtaaacaacagaagcaatgatagggtatccagcgtacataagagcagttccaagagcgaatgtagcaagaagctcgagaagtctgtaagccacatgggtgatagaaggcttgaagaatccatctctctcaagctcagcacgccatctagcgaaatcctcaagcataggagcatcctcagactcagatctcttgatctcagcaggtctagaaggcaaagctctaagcatcttccaagccttgagagaacgcatgtggaattctttgaaagcctcagtagcatcagcaccagtgttagcaagcatgtagaagatcacagatccaccagggtgcttgaagttagtcacatcgtactcaacgtcctcaactctaacccatctagtctcgaaagtagcagcaagctcatgaggctcaagagtcttaagatcaacaggagcagtagaagcatccttagcatcaagagcctcagcagaagatttagacctggtaagtggagatctaggagaagatcttccatcagtcttaggagggcacatggtatggtaattgtaaatgtaattgtaatgttgtttgttgtttgttgttgttggtaattgttgtaaaattaattaagtgggtatcttttggatggataagcaagtagtgatgatgttctaggtgaagtgatgggggtgttttatagcgggagatggtgaaatggatggtcgccacataagaaatggaggggaagggttcttgcgccattcttcagtttgcatggatgcatgggtttcattttgtaacacgtaataaggacaatgaagtgcaggtgtctctcaagtttcagaggggatatgtggacagaagaagaacggcgatgatattgatggaaatggccatctagtgtgaatctattcggttgataatactagtgcattttggccgttaatcccttcaattaactgcacaaacttcagttgagtattgattatttgattataggttctgtaaacacaataccaagtttatttagaggggagacatacaaatagtttcgatataaataatagagtggttaaacttagttattaaaactatatataaagtctaaaagttaaattatttttttaattgcaaatatataaagtctaaaggggttacattatttcttaagagatgtaactctgttggaatctgacttaatccgtctcatcactctggtttccagttctaatctaatgaattgttttctgccaaagaatttgaagcaagaagtaaattgatcaatgccgtcaacccacaccaaaccgtcaacccactaccatcgccgcggagacccccaaactcaacctccacccatcggtaagaagcacagggcagcccgcaccaccaccaatttggcgtgcatgacacctagggacttggcacgggaggcggcgcacgtggatgcaaatgacgggatatcagatgacaggaaacgacgttgagagaccatacgatgtagaatatgagctcaccatcaacgagaaactaggaaaatcacaaaaaaaacaactctcgtaattgtacgagtggcacagatgggtctgcctcaacatatctctaatacggcgaagcctgcccaacacgtagttgccggaatccggtgtggagctcacgactctgaaagataggcgcttcctgtttcgtttcgctcacccactggacgtccgtcatgtgatggatttcggtcattggtttgctgacaaccacattctgaagctccatgagatgagtcttcacaataggtcctgctcaataccgtggagttatggttgcaagtccataacttgccgttcgaatattttgcggagccagtcggacgggaattggcgagctcggctgacacctataaaggccatgacaagaagaaccaaaagttcttccctaatgctttcatgaggcttcgggtcgttatggatgtcggaaaacccctcttgaaggaacgagacgttattatgcatgacggtaagactattacttgtcagtataagtatgaaagattacctgtcttctgctttgtttgtggattgattggacacgttgaaaaaaaatgtgcacttcgatttcaatactcagagatcgacttcccttttctctaggagtattcgatcaaggcattaacatggaaggaagctcaagctctaaaggcttcacaatggaacctgaaaaatttcaacaagcctaaactgaaatcgaagtcaaatcacccaaccgggagctctaaatcagcaaacactcctcctccacagtatccaatcatcgtgcacgatgctccaggtattgcaagccaggtattgcaagctaggagtaggatagagaccttaaacgtcgttggtgtgaagagtcatcttcagacctaatggagatagatgtagacggcggcacgaagactctgaaacaccagaaaggctagtccaggataaggatctgctatcccaactgacctctcgttagtcccaaggcctctcaactagagcaggaggaaggatggtcacaagactaggataatgatgtttccaatatgaacctgaatgtccatagctaatttttttagtcttgcttctgcactttttgtttattatgttctggtgactatgttatttacccttgtccgtatgcttgagggtaccctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggcctctatcgctatccctgctgctcttgctggaactcttggatacgttacctacaatgtggctaaccctgatatcccagcttctgagaaagttcctgcttacttcatgcaggttgagtactggggacctactatcggaactattggatacctcctcttcatctacttcggaaagcgtatcatgcagaacagatctcaacctttcggactcaagaacgctatgctcgtttacaacttctaccagaccttcttcaacagctactgcatctaccttttcgttacttctcatagggctcagggacttaaggtttggggaaacatccctgatatgactgctaactcttggggaatctctcaggttatctggcttcactacaacaacaagtacgttgagcttctcgacaccttcttcatggtgatgaggaagaagttcgaccagctttctttccttcacatctaccaccacactcttctcatctggtcatggttcgttgttatgaagcttgagcctgttggagattgctacttcggatcttctgttaacaccttcgtgcacgtgatcatgtactcttactacggacttgctgctcttggagttaactgtttctggaagaagtacatcacccagatccagatgcttcagttctgtatctgtgcttctcactctatctacaccgcttacgttcagaataccgctttctggcttccttaccttcaactctgggttatggtgaacatgttcgttctcttcgccaacttctaccgtaagaggtacaagtctaagggtgctaagaagcagtgataaggcgcgcggcgcgccgggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagtctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcgatacaaacaagattaaaaacatacacgaggatcttttacaacaattaccaacaacaacaaacaacaaacaacattacaattacatttacaattaccataccatgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatgagatcgttcaaacatttggcaataaagtttcttaagattgaatcctgttgccggtcttgcgatgattatcatataatttctgttgaattacgttaagcacgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgatagaacccacacgagcatgtgctttccatttaattttaaaaaccaagaaacatacatacataacattccatcagcctctctctctttttattacggttaatgacttaaaacacatcttattatcccatccttaacacctagcagtgtctttatacgatctcatcgatcaccacttcaaaaccatgcagactgctgctgcccctggagctggcatcggctaggctgggtgccgcactgtcccggaaggtccctagcgacttgtttagattgatgggaccacctctcaacttcctgctgctgtccctgctgctggatgtcctgcctcatctggccgattgcacgctccagtcccctgcatgtgcactcgctcctcaattgcttaagatcatcgcagcagctatcgaagtgctggctctgttgccctcctccacggccttggttgtagtagtagctgccgccgcccttctggactttttcccacaggaaccgccgaataattcgatagaaccacacgagcatgtgctttcatttattttaaaaaccaagaaacatacataacatttcatcagcctctctctctctctctctctctctctctctctctctctctctctctctctctttattacagctgttacactaacttaaaacacattcatctcattattattattattatccatccttaacacctagcagtgtctttgtacgatctcataatcgatcaccccttcatcaggtatccttaggcttcactccaacgttgttgcagttacggaacatgtacacaccatcatggttctcaacgaactggcaagatctccaagttttccaaaggctaacccacatgttctcatcggtgtgtctgtagtgctctcccataactttcttgatgcactcggtagcttctctagcatggtagaatgggatccttgaaacgtagtgatggagcacatgagtctcgatgatgtcatggaagatgattccgaggattccgaactctctatcgatagtagcagcagcacccttagcgaaagtccactcttgagcatcgtaatgaggcatagaagaatcggtgtgctgaaggaaggtaacgaaaacaagccagtggttaacaaggatccaaggacagaaccatgtgatgaaagtaggccagaatccgaaaaccttgtaagcggtgtaaacagaagtgagggtagcaaggattccaagatcagaaagaacgatgtaccagtagtccttcttatcgaaaacagggctagaaggccagtagtgagacttgaagaacttagaaacaccagggtaaggttgtccagtagcgttagtagcaaggtaaagagaaagtcctccaagctgttggaacaagagagcgaaaacagagtagataggagtttcctcagcgatatcgtgaaggctggtaacttggtgcttctctttgaattcctcggcggtgtaaggaacgaaaaccatatctctggtcatgtgtccagtagccttatggtgcttagcatgagagaacttccagctgaagtaaggaaccataacaagagagtggagaacccatccaacggtatcgttaacccatccgtagttagagaaagcagaatgtccacactcatgtccaaggatccagattccgaatccgaaacaagagatagagaacacgtaagcagaccaagcagcgaatctaaggaattcgttagggagaagagggatgtaggtaagtccaacgtaagcgatagcagagatagccacgatatctctcaccacgtaagacatagacttcacgagagatctctcgtaacagtgcttagggatagcgtcaaggatatccttgatggtgtaatctggcaccttgaaaacgtttccgaaggtatcgatagcggtcttttgctgcttgaaagatgcaacgtttccagaacgcctaacggtcttagtagatccctcaaggatctcagatccagacacggtaaccttagacatggtatggtaattgtaaatgtaattgtaatgttgtttgttgtttgttgttgttggtaattgttgtaaaatttttggtggtgattggttctttaaggtgtgagagtgagttgtgagttgtgtggtgggtttggtgagattggggatggtgggtttatatagtggagactgaggaatggggtcgtgagtgttaactttgcatgggctacacgtgggttcttttgggcttacacgtagtattattcatgcaaatgcagccaatacatatacggtattttaataatgtgtgggaatacaatatgccgagtattttactaattttggcaatgacaagtgtacatttggattatcttacttggcctctcttgctttaatttggattatttttattctcttaccttggccgttcatattcacatccctaaaggcaagacagaattgaatggtggccaaaaattaaaacgatggatatgacctacatagtgtaggatcaattaacgtcgaaggaaaatactgattctctcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaatactcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacttttacaacaattaccaacaacaacaaacaacaaacaacattacaattacatttacaattaccataccatgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatgacaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctgagtcgaacgggcagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattttacaacaattaccaacaacaacaaacaacaaacaacattacaattacatttacaattaccataccatgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatgaaggggtgatcgattatgagatcgtacaaagacactgctaggtgttaaggatggataataataataataatgagatgaatgtgttttaagttagtgtaacagctgtaataaagagagagagagagagagagagagagagagagagagagagagagagagagaggctgatgaaatgttatgtatgtttcttggtttttaaaataaatgaaagcacatgctcgtgtggttctatcgaattattcggcggttcctgtgggaaaaagtccagaagggccgccgcagctactactacaaccaaggccgtggaggagggcaacagagccagcacttcgatagctgctgcgatgatcttaagcaattgaggagcgagtgcacatgcaggggactggagcgtgcaatcggccagatgaggcaggacatccagcagcagggacagcagcaggaagttgagaggtggtcccatcaatctaaacaagtcgctagggaccttccgggacagtgcggcacccagcctagccgatgccagctccaggggcagcagcagtctgcatggttttgaagtggtgatcgatgagatcgtataaagacactgctaggtgttaaggatgggataataagatgtgttttaagtcattaaccgtaataaaaagagagagaggctgatggaatgttatgtatgtatgtttcttggtttttaaaattaaatggaaagcacatgctcgtgtgggttctatctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtcctgcaggctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggaatttgctcaacctctcgttgctatggctcaagagcagtacgctgctatcgatgctgttgttgctcctgctatcttctctgctaccgactctattggatggggactcaagcctatctcttctgctactaaggatctccctctcgttgaatctcctacccctcttatcctttctctcctcgcttacttcgctatcgttggttctggactcgtttaccgtaaagtgttccctagaaccgttaagggacaggatcctttccttctcaaggctcttatgctcgctcacaacgttttccttatcggactcagcctttacatgtgcctcaagctcgtttacgaggcttacgtgaacaagtactccttctggggaaacgcttacaaccctgctcaaaccgagatggctaaggtgatctggatcttctacgtgtccaagatctacgagttcatggacaccttcatcatgcttctcaagggaaacgttaaccaggtttccttcctccatgtttaccaccacggatctatctctggaatctggtggatgatcacttatgctgctccaggtggagatgcttacttctctgctgctctcaactcttgggttcatgtgtgcatgtacacctactacttcatggctgctgttcttcctaaggacgaaaagaccaagagaaagtacctttggtggggaagataccttacccagatgcaaatgttccagttcttcatgaaccttctccaggctgtttacctcctctactcttcttctccttaccctaagttcattgctcaactcctcgttgtttacatggttaccctcctcatgcttttcggaaacttctactacatgaagcaccacgcttctaagtgataagggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagcctgcagggcggccgcggatcccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgaattaaatatgagccctgagaggcgtcctgttgaaatcagacctgctactgctgctgatatggctgctgtttgtgatatcgtgaaccactacatcgagacttctaccgttaacttcagaactgagcctcaaactcctcaagagtggatcgatgatcttgagagactccaagatagatacccttggcttgttgctgaggttgagggtgttgttgctggaatcgcttacgctggaccttggaaggctagaaacgcttacgattggactgttgagtctaccgtttacgtttcacacagacatcagagacttggacttggatctaccctttacactcaccttctcaagtctatggaagctcagggattcaagtctgttgttgctgttatcggactccctaacgatccttctgttagacttcatgaggctcttggatacactgctagaggaactcttagagctgctggatacaagcacggtggatggcatgatgttggattctggcaaagagatttcgagcttcctgctcctcctagacctgttagaccagttactcagatctgaatttgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 3 - DNA, 25787 bp, synthetic construct
  misc_feature 1..25787: note = pGA7- mod_C nucleotide sequence
  source 1..25787: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacgtgcttaacgtaattcaacagaaattatatgataatcatcgcaagaccggcaacaggattcaatcttaagaaactttattgccaaatgtttgaacgatctgcccggaagcggccaactcgaaaatttaattaatcatcagtgagccttctcagcctttccgttaacgtagtagtgctgtccaactttgtcgaggttgctgaaagtagccttccaagcaccgtagtaagagagcaccttgtagttgagtccccacttcttagcgaaagggacgaatcttcttgacacctcaggctgtctgaattgaggcatatcagggaagagatggtggataacctggcagttaaggtatcccataagccagttaacgtatccacgagaaggatcgatgtcaacggtgtgatcaacagcgtagttaacccagctaaggtgcttgtcagatggaacaacagggaggtgagtgtgagaagtagagaagtgagcgaagaggtacatgtaagcgatccagtttccgaaagtgaaccaccagtaagcaacaggccaagagtatccggtagcaagcttgataacagcggttctaacaacgtgagaaacgagcatccaagaagcttcctcgtagttcttcttcctgagcacctgtctaggatggagaacgtagatccagaaagcctgaacgagaagtccagaagtaacaggaacgaaggtccaagcttgaagtctagcccaagctctagagaatcccctaggtctattatcctccacagcggtgttgaagaaagccacagcaggagtggtatcaagatccatgtcgtgtctaactttctgaggggtagcatggtgcttgttatgcatctggttccacatctctccgctggtagaaagtccgaatccgcaagtcatagcctgaagtctcttatccacgtacacagatccggtaagagagttgtgtccaccctcatgttgaacccatccacatctagctccgaagaaagcaccgtacacaacgctagcaatgatagggtatccagcgtacataagagcggttccaagagcgaaagtagcaagaagctccaaaagacggtaagcaacatgggtgatagaaggcttgaagaatccgtccctctcaagttcagctctccacctagcgaaatcctcaagcataggagcatcctcagactcagatctcttgatctcagcaggtctagaaggcaaagctctaagcatcttccaagccttgaggctacgcatgtgaaattctttgaaagcctcagtagcatcagcaccagtgttagcaagcatgtagaagatcacgcttccaccaggatgtttgaagttggtcacgtcgtactcaacatcctcaaccctaacccatctagtctcgaaggtagcagcaagttcatgaggctcaagggtcttaagatcaacaggagcggtagaagcatccttagcatcaagagcctcagcagatgacttagacctggtgagaggagatctaggagaagatcttccatcggtcttaggaggacacatggcgcgccgattttcgagatggtaattgtaaatgtaattgtaatgttgtttgttgtttgttgttgttggtaattgttgtaaaattcgagttggccgcttccggggatcctcgtgtatgtttttaatcttgtttgtatcgatgagttttggtttgagtaaagagtgaagcggatgagttaatttataggctataaaggagatttgcatggcgatcacgtgtaataatgcatgcacgcatgtgattgtatgtgtgtgctgtgagagagaagctcttaggtgtttgaagggagtgacaagtggcgaagaaaaacaattctccgcggctgcatgctatgtgtaacgtgtagctaatgttctggcatggcatcttatgaacgattctttttaaaaacaaggtaaaaacttaacttcataaaattaaaaaaaaaaacgtttactaagttggtttaaaaggggatgagaggcgccccgcggaaagcttgctagccaattggggcccaacgttctcgagtttttctagaaggaaactgaaggcgggaaacgacaatctgctagtggatctcccagtcacgacgttgtaaaacgggcgccccgcggaaagcttgcggccgcggtaccgcccgttcgactcagatcttccaaggcctcgtctccgagtccgctgcttctcgccgcgccgatcacttctccgccgccaacaaggcttgtagttaataggaatcattcagggattgtgattccgggcagtagtaattaataatatagtattagtatagataatatgtttcgtttgggatctttggaacgttgctctgttccttgttgttcattttaaagcttttgagggatagttgcagaactgttcggtgatgcttcatcctctcaagaactagatttgggtaaagaaacatccatgcatggatatggaatgttgttcttccgattggagattattttataaaatttaaaattcatgatttaaaaaaacacataaaaaccacaaaattcatgatttattgacaatacgatacaaaattagcaccaccggctactggctcattacacatttccccttcccctcattctcactttgtggctttattattattattattacatatattttaccgttattatttcacgtcacataagcttgttaattaatcattagtgagccttctcagcctttccgttaacgtagtagtgctgtcccaccttatcaaggttagagaaagtagccttccaagcaccgtagtaagagagcaccttgtagttgagtccccacttcttagcgaaaggaacgaatcttctgctaacctcaggctgtctgaattgaggcatatcagggaagaggtggtggataacctgacagttaaggtatcccataagccagttcacgtatcctctagaaggatcgatatcaacggtgtgatcaacagcgtagttaacccaagaaaggtgcttatcagatggaacaacagggaggtgagtatgagaagtagagaagtgagcgaaaaggtacatgtaagcgatccagtttccgaaagtgaaccaccagtaagcaacaggccaagagtatccagtagcaagcttgataacagcggttctaacaacatgagaaacgagcatccaagaagcctcttcgtagttcttcttacggagaacttgtctagggtggagaacgtagatccagaaagcttgaacaagaagtccagaggtaacaggaacgaaagtccaagcttgaagtctagcccaagctctagagaatcctctaggtctgttatcctcaacagcagtgttgaagaaagccacagcaggagtggtatcaagatccatatcgtgtctaaccttttgaggggtagcatggtgcttgttatgcatctggttccacatctcaccagaagtagaaagtccgaatccacaagtcatagcctgaagtctcttgtccacgtaaacagatccggtaagagagttatgtccaccctcatgttgaacccatccacatctagctccgaagaaagcaccgtaaacaacagaagcaatgatagggtatccagcgtacataagagcagttccaagagcgaatgtagcaagaagctcgagaagtctgtaagccacatgggtgatagaaggcttgaagaatccatctctctcaagctcagcacgccatctagcgaaatcctcaagcataggagcatcctcagactcagatctcttgatctcagcaggtctagaaggcaaagctctaagcatcttccaagccttgagagaacgcatgtggaattctttgaaagcctcagtagcatcagcaccagtgttagcaagcatgtagaagatcacagatccaccagggtgcttgaagttagtcacatcgtactcaacgtcctcaactctaacccatctagtctcgaaagtagcagcaagctcatgaggctcaagagtcttaagatcaacaggagcagtagaagcatccttagcatcaagagcctcagcagaagatttagacctggtaagtggagatctaggagaagatcttccatcagtcttaggagggcacatggtatggtaattgtaaatgtaattgtaatgttgtttgttgtttgttgttgttggtaattgttgtaaaattaattaagtgggtatcttttggatggataagcaagtagtgatgatgttctaggtgaagtgatgggggtgttttatagcgggagatggtgaaatggatggtcgccacataagaaatggaggggaagggttcttgcgccattcttcagtttgcatggatgcatgggtttcattttgtaacacgtaataaggacaatgaagtgcaggtgtctctcaagtttcagaggggatatgtggacagaagaagaacggcgatgatattgatggaaatggccatctagtgtgaatctattcggttgataatactagtgcattttggccgttaatcccttcaattaactgcacaaacttcagttgagtattgattatttgattataggttctgtaaacacaataccaagtttatttagaggggagacatacaaatagtttcgatataaataatagagtggttaaacttagttattaaaactatatataaagtctaaaagttaaattatttttttaattgcaaatatataaagtctaaaggggttacattatttcttaagagatgtaactctgttggaatctgacttaatccgtctcatcactctggtttccagttctaatctaatgaattgttttctgccaaagaatttgaagcaagaagtaaattgatcaatgccgtcaacccacaccaaaccgtcaacccactaccatcgccgcggagacccccaaactcaacctccacccatcggtaagaagcacagggcagcccgcaccaccaccaatttggcgtgcatgacacctagggacttggcacgggaggcggcgcacgtggatgcaaatgacgggatatcagatgacaggaaacgacgttgagagaccatacgatgtagaatatgagctcaccatcaacgagaaactaggaaaatcacaaaaaaaacaactctcgtaattgtacgagtggcacagatgggtctgcctcaacatatctctaatacggcgaagcctgcccaacacgtagttgccggaatccggtgtggagctcacgactctgaaagataggcgcttcctgtttcgtttcgctcacccactggacgtccgtcatgtgatggatttcggtcattggtttgctgacaaccacattctgaagctccatgagatgagtcttcacaataggtcctgctcaataccgtggagttatggttgcaagtccataacttgccgttcgaatattttgcggagccagtcggacgggaattggcgagctcggctgacacctataaaggccatgacaagaagaaccaaaagttcttccctaatgctttcatgaggcttcgggtcgttatggatgtcggaaaacccctcttgaaggaacgagacgttattatgcatgacggtaagactattacttgtcagtataagtatgaaagattacctgtcttctgctttgtttgtggattgattggacacgttgaaaaaaaatgtgcacttcgatttcaatactcagagatcgacttcccttttctctaggagtattcgatcaaggcattaacatggaaggaagctcaagctctaaaggcttcacaatggaacctgaaaaatttcaacaagcctaaactgaaatcgaagtcaaatcacccaaccgggagctctaaatcagcaaacactcctcctccacagtatccaatcatcgtgcacgatgctccaggtattgcaagccaggtattgcaagctaggagtaggatagagaccttaaacgtcgttggtgtgaagagtcatcttcagacctaatggagatagatgtagacggcggcacgaagactctgaaacaccagaaaggctagtccaggataaggatctgctatcccaactgacctctcgttagtcccaaggcctctcaactagagcaggaggaaggatggtcacaagactaggataatgatgtttccaatatgaacctgaatgtccatagctaatttttttagtcttgcttctgcactttttgtttattatgttctggtgactatgttatttacccttgtccgtatgcttgagggtaccctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggcctctatcgctatccctgctgctcttgctggaactcttggatacgttacctacaatgtggctaaccctgatatcccagcttctgagaaagttcctgcttacttcatgcaggttgagtactggggacctactatcggaactattggatacctcctcttcatctacttcggaaagcgtatcatgcagaacagatctcaacctttcggactcaagaacgctatgctcgtttacaacttctaccagaccttcttcaacagctactgcatctaccttttcgttacttctcatagggctcagggacttaaggtttggggaaacatccctgatatgactgctaactcttggggaatctctcaggttatctggcttcactacaacaacaagtacgttgagcttctcgacaccttcttcatggtgatgaggaagaagttcgaccagctttctttccttcacatctaccaccacactcttctcatctggtcatggttcgttgttatgaagcttgagcctgttggagattgctacttcggatcttctgttaacaccttcgtgcacgtgatcatgtactcttactacggacttgctgctcttggagttaactgtttctggaagaagtacatcacccagatccagatgcttcagttctgtatctgtgcttctcactctatctacaccgcttacgttcagaataccgctttctggcttccttaccttcaactctgggttatggtgaacatgttcgttctcttcgccaacttctaccgtaagaggtacaagtctaagggtgctaagaagcagtgataaggcgcgcggcgcgccgggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagtctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcgatacaaacaagattaaaaacatacacgaggatcttttacaacaattaccaacaacaacaaacaacaaacaacattacaattacatttacaattaccataccatgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatgagatcgttcaaacatttggcaataaagtttcttaagattgaatcctgttgccggtcttgcgatgattatcatataatttctgttgaattacgttaagcacgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgatagaacccacacgagcatgtgctttccatttaattttaaaaaccaagaaacatacatacataacattccatcagcctctctctctttttattacggttaatgacttaaaacacatcttattatcccatccttaacacctagcagtgtctttatacgatctcatcgatcaccacttcaaaaccatgcagactgctgctgcccctggagctggcatcggctaggctgggtgccgcactgtcccggaaggtccctagcgacttgtttagattgatgggaccacctctcaacttcctgctgctgtccctgctgctggatgtcctgcctcatctggccgattgcacgctccagtcccctgcatgtgcactcgctcctcaattgcttaagatcatcgcagcagctatcgaagtgctggctctgttgccctcctccacggccttggttgtagtagtagctgccgccgcccttctggactttttcccacaggaaccgccgaataattcgatagaaccacacgagcatgtgctttcatttattttaaaaaccaagaaacatacataacatttcatcagcctctctctctctctctctctctctctctctctctctctctctctctctctctttattacagctgttacactaacttaaaacacattcatctcattattattattattatccatccttaacacctagcagtgtctttgtacgatctcataatcgatcaccccttcatcaggtatccttaggcttcactccaacgttgttgcagttacggaacatgtacacaccatcatggttctcaacgaactggcaagatctccaagttttccaaaggctaacccacatgttctcatcggtgtgtctgtagtgctctcccataactttcttgatgcactcggtagcttctctagcatggtagaatgggatccttgaaacgtagtgatggagcacatgagtctcgatgatgtcatggaagatgattccgaggattccgaactctctatcgatagtagcagcagcacccttagcgaaagtccactcttgagcatcgtaatgaggcatagaagaatcggtgtgctgaaggaaggtaacgaaaacaagccagtggttaacaaggatccaaggacagaaccatgtgatgaaagtaggccagaatccgaaaaccttgtaagcggtgtaaacagaagtgagggtagcaaggattccaagatcagaaagaacgatgtaccagtagtccttcttatcgaaaacagggctagaaggccagtagtgagacttgaagaacttagaaacaccagggtaaggttgtccagtagcgttagtagcaaggtaaagagaaagtcctccaagctgttggaacaagagagcgaaaacagagtagataggagtttcctcagcgatatcgtgaaggctggtaacttggtgcttctctttgaattcctcggcggtgtaaggaacgaaaaccatatctctggtcatgtgtccagtagccttatggtgcttagcatgagagaacttccagctgaagtaaggaaccataacaagagagtggagaacccatccaacggtatcgttaacccatccgtagttagagaaagcagaatgtccacactcatgtccaaggatccagattccgaatccgaaacaagagatagagaacacgtaagcagaccaagcagcgaatctaaggaattcgttagggagaagagggatgtaggtaagtccaacgtaagcgatagcagagatagccacgatatctctcaccacgtaagacatagacttcacgagagatctctcgtaacagtgcttagggatagcgtcaaggatatccttgatggtgtaatctggcaccttgaaaacgtttccgaaggtatcgatagcggtcttttgctgcttgaaagatgcaacgtttccagaacgcctaacggtcttagtagatccctcaaggatctcagatccagacacggtaaccttagacatggtatggtaattgtaaatgtaattgtaatgttgtttgttgtttgttgttgttggtaattgttgtaaaatttttggtggtgattggttctttaaggtgtgagagtgagttgtgagttgtgtggtgggtttggtgagattggggatggtgggtttatatagtggagactgaggaatggggtcgtgagtgttaactttgcatgggctacacgtgggttcttttgggcttacacgtagtattattcatgcaaatgcagccaatacatatacggtattttaataatgtgtgggaatacaatatgccgagtattttactaattttggcaatgacaagtgtacatttggattatcttacttggcctctcttgctttaatttggattatttttattctcttaccttggccgttcatattcacatccctaaaggcaagacagaattgaatggtggccaaaaattaaaacgatggatatgacctacatagtgtaggatcaattaacgtcgaaggaaaatactgattctctcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaatactcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacttttacaacaattaccaacaacaacaaacaacaaacaacattacaattacatttacaattaccataccatgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatgacaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctgagtcgaacgggcagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattttacaacaattaccaacaacaacaaacaacaaacaacattacaattacatttacaattaccataccatgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatgaaggggtgatcgattatgagatcgtacaaagacactgctaggtgttaaggatggataataataataataatgagatgaatgtgttttaagttagtgtaacagctgtaataaagagagagagagagagagagagagagagagagagagagagagagagagagaggctgatgaaatgttatgtatgtttcttggtttttaaaataaatgaaagcacatgctcgtgtggttctatcgaattattcggcggttcctgtgggaaaaagtccagaagggccgccgcagctactactacaaccaaggccgtggaggagggcaacagagccagcacttcgatagctgctgcgatgatcttaagcaattgaggagcgagtgcacatgcaggggactggagcgtgcaatcggccagatgaggcaggacatccagcagcagggacagcagcaggaagttgagaggtggtcccatcaatctaaacaagtcgctagggaccttccgggacagtgcggcacccagcctagccgatgccagctccaggggcagcagcagtctgcatggttttgaagtggtgatcgatgagatcgtataaagacactgctaggtgttaaggatgggataataagatgtgttttaagtcattaaccgtaataaaaagagagagaggctgatggaatgttatgtatgtatgtttcttggtttttaaaattaaatggaaagcacatgctcgtgtgggttctatctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtcctgcaggctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggaatttgctcaacctctcgttgctatggctcaagagcagtacgctgctatcgatgctgttgttgctcctgctatcttctctgctaccgactctattggatggggactcaagcctatctcttctgctactaaggatctccctctcgttgaatctcctacccctcttatcctttctctcctcgcttacttcgctatcgttggttctggactcgtttaccgtaaagtgttccctagaaccgttaagggacaggatcctttccttctcaaggctcttatgctcgctcacaacgttttccttatcggactcagcctttacatgtgcctcaagctcgtttacgaggcttacgtgaacaagtactccttctggggaaacgcttacaaccctgctcaaaccgagatggctaaggtgatctggatcttctacgtgtccaagatctacgagttcatggacaccttcatcatgcttctcaagggaaacgttaaccaggtttccttcctccatgtttaccaccacggatctatctctggaatctggtggatgatcacttatgctgctccaggtggagatgcttacttctctgctgctctcaactcttgggttcatgtgtgcatgtacacctactacttcatggctgctgttcttcctaaggacgaaaagaccaagagaaagtacctttggtggggaagataccttacccagatgcaaatgttccagttcttcatgaaccttctccaggctgtttacctcctctactcttcttctccttaccctaagttcattgctcaactcctcgttgtttacatggttaccctcctcatgcttttcggaaacttctactacatgaagcaccacgcttctaagtgataagggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagcctgcagggcggccgcggatcccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgaattaaatatgagccctgagaggcgtcctgttgaaatcagacctgctactgctgctgatatggctgctgtttgtgatatcgtgaaccactacatcgagacttctaccgttaacttcagaactgagcctcaaactcctcaagagtggatcgatgatcttgagagactccaagatagatacccttggcttgttgctgaggttgagggtgttgttgctggaatcgcttacgctggaccttggaaggctagaaacgcttacgattggactgttgagtctaccgtttacgtttcacacagacatcagagacttggacttggatctaccctttacactcaccttctcaagtctatggaagctcagggattcaagtctgttgttgctgttatcggactccctaacgatccttctgttagacttcatgaggctcttggatacactgctagaggaactcttagagctgctggatacaagcacggtggatggcatgatgttggattctggcaaagagatttcgagcttcctgctcctcctagacctgttagaccagttactcagatctgaatttgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 4 - DNA, 22824 bp, synthetic construct
  misc_feature 1..22824: note = pGA7- mod_D nucleotide sequence
  source 1..22824: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgcctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggcgccccgcggaaagcttgctagccaattggggcccaacgttctcgagtttttctagaaggaaactgaaggcgggaaacgacaatctgctagtggatctcccagtcacgacgttgtaaaacgggcgccccgcggaaagcttgcggccgcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacgtgcttaacgtaattcaacagaaattatatgataatcatcgcaagaccggcaacaggattcaatcttaagaaactttattgccaaatgtttgaacgatcggcgcgcctcattagtgagccttctcagcctttccgttaacgtagtagtgctgtcccaccttatcaaggttagagaaagtagccttccaagcaccgtagtaagagagcaccttgtagttgagtccccacttcttagcgaaaggaacgaatcttctgctaacctcaggctgtctgaattgaggcatatcagggaagaggtggtggataacctgacagttaaggtatcccataagccagttcacgtatcctctagaaggatcgatatcaacggtgtgatcaacagcgtagttaacccaagaaaggtgcttatcagatggaacaacagggaggtgagtatgagaagtagagaagtgagcgaaaaggtacatgtaagcgatccagtttccgaaagtgaaccaccagtaagcaacaggccaagagtatccagtagcaagcttgataacagcggttctaacaacatgagaaacgagcatccaagaagcctcttcgtagttcttcttacggagaacttgtctagggtggagaacgtagatccagaaagcttgaacaagaagtccagaggtaacaggaacgaaagtccaagcttgaagtctagcccaagctctagagaatcctctaggtctgttatcctcaacagcagtgttgaagaaagccacagcaggagtggtatcaagatccatatcgtgtctaaccttttgaggggtagcatggtgcttgttatgcatctggttccacatctcaccagaagtagaaagtccgaatccacaagtcatagcctgaagtctcttgtccacgtaaacagatccggtaagagagttatgtccaccctcatgttgaacccatccacatctagctccgaagaaagcaccgtaaacaacagaagcaatgatagggtatccagcgtacataagagcagttccaagagcgaatgtagcaagaagctcgagaagtctgtaagccacatgggtgatagaaggcttgaagaatccatctctctcaagctcagcacgccatctagcgaaatcctcaagcataggagcatcctcagactcagatctcttgatctcagcaggtctagaaggcaaagctctaagcatcttccaagccttgagagaacgcatgtggaattctttgaaagcctcagtagcatcagcaccagtgttagcaagcatgtagaagatcacagatccaccagggtgcttgaagttagtcacatcgtactcaacgtcctcaactctaacccatctagtctcgaaagtagcagcaagctcatgaggctcaagagtcttaagatcaacaggagcagtagaagcatccttagcatcaagagcctcagcagaagatttagacctggtaagtggagatctaggagaagatcttccatcagtcttaggagggcacatggtatggtaattgtaaatgtaattgtaatgttgtttgttgtttgttgttgttggtaattgttgtaaaagatcctcgtgtatgtttttaatcttgtttgtatcgatgagttttggtttgagtaaagagtgaagcggatgagttaatttataggctataaaggagatttgcatggcgatcacgtgtaataatgcatgcacgcatgtgattgtatgtgtgtgctgtgagagagaagctcttaggtgtttgaagggagtgacaagtggcgaagaaaaacaattctccgcggctgcatgctatgtgtaacgtgtagctaatgttctggcatggcatcttatgaacgattctttttaaaaacaaggtaaaaacttaacttcataaaattaaaaaaaaaaacgtttactaagttggtttaaaaggggatgagactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggaattcgcccagcctcttgttgctatggctcaagagcaatacgctgctatcgatgctgttgttgctcctgctatcttctctgctactgattctatcggatggggacttaagcctatctcttctgctactaaggacttgcctcttgttgagtctcctacacctctcatcctttctttgcttgcttacttcgctatcgttggatctggactcgtttacagaaaggttttccctagaaccgtgaagggacaagatccattccttttgaaggctcttatgcttgctcacaacgtgttccttatcggactttctctttacatgtgcctcaagcttgtgtacgaggcttacgttaacaagtactctttctggggaaacgcttacaaccctgctcaaactgagatggctaaggttatctggatcttctacgtgagcaagatctacgagttcatggataccttcatcatgctcctcaagggaaatgttaaccaggttagcttccttcacgtttaccatcacggatctatctctggaatctggtggatgattacttacgctgctcctggtggtgatgcttacttctctgctgctcttaactcttgggttcacgtgtgtatgtacacctactattttatggctgccgtgcttcctaaggacgagaaaactaagagaaagtacctctggtggggaagataccttactcaaatgcagatgttccagttcttcatgaaccttctccaggctgtttaccttctctactcttcatctccttaccctaagtttatcgctcagctcctcgtggtgtacatggttactcttctcatgcttttcggaaacttctactacatgaagcaccacgctagcaagtgatgaggcgcgccgggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagtctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcgatacaaacaagattaaaaacatacacgaggatcttttacaacaattaccaacaacaacaaacaacaaacaacattacaattacatttacaattaccataccatgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatgagatcgttcaaacatttggcaataaagtttcttaagattgaatcctgttgccggtcttgcgatgattatcatataatttctgttgaattacgttaagcacgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgatagaacccacacgagcatgtgctttccatttaattttaaaaaccaagaaacatacatacataacattccatcagcctctctctctttttattacggttaatgacttaaaacacatcttattatcccatccttaacacctagcagtgtctttatacgatctcatcgatcaccacttcaaaaccatgcagactgctgctgcccctggagctggcatcggctaggctgggtgccgcactgtcccggaaggtccctagcgacttgtttagattgatgggaccacctctcaacttcctgctgctgtccctgctgctggatgtcctgcctcatctggccgattgcacgctccagtcccctgcatgtgcactcgctcctcaattgcttaagatcatcgcagcagctatcgaagtgctggctctgttgccctcctccacggccttggttgtagtagtagctgccgccgcccttctggactttttcccacaggaaccgccgaataattcgatagaaccacacgagcatgtgctttcatttattttaaaaaccaagaaacatacataacatttcatcagcctctctctctctctctctctctctctctctctctctctctctctctctctctttattacagctgttacactaacttaaaacacattcatctcattattattattattatccatccttaacacctagcagtgtctttgtacgatctcataatcgatcaccccttcatcaggtatccttaggcttcactccaacgttgttgcagttacggaacatgtacacaccatcatggttctcaacgaactggcaagatctccaagttttccaaaggctaacccacatgttctcatcggtgtgtctgtagtgctctcccataactttcttgatgcactcggtagcttctctagcatggtagaatgggatccttgaaacgtagtgatggagcacatgagtctcgatgatgtcatggaagatgattccgaggattccgaactctctatcgatagtagcagcagcacccttagcgaaagtccactcttgagcatcgtaatgaggcatagaagaatcggtgtgctgaaggaaggtaacgaaaacaagccagtggttaacaaggatccaaggacagaaccatgtgatgaaagtaggccagaatccgaaaaccttgtaagcggtgtaaacagaagtgagggtagcaaggattccaagatcagaaagaacgatgtaccagtagtccttcttatcgaaaacagggctagaaggccagtagtgagacttgaagaacttagaaacaccagggtaaggttgtccagtagcgttagtagcaaggtaaagagaaagtcctccaagctgttggaacaagagagcgaaaacagagtagataggagtttcctcagcgatatcgtgaaggctggtaacttggtgcttctctttgaattcctcggcggtgtaaggaacgaaaaccatatctctggtcatgtgtccagtagccttatggtgcttagcatgagagaacttccagctgaagtaaggaaccataacaagagagtggagaacccatccaacggtatcgttaacccatccgtagttagagaaagcagaatgtccacactcatgtccaaggatccagattccgaatccgaaacaagagatagagaacacgtaagcagaccaagcagcgaatctaaggaattcgttagggagaagagggatgtaggtaagtccaacgtaagcgatagcagagatagccacgatatctctcaccacgtaagacatagacttcacgagagatctctcgtaacagtgcttagggatagcgtcaaggatatccttgatggtgtaatctggcaccttgaaaacgtttccgaaggtatcgatagcggtcttttgctgcttgaaagatgcaacgtttccagaacgcctaacggtcttagtagatccctcaaggatctcagatccagacacggtaaccttagacatggtatggtaattgtaaatgtaattgtaatgttgtttgttgtttgttgttgttggtaattgttgtaaaatttttggtggtgattggttctttaaggtgtgagagtgagttgtgagttgtgtggtgggtttggtgagattggggatggtgggtttatatagtggagactgaggaatggggtcgtgagtgttaactttgcatgggctacacgtgggttcttttgggcttacacgtagtattattcatgcaaatgcagccaatacatatacggtattttaataatgtgtgggaatacaatatgccgagtattttactaattttggcaatgacaagtgtacatttggattatcttacttggcctctcttgctttaatttggattatttttattctcttaccttggccgttcatattcacatccctaaaggcaagacagaattgaatggtggccaaaaattaaaacgatggatatgacctacatagtgtaggatcaattaacgtcgaaggaaaatactgattctctcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaatactcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacttttacaacaattaccaacaacaacaaacaacaaacaacattacaattacatttacaattaccataccatgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatgacaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctgagtcgaacgggcagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattttacaacaattaccaacaacaacaaacaacaaacaacattacaattacatttacaattaccataccatgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatgaaggggtgatcgattatgagatcgtacaaagacactgctaggtgttaaggatggataataataataataatgagatgaatgtgttttaagttagtgtaacagctgtaataaagagagagagagagagagagagagagagagagagagagagagagagagagaggctgatgaaatgttatgtatgtttcttggtttttaaaataaatgaaagcacatgctcgtgtggttctatcgaattattcggcggttcctgtgggaaaaagtccagaagggccgccgcagctactactacaaccaaggccgtggaggagggcaacagagccagcacttcgatagctgctgcgatgatcttaagcaattgaggagcgagtgcacatgcaggggactggagcgtgcaatcggccagatgaggcaggacatccagcagcagggacagcagcaggaagttgagaggtggtcccatcaatctaaacaagtcgctagggaccttccgggacagtgcggcacccagcctagccgatgccagctccaggggcagcagcagtctgcatggttttgaagtggtgatcgatgagatcgtataaagacactgctaggtgttaaggatgggataataagatgtgttttaagtcattaaccgtaataaaaagagagagaggctgatggaatgttatgtatgtatgtttcttggtttttaaaattaaatggaaagcacatgctcgtgtgggttctatctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtcctgcaggctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggcctctatcgctatccctgctgctcttgctggaactcttggatacgttacctacaatgtggctaaccctgatatcccagcttctgagaaagttcctgcttacttcatgcaggttgagtactggggacctactatcggaactattggatacctcctcttcatctacttcggaaagcgtatcatgcagaacagatctcaacctttcggactcaagaacgctatgctcgtttacaacttctaccagaccttcttcaacagctactgcatctaccttttcgttacttctcatagggctcagggacttaaggtttggggaaacatccctgatatgactgctaactcttggggaatctctcaggttatctggcttcactacaacaacaagtacgttgagcttctcgacaccttcttcatggtgatgaggaagaagttcgaccagctttctttccttcacatctaccaccacactcttctcatctggtcatggttcgttgttatgaagcttgagcctgttggagattgctacttcggatcttctgttaacaccttcgtgcacgtgatcatgtactcttactacggacttgctgctcttggagttaactgtttctggaagaagtacatcacccagatccagatgcttcagttctgtatctgtgcttctcactctatctacaccgcttacgttcagaataccgctttctggcttccttaccttcaactctgggttatggtgaacatgttcgttctcttcgccaacttctaccgtaagaggtacaagtctaagggtgctaagaagcagtgataagggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagcctgcagggcggccgcggatcccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgaattaaatatgagccctgagaggcgtcctgttgaaatcagacctgctactgctgctgatatggctgctgtttgtgatatcgtgaaccactacatcgagacttctaccgttaacttcagaactgagcctcaaactcctcaagagtggatcgatgatcttgagagactccaagatagatacccttggcttgttgctgaggttgagggtgttgttgctggaatcgcttacgctggaccttggaaggctagaaacgcttacgattggactgttgagtctaccgtttacgtttcacacagacatcagagacttggacttggatctaccctttacactcaccttctcaagtctatggaagctcagggattcaagtctgttgttgctgttatcggactccctaacgatccttctgttagacttcatgaggctcttggatacactgctagaggaactcttagagctgctggatacaagcacggtggatggcatgatgttggattctggcaaagagatttcgagcttcctgctcctcctagacctgttagaccagttactcagatctgaatttgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 5 - DNA, 24809 bp, synthetic construct
  misc_feature 1..24809: note = pga7- mod_e nucleotide sequence
  source 1..24809: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgcctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggcgccccgcggaaagcttgctagccaattggggcccaacgttctcgagtttttctagaaggaaactgaaggcgggaaacgacaatctgctagtggatctcccagtcacgacgttgtaaaacgggcgccccgcggaaagcttgcggccgcggtaccgcccgttcgactcagatcttccaaggcctcgtctccgagtccgctgcttctcgccgcgccgatcacttctccgccgccaacaaggcttgtagttaataggaatcattcagggattgtgattccgggcagtagtaattaataatatagtattagtatagataatatgtttcgtttgggatctttggaacgttgctctgttccttgttgttcattttaaagcttttgagggatagttgcagaactgttcggtgatgcttcatcctctcaagaactagatttgggtaaagaaacatccatgcatggatatggaatgttgttcttccgattggagattattttataaaatttaaaattcatgatttaaaaaaacacataaaaaccacaaaattcatgatttattgacaatacgatacaaaattagcaccaccggctactggctcattacacatttccccttcccctcattctcactttgtggctttattattattattattacatatattttaccgttattatttcacgtcacataagcttgttaattaatcattagtgagccttctcagcctttccgttaacgtagtagtgctgtcccaccttatcaaggttagagaaagtagccttccaagcaccgtagtaagagagcaccttgtagttgagtccccacttcttagcgaaaggaacgaatcttctgctaacctcaggctgtctgaattgaggcatatcagggaagaggtggtggataacctgacagttaaggtatcccataagccagttcacgtatcctctagaaggatcgatatcaacggtgtgatcaacagcgtagttaacccaagaaaggtgcttatcagatggaacaacagggaggtgagtatgagaagtagagaagtgagcgaaaaggtacatgtaagcgatccagtttccgaaagtgaaccaccagtaagcaacaggccaagagtatccagtagcaagcttgataacagcggttctaacaacatgagaaacgagcatccaagaagcctcttcgtagttcttcttacggagaacttgtctagggtggagaacgtagatccagaaagcttgaacaagaagtccagaggtaacaggaacgaaagtccaagcttgaagtctagcccaagctctagagaatcctctaggtctgttatcctcaacagcagtgttgaagaaagccacagcaggagtggtatcaagatccatatcgtgtctaaccttttgaggggtagcatggtgcttgttatgcatctggttccacatctcaccagaagtagaaagtccgaatccacaagtcatagcctgaagtctcttgtccacgtaaacagatccggtaagagagttatgtccaccctcatgttgaacccatccacatctagctccgaagaaagcaccgtaaacaacagaagcaatgatagggtatccagcgtacataagagcagttccaagagcgaatgtagcaagaagctcgagaagtctgtaagccacatgggtgatagaaggcttgaagaatccatctctctcaagctcagcacgccatctagcgaaatcctcaagcataggagcatcctcagactcagatctcttgatctcagcaggtctagaaggcaaagctctaagcatcttccaagccttgagagaacgcatgtggaattctttgaaagcctcagtagcatcagcaccagtgttagcaagcatgtagaagatcacagatccaccagggtgcttgaagttagtcacatcgtactcaacgtcctcaactctaacccatctagtctcgaaagtagcagcaagctcatgaggctcaagagtcttaagatcaacaggagcagtagaagcatccttagcatcaagagcctcagcagaagatttagacctggtaagtggagatctaggagaagatcttccatcagtcttaggagggcacatggtatggtaattgtaaatgtaattgtaatgttgtttgttgtttgttgttgttggtaattgttgtaaaattaattaagtgggtatcttttggatggataagcaagtagtgatgatgttctaggtgaagtgatgggggtgttttatagcgggagatggtgaaatggatggtcgccacataagaaatggaggggaagggttcttgcgccattcttcagtttgcatggatgcatgggtttcattttgtaacacgtaataaggacaatgaagtgcaggtgtctctcaagtttcagaggggatatgtggacagaagaagaacggcgatgatattgatggaaatggccatctagtgtgaatctattcggttgataatactagtgcattttggccgttaatcccttcaattaactgcacaaacttcagttgagtattgattatttgattataggttctgtaaacacaataccaagtttatttagaggggagacatacaaatagtttcgatataaataatagagtggttaaacttagttattaaaactatatataaagtctaaaagttaaattatttttttaattgcaaatatataaagtctaaaggggttacattatttcttaagagatgtaactctgttggaatctgacttaatccgtctcatcactctggtttccagttctaatctaatgaattgttttctgccaaagaatttgaagcaagaagtaaattgatcaatgccgtcaacccacaccaaaccgtcaacccactaccatcgccgcggagacccccaaactcaacctccacccatcggtaagaagcacagggcagcccgcaccaccaccaatttggcgtgcatgacacctagggacttggcacgggaggcggcgcacgtggatgcaaatgacgggatatcagatgacaggaaacgacgttgagagaccatacgatgtagaatatgagctcaccatcaacgagaaactaggaaaatcacaaaaaaaacaactctcgtaattgtacgagtggcacagatgggtctgcctcaacatatctctaatacggcgaagcctgcccaacacgtagttgccggaatccggtgtggagctcacgactctgaaagataggcgcttcctgtttcgtttcgctcacccactggacgtccgtcatgtgatggatttcggtcattggtttgctgacaaccacattctgaagctccatgagatgagtcttcacaataggtcctgctcaataccgtggagttatggttgcaagtccataacttgccgttcgaatattttgcggagccagtcggacgggaattggcgagctcggctgacacctataaaggccatgacaagaagaaccaaaagttcttccctaatgctttcatgaggcttcgggtcgttatggatgtcggaaaacccctcttgaaggaacgagacgttattatgcatgacggtaagactattacttgtcagtataagtatgaaagattacctgtcttctgctttgtttgtggattgattggacacgttgaaaaaaaatgtgcacttcgatttcaatactcagagatcgacttcccttttctctaggagtattcgatcaaggcattaacatggaaggaagctcaagctctaaaggcttcacaatggaacctgaaaaatttcaacaagcctaaactgaaatcgaagtcaaatcacccaaccgggagctctaaatcagcaaacactcctcctccacagtatccaatcatcgtgcacgatgctccaggtattgcaagccaggtattgcaagctaggagtaggatagagaccttaaacgtcgttggtgtgaagagtcatcttcagacctaatggagatagatgtagacggcggcacgaagactctgaaacaccagaaaggctagtccaggataaggatctgctatcccaactgacctctcgttagtcccaaggcctctcaactagagcaggaggaaggatggtcacaagactaggataatgatgtttccaatatgaacctgaatgtccatagctaatttttttagtcttgcttctgcactttttgtttattatgttctggtgactatgttatttacccttgtccgtatgcttgagggtaccctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggcctctatcgctatccctgctgctcttgctggaactcttggatacgttacctacaatgtggctaaccctgatatcccagcttctgagaaagttcctgcttacttcatgcaggttgagtactggggacctactatcggaactattggatacctcctcttcatctacttcggaaagcgtatcatgcagaacagatctcaacctttcggactcaagaacgctatgctcgtttacaacttctaccagaccttcttcaacagctactgcatctaccttttcgttacttctcatagggctcagggacttaaggtttggggaaacatccctgatatgactgctaactcttggggaatctctcaggttatctggcttcactacaacaacaagtacgttgagcttctcgacaccttcttcatggtgatgaggaagaagttcgaccagctttctttccttcacatctaccaccacactcttctcatctggtcatggttcgttgttatgaagcttgagcctgttggagattgctacttcggatcttctgttaacaccttcgtgcacgtgatcatgtactcttactacggacttgctgctcttggagttaactgtttctggaagaagtacatcacccagatccagatgcttcagttctgtatctgtgcttctcactctatctacaccgcttacgttcagaataccgctttctggcttccttaccttcaactctgggttatggtgaacatgttcgttctcttcgccaacttctaccgtaagaggtacaagtctaagggtgctaagaagcagtgataaggcgcgcggcgcgccgggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagtctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcgatacaaacaagattaaaaacatacacgaggatcttttacaacaattaccaacaacaacaaacaacaaacaacattacaattacatttacaattaccataccatgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatgagatcgttcaaacatttggcaataaagtttcttaagattgaatcctgttgccggtcttgcgatgattatcatataatttctgttgaattacgttaagcacgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgatagaacccacacgagcatgtgctttccatttaattttaaaaaccaagaaacatacatacataacattccatcagcctctctctctttttattacggttaatgacttaaaacacatcttattatcccatccttaacacctagcagtgtctttatacgatctcatcgatcaccacttcaaaaccatgcagactgctgctgcccctggagctggcatcggctaggctgggtgccgcactgtcccggaaggtccctagcgacttgtttagattgatgggaccacctctcaacttcctgctgctgtccctgctgctggatgtcctgcctcatctggccgattgcacgctccagtcccctgcatgtgcactcgctcctcaattgcttaagatcatcgcagcagctatcgaagtgctggctctgttgccctcctccacggccttggttgtagtagtagctgccgccgcccttctggactttttcccacaggaaccgccgaataattcgatagaaccacacgagcatgtgctttcatttattttaaaaaccaagaaacatacataacatttcatcagcctctctctctctctctctctctctctctctctctctctctctctctctctctttattacagctgttacactaacttaaaacacattcatctcattattattattattatccatccttaacacctagcagtgtctttgtacgatctcataatcgatcaccccttcatcaggtatccttaggcttcactccaacgttgttgcagttacggaacatgtacacaccatcatggttctcaacgaactggcaagatctccaagttttccaaaggctaacccacatgttctcatcggtgtgtctgtagtgctctcccataactttcttgatgcactcggtagcttctctagcatggtagaatgggatccttgaaacgtagtgatggagcacatgagtctcgatgatgtcatggaagatgattccgaggattccgaactctctatcgatagtagcagcagcacccttagcgaaagtccactcttgagcatcgtaatgaggcatagaagaatcggtgtgctgaaggaaggtaacgaaaacaagccagtggttaacaaggatccaaggacagaaccatgtgatgaaagtaggccagaatccgaaaaccttgtaagcggtgtaaacagaagtgagggtagcaaggattccaagatcagaaagaacgatgtaccagtagtccttcttatcgaaaacagggctagaaggccagtagtgagacttgaagaacttagaaacaccagggtaaggttgtccagtagcgttagtagcaaggtaaagagaaagtcctccaagctgttggaacaagagagcgaaaacagagtagataggagtttcctcagcgatatcgtgaaggctggtaacttggtgcttctctttgaattcctcggcggtgtaaggaacgaaaaccatatctctggtcatgtgtccagtagccttatggtgcttagcatgagagaacttccagctgaagtaaggaaccataacaagagagtggagaacccatccaacggtatcgttaacccatccgtagttagagaaagcagaatgtccacactcatgtccaaggatccagattccgaatccgaaacaagagatagagaacacgtaagcagaccaagcagcgaatctaaggaattcgttagggagaagagggatgtaggtaagtccaacgtaagcgatagcagagatagccacgatatctctcaccacgtaagacatagacttcacgagagatctctcgtaacagtgcttagggatagcgtcaaggatatccttgatggtgtaatctggcaccttgaaaacgtttccgaaggtatcgatagcggtcttttgctgcttgaaagatgcaacgtttccagaacgcctaacggtcttagtagatccctcaaggatctcagatccagacacggtaaccttagacatggtatggtaattgtaaatgtaattgtaatgttgtttgttgtttgttgttgttggtaattgttgtaaaatttttggtggtgattggttctttaaggtgtgagagtgagttgtgagttgtgtggtgggtttggtgagattggggatggtgggtttatatagtggagactgaggaatggggtcgtgagtgttaactttgcatgggctacacgtgggttcttttgggcttacacgtagtattattcatgcaaatgcagccaatacatatacggtattttaataatgtgtgggaatacaatatgccgagtattttactaattttggcaatgacaagtgtacatttggattatcttacttggcctctcttgctttaatttggattatttttattctcttaccttggccgttcatattcacatccctaaaggcaagacagaattgaatggtggccaaaaattaaaacgatggatatgacctacatagtgtaggatcaattaacgtcgaaggaaaatactgattctctcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaatactcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacttttacaacaattaccaacaacaacaaacaacaaacaacattacaattacatttacaattaccataccatgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatgacaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctgagtcgaacgggcagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattttacaacaattaccaacaacaacaaacaacaaacaacattacaattacatttacaattaccataccatgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatgaaggggtgatcgattatgagatcgtacaaagacactgctaggtgttaaggatggataataataataataatgagatgaatgtgttttaagttagtgtaacagctgtaataaagagagagagagagagagagagagagagagagagagagagagagagagagaggctgatgaaatgttatgtatgtttcttggtttttaaaataaatgaaagcacatgctcgtgtggttctatcgaattattcggcggttcctgtgggaaaaagtccagaagggccgccgcagctactactacaaccaaggccgtggaggagggcaacagagccagcacttcgatagctgctgcgatgatcttaagcaattgaggagcgagtgcacatgcaggggactggagcgtgcaatcggccagatgaggcaggacatccagcagcagggacagcagcaggaagttgagaggtggtcccatcaatctaaacaagtcgctagggaccttccgggacagtgcggcacccagcctagccgatgccagctccaggggcagcagcagtctgcatggttttgaagtggtgatcgatgagatcgtataaagacactgctaggtgttaaggatgggataataagatgtgttttaagtcattaaccgtaataaaaagagagagaggctgatggaatgttatgtatgtatgtttcttggtttttaaaattaaatggaaagcacatgctcgtgtgggttctatctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtcctgcaggctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggaatttgctcaacctctcgttgctatggctcaagagcagtacgctgctatcgatgctgttgttgctcctgctatcttctctgctaccgactctattggatggggactcaagcctatctcttctgctactaaggatctccctctcgttgaatctcctacccctcttatcctttctctcctcgcttacttcgctatcgttggttctggactcgtttaccgtaaagtgttccctagaaccgttaagggacaggatcctttccttctcaaggctcttatgctcgctcacaacgttttccttatcggactcagcctttacatgtgcctcaagctcgtttacgaggcttacgtgaacaagtactccttctggggaaacgcttacaaccctgctcaaaccgagatggctaaggtgatctggatcttctacgtgtccaagatctacgagttcatggacaccttcatcatgcttctcaagggaaacgttaaccaggtttccttcctccatgtttaccaccacggatctatctctggaatctggtggatgatcacttatgctgctccaggtggagatgcttacttctctgctgctctcaactcttgggttcatgtgtgcatgtacacctactacttcatggctgctgttcttcctaaggacgaaaagaccaagagaaagtacctttggtggggaagataccttacccagatgcaaatgttccagttcttcatgaaccttctccaggctgtttacctcctctactcttcttctccttaccctaagttcattgctcaactcctcgttgtttacatggttaccctcctcatgcttttcggaaacttctactacatgaagcaccacgcttctaagtgataagggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagcctgcagggcggccgcggatcccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgaattaaatatgagccctgagaggcgtcctgttgaaatcagacctgctactgctgctgatatggctgctgtttgtgatatcgtgaaccactacatcgagacttctaccgttaacttcagaactgagcctcaaactcctcaagagtggatcgatgatcttgagagactccaagatagatacccttggcttgttgctgaggttgagggtgttgttgctggaatcgcttacgctggaccttggaaggctagaaacgcttacgattggactgttgagtctaccgtttacgtttcacacagacatcagagacttggacttggatctaccctttacactcaccttctcaagtctatggaagctcagggattcaagtctgttgttgctgttatcggactccctaacgatccttctgttagacttcatgaggctcttggatacactgctagaggaactcttagagctgctggatacaagcacggtggatggcatgatgttggattctggcaaagagatttcgagcttcctgctcctcctagacctgttagaccagttactcagatctgaatttgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 6 - DNA, 26543 bp, synthetic construct
  misc_feature 1..26543: note = pGA7- mod_F nucleotide sequence
  source 1..26543: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgggcggccgcctagaatctaattattctattcagactaaattagtataagtatttttttaatcaataaatattaattaataatttattagtaggagtgattgaatttataatatattttttttaatcatttaaagaatcttatatctttaaattgacaagagttttaaatggggagagtgttatcatatgacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgcgatcgcgataattctcagtgcgccttctccgccttgccgttgacgtagtagtgctgcccgaccttatccaagttcgagaacgtcgccttccaggcgccgtaataggacagcaccttgtagttcagcccccacttcttcgcgaacgggacgaaccgccggctcacctccggctggcgaaactgcggcatgtccgggaacaggtgatgaatgacctggcagttcagatatcccatcaaccagttcacgtacccgcgcgacgggtcgatgtccacggtgtgatcgaccgcgtagttcacccagctcaggtgcttatccgagggcacgaccgggaggtgcgtgtggctcgtggagaagtgcgcgaagaggtacatgtacgcgatccagttgccgaaggtgaaccaccagtacgcgacgggccacgagtaccccgtcgcgagtttaatcaccgcggtcctgacgacgtgagagacgagcatccacgacgcctcctcgtagttcttctttcgcaacacctgccgcgggtgcaggacgtagatccagaacgcctggacgagcagcccggaggtcaccgggacgaacgtccacgcctgaagccgagcccacgcgcgggagaaccccctcggccggttgtcctccacggcggtgttaaaaaacgccaccgcgggggtcgtgtccaggtccatgtcgtgcctcactttctgcggcgtcgcgtggtgcttattgtgcatctggttccacatctccccgctcgtggacagcccgaacccgcacgtcatcgcttggaggcgcttgtcgacgtagacggaccccgtgagcgagttgtgcccgccctcgtgctggacccaaccgcaccgagcgccgaagaacgcgccgtacacgacggacgcgatgatcgggtacccggcgtacatgagggcggtgccgagggcgaaggtcgcgaggagctcgagtaaccgatacgcgacgtgcgttatcgagggcttaaagaacccgtcgcgttcgagctccgcgcgccaccgcgcgaaatcctccaacatcggcgcgtcctcgctctcgctgcgtttgatctccgcggggcgcgacggcagcgctctgagcatcttccacgccttaagcgatcgcatgtggaactccttgaacgcctccgtggcgtccgcgcccgtgttcgcgagcatgtagaatatcacgctgcctcccgggtgtttgaagtttgtgacgtcgtactcgacgtcctccacgcgcacccatcgcgtctcgaacgtcgccgcgagctcgtgcggctcgagcgttttgagatcgacgggcgcggtcgacgcgtccttggcgtcgagcgcctccgcggaggatttgctgcgcgtcagcggcgatcgcggggacgatcggccgtccgtcttcggcgggcacatcgtcgcgcgcgcgacttaaaccgacgacggacggacgaacctgcaacggcgaattatcaattgacgcgttgctctgtttgtgtcggaaaataatggaggaagctatgagatgtatttgcatggatcctcttttgtggtccgttacgttttgcaaagcatgcatttagacatgtgtatactcaaactcatgcatccatacacattgtaataaagtagtagtccactgaaagttgccaaaatagcaaaatagtacattgcagaaatatatatactaaattcatatatttcgatggaaaatttcggttttccacttaaaataaaatgtacgtataaacttagtcttcctttaacgccaaaaaaaagtctacaaatttctgtctttaatactacattatttttgaattgtgctaaactatgattacatgtgcattaaaatttgagggaatgtgtgttcttagatctgattggcataccctctgactaattatgggatcatgagtatttctcgggaatgtgtttattcgcaagtctattctctaacctcagtaaataatgagtggaagtttagttaaggccaaatagtctaaaatatattatttgtagttttgtacacagccaggattgaatcttaagagactctttaagtctaaaattacaatcagatcctagaccactttctatttgcatgaacgtaaccggatactgattttttaaatgttttctttggttcaaaatcgccactatctttgacaaaaacattctcgaataggattcaaacctaagagcctttcaaattcgttcttaattatgtcatcatctttagttctgctcccatctctcatcggtctcaagaaccactcctagtcatttaagtactacgttttatacgcattacatatacatacatttacacaactaggaattgagtagggaacaaagtttcaactttcaactaatagggtaagccttctggtacatggaaaccaaccaaccaatctactaggcggccgcccgtcgggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaactgaaggcgggaaacgacaatctgctagtggatctcccagtcacgacgttgtaaaacgggcgccccgcggaaagcttgcggccgcggtaccgcccgttcgactcagatcttccaaggcctcgtctccgagtccgctgcttctcgccgcgccgatcacttctccgccgccaacaaggcttgtagttaataggaatcattcagggattgtgattccgggcagtagtaattaataatatagtattagtatagataatatgtttcgtttgggatctttggaacgttgctctgttccttgttgttcattttaaagcttttgagggatagttgcagaactgttcggtgatgcttcatcctctcaagaactagatttgggtaaagaaacatccatgcatggatatggaatgttgttcttccgattggagattattttataaaatttaaaattcatgatttaaaaaaacacataaaaaccacaaaattcatgatttattgacaatacgatacaaaattagcaccaccggctactggctcattacacatttccccttcccctcattctcactttgtggctttattattattattattacatatattttaccgttattatttcacgtcacataagcttgttaattaatcattagtgagccttctcagcctttccgttaacgtagtagtgctgtcccaccttatcaaggttagagaaagtagccttccaagcaccgtagtaagagagcaccttgtagttgagtccccacttcttagcgaaaggaacgaatcttctgctaacctcaggctgtctgaattgaggcatatcagggaagaggtggtggataacctgacagttaaggtatcccataagccagttcacgtatcctctagaaggatcgatatcaacggtgtgatcaacagcgtagttaacccaagaaaggtgcttatcagatggaacaacagggaggtgagtatgagaagtagagaagtgagcgaaaaggtacatgtaagcgatccagtttccgaaagtgaaccaccagtaagcaacaggccaagagtatccagtagcaagcttgataacagcggttctaacaacatgagaaacgagcatccaagaagcctcttcgtagttcttcttacggagaacttgtctagggtggagaacgtagatccagaaagcttgaacaagaagtccagaggtaacaggaacgaaagtccaagcttgaagtctagcccaagctctagagaatcctctaggtctgttatcctcaacagcagtgttgaagaaagccacagcaggagtggtatcaagatccatatcgtgtctaaccttttgaggggtagcatggtgcttgttatgcatctggttccacatctcaccagaagtagaaagtccgaatccacaagtcatagcctgaagtctcttgtccacgtaaacagatccggtaagagagttatgtccaccctcatgttgaacccatccacatctagctccgaagaaagcaccgtaaacaacagaagcaatgatagggtatccagcgtacataagagcagttccaagagcgaatgtagcaagaagctcgagaagtctgtaagccacatgggtgatagaaggcttgaagaatccatctctctcaagctcagcacgccatctagcgaaatcctcaagcataggagcatcctcagactcagatctcttgatctcagcaggtctagaaggcaaagctctaagcatcttccaagccttgagagaacgcatgtggaattctttgaaagcctcagtagcatcagcaccagtgttagcaagcatgtagaagatcacagatccaccagggtgcttgaagttagtcacatcgtactcaacgtcctcaactctaacccatctagtctcgaaagtagcagcaagctcatgaggctcaagagtcttaagatcaacaggagcagtagaagcatccttagcatcaagagcctcagcagaagatttagacctggtaagtggagatctaggagaagatcttccatcagtcttaggagggcacatggtatggtaattgtaaatgtaattgtaatgttgtttgttgtttgttgttgttggtaattgttgtaaaattaattaagtgggtatcttttggatggataagcaagtagtgatgatgttctaggtgaagtgatgggggtgttttatagcgggagatggtgaaatggatggtcgccacataagaaatggaggggaagggttcttgcgccattcttcagtttgcatggatgcatgggtttcattttgtaacacgtaataaggacaatgaagtgcaggtgtctctcaagtttcagaggggatatgtggacagaagaagaacggcgatgatattgatggaaatggccatctagtgtgaatctattcggttgataatactagtgcattttggccgttaatcccttcaattaactgcacaaacttcagttgagtattgattatttgattataggttctgtaaacacaataccaagtttatttagaggggagacatacaaatagtttcgatataaataatagagtggttaaacttagttattaaaactatatataaagtctaaaagttaaattatttttttaattgcaaatatataaagtctaaaggggttacattatttcttaagagatgtaactctgttggaatctgacttaatccgtctcatcactctggtttccagttctaatctaatgaattgttttctgccaaagaatttgaagcaagaagtaaattgatcaatgccgtcaacccacaccaaaccgtcaacccactaccatcgccgcggagacccccaaactcaacctccacccatcggtaagaagcacagggcagcccgcaccaccaccaatttggcgtgcatgacacctagggacttggcacgggaggcggcgcacgtggatgcaaatgacgggatatcagatgacaggaaacgacgttgagagaccatacgatgtagaatatgagctcaccatcaacgagaaactaggaaaatcacaaaaaaaacaactctcgtaattgtacgagtggcacagatgggtctgcctcaacatatctctaatacggcgaagcctgcccaacacgtagttgccggaatccggtgtggagctcacgactctgaaagataggcgcttcctgtttcgtttcgctcacccactggacgtccgtcatgtgatggatttcggtcattggtttgctgacaaccacattctgaagctccatgagatgagtcttcacaataggtcctgctcaataccgtggagttatggttgcaagtccataacttgccgttcgaatattttgcggagccagtcggacgggaattggcgagctcggctgacacctataaaggccatgacaagaagaaccaaaagttcttccctaatgctttcatgaggcttcgggtcgttatggatgtcggaaaacccctcttgaaggaacgagacgttattatgcatgacggtaagactattacttgtcagtataagtatgaaagattacctgtcttctgctttgtttgtggattgattggacacgttgaaaaaaaatgtgcacttcgatttcaatactcagagatcgacttcccttttctctaggagtattcgatcaaggcattaacatggaaggaagctcaagctctaaaggcttcacaatggaacctgaaaaatttcaacaagcctaaactgaaatcgaagtcaaatcacccaaccgggagctctaaatcagcaaacactcctcctccacagtatccaatcatcgtgcacgatgctccaggtattgcaagccaggtattgcaagctaggagtaggatagagaccttaaacgtcgttggtgtgaagagtcatcttcagacctaatggagatagatgtagacggcggcacgaagactctgaaacaccagaaaggctagtccaggataaggatctgctatcccaactgacctctcgttagtcccaaggcctctcaactagagcaggaggaaggatggtcacaagactaggataatgatgtttccaatatgaacctgaatgtccatagctaatttttttagtcttgcttctgcactttttgtttattatgttctggtgactatgttatttacccttgtccgtatgcttgagggtaccctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggcctctatcgctatccctgctgctcttgctggaactcttggatacgttacctacaatgtggctaaccctgatatcccagcttctgagaaagttcctgcttacttcatgcaggttgagtactggggacctactatcggaactattggatacctcctcttcatctacttcggaaagcgtatcatgcagaacagatctcaacctttcggactcaagaacgctatgctcgtttacaacttctaccagaccttcttcaacagctactgcatctaccttttcgttacttctcatagggctcagggacttaaggtttggggaaacatccctgatatgactgctaactcttggggaatctctcaggttatctggcttcactacaacaacaagtacgttgagcttctcgacaccttcttcatggtgatgaggaagaagttcgaccagctttctttccttcacatctaccaccacactcttctcatctggtcatggttcgttgttatgaagcttgagcctgttggagattgctacttcggatcttctgttaacaccttcgtgcacgtgatcatgtactcttactacggacttgctgctcttggagttaactgtttctggaagaagtacatcacccagatccagatgcttcagttctgtatctgtgcttctcactctatctacaccgcttacgttcagaataccgctttctggcttccttaccttcaactctgggttatggtgaacatgttcgttctcttcgccaacttctaccgtaagaggtacaagtctaagggtgctaagaagcagtgataaggcgcgcggcgcgccgggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagtctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcgatacaaacaagattaaaaacatacacgaggatcttttacaacaattaccaacaacaacaaacaacaaacaacattacaattacatttacaattaccataccatgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatgagatcgttcaaacatttggcaataaagtttcttaagattgaatcctgttgccggtcttgcgatgattatcatataatttctgttgaattacgttaagcacgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgatagaacccacacgagcatgtgctttccatttaattttaaaaaccaagaaacatacatacataacattccatcagcctctctctctttttattacggttaatgacttaaaacacatcttattatcccatccttaacacctagcagtgtctttatacgatctcatcgatcaccacttcaaaaccatgcagactgctgctgcccctggagctggcatcggctaggctgggtgccgcactgtcccggaaggtccctagcgacttgtttagattgatgggaccacctctcaacttcctgctgctgtccctgctgctggatgtcctgcctcatctggccgattgcacgctccagtcccctgcatgtgcactcgctcctcaattgcttaagatcatcgcagcagctatcgaagtgctggctctgttgccctcctccacggccttggttgtagtagtagctgccgccgcccttctggactttttcccacaggaaccgccgaataattcgatagaaccacacgagcatgtgctttcatttattttaaaaaccaagaaacatacataacatttcatcagcctctctctctctctctctctctctctctctctctctctctctctctctctctttattacagctgttacactaacttaaaacacattcatctcattattattattattatccatccttaacacctagcagtgtctttgtacgatctcataatcgatcaccccttcatcaggtatccttaggcttcactccaacgttgttgcagttacggaacatgtacacaccatcatggttctcaacgaactggcaagatctccaagttttccaaaggctaacccacatgttctcatcggtgtgtctgtagtgctctcccataactttcttgatgcactcggtagcttctctagcatggtagaatgggatccttgaaacgtagtgatggagcacatgagtctcgatgatgtcatggaagatgattccgaggattccgaactctctatcgatagtagcagcagcacccttagcgaaagtccactcttgagcatcgtaatgaggcatagaagaatcggtgtgctgaaggaaggtaacgaaaacaagccagtggttaacaaggatccaaggacagaaccatgtgatgaaagtaggccagaatccgaaaaccttgtaagcggtgtaaacagaagtgagggtagcaaggattccaagatcagaaagaacgatgtaccagtagtccttcttatcgaaaacagggctagaaggccagtagtgagacttgaagaacttagaaacaccagggtaaggttgtccagtagcgttagtagcaaggtaaagagaaagtcctccaagctgttggaacaagagagcgaaaacagagtagataggagtttcctcagcgatatcgtgaaggctggtaacttggtgcttctctttgaattcctcggcggtgtaaggaacgaaaaccatatctctggtcatgtgtccagtagccttatggtgcttagcatgagagaacttccagctgaagtaaggaaccataacaagagagtggagaacccatccaacggtatcgttaacccatccgtagttagagaaagcagaatgtccacactcatgtccaaggatccagattccgaatccgaaacaagagatagagaacacgtaagcagaccaagcagcgaatctaaggaattcgttagggagaagagggatgtaggtaagtccaacgtaagcgatagcagagatagccacgatatctctcaccacgtaagacatagacttcacgagagatctctcgtaacagtgcttagggatagcgtcaaggatatccttgatggtgtaatctggcaccttgaaaacgtttccgaaggtatcgatagcggtcttttgctgcttgaaagatgcaacgtttccagaacgcctaacggtcttagtagatccctcaaggatctcagatccagacacggtaaccttagacatggtatggtaattgtaaatgtaattgtaatgttgtttgttgtttgttgttgttggtaattgttgtaaaatttttggtggtgattggttctttaaggtgtgagagtgagttgtgagttgtgtggtgggtttggtgagattggggatggtgggtttatatagtggagactgaggaatggggtcgtgagtgttaactttgcatgggctacacgtgggttcttttgggcttacacgtagtattattcatgcaaatgcagccaatacatatacggtattttaataatgtgtgggaatacaatatgccgagtattttactaattttggcaatgacaagtgtacatttggattatcttacttggcctctcttgctttaatttggattatttttattctcttaccttggccgttcatattcacatccctaaaggcaagacagaattgaatggtggccaaaaattaaaacgatggatatgacctacatagtgtaggatcaattaacgtcgaaggaaaatactgattctctcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaatactcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacttttacaacaattaccaacaacaacaaacaacaaacaacattacaattacatttacaattaccataccatgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatgacaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctgagtcgaacgggcagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattttacaacaattaccaacaacaacaaacaacaaacaacattacaattacatttacaattaccataccatgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatgaaggggtgatcgattatgagatcgtacaaagacactgctaggtgttaaggatggataataataataataatgagatgaatgtgttttaagttagtgtaacagctgtaataaagagagagagagagagagagagagagagagagagagagagagagagagagaggctgatgaaatgttatgtatgtttcttggtttttaaaataaatgaaagcacatgctcgtgtggttctatcgaattattcggcggttcctgtgggaaaaagtccagaagggccgccgcagctactactacaaccaaggccgtggaggagggcaacagagccagcacttcgatagctgctgcgatgatcttaagcaattgaggagcgagtgcacatgcaggggactggagcgtgcaatcggccagatgaggcaggacatccagcagcagggacagcagcaggaagttgagaggtggtcccatcaatctaaacaagtcgctagggaccttccgggacagtgcggcacccagcctagccgatgccagctccaggggcagcagcagtctgcatggttttgaagtggtgatcgatgagatcgtataaagacactgctaggtgttaaggatgggataataagatgtgttttaagtcattaaccgtaataaaaagagagagaggctgatggaatgttatgtatgtatgtttcttggtttttaaaattaaatggaaagcacatgctcgtgtgggttctatctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtcctgcaggctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggaatttgctcaacctctcgttgctatggctcaagagcagtacgctgctatcgatgctgttgttgctcctgctatcttctctgctaccgactctattggatggggactcaagcctatctcttctgctactaaggatctccctctcgttgaatctcctacccctcttatcctttctctcctcgcttacttcgctatcgttggttctggactcgtttaccgtaaagtgttccctagaaccgttaagggacaggatcctttccttctcaaggctcttatgctcgctcacaacgttttccttatcggactcagcctttacatgtgcctcaagctcgtttacgaggcttacgtgaacaagtactccttctggggaaacgcttacaaccctgctcaaaccgagatggctaaggtgatctggatcttctacgtgtccaagatctacgagttcatggacaccttcatcatgcttctcaagggaaacgttaaccaggtttccttcctccatgtttaccaccacggatctatctctggaatctggtggatgatcacttatgctgctccaggtggagatgcttacttctctgctgctctcaactcttgggttcatgtgtgcatgtacacctactacttcatggctgctgttcttcctaaggacgaaaagaccaagagaaagtacctttggtggggaagataccttacccagatgcaaatgttccagttcttcatgaaccttctccaggctgtttacctcctctactcttcttctccttaccctaagttcattgctcaactcctcgttgtttacatggttaccctcctcatgcttttcggaaacttctactacatgaagcaccacgcttctaagtgataagggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagcctgcagggcggccgcggatcccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgaattaaatatgagccctgagaggcgtcctgttgaaatcagacctgctactgctgctgatatggctgctgtttgtgatatcgtgaaccactacatcgagacttctaccgttaacttcagaactgagcctcaaactcctcaagagtggatcgatgatcttgagagactccaagatagatacccttggcttgttgctgaggttgagggtgttgttgctggaatcgcttacgctggaccttggaaggctagaaacgcttacgattggactgttgagtctaccgtttacgtttcacacagacatcagagacttggacttggatctaccctttacactcaccttctcaagtctatggaagctcagggattcaagtctgttgttgctgttatcggactccctaacgatccttctgttagacttcatgaggctcttggatacactgctagaggaactcttagagctgctggatacaagcacggtggatggcatgatgttggattctggcaaagagatttcgagcttcctgctcctcctagacctgttagaccagttactcagatctgaatttgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 7 - DNA, 23760 bp, synthetic construct
  misc_feature 1..23760: note = pGA7- mod_G nucleotide sequence
  source 1..23760: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgcggccgcggtaccgcccgttcgactcagatcttccaaggcctcgtctccgagtccgctgcttctcgccgcgccgatcacttctccgccgccaacaaggcttgtagttaataggaatcattcagggattgtgattccgggcagtagtaattaataatatagtattagtatagataatatgtttcgtttgggatctttggaacgttgctctgttccttgttgttcattttaaagcttttgagggatagttgcagaactgttcggtgatgcttcatcctctcaagaactagatttgggtaaagaaacatccatgcatggatatggaatgttgttcttccgattggagattattttataaaatttaaaattcatgatttaaaaaaacacataaaaaccacaaaattcatgatttattgacaatacgatacaaaattagcaccaccggctactggctcattacacatttccccttcccctcattctcactttgtggctttattattattattattacatatattttaccgttattatttcacgtcacataagcttgttaattaattatcactgcttcttagcacccttagacttgtacctcttacggtagaagttggcgaagagaacgaacatgttcaccataacccagagttgaaggtaaggaagccagaaagcggtattctgaacgtaagcggtgtagatagagtgagaagcacagatacagaactgaagcatctggatctgggtgatgtacttcttccagaaacagttaactccaagagcagcaagtccgtagtaagagtacatgatcacgtgcacgaaggtgttaacagaagatccgaagtagcaatctccaacaggctcaagcttcataacaacgaaccatgaccagatgagaagagtgtggtggtagatgtgaaggaaagaaagctggtcgaacttcttcctcatcaccatgaagaaggtgtcgagaagctcaacgtacttgttgttgtagtgaagccagataacctgagagattccccaagagttagcagtcatatcagggatgtttccccaaaccttaagtccctgagccctatgagaagtaacgaaaaggtagatgcagtagctgttgaagaaggtctggtagaagttgtaaacgagcatagcgttcttgagtccgaaaggttgagatctgttctgcatgatacgctttccgaagtagatgaagaggaggtatccaatagttccgatagtaggtccccagtactcaacctgcatgaagtaagcaggaactttctcagaagctgggatatcagggttagccacattgtaggtaacgtatccaagagttccagcaagagcagcagggatagcgatagaggccatggtatggtaattgtaaatgtaattgtaatgttgtttgttgtttgttgttgttggtaattgttgtaaaattaattaagtgggtatcttttggatggataagcaagtagtgatgatgttctaggtgaagtgatgggggtgttttatagcgggagatggtgaaatggatggtcgccacataagaaatggaggggaagggttcttgcgccattcttcagtttgcatggatgcatgggtttcattttgtaacacgtaataaggacaatgaagtgcaggtgtctctcaagtttcagaggggatatgtggacagaagaagaacggcgatgatattgatggaaatggccatctagtgtgaatctattcggttgataatactagtgcattttggccgttaatcccttcaattaactgcacaaacttcagttgagtattgattatttgattataggttctgtaaacacaataccaagtttatttagaggggagacatacaaatagtttcgatataaataatagagtggttaaacttagttattaaaactatatataaagtctaaaagttaaattatttttttaattgcaaatatataaagtctaaaggggttacattatttcttaagagatgtaactctgttggaatctgacttaatccgtctcatcactctggtttccagttctaatctaatgaattgttttctgccaaagaatttgaagcaagaagtaaattgatcaatgccgtcaacccacaccaaaccgtcaacccactaccatcgccgcggagacccccaaactcaacctccacccatcggtaagaagcacagggcagcccgcaccaccaccaatttggcgtgcatgacacctagggacttggcacgggaggcggcgcacgtggatgcaaatgacgggatatcagatgacaggaaacgacgttgagagaccatacgatgtagaatatgagctcaccatcaacgagaaactaggaaaatcacaaaaaaaacaactctcgtaattgtacgagtggcacagatgggtctgcctcaacatatctctaatacggcgaagcctgcccaacacgtagttgccggaatccggtgtggagctcacgactctgaaagataggcgcttcctgtttcgtttcgctcacccactggacgtccgtcatgtgatggatttcggtcattggtttgctgacaaccacattctgaagctccatgagatgagtcttcacaataggtcctgctcaataccgtggagttatggttgcaagtccataacttgccgttcgaatattttgcggagccagtcggacgggaattggcgagctcggctgacacctataaaggccatgacaagaagaaccaaaagttcttccctaatgctttcatgaggcttcgggtcgttatggatgtcggaaaacccctcttgaaggaacgagacgttattatgcatgacggtaagactattacttgtcagtataagtatgaaagattacctgtcttctgctttgtttgtggattgattggacacgttgaaaaaaaatgtgcacttcgatttcaatactcagagatcgacttcccttttctctaggagtattcgatcaaggcattaacatggaaggaagctcaagctctaaaggcttcacaatggaacctgaaaaatttcaacaagcctaaactgaaatcgaagtcaaatcacccaaccgggagctctaaatcagcaaacactcctcctccacagtatccaatcatcgtgcacgatgctccaggtattgcaagccaggtattgcaagctaggagtaggatagagaccttaaacgtcgttggtgtgaagagtcatcttcagacctaatggagatagatgtagacggcggcacgaagactctgaaacaccagaaaggctagtccaggataaggatctgctatcccaactgacctctcgttagtcccaaggcctctcaactagagcaggaggaaggatggtcacaagactaggataatgatgtttccaatatgaacctgaatgtccatagctaatttttttagtcttgcttctgcactttttgtttattatgttctggtgactatgttatttacccttgtccgtatgcttgagggtaccctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagcattttacaacaattaccaacaacaacaaacaacaaacaacattacaattacatttacaattaccataccatggaattcgcccagcctcttgttgctatggctcaagagcaatacgctgctatcgatgctgttgttgctcctgctatcttctctgctactgattctatcggatggggacttaagcctatctcttctgctactaaggacttgcctcttgttgagtctcctacacctctcatcctttctttgcttgcttacttcgctatcgttggatctggactcgtttacagaaaggttttccctagaaccgtgaagggacaagatccattccttttgaaggctcttatgcttgctcacaacgtgttccttatcggactttctctttacatgtgcctcaagcttgtgtacgaggcttacgttaacaagtactctttctggggaaacgcttacaaccctgctcaaactgagatggctaaggttatctggatcttctacgtgagcaagatctacgagttcatggataccttcatcatgctcctcaagggaaatgttaaccaggttagcttccttcacgtttaccatcacggatctatctctggaatctggtggatgattacttacgctgctcctggtggtgatgcttacttctctgctgctcttaactcttgggttcacgtgtgtatgtacacctactattttatggctgccgtgcttcctaaggacgagaaaactaagagaaagtacctctggtggggaagataccttactcaaatgcagatgttccagttcttcatgaaccttctccaggctgtttaccttctctactcttcatctccttaccctaagtttatcgctcagctcctcgtggtgtacatggttactcttctcatgcttttcggaaacttctactacatgaagcaccacgctagcaagtgatgaggcgcgccgggccgccgccatgtgacagatcgaaggaagaaagtgtaataagacgactctcactactcgatcgctagtgattgtcattgttatatataataatgttatctttcacaacttatcgtaatgcatgtgaaactataacacattaatcctacttgtcatatgataacactctccccatttaaaactcttgtcaatttaaagatataagattctttaaatgattaaaaaaaatatattataaattcaatcactcctactaataaattattaattattatttattgattaaaaaaatacttatactaatttagtctgaatagaataattagattctagtctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcgatacaaacaagattaaaaacatacacgaggatcttttacaacaattaccaacaacaacaaacaacaaacaacattacaattacatttacaattaccataccatgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatgagatcgttcaaacatttggcaataaagtttcttaagattgaatcctgttgccggtcttgcgatgattatcatataatttctgttgaattacgttaagcacgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgatagaacccacacgagcatgtgctttccatttaattttaaaaaccaagaaacatacatacataacattccatcagcctctctctctttttattacggttaatgacttaaaacacatcttattatcccatccttaacacctagcagtgtctttatacgatctcatcgatcaccacttcaaaaccatgcagactgctgctgcccctggagctggcatcggctaggctgggtgccgcactgtcccggaaggtccctagcgacttgtttagattgatgggaccacctctcaacttcctgctgctgtccctgctgctggatgtcctgcctcatctggccgattgcacgctccagtcccctgcatgtgcactcgctcctcaattgcttaagatcatcgcagcagctatcgaagtgctggctctgttgccctcctccacggccttggttgtagtagtagctgccgccgcccttctggactttttcccacaggaaccgccgaataattcgatagaaccacacgagcatgtgctttcatttattttaaaaaccaagaaacatacataacatttcatcagcctctctctctctctctctctctctctctctctctctctctctctctctctctttattacagctgttacactaacttaaaacacattcatctcattattattattattatccatccttaacacctagcagtgtctttgtacgatctcataatcgatcaccccttcatcaggtatccttaggcttcactccaacgttgttgcagttacggaacatgtacacaccatcatggttctcaacgaactggcaagatctccaagttttccaaaggctaacccacatgttctcatcggtgtgtctgtagtgctctcccataactttcttgatgcactcggtagcttctctagcatggtagaatgggatccttgaaacgtagtgatggagcacatgagtctcgatgatgtcatggaagatgattccgaggattccgaactctctatcgatagtagcagcagcacccttagcgaaagtccactcttgagcatcgtaatgaggcatagaagaatcggtgtgctgaaggaaggtaacgaaaacaagccagtggttaacaaggatccaaggacagaaccatgtgatgaaagtaggccagaatccgaaaaccttgtaagcggtgtaaacagaagtgagggtagcaaggattccaagatcagaaagaacgatgtaccagtagtccttcttatcgaaaacagggctagaaggccagtagtgagacttgaagaacttagaaacaccagggtaaggttgtccagtagcgttagtagcaaggtaaagagaaagtcctccaagctgttggaacaagagagcgaaaacagagtagataggagtttcctcagcgatatcgtgaaggctggtaacttggtgcttctctttgaattcctcggcggtgtaaggaacgaaaaccatatctctggtcatgtgtccagtagccttatggtgcttagcatgagagaacttccagctgaagtaaggaaccataacaagagagtggagaacccatccaacggtatcgttaacccatccgtagttagagaaagcagaatgtccacactcatgtccaaggatccagattccgaatccgaaacaagagatagagaacacgtaagcagaccaagcagcgaatctaaggaattcgttagggagaagagggatgtaggtaagtccaacgtaagcgatagcagagatagccacgatatctctcaccacgtaagacatagacttcacgagagatctctcgtaacagtgcttagggatagcgtcaaggatatccttgatggtgtaatctggcaccttgaaaacgtttccgaaggtatcgatagcggtcttttgctgcttgaaagatgcaacgtttccagaacgcctaacggtcttagtagatccctcaaggatctcagatccagacacggtaaccttagacatggtatggtaattgtaaatgtaattgtaatgttgtttgttgtttgttgttgttggtaattgttgtaaaatttttggtggtgattggttctttaaggtgtgagagtgagttgtgagttgtgtggtgggtttggtgagattggggatggtgggtttatatagtggagactgaggaatggggtcgtgagtgttaactttgcatgggctacacgtgggttcttttgggcttacacgtagtattattcatgcaaatgcagccaatacatatacggtattttaataatgtgtgggaatacaatatgccgagtattttactaattttggcaatgacaagtgtacatttggattatcttacttggcctctcttgctttaatttggattatttttattctcttaccttggccgttcatattcacatccctaaaggcaagacagaattgaatggtggccaaaaattaaaacgatggatatgacctacatagtgtaggatcaattaacgtcgaaggaaaatactgattctctcaagcatacggacaagggtaaataacatagtcaccagaacataataaacaaaaagtgcagaagcaagactaaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaatactcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcagacccatctgtgccactcgtacaattacgagagttgttttttttgtgattttcctagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacttttacaacaattaccaacaacaacaaacaacaaacaacattacaattacatttacaattaccataccatgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatgacaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctgagtcgaacgggcagaatcagtattttccttcgacgttaattgatcctacactatgtaggtcatatccatcgttttaatttttggccaccattcaattctgtcttgcctttagggatgtgaatatgaacggccaaggtaagagaataaaaataatccaaattaaagcaagagaggccaagtaagataatccaaatgtacacttgtcattgccaaaattagtaaaatactcggcatattgtattcccacacattattaaaataccgtatatgtattggctgcatttgcatgaataatactacgtgtaagcccaaaagaacccacgtgtagcccatgcaaagttaacactcacgaccccattcctcagtctccactatataaacccaccatccccaatctcaccaaacccaccacacaactcacaactcactctcacaccttaaagaaccaatcaccaccaaaaattttacaacaattaccaacaacaacaaacaacaaacaacattacaattacatttacaattaccataccatgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatgaaggggtgatcgattatgagatcgtacaaagacactgctaggtgttaaggatggataataataataataatgagatgaatgtgttttaagttagtgtaacagctgtaataaagagagagagagagagagagagagagagagagagagagagagagagagagaggctgatgaaatgttatgtatgtttcttggtttttaaaataaatgaaagcacatgctcgtgtggttctatcgaattattcggcggttcctgtgggaaaaagtccagaagggccgccgcagctactactacaaccaaggccgtggaggagggcaacagagccagcacttcgatagctgctgcgatgatcttaagcaattgaggagcgagtgcacatgcaggggactggagcgtgcaatcggccagatgaggcaggacatccagcagcagggacagcagcaggaagttgagaggtggtcccatcaatctaaacaagtcgctagggaccttccgggacagtgcggcacccagcctagccgatgccagctccaggggcagcagcagtctgcatggttttgaagtggtgatcgatgagatcgtataaagacactgctaggtgttaaggatgggataataagatgtgttttaagtcattaaccgtaataaaaagagagagaggctgatggaatgttatgtatgtatgtttcttggtttttaaaattaaatggaaagcacatgctcgtgtgggttctatctcgattaaaaatcccaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtcctgcaggctagtagattggttggttggtttccatgtaccagaaggcttaccctattagttgaaagttgaaactttgttccctactcaattcctagttgtgtaaatgtatgtatatgtaatgcgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaggaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgcaaaacgtaacggaccacaaaagaggatccatgcaaatacatctcatagcttcctccattattttccgacacaaacagagcagactctagaggatccccccgttttacaacaattaccaacaacaacaaacaacaaacaacattacaattacatttacaattaccatcccaaatcggcgcgccatgtgtcctcctaagaccgatggaagatcttctcctagatctcctctcaccaggtctaagtcatctgctgaggctcttgatgctaaggatgcttctaccgctcctgttgatcttaagacccttgagcctcatgaacttgctgctaccttcgagactagatgggttagggttgaggatgttgagtacgacgtgaccaacttcaaacatcctggtggaagcgtgatcttctacatgcttgctaacactggtgctgatgctactgaggctttcaaagaatttcacatgcgtagcctcaaggcttggaagatgcttagagctttgccttctagacctgctgagatcaagagatctgagtctgaggatgctcctatgcttgaggatttcgctaggtggagagctgaacttgagagggacggattcttcaagccttctatcacccatgttgcttaccgtcttttggagcttcttgctactttcgctcttggaaccgctcttatgtacgctggataccctatcattgctagcgttgtgtacggtgctttcttcggagctagatgtggatgggttcaacatgagggtggacacaactctcttaccggatctgtgtacgtggataagagacttcaggctatgacttgcggattcggactttctaccagcggagagatgtggaaccagatgcataacaagcaccatgctacccctcagaaagttagacacgacatggatcttgataccactcctgctgtggctttcttcaacaccgctgtggaggataatagacctaggggattctctagagcttgggctagacttcaagcttggaccttcgttcctgttacttctggacttctcgttcaggctttctggatctacgttctccatcctagacaggtgctcaggaagaagaactacgaggaagcttcttggatgctcgtttctcacgttgttagaaccgctgttatcaagcttgctaccggatactcttggcctgttgcttactggtggttcactttcggaaactggatcgcttacatgtacctcttcgctcacttctctacttctcacactcacctccctgttgttccatctgacaagcaccttagctgggttaactacgctgttgatcacaccgttgacatcgatccttctcgtggatacgttaactggcttatgggataccttaactgccaggttatccaccatctcttccctgatatgcctcaattcagacagcctgaggtgtcaagaagattcgtccctttcgctaagaagtggggactcaactacaaggtgctctcttactacggtgcttggaaggctactttcagcaacctcgacaaagttggacagcactactacgttaacggaaaggctgagaaggctcactgatgattaattaaatttgggctcgaaccggttcgagcaagcttatgtgacgtgaaataataacggtaaaatatatgtaataataataataataaagccacaaagtgagaatgaggggaaggggaaatgtgtaatgagccagtagccggtggtgctaattttgtatcgtattgtcaataaatcatgaattttgtggtttttatgtgtttttttaaatcatgaattttaaattttataaaataatctccaatcggaagaacaacattccatatccatgcatggatgtttctttacccaaatctagttcttgagaggatgaagcatcaccgaacagttctgcaactatccctcaaaagctttaaaatgaacaacaaggaacagagcaacgttccaaagatcccaaacgaaacatattatctatactaatactatattattaattactactgcccggaatcacaatccctgaatgattcctattaactacaagccttgttggcggcggagaagtgatcggcgcggcgagaagcagcggactcggagacgaggccttggaagatctcctgcagggcggccgcggatcccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactgaattaaatatgagccctgagaggcgtcctgttgaaatcagacctgctactgctgctgatatggctgctgtttgtgatatcgtgaaccactacatcgagacttctaccgttaacttcagaactgagcctcaaactcctcaagagtggatcgatgatcttgagagactccaagatagatacccttggcttgttgctgaggttgagggtgttgttgctggaatcgcttacgctggaccttggaaggctagaaacgcttacgattggactgttgagtctaccgtttacgtttcacacagacatcagagacttggacttggatctaccctttacactcaccttctcaagtctatggaagctcagggattcaagtctgttgttgctgttatcggactccctaacgatccttctgttagacttcatgaggctcttggatacactgctagaggaactcttagagctgctggatacaagcacggtggatggcatgatgttggattctggcaaagagatttcgagcttcctgctcctcctagacctgttagaccagttactcagatctgaatttgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcactagtgatgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 8 - DNA, 11042 bp, synthetic construct
  misc_feature 1..11042: note = pORE04+11ABGBEC_Cowpea_EPA_insert nucleotide sequence
  source 1..11042: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccctagaatctaattattctattcagactaaattagtataagtatttttttaatcaataaataataattaataatttattagtaggagtgattgaatttataatatattttttttaatcatttaaagaatcttatatctttaaattgacaagagttttaaatggggagagtgttatcatatcacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgaattctcatcacttagaagcgtggtgcttcatgtagtagaagtttccgaaaagcatgagaagagtcaccatgtacaccacgaggagctgagcgataaacttagggtaaggtgaagaagagtagaggaggtacacagcctggagaaggttcatgaagaactggaacatttgcatctgggtgaggtatcttccccaccagaggtactttctcttagttttctcatccttagggagcacagcagccataaagtagtaggtgtacatgcacacgtgcacccaagagttgagagcagcagagaaataagcatcaccacctggagcagcgtaggtgatcatccaccagattccagagatagatccgtgatggtacacgtggaggaatgacacctggttcacatttcccttgaggagcatgatgaaggtatccatgaactcgtagatctttgacacgtagaaaatccagatcaccttagccatctcggtctgagcagggttgtaagcgtttccccagaaagagtacttgttcacgtaagcctcgtacacgagcttgaggcacatgtagagtgagagtccgatgaggaacacgttatgagcgagcatgagagccttgagcaagaatggatcctgtcccttcacagttctagggaacacctttctgtacacgagtccagatcccacgatagcgaagtaagcgaggagagacaagataagaggggtaggagattccacgagagggagatccttagtagcagaagagataggcttgagtccccatccgatagaatcggtagcagagaagatagcaggagccacaacagcatcgatagcagcgtattgttcttgagccatagccacgagaggctgagcaaattccatgaattctgttcttctttactctttgtgtgactgaggtttggtctagtgctttggtcatctatatataatgataacaacaatgagaacaagctttggagtgatcggagggtctaggatacatgagattcaagtggactaggatctacaccgttggattttgagtgtggatatgtgtgaggttaattttacttggtaacggccacaaaggcctaaggagaggtgttgagacccttatcggcttgaaccgctggaataatgccacgtggaagataattccatgaatcttatcgttatctatgagtgaaattgtgtgatggtggagtggtgcttgctcattttacttgcctggtggacttggccctttccttatggggaatttatattttacttactatagagctttcataccttttttttaccttggatttagttaatatataatggtatgattcatgaataaaaatgggaaatttttgaatttgtactgctaaatgcataagattaggtgaaactgtggaatatatatttttttcatttaaaagcaaaatttgccttttactagaattataaatatagaaaaatatataacattcaaataaaaatgaaaataagaactttcaaaaaacagaactatgtttaatgtgtaaagattagtcgcacatcaagtcatctgttacaatatgttacaacaagtcataagcccaacaaagttagcacgtctaaataaactaaagagtccacgaaaatattacaaatcataagcccaacaaagttattgatcaaaaaaaaaaaacgcccaacaaagctaaacaaagtccaaaaaaaacttctcaagtctccatcttcctttatgaacattgaaaactatacacaaaacaagtcagataaatctctttctgggcctgtcttcccaacctcctacatcacttccctatcggattgaatgttttacttgtaccttttccgttgcaatgatattgatagtatgtttgtgaaaactaatagggttaacaatcgaagtcatggaatatggatttggtccaagattttccgagagctttctagtagaaagcccatcaccagaaatttactagtaaaataaatcaccaattaggtttcttattatgtgccaaattcaatataattatagaggatatttcaaatgaaaacgtatgaatgttattagtaaatggtcaggtaagacattaaaaaaatcctacgtcagatattcaactttaaaaattcgatcagtgtggaattgtacaaaaatttgggatctactatatatatataatgctttacaacacttggatttttttttggaggctggaatttttaatctacatatttgttttggccatgcaccaactcattgtttagtgtaatactttgattttgtcaaatatatgtgttcgtgtatatttgtataagaatttctttgaccatatacacacacacatatatatatatatatatatattatatatcatgcacttttaattgaaaaaataatatatatatatatagtgcattttttctaacaaccatatatgttgcgattgatctgcaaaaatactgctagagtaatgaaaaatataatctattgctgaaattatctcagatgttaagattttcttaaagtaaattctttcaaattttagctaaaagtcttgtaataactaaagaataatacacaatctcgaccacggaaaaaaaacacataataaatttggggcccctagaatctaattattctattcagactaaattagtataagtatttttttaatcaataaataataattaataatttattagtaggagtgattgaatttataatatattttttttaatcatttaaagaatcttatatctttaaattgacaagagttttaaatggggagagtgttatcatatcacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgcggccgctcatcagtgagccttctcagcctttccgttcacgtagtagtgctgtcccaccttatcgaggtttgagaaggtagccttccaagcaccgtagtaagagagcaccttgtagttgagtccccacttcttagcgaaaggaacgaatcttcttgacacctcaggctgtctgaactgtggcatatctgggaagaggtgatggatcacctggcagttgaggtatcccatgagccagttcacgtaacccctagaaggatcgatatccacggtgtgatccacagcgtagttcacccaagaaaggtgcttatcagatggcaccactgggagatgggtatgagaggtagagaagtgagcgaagaggtacatgtaagcgatccagtttccgaaggtgaaccaccaataagcaacaggccaagagtatccggtagcgagcttgataacagcggttctcacaacgtgagacacgagcatccaagaagcctcttcgtagttcttctttctgagcacctgtctaggatggagaacgtagatccagaaagcctgcacgagaagtccagaagtcacaggaacgaaagtccaagcctgaagtctagcccaagctctagaaaatcccctaggcctgttatcctcaacagcggtgttgaagaaagccacagcaggagtggtatcgagatccatatcatgcctcaccttttgtggggttgcgtggtgcttgttgtgcatctggttccacatctcaccagaggtagaaagtccgaatccgcaagtcatagcctggagcctcttatccacatacacagatccggtgagagagttatgaccaccctcgtgttgaacccatccacatctagctccgaagaaagcaccgtacaccacagaagcgataatagggtatccagcatacatgagagcagttccgagagcgaaagtagcaagaagctcgagaagtctgtatgccacgtgggtgatagaaggcttgaagaatccatccctctcaagctcagctctccacctagcgaaatcttcgagcataggagcatcctcagactcagacctcttgatctcagctggtctagaaggcaaagccctaagcatcttccaagccttgagagatctcatgtgaaattctttgaaagcctcagtagcatcagcaccggtgttagcgagcatgtagaagatcacagaaccaccagggtgcttgaagttagtaacatcgtactcaacatcctcaactctcacccatctagtctcgaaggtagcagccaactcatgaggctcaagagtcttgagatccacaggagcagtagaagcatccttagcatcgagagcctcagcagatgacttagacctggtaagaggtgacctaggagaagatcttccatcagtctttggagggcacatgcggccgctgttcttctttactctttgtgtgactgaggtttggtctagtgctttggtcatctatatataatgataacaacaatgagaacaagctttggagtgatcggagggtctaggatacatgagattcaagtggactaggatctacaccgttggattttgagtgtggatatgtgtgaggttaattttacttggtaacggccacaaaggcctaaggagaggtgttgagacccttatcggcttgaaccgctggaataatgccacgtggaagataattccatgaatcttatcgttatctatgagtgaaattgtgtgatggtggagtggtgcttgctcattttacttgcctggtggacttggccctttccttatggggaatttatattttacttactatagagctttcataccttttttttaccttggatttagttaatatataatggtatgattcatgaataaaaatgggaaatttttgaatttgtactgctaaatgcataagattaggtgaaactgtggaatatatatttttttcatttaaaagcaaaatttgccttttactagaattataaatatagaaaaatatataacattcaaataaaaatgaaaataagaactttcaaaaaacagaactatgtttaatgtgtaaagattagtcgcacatcaagtcatctgttacaatatgttacaacaagtcataagcccaacaaagttagcacgtctaaataaactaaagagtccacgaaaatattacaaatcataagcccaacaaagttattgatcaaaaaaaaaaaacgcccaacaaagctaaacaaagtccaaaaaaaacttctcaagtctccatcttcctttatgaacattgaaaactatacacaaaacaagtcagataaatctctttctgggcctgtcttcccaacctcctacatcacttccctatcggattgaatgttttacttgtaccttttccgttgcaatgatattgatagtatgtttgtgaaaactaatagggttaacaatcgaagtcatggaatatggatttggtccaagattttccgagagctttctagtagaaagcccatcaccagaaatttactagtaaaataaatcaccaattaggtttcttattatgtgccaaattcaatataattatagaggatatttcaaatgaaaacgtatgaatgttattagtaaatggtcaggtaagacattaaaaaaatcctacgtcagatattcaactttaaaaattcgatcagtgtggaattgtacaaaaatttgggatctactatatatatataatgctttacaacacttggatttttttttggaggctggaatttttaatctacatatttgttttggccatgcaccaactcattgtttagtgtaatactttgattttgtcaaatatatgtgttcgtgtatatttgtataagaatttctttgaccatatacacacacacatatatatatatatatatatattatatatcatgcacttttaattgaaaaaataatatatatatatatagtgcattttttctaacaaccatatatgttgcgattgatctgcaaaaatactgctagagtaatgaaaaatataatctattgctgaaattatctcagatgttaagattttcttaaagtaaattctttcaaattttagctaaaagtcttgtaataactaaagaataatacacaatctcgaccacggaaaaaaaacacataataaatttgggcgcgccgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctgcgatcgcatgcctcctagggattcttactcttacgctgctcctccatctgctcagctccatgaagttgatactcctcaagagcacgataagaaagaactcgtgatcggagatagggcttacgatgtgaccaacttcgtgaagagacaccctggtggaaagattatcgcttaccaggttggaactgatgctaccgatgcttacaagcagttccacgtgagatctgctaaggctgataagatgctcaagtctctcccatctaggcctgtgcacaagggatattctccaagaagggctgatcttatcgctgatttccaagagttcaccaagcagcttgaggctgagggaatgttcgaaccttctctccctcatgtggcttacagactcgctgaggttatcgctatgcatgttgctggtgctgctctcatctggcacggatatactttcgctggaatcgctatgctcggagtggttcagggaagatgtggatggcttatgcatgagggtggacactactctctcaccggaaacattgctttcgatagggctatccaggtggcatgctatggacttggatgtggaatgtctggtgcttggtggagaaaccagcataacaagcaccatgctacccctcaaaagctccagcatgatgtggatctcgatactctccctctcgtggctttccatgagagaatcgctgctaaggtgaagtctcctgctatgaaggcttggctctctatgcaggctaagcttttcgctcctgtgactactcttctcgttgctcttggatggcagctctacctccatcctagacacatgctcaggaccaagcactacgatgagcttgctatgctcggtatcagatacggactcgttggatacctcgctgctaattacggtgctggatacgttctcgcttgctaccttctttacgttcagctcggagctatgtacatcttctgcaacttcgctgtgtctcacactcatctccctgtggttgaacctaacgagcatgctacttgggttgagtacgctgctaaccacactaccaactgctctccatcttggtggtgtgattggtggatgtcttacctcaactaccagatcgagcaccacctctacccttctatgcctcagttcagacaccctaagatcgctcctagagtgaagcagcttttcgagaagcacggactccactacgatgtgagaggatactttgaggctatggctgataccttcgctaacctcgataatgtggctcacgctcctgagaagaaaatgcagtgatgagcgatcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcagggcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgtccgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 9 - DNA, 1254 bp, synthetic construct
  misc_feature 1..1254: note = Codon-optimized open reading frame for expression of Lachanceakluyveri12 desaturase in plants
  source 1..1254: mol_type = other DNA
atgagcgctgttaccgttactggatctgatcctaagaacagaggatcttctagcaacaccgagcaagaggttccaaaagttgctatcgataccaacggaaacgtgttctctgttcctgatttcaccatcaaggacatccttggagctatccctcatgagtgttacgagagaagattggctacctctctctactacgtgttcagagatatcttctgcatgcttaccaccggataccttacccataagatcctttaccctctcctcatctcttacacctctaacagcatcatcaagttcactttctgggccctttacacttacgttcaaggacttttcggaaccggaatctgggttctcgctcatgagtgtggacatcaagctttctctgattacggaatcgtgaacgatttcgttggatggacccttcactcttaccttatggttccttacttcagctggaagtactctcatggaaagcaccataaggctactggacacatgaccagagatatggttttcgttcctgccaccaaagaggaattcaagaagtctaggaacttcttcggtaacctcgctgagtactctgaggattctccacttagaaccctttacgagcttcttgttcaacaacttggaggatggatcgcttacctcttcgttaacgttacaggacaaccttaccctgatgttccttcttggaaatggaaccacttctggcttacctctccacttttcgagcaaagagatgctctctacatcttcctttctgatcttggaatcctcacccagggaatcgttcttactctttggtacaagaaattcggaggatggtcccttttcatcaactggttcgttccttacatctgggttaaccactggctcgttttcatcacattccttcagcacactgatcctactatgcctcattacaacgctgaggaatggactttcgctaagggtgctgctgctactatcgatagaaagttcggattcatcggacctcacatcttccatgatatcatcgagactcatgtgcttcaccactactgttctaggatcccattctacaacgctagacctgcttctgaggctatcaagaaagttatgggaaagcactacaggtctagcgacgagaacatgtggaagtcactttggaagtctttcaggtcttgccaatacgttgacggtgataacggtgttctcatgttccgtaacatcaacaactgcggagttggagctgctgagaagtaatga

SEQ ID NO: 10 - protein, 416 aa, Lachancea kluyveri
MSAVTVTGSDPKNRGSSSNTEQEVPKVAIDTNGNVFSVPDFTIKDILGAIPHECYERRLATSLYYVFRDIFCMLTTGYLTHKILYPLLISYTSNSIIKFTFWALYTYVQGLFGTGIWVLAHECGHQAFSDYGIVNDFVGWTLHSYLMVPYFSWKYSHGKHHKATGHMTRDMVFVPATKEEFKKSRNFFGNLAEYSEDSPLRTLYELLVQQLGGWIAYLFVNVTGQPYPDVPSWKWNHFWLTSPLFEQRDALYIFLSDLGILTQGIVLTLWYKKFGGWSLFINWFVPYIWVNHWLVFITFLQHTDPTMPHYNAEEWTFAKGAAATIDRKFGFIGPHIFHDIIETHVLHHYCSRIPFYNARPASEAIKKVMGKHYRSSDENMWKSLWKSFRSCQYVDGDNGVLMFRNINNCGVGAAEK

SEQ ID NO: 11 - DNA, 1251 bp, Pichia pastoris
  source 1..1251: mol_type = other DNA
atgtctaaggttaccgtgtctggatctgagatccttgagggatctactaagaccgttaggcgttctggaaacgttgcatctttcaagcagcaaaagaccgctatcgataccttcggaaacgttttcaaggtgccagattacaccatcaaggatatccttgacgctatccctaagcactgttacgagagatctctcgtgaagtctatgtcttacgtggtgagagatatcgtggctatctctgctatcgcttacgttggacttacctacatccctcttctccctaacgaattccttagattcgctgcttggtctgcttacgtgttctctatctcttgtttcggattcggaatctggatccttggacatgagtgtggacattctgctttctctaactacggatgggttaacgataccgttggatgggttctccactctcttgttatggttccttacttcagctggaagttctctcatgctaagcaccataaggctactggacacatgaccagagatatggttttcgttccttacaccgccgaggaattcaaagagaagcaccaagttaccagccttcacgatatcgctgaggaaactcctatctactctgttttcgctctcttgttccaacagcttggaggactttctctttaccttgctactaacgctactggacaaccttaccctggtgtttctaagttcttcaagtctcactactggccttctagccctgttttcgataagaaggactactggtacatcgttctttctgatcttggaatccttgctaccctcacttctgtttacaccgcttacaaggttttcggattctggcctactttcatcacatggttctgtccttggatccttgttaaccactggcttgttttcgttaccttccttcagcacaccgattcttctatgcctcattacgatgctcaagagtggactttcgctaagggtgctgctgctactatcgatagagagttcggaatcctcggaatcatcttccatgacatcatcgagactcatgtgctccatcactacgtttcaaggatcccattctaccatgctagagaagctaccgagtgcatcaagaaagttatgggagagcactacagacacaccgatgagaacatgtgggttagcctttggaaaacttggagatcttgccagttcgttgagaaccatgatggtgtgtacatgttccgtaactgcaacaacgttggagtgaagcctaaggatacctgatga

SEQ ID NO: 12 - protein, 415 aa, Pichia pastoris
MSKVTVSGSEILEGSTKTVRRSGNVASFKQQKTAIDTFGNVFKVPDYTIKDILDAIPKHCYERSLVKSMSYVVRDIVAISAIAYVGLTYIPLLPNEFLRFAAWSAYVFSISCFGFGIWILGHECGHSAFSNYGWVNDTVGWVLHSLVMVPYFSWKFSHAKHHKATGHMTRDMVFVPYTAEEFKEKHQVTSLHDIAEETPIYSVFALLFQQLGGLSLYLATNATGQPYPGVSKFFKSHYWPSSPVFDKKDYWYIVLSDLGILATLTSVYTAYKVFGFWPTFITWFCPWILVNHWLVFVTFLQHTDSSMPHYDAQEWTFAKGAAATIDREFGILGIIFHDIIETHVLHHYVSRIPFYHAREATECIKKVMGEHYRHTDENMWVSLWKTWRSCQFVENHDGVYMFRNCNNVGVKPKDT

SEQ ID NO: 13 - DNA, 1392 bp, Micromonas pusilla
  source 1..1392: mol_type = other DNA
atgtgcccgccgaagacggacggccgatcgtccccgcgatcgccgctgacgcgcagcaaatcctccgcggaggcgctcgacgccaaggacgcgtcgaccgcgcccgtcgatctcaaaacgctcgagccgcacgagctcgcggcgacgttcgagacgcgatgggtgcgcgtggaggacgtcgagtacgacgtcacaaacttcaaacacccgggaggcagcgtgatattctacatgctcgcgaacacgggcgcggacgccacggaggcgttcaaggagttccacatgcgatcgcttaaggcgtggaagatgctcagagcgctgccgtcgcgccccgcggagatcaaacgcagcgagagcgaggacgcgccgatgttggaggatttcgcgcggtggcgcgcggagctcgaacgcgacgggttctttaagccctcgataacgcacgtcgcgtatcggttactcgagctcctcgcgaccttcgccctcggcaccgccctcatgtacgccgggtacccgatcatcgcgtccgtcgtgtacggcgcgttcttcggcgctcggtgcggttgggtccagcacgagggcgggcacaactcgctcacggggtccgtctacgtcgacaagcgcctccaagcgatgacgtgcgggttcgggctgtccacgagcggggagatgtggaaccagatgcacaataagcaccacgcgacgccgcagaaagtgaggcacgacatggacctggacacgacccccgcggtggcgttttttaacaccgccgtggaggacaaccggccgagggggttctcccgcgcgtgggctcggcttcaggcgtggacgttcgtcccggtgacctccgggctgctcgtccaggcgttctggatctacgtcctgcacccgcggcaggtgttgcgaaagaagaactacgaggaggcgtcgtggatgctcgtctctcacgtcgtcaggaccgcggtgattaaactcgcgacggggtactcgtggcccgtcgcgtactggtggttcaccttcggcaactggatcgcgtacatgtacctcttcgcgcacttctccacgagccacacgcacctcccggtcgtgccctcggataagcacctgagctgggtgaactacgcggtcgatcacaccgtggacatcgacccgtcgcgcgggtacgtgaactggttgatgggatatctgaactgccaggtcattcatcacctgttcccggacatgccgcagtttcgccagccggaggtgagccggcggttcgtcccgttcgcgaagaagtgggggctgaactacaaggtgctgtcctattacggcgcctggaaggcgacgttctcgaacttggataaggtcgggcagcactactacgtcaacggcaaggcggagaaggcgcactga

SEQ ID NO: 14 - DNA, 1395 bp, synthetic construct
  misc_feature 1..1395: note = Codon-optimized open reading frame for expression of Micromonaspusilla6-desaturase in plants (version 1)
  source 1..1395: mol_type = other DNA
atgtgccctcctaagactgatggaagatcttctcctagatctccacttaccaggtctaaatcttctgctgaggctcttgatgctaaggatgcttctactgctcctgttgatcttaagactcttgagcctcatgagcttgctgctactttcgagactagatgggttagagttgaggacgttgagtacgatgtgactaacttcaagcaccctggtggatctgtgatcttctacatgcttgctaacactggtgctgatgctactgaggctttcaaagaattccacatgcgttctctcaaggcttggaagatgcttagagctttgccttctagacctgctgagatcaagagatctgagtctgaggatgctcctatgcttgaggatttcgctagatggcgtgctgagcttgagagagatggattcttcaagccttctatcacccatgtggcttacagacttctcgagcttcttgctacattcgctcttggaactgctcttatgtacgctggataccctatcattgcttctgttgtttacggtgctttcttcggagctagatgtggatgggttcaacatgagggtggacataactctcttaccggatctgtttacgtggacaagagacttcaggctatgacttgtggattcggactttctacttctggtgagatgtggaaccagatgcataacaagcaccatgctacccctcaaaaggttagacacgatatggatcttgataccactcctgctgtggctttcttcaacactgctgttgaggataacagacctagaggattctctagagcttgggctagacttcaagcttggactttcgttcctgttacctctggacttcttgttcaagctttctggatctacgttctccaccctagacaagttctccgtaagaagaactacgaagaggcttcttggatgctcgtttctcatgttgttagaaccgctgttatcaagcttgctactggatactcttggcctgttgcttactggtggttcactttcggaaactggatcgcttacatgtaccttttcgctcacttctctacttctcatactcacctccctgttgttccatctgataagcacctttcttgggttaactacgctgttgatcacaccgttgatatcgatccttctagaggatacgtgaactggcttatgggataccttaactgtcaggttatccaccacctcttccctgatatgcctcaattcagacagcctgaggttagcagaagattcgttcctttcgctaagaagtggggactcaactacaaggtgctctcttactacggtgcttggaaggctactttctctaaccttgataaggtgggacagcactactacgttaacggaaaggctgagaaggctcactaatga

SEQ ID NO: 15 - DNA, 1395 bp, synthetic construct
  misc_feature 1..1395: note = Codon-optimized open reading frame for expression of Micromonaspusilla6-desaturase in plants (version 2)
  source 1..1395: mol_type = other DNA
atgtgtcctcctaagaccgatggaagatcttctcctagatctcctctcaccaggtctaagtcatctgctgaggctcttgatgctaaggatgcttctaccgctcctgttgatcttaagacccttgagcctcatgaacttgctgctaccttcgagactagatgggttagggttgaggatgttgagtacgacgtgaccaacttcaaacatcctggtggaagcgtgatcttctacatgcttgctaacactggtgctgatgctactgaggctttcaaagaatttcacatgcgtagcctcaaggcttggaagatgcttagagctttgccttctagacctgctgagatcaagagatctgagtctgaggatgctcctatgcttgaggatttcgctaggtggagagctgaacttgagagggacggattcttcaagccttctatcacccatgttgcttaccgtcttttggagcttcttgctactttcgctcttggaaccgctcttatgtacgctggataccctatcattgctagcgttgtgtacggtgctttcttcggagctagatgtggatgggttcaacatgagggtggacacaactctcttaccggatctgtgtacgtggataagagacttcaggctatgacttgcggattcggactttctaccagcggagagatgtggaaccagatgcataacaagcaccatgctacccctcagaaagttagacacgacatggatcttgataccactcctgctgtggctttcttcaacaccgctgtggaggataatagacctaggggattctctagagcttgggctagacttcaagcttggaccttcgttcctgttacttctggacttctcgttcaggctttctggatctacgttctccatcctagacaggtgctcaggaagaagaactacgaggaagcttcttggatgctcgtttctcacgttgttagaaccgctgttatcaagcttgctaccggatactcttggcctgttgcttactggtggttcactttcggaaactggatcgcttacatgtacctcttcgctcacttctctacttctcacactcacctccctgttgttccatctgacaagcaccttagctgggttaactacgctgttgatcacaccgttgacatcgatccttctcgtggatacgttaactggcttatgggataccttaactgccaggttatccaccatctcttccctgatatgcctcaattcagacagcctgaggtgtcaagaagattcgtccctttcgctaagaagtggggactcaactacaaggtgctctcttactacggtgcttggaaggctactttcagcaacctcgacaaagttggacagcactactacgttaacggaaaggctgagaaggctcactgatga

SEQ ID NO: 16 - protein, 463 aa, Micromonas pusilla
MCPPKTDGRSSPRSPLTRSKSSAEALDAKDASTAPVDLKTLEPHELAATFETRWVRVEDVEYDVTNFKHPGGSVIFYMLANTGADATEAFKEFHMRSLKAWKMLRALPSRPAEIKRSESEDAPMLEDFARWRAELERDGFFKPSITHVAYRLLELLATFALGTALMYAGYPIIASVVYGAFFGARCGWVQHEGGHNSLTGSVYVDKRLQAMTCGFGLSTSGEMWNQMHNKHHATPQKVRHDMDLDTTPAVAFFNTAVEDNRPRGFSRAWARLQAWTFVPVTSGLLVQAFWIYVLHPRQVLRKKNYEEASWMLVSHVVRTAVIKLATGYSWPVAYWWFTFGNWIAYMYLFAHFSTSHTHLPVVPSDKHLSWVNYAVDHTVDIDPSRGYVNWLMGYLNCQVIHHLFPDMPQFRQPEVSRRFVPFAKKWGLNYKVLSYYGAWKATFSNLDKVGQHYYVNGKAEKAH

SEQ ID NO: 17 - DNA, 1449 bp, Ostreococcus lucimarinus
  source 1..1449: mol_type = other DNA
atgtgcgtcgaaacgaccgaaggcacatcgcgaacgatggcgaacgaacgcacgagctcgtcgtcgtcgctgagcgaaggcggaacgccgacggtgacggtcgggatgggaagcgaagacgcggggaagaagactcgaaacgcgagcgtcacggcgtggacgaaagagttggagccgcacgcgatcgcgaagacgttcgaacggcggtacgtgacgatcgaaggcgtggaatacgatgtgacggattttaagcatcccggaggatcggttatttattacatgctgtcgaacacgggagcggacgcgacggaggcttttaaagagtttcattatcggtcgaaaaaggcgcgcaaggcgttggcggcgttgccgcataagccagtggacgcggcgacgcgggaaccgatcgaagatgaggcgatgctgaaggatttcgcgcagtggcgcaaggaattggagcgtgagggattttttaagccctcgccggcgcacgtggcgtatcgattcgccgagctcgcggcgatgttcgcgctcggcacggcgttgatgcacgcgcgttggcacgtcgcttccgtgatcgtgtactcgtgtttcttcggcgcgcgatgcggttgggtgcagcacgagggtgggcacaattcgttgactggaaacatttggtgggacaagcgaatccaagccttcgccgcggggttcggcttggcgtcgagtggcgacatgtggaacaacatgcacaacaagcatcacgcgacgccccaaaaggtgcgacacgatatggatctcgacaccactcccacggtggcgttcttcaactccgcggttgaagaaaatcgcccgcggggattcagtaagttgtggttgcgccttcaagcgtggaccttcgtgcccgtgacgtccggtatggttttgttcttctggatgttcgtcttgcacccgcgtaacgcgctgcgacgcaaaagcttcgaagaagcggcttggatgttttccgcgcacgtcattcgcacggcggttatcaaagccgtcaccggctactcctggatcgcctcgtacggcttgttcgcggcgacgatgtgggcgagcggatgttacttgttcgcgcacttttccacgtctcacacgcacttggatgtcgtgccgagcgataaacacctctcgtgggtgcgatacgccgtcgatcacacgatcgacatcaatccgaacaacagcgtcgtcaactggttgatgggctacttgaactgccaagtcatccatcacctgttcccggatatgcctcagttccgccaacccgaagtctcccgccgattcgtcccgtttgcgaagaagtggaacttaaactacaaggtcttgacgtattatggggcctggaaggcgacgttcggcaacttgaacgacgtcgggaagcactattacgtgcacggatctcagcgcgtcaaatcaaagtcggcgtga

SEQ ID NO: 18 - DNA, 1449 bp, synthetic construct
  misc_feature 1..1449: note = Codon-optimized open reading frame for expression of Ostreococcuslucimarinus6-desaturase in plants
  source 1..1449: mol_type = other DNA
atgtgtgttgagactactgagggaacctctagaactatggctaacgagaggacctcttcttcttcttcactctctgagggtggaactcctactgttactgtgggaatgggatctgaggatgctggaaagaaaaccagaaacgcttctgttactgcttggaccaaagagcttgagcctcacgctatcgctaagaccttcgagagaagatacgttaccatcgagggtgttgagtacgatgtgaccgatttcaaacaccctggtggatctgtgatctactacatgctctctaacactggtgctgatgctactgaggctttcaaagagttccactaccgttctaagaaggctagaaaggctcttgctgctcttcctcacaagcctgttgatgctgctactagagagcctattgaggacgaggctatgcttaaggatttcgctcagtggagaaaagagttggagagagagggattcttcaagccttctcctgctcatgttgcttaccgtttcgctgaactcgctgctatgttcgctcttggaaccgctcttatgcatgctagatggcacgttgctagcgttatcgtgtactcctgtttcttcggagctagatgtggatgggttcaacatgagggtggacacaactctcttaccggaaacatctggtgggataagagaatccaagctttcgctgctggattcggacttgcttcttctggtgacatgtggaacaacatgcacaacaagcaccatgctactcctcagaaagtgagacacgatatggatcttgataccacccctaccgttgctttcttcaactctgctgtggaggaaaacagacctaggggattctctaagctttggctcagacttcaagcttggaccttcgttcctgttacctctggaatggtgctcttcttctggatgttcgttctccatcctagaaacgctctccgtcgtaagtctttcgaagaggctgcttggatgttctctgctcacgttatcagaaccgctgttatcaaggctgttaccggatactcttggatcgctagctacggacttttcgctgctactatgtgggcttctggatgctaccttttcgctcacttctctacttctcacacccacctcgatgttgttccatctgataagcaccttagctgggttaggtacgctgttgatcacaccatcgacatcaaccctaacaactctgttgtgaactggcttatgggataccttaactgccaggttatccaccatctcttccctgatatgcctcaattcagacagcctgaggtgtcaagaagattcgtccctttcgctaagaagtggaacctcaactacaaggtgctcacttactacggtgcttggaaggctactttcggaaacctcaacgatgttggaaagcactactacgttcacggatctcagagagtgaagagcaagagcgcttga

SEQ ID NO: 19 - protein, 482 aa, Ostreococcus lucimarinus
MCVETTEGTSRTMANERTSSSSSLSEGGTPTVTVGMGSEDAGKKTRNASVTAWTKELEPHAIAKTFERRYVTIEGVEYDVTDFKHPGGSVIYYMLSNTGADATEAFKEFHYRSKKARKALAALPHKPVDAATREPIEDEAMLKDFAQWRKELEREGFFKPSPAHVAYRFAELAAMFALGTALMHARWHVASVIVYSCFFGARCGWVQHEGGHNSLTGNIWWDKRIQAFAAGFGLASSGDMWNNMHNKHHATPQKVRHDMDLDTTPTVAFFNSAVEENRPRGFSKLWLRLQAWTFVPVTSGMVLFFWMFVLHPRNALRRKSFEEAAWMFSAHVIRTAVIKAVTGYSWIASYGLFAATMWASGCYLFAHFSTSHTHLDVVPSDKHLSWVRYAVDHTIDINPNNSVVNWLMGYLNCQVIHHLFPDMPQFRQPEVSRRFVPFAKKWNLNYKVLTYYGAWKATFGNLNDVGKHYYVHGSQRVKSKSA

SEQ ID NO: 20 - protein, 456 aa, Ostreococcus lucimarinus
MCVETENNDGIPTVEIAFDGERERAEANVKLSAEKMEPAALAKTFARRYVVIEGVEYDVTDFKHPGGTVIFYALSNTGADATEAFKEFHHRSRKARKALAALPSRPAKTAKVDDAEMLQDFAKWRKELERDGFFKPSPAHVAYRFAELAAMYALGTYLMYARYVVSSVLVYACFFGARCGWVQHEGGHSSLTGNIWWDKRIQAFTAGFGLAGSGDMWNSMHNKHHATPQKVRHDMDLDTTPAVAFFNTAVEDNRPRGFSKYWLRLQAWTFIPVTSGLVLLFWMFFLHPSKALKGGKYEELVWMLAAHVIRTWTIKAVTGFTAMQSYGLFLATSWVSGCYLFAHFSTSHTHLDVVPADEHLSWVRYAVDHTIDIDPSQGWVNWLMGYLNCQVIHHLFPSMPQFRQPEVSRRFVAFAKKWNLNYKVMTYAGAWKATLGNLDNVGKHYYVHGQHSGKTA

SEQ ID NO: 21 - DNA, 894 bp, Pyramimonas cordata
  source 1..894: mol_type = other DNA
atggagttcgctcagcctcttgtggctatggcacaggagcagtatgccgcaattgacgcggtggtagcccctgcaattttctcagctaccgacagcatcggttggggtcttaagcccattagcagcgcgacaaaggatcttcctctcgttgagagtccgacgccgctcatactgagcctgttggcctattttgcgatcgtcggctctgggctggtgtaccgcaaagtattccctcgcacagtaaaggggcaagaccccttcctgctgaaggcgctcatgcttgcgcacaacgtgttcctcattggcctcagtctatacatgtgcttgaagcttgtctacgaggcttacgtcaacaagtactccttctggggaaacgcctacaaccccgcacagaccgagatggcgaaggtcatctggattttctacgtctccaagatctatgagttcatggacacgttcatcatgctcttgaagggcaacgtcaaccaggtctctttcctgcatgtgtaccatcatggctccatctctggtatctggtggatgatcacctacgctgcccctggcggtgacgcgtacttctcggcggcgctcaactcgtgggtgcacgtgtgcatgtacacgtactacttcatggcggcggtgctgcccaaggacgagaagaccaagcgcaagtacctctggtggggccgctacctgacccagatgcagatgttccagttcttcatgaacctgctccaggcggtctacctcctctactcctctagcccctaccccaagttcatcgcccagctgctggtggtgtacatggtcacgctgctgatgctcttcggcaacttctactacatgaagcaccacgcgagcaagaagcagaagctggccagcaagaagcagtag

SEQ ID NO: 22 - DNA, 870 bp, synthetic construct
  misc_feature 1..870: note = Codon-optimized open reading frame for expression of Pyramimonascordata6-elongase in plants (truncated at 3' end and encodingfunctional elongase) (version 1)
  source 1..870: mol_type = other DNA
atggaattcgcccagcctcttgttgctatggctcaagagcaatacgctgctatcgatgctgttgttgctcctgctatcttctctgctactgattctatcggatggggacttaagcctatctcttctgctactaaggacttgcctcttgttgagtctcctacacctctcatcctttctttgcttgcttacttcgctatcgttggatctggactcgtttacagaaaggttttccctagaaccgtgaagggacaagatccattccttttgaaggctcttatgcttgctcacaacgtgttccttatcggactttctctttacatgtgcctcaagcttgtgtacgaggcttacgttaacaagtactctttctggggaaacgcttacaaccctgctcaaactgagatggctaaggttatctggatcttctacgtgagcaagatctacgagttcatggataccttcatcatgctcctcaagggaaatgttaaccaggttagcttccttcacgtttaccatcacggatctatctctggaatctggtggatgattacttacgctgctcctggtggtgatgcttacttctctgctgctcttaactcttgggttcacgtgtgtatgtacacctactattttatggctgccgtgcttcctaaggacgagaaaactaagagaaagtacctctggtggggaagataccttactcaaatgcagatgttccagttcttcatgaaccttctccaggctgtttaccttctctactcttcatctccttaccctaagtttatcgctcagctcctcgtggtgtacatggttactcttctcatgcttttcggaaacttctactacatgaagcaccacgctagcaagtgatga

SEQ ID NO: 23 - DNA, 870 bp, synthetic construct; identical to SEQ ID NO: 22
  misc_feature 1..870: note = Codon-optimized open reading frame for expression of Pyramimonascordata6-elongase in plants (truncated at 3' end and encodingfunctional elongase) (version 2)
  source 1..870: mol_type = other DNA

SEQ ID NO: 24 - DNA, 870 bp, synthetic construct
  misc_feature 1..870: note = Codon-optimized open reading frame for expression of Pyramimonascordata6-elongase in plants (truncated at 3' end and encodingfunctional elongase) (version 3)
  source 1..870: mol_type = other DNA
atggaatttgctcaacctctcgttgctatggctcaagagcagtacgctgctatcgatgctgttgttgctcctgctatcttctctgctaccgactctattggatggggactcaagcctatctcttctgctactaaggatctccctctcgttgaatctcctacccctcttatcctttctctcctcgcttacttcgctatcgttggttctggactcgtttaccgtaaagtgttccctagaaccgttaagggacaggatcctttccttctcaaggctcttatgctcgctcacaacgttttccttatcggactcagcctttacatgtgcctcaagctcgtttacgaggcttacgtgaacaagtactccttctggggaaacgcttacaaccctgctcaaaccgagatggctaaggtgatctggatcttctacgtgtccaagatctacgagttcatggacaccttcatcatgcttctcaagggaaacgttaaccaggtttccttcctccatgtttaccaccacggatctatctctggaatctggtggatgatcacttatgctgctccaggtggagatgcttacttctctgctgctctcaactcttgggttcatgtgtgcatgtacacctactacttcatggctgctgttcttcctaaggacgaaaagaccaagagaaagtacctttggtggggaagataccttacccagatgcaaatgttccagttcttcatgaaccttctccaggctgtttacctcctctactcttcttctccttaccctaagttcattgctcaactcctcgttgtttacatggttaccctcctcatgcttttcggaaacttctactacatgaagcaccacgcttctaagtgataa

SEQ ID NO: 25 - protein, 297 aa, Pyramimonas cordata
MEFAQPLVAMAQEQYAAIDAVVAPAIFSATDSIGWGLKPISSATKDLPLVESPTPLILSLLAYFAIVGSGLVYRKVFPRTVKGQDPFLLKALMLAHNVFLIGLSLYMCLKLVYEAYVNKYSFWGNAYNPAQTEMAKVIWIFYVSKIYEFMDTFIMLLKGNVNQVSFLHVYHHGSISGIWWMITYAAPGGDAYFSAALNSWVHVCMYTYYFMAAVLPKDEKTKRKYLWWGRYLTQMQMFQFFMNLLQAVYLLYSSSPYPKFIAQLLVVYMVTLLMLFGNFYYMKHHASKKQKLASKKQ

SEQ ID NO: 26 - protein, 288 aa, Pyramimonas cordata
MEFAQPLVAMAQEQYAAIDAVVAPAIFSATDSIGWGLKPISSATKDLPLVESPTPLILSLLAYFAIVGSGLVYRKVFPRTVKGQDPFLLKALMLAHNVFLIGLSLYMCLKLVYEAYVNKYSFWGNAYNPAQTEMAKVIWIFYVSKIYEFMDTFIMLLKGNVNQVSFLHVYHHGSISGIWWMITYAAPGGDAYFSAALNSWVHVCMYTYYFMAAVLPKDEKTKRKYLWWGRYLTQMQMFQFFMNLLQAVYLLYSSSPYPKFIAQLLVVYMVTLLMLFGNFYYMKHHASK

SEQ ID NO: 27 - DNA, 1278 bp, Pavlova salina
  source 1..1278: mol_type = other DNA
atgccgccgcgcgatagctactcgtacgccgccccgccgtcggcccagctgcacgaggtcgataccccgcaggagcatgataagaaggagctcgtcatcggtgaccgcgcgtacgacgtgaccaactttgtgaagcgccacccgggtggcaagatcatcgcataccaggttggcacagatgcgacggacgcgtacaagcagttccatgtgcggtctgccaaggcggacaagatgctcaagtcgctgccttcgcgcccggtgcacaagggctactcgccccgccgcgctgacctcattgccgacttccaggagttcaccaagcagctggaggcggagggcatgtttgagccgtcgctgccgcacgtggcataccgcctggcggaggtgatcgcgatgcacgtggccggcgccgcgctcatctggcacgggtacaccttcgcgggcattgccatgctcggcgttgtgcagggccgctgcggctggctcatgcacgagggcggccactactcgctcacgggcaacattgcttttgaccgtgccatccaagtcgcgtgctacggccttggctgcggcatgtcgggcgcgtggtggcgcaaccagcacaacaagcaccacgcgacgccgcagaagttgcagcacgacgtcgacctcgacaccctcccgctcgtcgccttccacgagcggatagccgccaaggtgaagagccccgcgatgaaggcgtggcttagtatgcaggcgaagctcttcgcgccagtgaccacgctgctggtcgcgctgggctggcagctgtacctgcacccgcgccatatgctgcgcaccaagcactacgacgagctcgcgatgctcggcattcgctacggccttgtcggctacctcgcggcgaactacggcgcggggtacgtgctcgcgtgctacctgctgtacgtgcagctcggcgccatgtacatcttctgcaactttgccgtgtcgcacacacacctgccggttgtcgagcctaacgagcacgcaacgtgggtggagtacgccgcgaaccacacgaccaactgctcgccctcgtggtggtgcgactggtggatgtcgtacctcaactaccagatcgagcaccacctctacccgtccatgccgcagttccgccacccgaagattgcgccgcgggtgaagcagctcttcgagaagcacggcctgcactacgacgtgcgtggctacttcgaggccatggcggacacgtttgccaaccttgacaacgtcgcgcacgcgccggagaagaagatgcagtga

SEQ ID NO: 28 - DNA, 1281 bp, synthetic construct
  misc_feature 1..1281: note = Codon-optimized open reading frame for expression of Pavlovasalina5-desaturase in plants (version 1)
  source 1..1281: mol_type = other DNA
atgcctccaagggactcttactcttatgctgctcctccttctgctcaacttcacgaagttgatactcctcaagagcacgacaagaaagagcttgttatcggagatagggcttacgatgttaccaacttcgttaagagacaccctggtggaaagatcattgcttaccaagttggaactgatgctaccgatgcttacaagcagttccatgttagatctgctaaggctgacaagatgcttaagtctcttccttctcgtcctgttcacaagggatactctccaagaagggctgatcttatcgctgatttccaagagttcaccaagcaacttgaggctgagggaatgttcgagccttctcttcctcatgttgcttacagacttgctgaggttatcgctatgcatgttgctggtgctgctcttatctggcatggatacactttcgctggaatcgctatgcttggagttgttcagggaagatgtggatggcttatgcatgagggtggacattactctctcactggaaacattgctttcgacagagctatccaagttgcttgttacggacttggatgtggaatgtctggtgcttggtggcgtaaccagcataacaagcaccatgctactcctcaaaagcttcagcacgatgttgatcttgatacccttcctctcgttgctttccatgagagaatcgctgctaaggttaagtctcctgctatgaaggcttggctttctatgcaagctaagcttttcgctcctgttaccactcttcttgttgctcttggatggcagctttaccttcatcctagacacatgctcaggactaagcactacgatgagcttgctatgctcggaatcagatacggacttgttggataccttgctgctaactacggtgctggatacgttctcgcttgttaccttctttacgttcagcttggagctatgtacatcttctgcaacttcgctgtttctcatactcacctccctgttgttgagcctaacgagcatgctacttgggttgagtacgctgctaaccacactactaactgttctccatcttggtggtgtgattggtggatgtcttaccttaactaccagatcgagcaccacctttacccttctatgcctcaattcagacaccctaagatcgctcctagagttaagcagcttttcgagaagcacggacttcactacgatgttagaggatacttcgaggctatggctgatactttcgctaaccttgataacgttgcccatgctcctgagaagaaaatgcagtaatga

SEQ ID NO: 29 - DNA, 1281 bp, synthetic construct
  misc_feature 1..1281: note = Codon-optimized open reading frame for expression of Pavlovasalina5-desaturase in plants (version 2)
  source 1..1281: mol_type = other DNA
atgcctcctagggactcttactcttacgctgctcctccttctgctcaacttcacgaggttgacactcctcaagagcacgacaagaaagagcttgttatcggagatagggcttacgatgtgaccaacttcgttaagagacaccctggtggaaagatcattgcttaccaagtgggaactgatgctaccgatgcttacaagcagttccatgtgagatctgctaaggctgacaagatgctcaagtctctcccttctagacctgttcacaagggatactctcctagaagagctgatcttatcgctgacttccaagagttcactaagcaacttgaggctgagggaatgttcgaaccttctctccctcatgttgcttaccgtcttgctgaggttatcgctatgcatgttgctggtgctgctcttatctggcacggatacactttcgctggaatcgctatgcttggagttgttcagggaagatgcggatggcttatgcatgagggtggacactactctcttaccggaaacattgctttcgatagggctatccaagttgcttgttacggacttggatgcggaatgtctggtgcttggtggagaaaccagcataacaagcaccatgctactcctcaaaagctccagcacgatgttgatcttgataccctccctctcgttgctttccatgagagaatcgctgctaaggttaagtctcctgctatgaaggcttggctctccatgcaagctaaactcttcgctcctgttaccactcttcttgttgctcttggatggcagctttaccttcaccctagacacatgctcagaactaagcactacgacgagcttgctatgcttggtatcagatacggacttgtgggataccttgctgctaactacggtgctggatacgttcttgcttgctaccttctctacgttcagcttggagctatgtacatcttctgcaacttcgctgtttctcacactcatctccctgttgttgagcctaacgagcatgctacttgggttgagtacgctgctaaccacactactaactgctctccatcttggtggtgtgattggtggatgagctacctcaactaccagatcgagcatcacctttacccttctatgcctcagttcaggcatcctaagatcgctcctagagtgaagcaactcttcgagaagcacggacttcactacgatgtgcgtggatacttcgaggctatggctgatactttcgctaacctcgataacgttgctcatgctcctgagaagaaaatgcaatgatga

SEQ ID NO: 30 - protein, 425 aa, Pavlova salina
MPPRDSYSYAAPPSAQLHEVDTPQEHDKKELVIGDRAYDVTNFVKRHPGGKIIAYQVGTDATDAYKQFHVRSAKADKMLKSLPSRPVHKGYSPRRADLIADFQEFTKQLEAEGMFEPSLPHVAYRLAEVIAMHVAGAALIWHGYTFAGIAMLGVVQGRCGWLMHEGGHYSLTGNIAFDRAIQVACYGLGCGMSGAWWRNQHNKHHATPQKLQHDVDLDTLPLVAFHERIAAKVKSPAMKAWLSMQAKLFAPVTTLLVALGWQLYLHPRHMLRTKHYDELAMLGIRYGLVGYLAANYGAGYVLACYLLYVQLGAMYIFCNFAVSHTHLPVVEPNEHATWVEYAANHTTNCSPSWWCDWWMSYLNYQIEHHLYPSMPQFRHPKIAPRVKQLFEKHGLHYDVRGYFEAMADTFANLDNVAHAPEKKMQ

SEQ ID NO: 31 - DNA, 1329 bp, Pyramimonas cordata
  source 1..1329: mol_type = other DNA
atgggaaagggaggcaatgctagcgctcctactgcgaagaaggaggtgttgatcgaggggaagttttacgatgtcaccgacttcaggcaccccggtggttcgatcatcaagtttctctcgggttctggtgctgacgccaccgcttcctaccgcgagttccacgttaggtcagcgaaggcagacaagttcttgaagacgctgccctcccgcgaagccactccccaggagctgaagcaggcggttgagttctccaagctcaacccgccctccgcggagagtgcctctgctcccctgaccgaccttgccaaggtggaagcgctgaacaaggacttcgaggctttccgtgagcagctcattcaggagggcttctttaagcccaatatcccgcatgtggtcaagcgcatcacggaagtcgtggcgatgatggccgtagcctcctggatgatggtgcagaccaacgctcttgttgtgaccctcggagttctgatccgcggcattgcacagggccggtgcggttggcttatgcacgagggcggccactatagtcttactgggaagatctccattgataggcgtctgcaggagtcaatttacggattcggctgtggaatgtccggcgcctggtggcgcaaccagcacaacaagcaccacgcaaccccacagaagctgcagcatgacgtcgacctggagacccttcctctgatggctttcaacaacgctgttaccgatagacgcaaggtgaagcctggtagtctccaggctctgtggctcaagtaccaggccttcctcttcttccccgtgacctcccttctggtcggcctcggttggaccaccgtcctccaccccaggcacagcttgcgcaccaagcactatttcgagctgctctgcatggctgctcgttacgcgagtttcgctgctcttttcgctcccaagtacggacttgcaggagctgccgggctctacctcgccaccttcgctgtcgggtgcaactatattttcatcaacttctcggtctctcacactcacctgcccgtgagcggtgcgagcgagtacctgcattgggtcgtgtattcggccatccacaccactaacatcaaatccagcatgctgtgcgattggtggatgtcattcctcaacttccagatcgagcatcacctgttcccttcaatgccccagttccgccacaagattatctccccgcgtgtaaaggccttgtttgagaagcacggtcttgtgtatgatgtgcgcccctattggggggccatggctgacaccttcaagaacttgaatgacgttggcactcacgcatctcactccaaggcgcactag

SEQ ID NO: 32 - protein, 442 aa, Pyramimonas cordata
MGKGGNASAPTAKKEVLIEGKFYDVTDFRHPGGSIIKFLSGSGADATASYREFHVRSAKADKFLKTLPSREATPQELKQAVEFSKLNPPSAESASAPLTDLAKVEALNKDFEAFREQLIQEGFFKPNIPHVVKRITEVVAMMAVASWMMVQTNALVVTLGVLIRGIAQGRCGWLMHEGGHYSLTGKISIDRRLQESIYGFGCGMSGAWWRNQHNKHHATPQKLQHDVDLETLPLMAFNNAVTDRRKVKPGSLQALWLKYQAFLFFPVTSLLVGLGWTTVLHPRHSLRTKHYFELLCMAARYASFAALFAPKYGLAGAAGLYLATFAVGCNYIFINFSVSHTHLPVSGASEYLHWVVYSAIHTTNIKSSMLCDWWMSFLNFQIEHHLFPSMPQFRHKIISPRVKALFEKHGLVYDVRPYWGAMADTFKNLNDVGTHASHSKAH

SEQ ID NO: 33 - DNA, 804 bp, Pyramimonas cordata
  source 1..804: mol_type = other DNA
atggcgtctattgcgattccggctgcgctggcagggactcttggttatgtgacgtacaatgtcgcaaacccagatattcctgcatccgagaaggtgcctgcttactttatgcaggtcgagtattgggggccaacgattgggaccatcggttatcttctgttcatctactttggtaaacggattatgcaaaacaggagccagccgtttggcctgaagaacgctatgctggtgtacaacttctatcagactttcttcaactcgtactgcatatacctttttgtcacgtcgcaccgcgctcaggggctgaaagtttggggaaacatccccgatatgactgccaacagctgggggatctcacaggtgatctggctgcactacaacaacaagtacgttgagctgctggacacgttcttcatggtcatgcgcaagaagtttgaccagctttcgttcctgcacatttaccatcataccctgttgatctggtcttggttcgtggtgatgaaattggagcccgttggggactgctactttggctctagcgtcaacacgtttgtgcacgtcattatgtactcgtactatggccttgccgcgctcggggtgaattgcttctggaagaagtacattacgcagattcagatgctgcagttctgtatctgcgcttcgcactcgatttataccgcctatgtgcagaacaccgcgttctggttgccttacttgcagctgtgggtgatggtgaacatgttcgtgttgttcgccaacttctatcgcaagcgctacaagagcaagggtgccaagaagcagtaa

SEQ ID NO: 34 - DNA, 807 bp, synthetic construct
  misc_feature 1..807: note = Codon-optimized open reading frame for expression of Pyramimonascordata5-elongase in plants (version 1)
  source 1..807: mol_type = other DNA
atggcctctatcgctatccctgctgctcttgctggaactcttggatacgttacctacaatgtggctaaccctgatatcccagcttctgagaaagttcctgcttacttcatgcaggttgagtactggggacctactatcggaactattggatacctcctcttcatctacttcggaaagcgtatcatgcagaacagatctcaacctttcggactcaagaacgctatgctcgtttacaacttctaccagaccttcttcaacagctactgcatctaccttttcgttacttctcatagggctcagggacttaaggtttggggaaacatccctgatatgactgctaactcttggggaatctctcaggttatctggcttcactacaacaacaagtacgttgagcttctcgacaccttcttcatggtgatgaggaagaagttcgaccagctttctttccttcacatctaccaccacactcttctcatctggtcatggttcgttgttatgaagcttgagcctgttggagattgctacttcggatcttctgttaacaccttcgtgcacgtgatcatgtactcttactacggacttgctgctcttggagttaactgtttctggaagaagtacatcacccagatccagatgcttcagttctgtatctgtgcttctcactctatctacaccgcttacgttcagaataccgctttctggcttccttaccttcaactctgggttatggtgaacatgttcgttctcttcgccaacttctaccgtaagaggtacaagtctaagggtgctaagaagcagtgataa

SEQ ID NO: 35 - DNA, 867 bp, synthetic construct
  misc_feature 1..867: note = Codon-optimized open reading frame for expression of Pyramimonascordata5-elongase in plants (version 2)
  source 1..867: mol_type = other DNA
atggaatttgctcaacctctcgttgctatggctcaagagcagtacgctgctatcgatgctgttgttgctcctgctatcttctctgctaccgactctattggatggggactcaagcctatctcttctgctactaaggatctccctctcgttgaatctcctacccctcttatcctttctctcctcgcttacttcgctatcgttggttctggactcgtttaccgtaaagtgttccctagaaccgttaagggacaggatcctttccttctcaaggctcttatgctcgctcacaacgttttccttatcggactcagcctttacatgtgcctcaagctcgtttacgaggcttacgtgaacaagtactccttctggggaaacgcttacaaccctgctcaaaccgagatggctaaggtgatctggatcttctacgtgtccaagatctacgagttcatggacaccttcatcatgcttctcaagggaaacgttaaccaggtttccttcctccatgtttaccaccacggatctatctctggaatctggtggatgatcacttatgctgctccaggtggagatgcttacttctctgctgctctcaactcttgggttcatgtgtgcatgtacacctactacttcatggctgctgttcttcctaaggacgaaaagaccaagagaaagtacctttggtggggaagataccttacccagatgcaaatgttccagttcttcatgaaccttctccaggctgtttacctcctctactcttcttctccttaccctaagttcattgctcaactcctcgttgtttacatggttaccctcctcatgcttttcggaaacttctactacatgaagcaccacgcttctaagtga

SEQ ID NO: 36 - DNA, 807 bp, synthetic construct
  misc_feature 1..807: note = Codon-optimized open reading frame for expression of Pyramimonascordata5-elongase in plants (version 3)
  source 1..807: mol_type = other DNA
atggcttctatcgctatccctgctgctcttgctggaactcttggatacgtgacctacaacgtggctaaccctgatattcctgcttctgagaaggttccagcttacttcatgcaagtggagtactggggacctactatcggaactatcggttacctcctcttcatctacttcggaaagcgtatcatgcaaaacagaagccagcctttcggacttaagaacgctatgctcgtgtacaacttctaccagaccttcttcaacagctactgcatctacctcttcgttacctctcatagggctcagggacttaaagtttggggaaacatccctgatatgaccgctaactcttggggaatctctcaggttatctggctccactacaacaacaagtacgtggagcttctcgataccttcttcatggtgatgaggaagaagttcgaccagctttctttccttcacatctaccaccacactcttctcatctggtcatggttcgtggttatgaagctcgagcctgttggagattgctacttcggatctagcgttaacaccttcgtgcacgtgatcatgtactcttactacggacttgctgctcttggagttaactgcttctggaagaagtacatcacccagatccagatgcttcagttctgtatctgcgcttctcactctatctacaccgcttacgttcagaacactgctttctggcttccttaccttcagctctgggtgatggttaacatgttcgtgctcttcgctaacttctaccgtaaaaggtacaagagcaagggtgctaagaagcagtgataa

SEQ ID NO: 37 - protein, 267 aa, Pyramimonas cordata
MASIAIPAALAGTLGYVTYNVANPDIPASEKVPAYFMQVEYWGPTIGTIGYLLFIYFGKRIMQNRSQPFGLKNAMLVYNFYQTFFNSYCIYLFVTSHRAQGLKVWGNIPDMTANSWGISQVIWLHYNNKYVELLDTFFMVMRKKFDQLSFLHIYHHTLLIWSWFVVMKLEPVGDCYFGSSVNTFVHVIMYSYYGLAALGVNCFWKKYITQIQMLQFCICASHSIYTAYVQNTAFWLPYLQLWVMVNMFVLFANFYRKRYKSKGAKKQ

SEQ ID NO: 38 - DNA, 1344 bp, Pavlova salina
  source 1..1344: mol_type = other DNA
atgcctccgagcgcggcgaagcagatgggcgcgagcacgggcgtgcatgcgggcgtcacagattcgtcggccttcacgcgcaaggatgtcgccgacaggccggacctcacgatcgtgggtgacagcgtgtacgatgcgaaggcgttccgctccgagcatccgggtggcgcgcactttgtgtcgctgttcggcgggcgcgatgccacggaggcgttcatggagtaccaccggcgcgcctggcccaagtcgcgcatgtcgcgcttccacgtcggctctctggcatcgaccgaggagcccgtcgccgccgatgagggctacctccagctgtgcgctcgcatcgccaagatggtgccgtcggtcagcagcgggttcgcgccggcgtcgtactgggtgaaggccgggctgatcctcggctccgcgatcgcgctcgaggcgtacatgctgtacgcgggcaagcgcctgctcccgtcgatcgtgctcgggtggctgtttgcgctgattggcctgaacatccagcacgatgccaaccacggcgcgctctccaagtcggcctcggtcaacctggcgctcgggttgtgccaggactggatcggcgggagcatgatcctctggctgcaggagcacgttgtcatgcaccacttgcacaccaacgacgttgacaaggacccggaccagaaggcgcacggcgccctgcggctcaagccgaccgacgcgtggagcccgatgcactggctgcagcacctctacctgctgcctggggagacgatgtacgccttcaagctgctgtttctcgacatcagcgagctggtgatgtggcggtgggagggcgagcccatcagcaagctggccgggtacctcttcatgccctcgctgctcctcaagctcaccttctgggcgcgctttgtcgcgctgccgctgtacctcgcgcccagcgtgcacacggcggtgtgcatcgcggcgacggtaatgacggggagcttctacctcgccttcttcttcttcatctcgcacaacttcgagggcgtggcgagcgtcggaccggacggcagcatcaccagcatgacgcgcggcgcatccttcctcaagcggcaggccgagacctcgtccaacgtgggcggcccgctgctcgccacgctcaacggcggcctcaactaccaaatcgagcaccacctcttccccagggtgcaccacggcttctaccctcgcctcgcgccgttggtcaaggcggagctcgaggcgcgcggcattgagtacaagcactaccccaccatatggagcaacctggcatccacgctgaggcacatgtacgcgctcggccgcaggccgcgcagcaaggcggagtga

SEQ ID NO: 39 - DNA, 1347 bp, synthetic construct
  misc_feature 1..1347: note = Codon-optimized open reading frame for expression of Pavlovasalina4-desaturase in plants (version 1)
  source 1..1347: mol_type = other DNA
atgccacctagcgctgctaagcaaatgggagcttctactggtgttcatgctggtgttactgactcttctgctttcaccagaaaggatgttgctgatagacctgatctcaccatcgttggagattctgtttacgatgctaaggctttcagatctgagcatcctggtggtgctcatttcgtttctttgttcggaggaagagatgctactgaggctttcatggaataccatagaagggcttggcctaagtctagaatgtctagattccacgttggatctcttgcttctactgaggaacctgttgctgctgatgagggataccttcaactttgtgctaggatcgctaagatggtgccttctgtttcttctggattcgctcctgcttcttactgggttaaggctggacttatccttggatctgctatcgctcttgaggcttacatgctttacgctggaaagagacttctcccttctatcgttcttggatggcttttcgctcttatcggtcttaacatccagcatgatgctaaccatggtgctttgtctaagtctgcttctgttaaccttgctcttggactttgtcaggattggatcggaggatctatgatcctttggcttcaagagcatgttgttatgcaccacctccacactaacgatgttgataaggatcctgatcaaaaggctcacggtgctcttagactcaagcctactgatgcttggtcacctatgcattggcttcagcatctttaccttttgcctggtgagactatgtacgctttcaagcttttgttcctcgacatctctgagcttgttatgtggcgttgggagggtgagcctatctctaagcttgctggatacctctttatgccttctttgcttctcaagcttaccttctgggctagattcgttgctttgcctctttaccttgctccttctgttcatactgctgtgtgtatcgctgctactgttatgactggatctttctacctcgctttcttcttcttcatctcccacaacttcgagggtgttgcttctgttggacctgatggatctatcacttctatgactagaggtgctagcttccttaagagacaagctgagacttcttctaacgttggaggacctcttcttgctactcttaacggtggactcaactaccaaattgagcatcacttgttccctagagttcaccatggattctaccctagacttgctcctcttgttaaggctgagcttgaggctagaggaatcgagtacaagcactaccctactatctggtctaaccttgcttctaccctcagacatatgtacgctcttggaagaaggcctagatctaaggctgagtaatga

SEQ ID NO: 40 - DNA, 1347 bp, synthetic construct
  misc_feature 1..1347: note = Codon-optimized open reading frame for expression of Pavlovasalina4-desaturase in plants (version 2)
  source 1..1347: mol_type = other DNA
atgcctccatctgctgctaaacagatgggagcttctactggtgttcacgctggtgttaccgattcttctgctttcaccagaaaggatgtggctgatagacctgatcttaccatcgttggtgactctgtgtacgatgctaaggctttcagatctgagcatcctggtggtgctcatttcgtttcactcttcggaggaagagatgctactgaggctttcatggaataccacagaagagcttggcctaagtctaggatgtctaggttccatgttggatctcttgcttctaccgaggaacctgttgctgctgatgagggataccttcagctttgtgctaggatcgctaagatggtgccttctgtgtcatctggattcgctccagcttcttactgggttaaggctggacttatcctcggatctgctatcgctcttgaggcttacatgctctacgctggaaagagacttctcccttctatcgttcttggatggctcttcgctcttatcggacttaacatccagcatgacgctaaccatggtgctttgtctaagtctgctagcgttaaccttgctcttggactttgtcaggattggatcggaggatctatgatcctttggctccaagagcatgttgttatgcaccacctccacaccaacgatgttgataaggaccctgatcaaaaggctcatggtgctcttagactcaagcctaccgatgcttggtcacctatgcattggcttcagcacctttaccttctccctggtgaaactatgtacgctttcaagctcctcttcctcgatatctctgagcttgtgatgtggagatgggagggtgaacctatctctaagctcgctggatacctcttcatgccttctcttctcctcaagcttaccttctgggctagattcgttgctcttcctctttacctcgctccttctgttcatactgctgtgtgtatcgctgctactgttatgaccggaagcttctaccttgctttcttcttcttcatcagccacaacttcgagggtgttgcttctgttggacctgatggatctatcacctctatgaccaggggagcttctttccttaagaggcaggctgagacttcttctaatgtgggaggacctcttcttgctactcttaacggtggactcaactaccaaatcgagcaccaccttttccctagagttcaccacggattctaccctagacttgctcctcttgtgaaggctgaacttgaggctagaggaatcgagtacaagcactaccctaccatctggtctaacctcgcttctaccctcagacatatgtacgctcttggaagaaggcctagatctaaggctgagtgatga

SEQ ID NO: 41 - protein, 447 aa, Pavlova salina
MPPSAAKQMGASTGVHAGVTDSSAFTRKDVADRPDLTIVGDSVYDAKAFRSEHPGGAHFVSLFGGRDATEAFMEYHRRAWPKSRMSRFHVGSLASTEEPVAADEGYLQLCARIAKMVPSVSSGFAPASYWVKAGLILGSAIALEAYMLYAGKRLLPSIVLGWLFALIGLNIQHDANHGALSKSASVNLALGLCQDWIGGSMILWLQEHVVMHHLHTNDVDKDPDQKAHGALRLKPTDAWSPMHWLQHLYLLPGETMYAFKLLFLDISELVMWRWEGEPISKLAGYLFMPSLLLKLTFWARFVALPLYLAPSVHTAVCIAATVMTGSFYLAFFFFISHNFEGVASVGPDGSITSMTRGASFLKRQAETSSNVGGPLLATLNGGLNYQIEHHLFPRVHHGFYPRLAPLVKAELEARGIEYKHYPTIWSNLASTLRHMYALGRRPRSKAE

SEQ ID NO: 42 - DNA, 792 bp, Isochrysis galbana
  source 1..792: mol_type = other DNA
atggccctcgcaaacgacgcgggagagcgcatctgggcggctgtgaccgacccggaaatcctcattggcaccttctcgtacttgctactcaaaccgctgctccgcaattccgggctggtggatgagaagaagggcgcatacaggacgtccatgatctggtacaacgttctgctggcgctcttctctgcgctgagcttctacgtgacggcgaccgccctcggctgggactatggtacgggcgcgtggctgcgcaggcaaaccggcgacacaccgcagccgctcttccagtgcccgtccccggtttgggactcgaagctcttcacatggaccgccaaggcattctattactccaagtacgtggagtacctcgacacggcctggctggtgctcaagggcaagagggtctcctttctccaggccttccaccactttggcgcgccgtgggatgtgtacctcggcattcggctgcacaacgagggcgtatggatcttcatgtttttcaactcgttcattcacaccatcatgtacacctactacggcctcaccgccgccgggtataagttcaaggccaagccgctcatcaccgcgatgcagatctgccagttcgtgggcggcttcctgttggtctgggactacatcaacgtcccctgcttcaactcggacaaagggaagttgttcagctgggctttcaactatgcatacgtcggctcggtcttcttgctcttctgccactttttctaccaggacaacttggcaacgaagaaatcggccaaggcgggcaagcagctctag

SEQ ID NO: 43 - protein, 263 aa, Isochrysis galbana
MALANDAGERIWAAVTDPEILIGTFSYLLLKPLLRNSGLVDEKKGAYRTSMIWYNVLLALFSALSFYVTATALGWDYGTGAWLRRQTGDTPQPLFQCPSPVWDSKLFTWTAKAFYYSKYVEYLDTAWLVLKGKRVSFLQAFHHFGAPWDVYLGIRLHNEGVWIFMFFNSFIHTIMYTYYGLTAAGYKFKAKPLITAMQICQFVGGFLLVWDYINVPCFNSDKGKLFSWAFNYAYVGSVFLLFCHFFYQDNLATKKSAKAGKQL

SEQ ID NO: 44 - DNA, 801 bp, Emiliania huxleyi
  source 1..801: mol_type = other DNA
atgctcgatcgcgcctcgtccgacgcggccatctggtctgcggtgtccgatccggaaatcctgatcggcactttctcctacctgctgctcaagccgctgctacgcaactcagggctcgtggacgagcggaaaggcgcctaccggacctcgatgatctggtacaacgtggtgctcgcgctcttctccgcgacgagcttctacgtgactgcgaccgcgctcgggtgggacaagggcaccggcgagtggctccgcagtctcacgggcgacagcccgcagcagctgtggcaatgcccgtcgagggtatgggactccaagctgttcctgtggacggccaaggccttctactactcaaagtacgtggagtacctcgacacggcgtggctcgtcctcaaggggaagaaggtctccttcctgcagggcttccaccactttggcgcgccgtgggacgtgtacctgggcattcggctgaagaacgagggcgtgtggatcttcatgttcttcaactcgttcatccacacggtcatgtacacgtactacggcctcaccgccgcgggctacaagatccgcggcaagccgatcatcaccgcgatgcaaataagccagttcgtcggcggctttgtcctagtgtgggactacatcaacgtgccgtgcttccacgccgacgccgggcaggtcttcagctgggtctttaactatgcttacgtcggctccgtctttctgctgttctgccacttcttctacatggacaacatcgcgaaggccaaggccaagaaggccgtcgctacccgcaaggcgctgtga

SEQ ID NO: 45 - DNA, 801 bp, synthetic construct
  misc_feature 1..801: note = Codon-optimized open reading frame for expression of Emilianiahuxleyi9-elongase in plants
  source 1..801: mol_type = other DNA
atgcttgatagagcttcatctgatgctgctatttggagcgctgtttctgatcctgagatccttatcggaaccttctcttaccttttgcttaagcctctcctcagaaactctggacttgtggatgagagaaagggagcttaccgtacttctatgatctggtacaacgttgttcttgctcttttctctgctacctctttctacgttactgctactgctcttggatgggataagggaactggtgagtggcttagatctcttactggtgattctcctcaacaactttggcagtgcccttctagagtttgggacagcaaactcttcttgtggactgctaaagccttctactactccaagtacgttgagtaccttgatactgcttggcttgttctcaagggaaagaaggtttcattcctccagggattccatcatttcggtgctccatgggatgtttaccttggaatcaggcttaagaacgagggagtttggatcttcatgttcttcaacagcttcatccacactgttatgtacacttactacggacttactgctgctggatacaagatcagaggaaagcctatcatcaccgctatgcaaatctctcaattcgttggtggattcgttcttgtgtgggactacatcaacgttccttgtttccatgctgatgctggacaagttttctcttgggtgttcaactacgcttatgtgggatctgttttccttcttttctgccacttcttctacatggacaacattgctaaggctaaggctaaaaaggctgttgctaccagaaaggctctttga

SEQ ID NO: 46 - protein, 266 aa, Emiliania huxleyi
MLDRASSDAAIWSAVSDPEILIGTFSYLLLKPLLRNSGLVDERKGAYRTSMIWYNVVLALFSATSFYVTATALGWDKGTGEWLRSLTGDSPQQLWQCPSRVWDSKLFLWTAKAFYYSKYVEYLDTAWLVLKGKKVSFLQGFHHFGAPWDVYLGIRLKNEGVWIFMFFNSFIHTVMYTYYGLTAAGYKIRGKPIITAMQISQFVGGFVLVWDYINVPCFHADAGQVFSWVFNYAYVGSVFLLFCHFFYMDNIAKAKAKKAVATRKAL

SEQ ID NO: 47 - DNA, 819 bp, Pavlova pinguis
  source 1..819: mol_type = other DNA
atggttgcgccacccatcacgctcgagtggctgctttcgccgaagctcaaggatgcagtgttcggtggggaggtgctctacttctccattgcctacctgtttcttgcgcccattttgaagcgcaccccgttggtggacacgcggaagggcgcgtataagagtggtatgatcgcgtacaacgtgatcatgtgcgtgttctcgctggtgtgcttcatctgccagctcgcagccctgggctatgacatgggctacttgcagtgggtgcgtgacctcacaggggacgagattgtccccctctaccaggacgtgtccccgtcccccgccttctccaacaagctcttcaagtattcgtctattgccttccactactccaagtatgttgagtacatggacaccgcatggctggtgatgaagggcaagcccgtgtccttgctccagggcttccaccactttggcgccgcctgggacacctactttggcatcaccttccagaacgagggcatctacgtgttcgtggtgctcaacgccttcatccacacgatcatgtacgcatactacgcggccactgcggcgggtctcaagttctcactgaagttcgtcatcacgctcatgcagatcacccaattcaacgtgggcttcgtaatggtgtatcactacatcaccctggagtacttccgcaactcaccggagctcgtcttctcctaccttttcaactatgcgtacgtctgcacggttctcctcctcttcatgcagttcttctacatggacaactttggcaagaagaaggccgctgccgccgcgggcaagaagaagaagtag

SEQ ID NO: 48 - protein, 272 aa, Pavlova pinguis
MVAPPITLEWLLSPKLKDAVFGGEVLYFSIAYLFLAPILKRTPLVDTRKGAYKSGMIAYNVIMCVFSLVCFICQLAALGYDMGYLQWVRDLTGDEIVPLYQDVSPSPAFSNKLFKYSSIAFHYSKYVEYMDTAWLVMKGKPVSLLQGFHHFGAAWDTYFGITFQNEGIYVFVVLNAFIHTIMYAYYAATAAGLKFSLKFVITLMQITQFNVGFVMVYHYITLEYFRNSPELVFSYLFNYAYVCTVLLLFMQFFYMDNFGKKKAAAAAGKKKK

SEQ ID NO: 49 - DNA, 840 bp, Pavlova salina
  source 1..840: mol_type = other DNA
atggcgactgaagggatgccggcgataacgctggactggctgctctcgcccgggctgaaggatgccgtaattggcggggaggtgctctacttttcgcttgggtatctgctgctcgagcccatcctcaagcgctcaccgtttgtggacaagcgcaagggcgcataccgcaacggcatgatcgcgtacaacatcctcatgtgcggtttctcgctggtatgcttcgtgtgccagatggcggcgctcggccttgatcgcggccacctgcagtttgtccgcgacctcacgggcgacagcgtggtgcagctctaccaggacgtgagcccatcccctgcattcgcgaacaagctcttccggtactcagcggtggcgttccactactcaaagtacgtggagtacatggacacagcgtggcttgtgctgaagggcaagcccgtctcgttcctgcagggcttccaccacttcggcgccgcgtgggacacctactttggcatcacgtttcagaacgagggcacctacgtctttgtgctgctcaacgcattcatccacacaatcatgtacacctactacggcgcgacggcagcgggcatcaaaatctcgatgaagccgctgatcaccctcatgcagatcacgcagttcctgctgggcttcgcgctcgtctacccgtacattgacctcggctacttccgtgcgtcgcccgagctcgtgtggagctacctgttcaactatgcgtacgtactcatggtgctcttcctcttcatgcgcttcttctaccacgacaactttagcaagcacaagccaatctcgcgcatcgactccagcaaccgcatgaaaaccgagtag

SEQ ID NO: 50 - protein, 279 aa, Pavlova salina
MATEGMPAITLDWLLSPGLKDAVIGGEVLYFSLGYLLLEPILKRSPFVDKRKGAYRNGMIAYNILMCGFSLVCFVCQMAALGLDRGHLQFVRDLTGDSVVQLYQDVSPSPAFANKLFRYSAVAFHYSKYVEYMDTAWLVLKGKPVSFLQGFHHFGAAWDTYFGITFQNEGTYVFVLLNAFIHTIMYTYYGATAAGIKISMKPLITLMQITQFLLGFALVYPYIDLGYFRASPELVWSYLFNYAYVLMVLFLFMRFFYHDNFSKHKPISRIDSSNRMKTE

SEQ ID NO: 51 - DNA, 1284 bp, Pavlova salina
  source 1..1284: mol_type = other DNA
atgggacgcggcggagacagcagtgggcaggcgcatccggcggcggagctggcggtcccgagcgaccgcgcggaggtgagcaacgctgacagcaaagcgctgcacatcgtgctgtatggcaagcgcgtggatgtgaccaagttccaacgcacgcacccgggtggtagcaaggtcttccggatcttccaggaccgcgatgcgacggagcagttcgagtcctaccactcgaagcgcgcgatcaagatgatggagggcatgctcaagaagtctgaggatgctcccgccgacacgcccttgccctcccagtcaccgatggggaaggacttcaaggcgatgatcgagcggcacgttgcagcgggttactacgatccatgcccgctcgatgagctgttcaagctcagcctcgtgctcctcccgacctttgcgggcatgtacatgctcaaggcgggcgtcggctccccgctctgcggcgccctcatggtgagctttggctggtacctcgatggctggctcgcgcacgactatctgcaccactccgtcttcaaggggtccgtcgcacgcaccgtcgggtggaacaacgcggcgggctacttcctcggcttcgtgcaggggtatgcggtcgagtggtggcgcgcgcggcataacacgcaccacgtgtgcaccaatgaggacggctcggaccccgacatcaaaacggcgccgctgctcatatacgtgcgcaacaagccgagcatcgccaagcgcctgaacgccttccagcgctaccagcagtactactatgtgccggtgatggcaatcctcgacctgtactggcggctcgagtcgatcgcctacgtcgcgatgcgcctgccgaagatgctgccgcaggccctcgcactcgtcgcgcactacgccatcgtcgcgtgggtctttgcgggcaactaccacctgctcccgctcgtgacggttctgcgcgggtttggcactgggatcaccgttttcgcgacgcactacggtgaggacattctcgacgcggaccaggtgcgtcacatgacgctcgtcgagcagacggcactcacctcgcgcaacatctcgggcggctggctcgtgaacgtgctcaccggcttcatctcactgcagacggagcaccacctgttcccgatgatgccaaccggcaacctcatgactatccagcccgaggtgcgcgccttcttcaagaagcacggacttgagtaccgcgagggcaacctcattgagtgcgtgcggcagaacatccgtgcgcttgcattcgagcacctgctttga

SEQ ID NO: 52 - protein, 427 aa, Pavlova salina
MGRGGDSSGQAHPAAELAVPSDRAEVSNADSKALHIVLYGKRVDVTKFQRTHPGGSKVFRIFQDRDATEQFESYHSKRAIKMMEGMLKKSEDAPADTPLPSQSPMGKDFKAMIERHVAAGYYDPCPLDELFKLSLVLLPTFAGMYMLKAGVGSPLCGALMVSFGWYLDGWLAHDYLHHSVFKGSVARTVGWNNAAGYFLGFVQGYAVEWWRARHNTHHVCTNEDGSDPDIKTAPLLIYVRNKPSIAKRLNAFQRYQQYYYVPVMAILDLYWRLESIAYVAMRLPKMLPQALALVAHYAIVAWVFAGNYHLLPLVTVLRGFGTGITVFATHYGEDILDADQVRHMTLVEQTALTSRNISGGWLVNVLTGFISLQTEHHLFPMMPTGNLMTIQPEVRAFFKKHGLEYREGNLIECVRQNIRALAFEHLL

SEQ ID NO: 53 - protein, 172 aa, Tomato bushy stunt virus
MERAIQGNDAREQANSERWDGGSGGTTSPFKLPDESPSWTEWRLHNDETNSNQDNPLGFKESWGFGKVVFKRYLRYDRTEASLHRVLGSWTGDSVNYAASRFFGFDQIGCTYSIRFRGVSITVSGGSRTLQHLCEMAIRSKQELLQLAPIEVESNVSRGCPEGTETFEKESE

SEQ ID NO: 54 - protein, 116 aa, Tomato yellow leaf curl virus
MWDPLLNEFPESVHGFRCMLAIKYLQSVEETYEPNTLGHDLIRDLISVVRARDYVEATRRYNHFHARLEGSPKAELRQPIQQPCCCPHCPRHKQATIMDVQAHVPEAQNIQNVSKP

SEQ ID NO: 55 - protein, 351 aa, Turnip crinkle virus
MENDPRVRKFASEGAQWAIKWQKKGWSSLTSRQKQTARAAMGIKLSPVAQPVQKVTRLSAPVALAYREVSTQPRVSTARDGITRSGSELITTLKKNTDTEPKYTTAVLNPSEPGTFNQLIKEAAQYEKYRFTSLRFRYSPMSPSTTGGKVALAFDRDAAKPPPNDLASLYNIEGCVSSVPWTGFILTVPTDSTDRFVADGISDPKLVNFGKLIMATYGQGANDAAQLGEVRVEYTVQLKNRTGSTSDAQIGDFAGVKDGPRLVSWSKTKGTAGWEHDCHFLGTGNFSLTLFYEKAPVSGLENADASDFSVLGEAAAGSVQWAGVKVAERGQSVKMVTTEEQPRGKWQALRI

SEQ ID NO: 56 - protein, 303 aa, Pea mosaic virus
MHGIEQPQLPLDYVHRCASTSFLLASLDGLLSEARELSGPLALITSSYYLLVSIALCWAIPGSFWYRPGCWLQPVSGRNLIFCGPTEALQRFRLYAARLGLVLSENCPRHGQSAAITLQSYWALPNNIWMDMAQLDLLTFSMPIANTFAYLADCEARFPPIVEGVGSAYYVPTLLGLTHQDPRLYLALRRRNLDLSGEPHRVRPGVLESMALLCSSVRSTSRSRQIPPLYGSVLHHVLGLAERDCILFDTDSNYSSYTHRVLEQDRNRADQSLFSIDLEYVHDLELIALGYSDEDDEDLDNFF

SEQ ID NO: 57 - protein, 256 aa, Cereal yellow dwarf virus
MFIAQPCGRVLVFDVASRTPSFFTRYSVELSLRVLDPFFTRAVTDFRYTQNEIDLFCVSLGFLLPILLTGESYSWRGHLNLPLSYTELLVRWGLAVGYFPTFSTDGDIRQNPELRIDLSTMSTRSFYEQFLLRYNTSGLAKAIVGQQECFQSGMESFKRFLHYRLTCFESCLPRPRWESPLAPGPYLDRAFEATLLGRMVGHNQLLFTGLSSDITRYYNELVVEGVPVAFWDAAGITLHHAGEEYFSNSYIQKILQ

SEQ ID NO: 58 - DNA, 519 bp, Tomato bushy stunt virus
  source 1..519: mol_type = other DNA
atggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactgagaccttcgaaaaagaaagcgagtaa

SEQ ID NO: 59 - DNA, 351 bp, Tomato yellow leaf curl virus
  source 1..351: mol_type = other DNA
atgtgggatccacttctaaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgcctcgaaggttcgccgaaggctgaacttcgacagcccatacagcagccgtgctgctgtccccattgtccaaggcacaaacaagcgacgatcatggacgtacaggcccatgtaccggaagcccagaatatacagaatgtatcgaagccctga

SEQ ID NO: 60 - DNA, 1056 bp, Turnip crinkle virus
  source 1..1056: mol_type = other DNA
atggaaaatgatcctagagtccgaaagttcgcatccgagggcgcccaatgggcgataaagtggcagaagaagggctggtcatccctaaccagcagacagaaacagaccgcccgcgcagcgatggggatcaagctctcccctgtggcgcaacctgtgcagaaagtgactcgactgagtgctccggtggctctcgcctaccgcgaggtttccacccagcctcgggtttctactgccagggacggcataaccagaagcggttctgaactgatcacaaccctgaagaagaacactgacactgaacctaagtacaccacagctgtgcttaacccaagcgaacccggaacattcaaccaactcatcaaggaggcggcccagtatgaaaaataccgattcacgtcactcagatttaggtactctcccatgagcccttcaaccaccgggggcaaggtggctctggcattcgaccgagacgctgccaaacctccgcccaacgacctcgcttccctctacaacatagagggttgtgtatctagcgtgccctggacagggtttattttgaccgtcccaacagattctactgaccgctttgtggcggatggtatcagcgatccaaagcttgtcaatttcggcaagctcatcatggccacctatggccaaggagccaatgatgccgcccaactcggtgaagtgcgagtcgagtacaccgtgcagctcaagaacagaactggctcaaccagcgacgcccagattggggacttcgcgggtgttaaggacggacccaggttggtctcgtggtccaagaccaagggaacagctgggtgggagcacgattgtcattttctcggaaccggaaacttctcgttgacactgttctacgagaaggcgcccgtttcggggctagaaaacgcagacgcctctgacttctcggtcctgggagaagccgcagcaggtagtgtccaatgggctggagtgaaggtagcagaaaggggacaaagcgtgaaaatggtcacaactgaggagcagccaaggggaaaatggcaagcactcaggatttag

SEQ ID NO: 61 - DNA, 912 bp, Pea mosaic virus
  source 1..912: mol_type = other DNA
atgcacggaattgagcagcctcaactaccgctagattacgttcaccgttgcgcatcaacctccttcttgctcgcatcactagatggcctcctttctgaagcccgtgaactctcagggcctctggctctcattacttctagctattacttacttgtttctattgccctctgctgggcaatccctggatccttctggtataggcctggctgctggttgcagccagtctcagggcggaatctcatcttttgcggccctaccgaggccttgcaacgattccgtctgtacgctgccagacttgggttggtcctgtcagagaactgcccaagacacggccaatcagcagcaatcacccttcaatcatactgggcacttcctaacaacatctggatggacatggcccaattggacttgctcaccttctcaatgccaattgctaatacatttgcctacttggcagattgtgaagcaagatttcctcctattgttgaaggagtgggatctgcttactatgtgccaacgctgctcggacttactcaccaagaccccaggctttatcttgcgcttcgcaggagaaaccttgatcttagtggcgaacctcatagagttcgtcctggtgtcctggagtctatggctttgctctgttctagtgtacgtagcacaagccgttccaggcaaattcctcctttatatggcagcgttttgcaccacgttttgggcctggccgagagagactgcatcctctttgatacggatagtaactactcctcttacactcatcgggttcttgaacaagaccggaatcgggctgatcagtcattgtttagcattgacttggaatatgttcatgacctggagcttattgccctgggttactctgatgaagatgatgaagatcttgataacttcttctag

SEQ ID NO: 62 - DNA, 771 bp, Cereal yellow dwarf virus
  source 1..771: mol_type = other DNA
atgttcatcgcccaaccttgcgggcgagttcttgtgttcgacgtcgcctcccgcacgccatcgttcttcactcgttatagtgttgaactctcgctccgtgttctagacccattcttcacgcgagcagtaacagatttccgatacacccaaaatgaaatcgatttattttgtgtgtctcttggctttctgttgccaattctcctcacaggagaatcttactcttggcgcggtcacctcaacctccccctttcttacaccgaattacttgttcgatgggggctcgcagtggggtacttccctaccttctccactgatggtgacattcgacagaacccagaactccgcatcgacctgtccaccatgtcaacccgctctttctacgagcagttcctactcagatataacacgagtgggttggcaaaagctatcgtcggacagcaagagtgctttcaaagcggcatggagtcttttaaaagattcctacactaccgcctcacgtgctttgaaagctgccttccacgacctcgttgggaaagtcctttggctcctggtccttatctggacagggcttttgaggcaactcttctcggccgtatggtcggtcataaccaactactctttaccggtttgtcttctgatatcactaggtattataacgagttggttgtggaaggcgtgccggtggctttttgggacgctgccggcattactttgcatcacgctggtgaagaatatttttcgaattcttacattcaaaagattcttcaatga

SEQ ID NO: 63 - protein, 389 aa, Arabidopsis thaliana
MVIAAAVIVPLGLLFFISGLAVNLFQAVCYVLIRPLSKNTYRKINRVVAETLWLELVWIVDWWAGVKIQVFADNETFNRMGKEHALVVCNHRSDIDWLVGWILAQRSGCLGSALAVMKKSSKFLPVIGWSMWFSEYLFLERNWAKDESTLKSGLQRLSDFPRPFWLALFVEGTRFTEAKLKAAQEYAASSELPIPRNVLIPRTKGFVSAVSNMRSFVPAIYDMTVTIPKTSPPPTMLRLFKGQPSVVHVHIKCHSMKDLPESDDAIAQWCRDQFVAKDALLDKHIAADTFPGQQEQNIGRPIKSLAVVLSWACVLTLGAIKFLHWAQLFSSWKGITISALGLGIITLCMQILIRSSQSERSTPAKVVPAKPKDNHHPESSSQTETEKEK

SEQ ID NO: 64 - protein, 281 aa, Limnanthes alba
MAKTRTSSLRNRRQLKTAVAATADDDKDGIFMVLLSCFKIFVCFAIVLITAVAWGLIMVLLLPWPYMRIRLGNLYGHIIGGLVIWLYGIPIEIQGSEHTKKRAIYISNHASPIDAFFVMWLAPIGTVGVAKKEVIWYPLLGQLYTLAHHIRIDRSNPAAAIQSMKEAVRVITEKNLSLIMFPEGTRSGDGRLLPFKKGFVHLALQSHLPIVPMILTGTHLAWRKGTFRVRPVPITVKYLPPINTDDWTVDKIDDYVKMIHDIYVRNLPASQKPLGSTNRSK

SEQ ID NO: 65 - protein, 303 aa, Saccharomyces cerevisiae
MSVIGRFLYYLRSVLVVLALAGCGFYGVIASILCTLIGKQHLAQWITARCFYHVMKLMLGLDVKVVGEENLAKKPYIMIANHQSTLDIFMLGRIFPPGCTVTAKKSLKYVPFLGWFMALSGTYFLDRSKRQEAIDTLNKGLENVKKNKRALWVFPEGTRSYTSELTMLPFKKGAFHLAQQGKIPIVPVVVSNTSTLVSPKYGVFNRGCMIVRILKPISTENLTKDKIGEFAEKVRDQMVDTLKEIGYSPAINDTTLPPQAIEYAALQHDKKVNKKIKNEPVPSVSISNDVNTHNEGSSVKKMH

SEQ ID NO: 66 - protein, 373 aa, Micromonas pusilla
MTPYQWFNVVSSLGYVLFTATTSTVTMLVPAIILLRPVSANLYARCTSWIFACWWTSCLFITERLNGVKVRVTGDALPLNAPLLIMSNHKCNLDWMFLWSSAIRTGSMFHVGVFKAVAKSEIRVIPIFGWGCKLNGFAYVRRRWSSDASHLTSWIQSQIRRRLNANWTLIFPEGTRYTDRNKERSDLSCAKDGLEPMAGEILRPRTKGLALLLRESAKGGGYYRKIVDMTIQYTDADGKPLKGAALGTRCFGQLAKGQLPVATCHVHFDVFSHKDVPAGEDEDEVEAWVWKRWRKKANMLEACASAGQFEGVREWSTSGTAVPLKTQTALRCFFVLQGLVCVGVACSSTAFLAYVACAAVGAAVIAQTDPAWW

SEQ ID NO: 67 - protein, 314 aa, Mortierella alpina
MSIGSSNPVLLAAIPFVYLFVLPRVLAFLPQKAQFLAKCIVVLIATLIMSVAGCFISIVCALLDKRYVINYVVSRLFSFLAARPCGVTYKIVGEEHLDKYPAIVVCNHQSSMDMMVLGRVFPKHCVVMAKKELLYFPFLGMFMKLSNAIFIDRKNHKKAIESTTQAVADMKKHNSGIWIFPEGTRSRLDKADLLPFKKGAFHLAIQAQLPILPIISQGYSHIYDSSKRYFPGGELEIRVLEPIPTTGLTTDDVNDLMDKTRNLMLKHLKEMDSQYSSSTAENGSTHIDADIAKSTATSIGNTDDAITKRRTPKE

SEQ ID NO: 68 - protein, 391 aa, Braccisa napus
MAMAAAAVIVPLGILFFISGLVVNLLQAVCYVLIRPLSKNTYRKINRVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSQLPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQKEHNIGRPIKSLAVVVSWACLLTLGAMKFLHWSNLFSSLKGIALSALGLGIITLCMQILIRSSQSERSTPAKVAPAKPKDKHQSGSSSQTEVEEKQK

SEQ ID NO: 69 - protein, 390 aa, Braccisa napus
MAMAAAVIVPLGILFFISGLVVNLLQAICYVLIRPLSKNTYRKINRVVAETLWLELVWIVDWWAGVKIQVFADNETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTEVEKQK

SEQ ID NO: 70 - protein, 361 aa, Phytophthora infestans
MATKEAYVFPTLTEIKRSLPKDCFEASVPLSLYYTVRCLVIAVALTFGLNYARALPEVESFWALDAALCTGYILLQGIVFWGFFTVGHDAGHGAFSRYHLLNFVVGTFMHSLILTPFESWKLTHRHHHKNTGNIDRDEVFYPQRKADDHPLSRNLILALGAAWLAYLVEGFPPRKVNHFNPFEPLFVRQVSAVVISLLAHFFVAGLSIYLSLQLGLKTMAIYYYGPVFVFGSMLVITTFLHHNDEETPWYADSEWTYVKGNLSSVDRSYGALIDNLSHNIGTHQIHHLFPIIPHYKLKKATAAFHQAFPELVRKSDEPIIKAFFRVGRLYANYGVVDQEAKLFTLKEAKAATEAAAKTKST

SEQ ID NO: 71 - protein, 418 aa, Thalassiosira pseudonana
MYRLTSTFLIALAFSSSINAFSPQRPPRTITKSKVQSTVLPIPTKDDLNFLQPQLDENDLYLDDVNTPPRAGTIMKMLPKETFNIDTATSLGYFGMDMAAVVSSMTLLNAIVTSDQYHALPLPLQAATVIPFQLLAGFAMWCMWCIGHDAGHSTVSKTKWINRVVGEVAHSVVCLTPFVPWQMSHRKHHLNHNHIEKDYSHKWYSRDEFDDIPQLYKTFGYNPRMMQLPFLYFMYLALGIPDGGHVVFYGRMWEGVSLQKKFDAAISVAVSCATAGSLWMNMGTADFTVVCMVPWLVLSWWLFMVTYLQHHSEDGKLYTDETFTFEKGAFETVDRSYGKLINRMSHHMMDGHVVHHLFFERVPHYRLEAATEALVKGMDETGQKHLYKYIDTPDFNAEIVNGFRDNWFLVEEENIKRE

SEQ ID NO: 72 - protein, 363 aa, Pythium irregulare
MASTSAAQDAAPYEFPSLTEIKRALPSECFEASVPLSLYYTARSLALAGSLAVALSYARALPLVQANALLDATLCTGYVLLQGIVFWGFFTVGHDCGHGAFSRSHVLNFSVGTLMHSIILTPFESWKLSHRHHHKNTGNIDKDEIFYPQREADSHPVSRHLVMSLGSAWFAYLFAGFPPRTMNHFNPWEAMYVRRVAAVIISLGVLFAFAGLYSYLTFVLGFTTMAIYYFGPLFIFATMLVVTTFLHHNDEETPWYADSEWTYVKGNLSSVDRSYGALIDNLSHNIGTHQIHHLFPIIPHYKLNDATAAFAKAFPELVRKNAAPIIPTFFRMAAMYAKYGVVDTDAKTFTLKEAKAAAKTKSS

(19) United States
(12) Patent Application Publication
(10) Pub. No.: US 2024/0084214 A1
(43) Pub. Date: Mar. 14, 2024

(54) LIPID COMPRISING POLYUNSATURATED FATTY ACIDS

(71) Applicants: Commonwealth Scientific and Industrial Research Organisation, Acton (AU); Grains Research and Development Corporation, Barton (AU); NUSEED NUTRITIONAL AUSTRALIA PTY LTD., Laverton North (AU)

(72) Inventors: James Robertson Petrie, Goulburn (AU); Surinder Pal Singh, Downer (AU); Robert Charles de Feyter, Monash (AU)

(73) Assignees: Commonwealth Scientific and Industrial Research Organisation, Acton (AU); Grains Research and Development Corporation, Barton (AU); NUSEED NUTRITIONAL AUSTRALIA PTY LTD., Laverton North (AU)

(21) Appl. No.: 18/503,775

(22) Filed: Nov. 7, 2023

Related U.S. Application Data
(63) Continuation of application No. 17/150,898, filed on Jan. 15, 2021, now Pat. No. 11,834,621.
Application No. 17/150,898 is a continuation of application No. 15/975,497, filed on May 9, 2018, now Pat. No. 10,899,992.
Application No. 15/975,497 is a continuation of application No. 15/373,145, filed on Dec. 8, 2016, now Pat. No. 10,335,386.
Application No. 15/373,145 is a continuation of application No. 14/464,510, filed on Aug. 20, 2014, now Pat. No. 9,550,718.
Application No. 14/464,510 is a continuation of application No. 13/918,399, filed on Jun. 14, 2013, now Pat. No. 8,816,111.
(60) Provisional application No. 61/782,680, filed on Mar. 14, 2013.
(60) Provisional application No. 61/697,676, filed on Sep. 6, 2012.
(60) Provisional application No. 61/663,344, filed on Jun. 22, 2012.
(60) Provisional application No. 61/660,392, filed on Jun. 15, 2012.

(51) Int. Cl. C11B 1/10; A01H 1/00; A01H 5/10; A01H 6/20; A61K 36/31; C07C 57/02; C07C 57/03; C07C 67/02; C11B 1/00; C11C 1/00; C11C 3/00; C11C 3/06; C12N 9/10; C12N 15/82

(52) U.S. Cl. CPC C11B 1/10; A01H 1/104; A01H 5/10; A01H 6/20; A61K 36/31; C07C 57/02; C07C 57/03; C07C 67/02; C11B 1/00; C11C 1/002; C11C 3/00; C11C 3/003; C11C 3/06; C12N 9/1025; C12N 15/8247; A23L 33/12

(57) ABSTRACT

The present invention relates to extracted plant lipid, comprising fatty acids in an esterified form.

### CROSS-REFERENCE TO RELATED APPLICATIONS

This application claims benefit of U.S. Provisional Patent Application No. 61/782,680, filed Mar. 14, 2013, U.S. Provisional Patent Application No. 61/697,676, filed Sep. 6, 2012, U.S. Provisional Patent Application No. 61/663,344, filed Jun. 22, 2012, and U.S. Provisional Patent Application No. 61/660,392, filed Jun. 15, 2012, the entire contents of each of which are hereby incorporated by reference into the subject application.

### REFERENCE TO SEQUENCE LISTING

This application incorporates-by-reference nucleotide and/or amino acid sequences which are present in the file named “130614_2251_84199_A_Sequence_Listing_REB.txt,” which is 369 kilobytes in size, and which was created Jun. 14, 2013 in the IBM-PC machine format, having an operating system compatibility with MS-Windows, which is contained in the text file filed Jun. 14, 2013 as part of this application.

### FIELD OF THE INVENTION

The present invention relates to extracted plant lipid, comprising fatty acids in an esterified form.

### BACKGROUND OF THE INVENTION

Omega-3 long-chain polyunsaturated fatty acids (LC-PUFA) are now widely recognized as important compounds for human and animal health. These fatty acids may be obtained from dietary sources or by conversion of linoleic (LA, 18:2ω6) or α-linolenic (ALA, 18:3ω3) fatty acids, both of which are regarded as essential fatty acids in the human diet. While humans and many other vertebrate animals are able to convert LA or ALA, obtained from plant sources to C22 they carry out this conversion at a very low rate. Moreover, most modern societies have imbalanced diets in which at least 90% of polyunsaturated fatty acids (PUFA) are of the ω6 fatty acids, instead of the 4:1 ratio or less for ω6:ω3 fatty acids that is regarded as ideal (Trautwein, 2001). The immediate dietary source of LC-PUFAs such as eicosapentaenoic acid (EPA, 20:5ω3) and docosahexaenoic acid (DHA, 22:6ω3) for humans is mostly from fish or fish oil. Health professionals have therefore recommended the regular inclusion of fish containing significant levels of LC-PUFA into the human diet. Increasingly, fish-derived LC-PUFA oils are being incorporated into food products and in infant formula, for example. However, due to a decline in global and national fisheries, alternative sources of these beneficial health-enhancing oils are needed.

Flowering plants, in contrast to animals, lack the capacity to synthesise polyunsaturated fatty acids with chain lengths longer than 18 carbons. In particular, crop and horticultural plants along with other angiosperms do not have the enzymes needed to synthesize the longer chain ω3 fatty acids such as EPA, docosapentaenoic acid (DPA, 22:5ω3) and DHA that are derived from ALA. An important goal in plant biotechnology is therefore the engineering of crop plants which produce substantial quantities of LC-PUFA, thus providing an alternative source of these compounds.

### LC-PUFA Biosynthesis Pathways

Biosynthesis of LC-PUFAs in organisms such as microalgae, mosses and fungi usually occurs as a series of oxygen-dependent desaturation and elongation reactions (FIG. 1). The most common pathway that produces EPA in these organisms includes a Δ6-desaturation, Δ6-elongation and Δ5-desaturation (termed the Δ6-desaturation pathway) whilst a less common pathway uses a Δ9-elongation, Δ8-desaturation and Δ5-desaturation (termed the Δ9-desaturation pathway). These consecutive desaturation and elongation reactions can begin with either the ω6 fatty acid substrate LA, shown schematically as the upper left part of FIG. 1 (ω6) or the ω3 substrate ALA through to EPA, shown as the lower right part of FIG. 1 (ω3). If the initial Δ6-desaturation is performed on the ω6 substrate LA, the LC-PUFA product of the series of three enzymes will be the ω6 fatty acid ARA. LC-PUFA synthesising organisms may convert ω6 fatty acids to ω3 fatty acids using an ω3-desaturase, shown as the Δ17-desaturase step in FIG. 1 for conversion of arachidonic acid (ARA, 20:4ω6) to EPA. Some members of the ω3-desaturase family can act on a variety of substrates ranging from LA to ARA. Plant ω3-desaturases often specifically catalyse the Δ15-desaturation of LA to ALA, while fungal and yeast ω3-desaturases may be specific for the Δ17-desaturation of ARA to EPA (Pereira et al., 2004a; Zank et al., 2005). Some reports suggest that non-specific ω3-desaturases may exist which can convert a wide variety of ω6 substrates to their corresponding ω3 products (Zhang et al., 2008).

The conversion of EPA to DHA in these organisms occurs by a Δ5-elongation of EPA to produce DPA, followed by a Δ4-desaturation to produce DHA (FIG. 1). In contrast, mammals use the so-called “Sprecher” pathway which converts DPA to DHA by three separate reactions that are independent of a Δ4-desaturase (Sprecher et al., 1995).

The front-end desaturases generally found in plants, mosses, microalgae, and lower animals such as Caenorhabditis elegans predominantly accept fatty acid substrates esterified to the sn-2 position of a phosphatidylcholine (PC) substrate. These desaturases are therefore known as acyl-PC, lipid-linked, front-end desaturases (Domergue et al., 2003). In contrast, higher animal front-end desaturases generally accept acyl-CoA substrates where the fatty acid substrate is linked to CoA rather than PC (Domergue et al., 2005). Some microalgal desaturases and one plant desaturase are known to use fatty acid substrates esterified to CoA (Table 2).

Each PUFA elongation reaction consists of four steps catalysed by a multi-component protein complex: first, a condensation reaction results in the addition of a 2C unit from malonyl-CoA to the fatty acid, resulting in the formation of a β-ketoacyl intermediate. This is then reduced by NADPH, followed by a dehydration to yield an enoyl intermediate. This intermediate is finally reduced a second time to produce the elongated fatty acid. It is generally thought that the condensation step of these four reactions is substrate specific whilst the other steps are not. In practice, this means that native plant elongation machinery is capable of elongating PUFA providing that the condensation enzyme (typically called an ‘elongase’) specific to the PUFA is introduced, although the efficiency of the native plant elongation machinery in elongating the non-native PUFA substrates may be low. In 2007 the identification and characterisation of the yeast elongation cycle dehydratase was published (Denic and Weissman, 2007).

PUFA desaturation in plants, mosses and microalgae naturally occurs to fatty acid substrates predominantly in the acyl-PC pool whilst elongation occurs to substrates in the acyl-CoA pool. Transfer of fatty acids from acyl-PC molecules to a CoA carrier is performed by phospholipases (PLAs) whilst the transfer of acyl-CoA fatty acids to a PC carrier is performed by lysophosphatidyl-choline acyltransferases (LPCATs) (FIG. 21) (Singh et al., 2005).

### Engineered Production of LC-PUFA

Most LC-PUFA metabolic engineering has been performed using the aerobic Δ6-desaturation/elongation pathway. The biosynthesis of γ-linolenic acid (GLA, 18:3ω6) in tobacco was first reported in 1996 using a Δ6-desaturase from the cyanobacterium Synechocystis (Reddy and Thomas, 1996). More recently, GLA has been produced in crop plants such as safflower (73% GLA in seedoil; Knauf et al., 2006) and soybean (28% GLA; Sato et al., 2004). The production of LC-PUFA such as EPA and DHA involves more complicated engineering due to the increased number of desaturation and elongation steps involved. EPA production in a land plant was first reported by Qi et al. (2004) who introduced genes encoding a Δ9-elongase from Isochrysis galbana, a Δ8-desaturase from Euglena gracilis and a Δ5-desaturase from Mortierella alpina into Arabidopsis yielding up to 3% EPA. This work was followed by Abbadi et al. (2004) who reported the production of up to 0.8% EPA in flax seed using genes encoding a Δ6-desaturase and Δ6-elongase from Physcomitrella patens and a Δ5-desaturase from Phaeodactylum tricornutum.

The first report of DHA production, and to date the highest levels of VLC-PUFA production reported, was in WO 04/017467 where the production of 3% DHA in soybean embryos is described, but not seed, by introducing genes encoding the Saprolegnia diclina Δ6-desaturase, Mortierella alpina Δ6-desaturase, Mortierella alpina Δ5-desaturase, Saprolegnia diclina Δ4-desaturase, Saprolegnia diclina Δ17-desaturase, Mortierella alpina Δ6-elongase and Pavlova lutheri Δ5-elongase. The maximal EPA level in embryos also producing DHA was 19.6%, indicating that the efficiency of conversion of EPA to DHA was poor (WO 2004/071467). This finding was similar to that published by Robert et al. (2005), where the flux from EPA to DHA was low, with the production of 3% EPA and 0.5% DHA in Arabidopsis using the Danio rerio Δ5/6-desaturase, the Caenorhabditis elegans Δ6-elongase, and the Pavlova salina Δ5-elongase and Δ4-desaturase. Also in 2005, Wu et al. published the production of 25% ARA, 15% EPA, and 1.5% DHA in Brassica juncea using the Pythium irregulare Δ6-desaturase, a Thraustochytrid Δ5-desaturase, the Physcomitrella patens Δ6-elongase, the Calendula officianalis Δ12-desaturase, a Thraustochytrid Δ5-elongase, the Phytophthora infestans Δ17-desaturase, the Oncorhyncus mykiss LC-PUFA elongase, a Thraustochytrid Δ4-desaturase and a Thraustochytrid LPCAT (Wu et al., 2005). Summaries of efforts to produce oil-seed crops which synthesize ω3 LC-PUFAs is provided in Venegas-Caleron et al. (2010) and Ruiz-Lopez et al. (2012). As indicated by Ruiz-Lopez et al. (2012), results obtained to date for the production of DHA in transgenic plants has been no where near the levels seen in fish oils.

There therefore remains a need for more efficient production of LC-PUFA in recombinant cells, in particular of DHA in seeds of oilseed plants.

### SUMMARY OF THE INVENTION

The present inventors have identified methods and plants for producing lipid with high levels of DHA.

In a first aspect, the present invention provides extracted plant lipid, comprising fatty acids in an esterified form, the fatty acids comprising oleic acid, palmitic acid, ω6 fatty acids which comprise linoleic acid (LA), ω3 fatty acids which comprise α-linolenic acid (ALA), and docosahexaenoic acid (DHA), and optionally one or more of stearidonic acid (SDA), eicosapentaenoic acid (EPA), docosapentaenoic acid (DPA) and eicosatetraenoic acid (ETA), wherein the level of DHA in the total fatty acid content of the extracted lipid is about 7% to 20%.

In an embodiment, the extracted lipid has one or more or all of the following features
i) the level of palmitic acid in the total fatty acid content of the extracted lipid is between about 2% and 18%, between about 2% and 16%, or between about 2% and 15%,
ii) the level of myristic acid (C14:0) in the total fatty acid content of the extracted lipid is less than about 6%, less than about 3%, less than about 2%, or less than about 1%,
iii) the level of oleic acid in the total fatty acid content of the extracted lipid is between about 1% and about 30%, between about 3% and about 30%, between about 6% and about 30%, between 1% and about 20%, between about 30% and about 60%, between about 45% to about 60%, or is about 30%,
iv) the level of linoleic acid (LA) in the total fatty acid content of the extracted lipid is between about 4% and about 35%, between about 4% and about 20%, or between about 4% and 17%,
v) the level of α-linolenic acid (ALA) in the total fatty acid content of the extracted lipid is between about 4% and about 40%, between about 7% and about 40%, between about 10% and about 35%, between about 20% and about 35%, between about 4% and about 16%, or between about 2% and about 16%,
vi) the level of γ-linolenic acid (GLA) in the total fatty acid content of the extracted lipid is less than about 4%, less than about 3%, less than about 2%, less than about 1%, less than about 0.5%, between 0.05% and about 7%, between 0.05% and about 4%, between 0.05% and about 3%, or between 0.05% and about 2%,
vii) the level of stearidonic acid (SDA) in the total fatty acid content of the extracted lipid is less than about 7%, less than about 6%, less than about 4%, less than about 3%, between about 0.05% and about 7%, between about 0.05% and about 6%, between about 0.05% and about 4%, between about 0.05% and about 3%, or between 0.05% and about 2%.
viii) the level of eicosatetraenoic acid (ETA) in the total fatty acid content of the extracted lipid is less than about 6%, less than about 5%, less than about 4%, less than about 1%, less than about 0.5%, between about 0.05% and about 6%, between about 0.05% and about 5%, between about 0.05% and about 4%, between about 0.05% and about 3%, or between about 0.05% and about 2%,
ix) the level of eicosatrienoic acid (ETrA) in the total fatty acid content of the extracted lipid is less than about 4%, less than about 2%, less than about 1%, between about 0.05% and about 4%, between about 0.05% and about 3%, between about 0.05% and about 2%, or between about 0.05% and about 1%,
x) the level of eicosapentaenoic acid (EPA) in the total fatty acid content of the extracted lipid is less than about 4%, less than about 3%, less than about 2%, between about 0.05% and about 10%, between about 0.05% and about 5%, between about 0.05% and about 3%, or between about 0.05% and about 2%,
xi) the level of docosapentaenoic acid (DPA) in the total fatty acid content of the extracted lipid is less than about 4%, less than about 3%, less than about 2%, between about 0.05% and about 8%, between about 0.05% and about 5%, between about 0.05% and about 3%, or between about 0.05% and about 2%,
xii) the level of DHA in the total fatty acid content of the extracted lipid is about 8%, about 9%, about 10%, about 11%, about 12%, about 13%, about 14%, about 15%, about 16%, about 17%, about 18%, between about 8% and 20%, between about 10% and 20%, between about 11% and 20%, between about 10% and about 16%, or between about 14% and 20%,
xiii) the lipid comprises ω6-docosapentaenoic acid (22:5^{Δ4,7,10,13,16}) in its fatty acid content,
xiv) the lipid is essentially free of ω6-docosapentaenoic acid (22:5^{Δ4,7,10,13,16}) in its fatty acid content,
xv) the lipid is essentially free of SDA, EPA and ETA in its fatty acid content,
xvi) the level of total saturated fatty acids in the total fatty acid content of the extracted lipid is between about 4% and about 25%, between about 4% and about 20%, between about 6% and about 20%, between about 4% and about 60%, between about 30% and about 60%, or between about 45% and about 60%.
xvii) the level of total monounsaturated fatty acids in the total fatty acid content of the extracted lipid is between about 4% and about 35%, between about 8% and about 25%, or between 8% and about 22%.
xviii) the level of total polyunsaturated fatty acids in the total fatty acid content of the extracted lipid is between about 20% and about 75%, between about 50% and about 75%, or between about 60% and about 75%,
xix) the level of total (ω6 fatty acids in the total fatty acid content of the extracted lipid is between about 35% and about 50%, between about 20% and about 35%, between about 6% and 20%, less than about 20%, less than about 16%, less than about 10%, between about 1% and about 16%, between about 2% and about 10%, or between about 4% and about 10%,
xx) the level of new ω6 fatty acids in the total fatty acid content of the extracted lipid is less than about 10%, less than about 8%, less than about 6%, less than 4%, between about 1% and about 20%, between about 1% and about 10%, between about 0.5% and about 8%, or between about 0.5% and 4%,
xxi) the level of total ω3 fatty acids in the total fatty acid content of the extracted lipid is between 36% and about 65%, between about 40% and about 60%, between about 20% and about 35%, between about 10% and about 20%, about 25%, about 30%, about 35% or about 40%,
xxii) the level of new ω3 fatty acids in the total fatty acid content of the extracted lipid is between about 9% and about 33%, between about 10% and about 20%, between about 20% and about 30%, between about 12% and about 25%, about 13%, about 15%, about 17% or about 20%,
xxiii) the ratio of total ω6 fatty acids: total ω3 fatty acids in the fatty acid content of the extracted lipid is between about 1.0 and about 3.0, between about 0.1 and about 1, between about 0.1 and about 0.5, less than about 0.50, less than about 0.40, less than about 0.30, less than about 0.20, less than about 0.15, about 1.0, about 0.1 or about 0.2,
xxiv) the ratio of new ω6 fatty acids: new ω3 fatty acids in the fatty acid content of the extracted lipid is between about 1.0 and about 3.0, between about 0.1 and about 1, between about 0.1 and about 0.5, less than about 0.50, less than about 0.40, less than about 0.30, less than about 0.20, less than about 0.15, about 0.1, about 0.2 or about 1.0,
xxv) the fatty acid composition of the lipid is based on an efficiency of conversion of oleic acid to LA by Δ12-desaturase of at least about 60%, at least about 70%, at least about 80%, between about 60% and about 98%, between about 70% and about 95%, or between about 75% and about 90%,
xxvi) the fatty acid composition of the lipid is based on an efficiency of conversion of ALA to SDA by Δ6-desaturase of at least about 30%, at least about 40%, at least about 50%, at least about 60%, at least about 70%, between about 30% and about 70%, between about 35% and about 60%, or between about 50% and about 70%,
xxvii) the fatty acid composition of the lipid is based on an efficiency of conversion of SDA to ETA acid by Δ6-elongase of at least about 60%, at least about 70%, at least about 75%, between about 60% and about 95%, between about 70% and about 88%, or between about 75% and about 85%,
xxviii) the fatty acid composition of the lipid is based on an efficiency of conversion of ETA to EPA by Δ5-desaturase of at least about 60%, at least about 70%, at least about 75%, between about 60% and about 99%, between about 70% and about 99%, or between about 75% and about 98%,
xxix) the fatty acid composition of the lipid is based on an efficiency of conversion of EPA to DPA by Δ5-elongase of at least about 80%, at least about 85%, at least about 90%, between about 50% and about 95%, or between about 85% and about 95%.
xxx) the fatty acid composition of the lipid is based on an efficiency of conversion of DPA to DHA by Δ4-desaturase of at least about 80%, at least about 90%, at least about 93%, between about 50% and about 95%, between about 80% and about 95%, or between about 85% and about 95%,
xxxi) the fatty acid composition of the lipid is based on an efficiency of conversion of oleic acid to DHA of at least about 10%, at least about 15%, at least about 20%, between about 10% and about 50%, between about 10% and about 30%, or between about 10% and about 25%,
xxxii) the fatty acid composition of the lipid is based on an efficiency of conversion of LA to DHA of at least about 15%, at least about 20%, at least about 22%, at least about 25%, between about 15% and about 50%, between about 20% and about 40%, or between about 20% and about 30%,
xxxiii) the fatty acid composition of the lipid is based on an efficiency of conversion of ALA to DHA of at least about 17%, at least about 22%, at least about 24%, between about 17% and about 55%, between about 22% and about 35%, or between about 24% and about 35%,
xxxiv) the total fatty acid in the extracted lipid has less than 1% C20:1, xxxv) the triacylglycerol (TAG) content of the lipid is at least about 70%, at least about 80%, at least about 90%, at least 95%, between about 70% and about 99%, or between about 90° % and about 99%,
xxxvi) the lipid comprises diacylglycerol (DAG),
xxxvii) the lipid comprises less than about 10%, less than about 5%, less than about 1%, or between about 0.001% and about 5%, free (non-esterified) fatty acids and/or phospholipid, or is essentially free thereof.
xxxviii) at least 70%, or at least 80%, of the DHA esterified in the form of TAG is in the sn-1 or sn-3 position of the TAG,
xxxix) the most abundant DHA-containing TAG species in the lipid is DHA/18:3/18:3 (TAG 58:12), and
xl) the lipid comprises tri-DHA TAG (TAG 66:18).

In another embodiment, the extracted lipid is in the form of an oil, wherein at least about 90%, or least about 95%, at least about 98%, or between about 95% and about 98%, by weight of the oil is the lipid.

In a preferred embodiment, the lipid or oil, preferably a seedoil, has the following features: in the total fatty acid content of the lipid or oil, the level of DHA is between about 7% and 20%, the level of palmitic acid is between about 2% and about 16%, the level of myristic acid is less than about 6%, the level of oleic acid is between about 1% and about 30%, the level of LA is between about 4% and about 35%, ALA is present. GLA is present, the level of SDA is between about 0.05% and about 7%, the level of ETA is less than about 4%, the level of EPA is between about 0.05% and about 10%, the level of DPA is between about 0.05% and about 8%, the level of total saturated fatty acids in the total fatty acid content of the extracted lipid is between about 4% and about 25%, the level of total monounsaturated fatty acids in the total fatty acid content of the extracted lipid is between about 4% and about 35%, the level of total polyunsaturated fatty acids in the total fatty acid content of the extracted lipid is between about 20% and about 75%, the ratio of total ω6 fatty acids: total ω3 fatty acids in the fatty acid content of the extracted lipid is between about 0.05 and about 3.0, the ratio of new ω6 fatty acids: new ω3 fatty acids in the fatty acid content of the extracted lipid is between about 0.03 and about 3.0, preferably less than about 0.50, the fatty acid composition of the lipid is based on: an efficiency of conversion of oleic acid to LA by Δ12-desaturase of at least about 60%, an efficiency of conversion of SDA to ETA acid by Δ6-elongase of at least about 60%, an efficiency of conversion of EPA to DPA by Δ5-elongase of between about 50% and about 95%, an efficiency of conversion of DPA to DHA by Δ4-desaturase of between about 50% and about 95%, an efficiency of conversion of oleic acid to DHA of at least about 10%, and the triacylglycerol (TAG) content of the lipid is at least about 70%, and optionally the lipid is essentially free of cholesterol and/or the lipid comprises tri-DHA TAG (TAG 66:18).

In a more preferred embodiment, the lipid or oil, preferably a seedoil, has the following features: in the total fatty acid content of the lipid, the level of DHA is between about 7% and 20%, the level of palmitic acid is between about 2% and about 16%, the level of myristic acid is less than about 2%, the level of oleic acid is between about 1% and about 30%, the level of LA is between about 4% and about 35%, the level of ALA is between about 7% and about 40%, the level of GLA is less than about 4%, the level of SDA is between about 0.05% and about 7%, the level of ETA is less than about 4%, the level of ETrA is between about 0.05% and about 4%, the level of EPA is between about 0.05% and about 10%, the level of DPA is between about 0.05% and about 8%, the level of total saturated fatty acids in the total fatty acid content of the extracted lipid is between about 4% and about 25%, the level of total monounsaturated fatty acids in the total fatty acid content of the extracted lipid is between about 4% and about 35%, the level of total polyunsaturated fatty acids in the total fatty acid content of the extracted lipid is between about 20% and about 75%, the level of new ω6 fatty acids in the total fatty acid content of the extracted lipid is between about 0.5% and about 10%, the level of total ω3 fatty acids in the total fatty acid content of the extracted lipid is between 36% and about 75%, the level of new ω3 fatty acids in the total fatty acid content of the extracted lipid is between about 9% and about 33%, the ratio of total ω6 fatty acids: total ω3 fatty acids in the fatty acid content of the extracted lipid is between about 0.05 and about 3.0, the ratio of new ω6 fatty acids: new ω3 fatty acids in the fatty acid content of the extracted lipid is between about 0.03 and about 3.0, the fatty acid composition of the lipid is based on: an efficiency of conversion of oleic acid to LA by Δ12-desaturase of at least about 6(0%, an efficiency of conversion of SDA to ETA acid by Δ6-elongase of at least about 60%, an efficiency of conversion of ETA to EPA by Δ5-desaturase of at least about 60%, an efficiency of conversion of EPA to DPA by Δ5-elongase of between about 50% and about 95%, an efficiency of conversion of DPA to DHA by Δ4-desaturase of between about 50% and about 95%, an efficiency of conversion of oleic acid to DHA of at least about 10%, an efficiency of conversion of LA to DHA of at least about 15%, an efficiency of conversion of ALA to DHA of at least about 17%, and the total fatty acid content in the extracted lipid has less than 1% C20:1, the triacylglycerol (TAG) content of the lipid is at least about 70%, the lipid is essentially free of cholesterol, and the lipid comprises tri-DHA TAG (TAG 66:18). Preferably, the lipid or oil is canola oil and/or has not been treated with a transesterification process after it was extracted from the plant or plant part. In a particular embodiment, the lipid or canola oil may subsequently be treated to convert the fatty acids in the oil to alkyl esters such as methyl or ethyl esters. Further treatment may be applied to enrich the lipid or oil for the DHA.

In an embodiment, the lipid or oil, preferably a seedoil, has the following features: in the total fatty acid content of the lipid, the level of DHA is between about 7% and 20%, the level of palmitic acid is between about 2% and about 16%, the level of myristic acid is less than about 2%, the level of oleic acid is between about 30% and about 6(0%, preferably between about 45% and about 60%, the level of LA is between about 4% and about 20%, the level of ALA is between about 2% and about 16%, the level of GLA is less than about 3%, the level of SDA is less than about 3%, the level of ETA is less than about 4%, the level of ETrA less than about 2%, the level of EPA is less than about 4%, the level of DPA is less than about 4%, the level of total saturated fatty acids in the total fatty acid content of the extracted lipid is between about 4% and about 25%, the level of total monounsaturated fatty acids in the total fatty acid content of the extracted lipid is between about 30% and about 60%, or between about 40% and about 60%, the level of total polyunsaturated fatty acids in the total fatty acid content of the extracted lipid is between about 20% and about 75%, the level of new ω6 fatty acids in the total fatty acid content of the extracted lipid is between about 0.5% and about 10%, the level of total ω3 fatty acids in the total fatty acid content of the extracted lipid is between about 10% and about 20%, the level of new ω3 fatty acids in the total fatty acid content of the extracted lipid is between about 9% and about 20%, the ratio of total ω6 fatty acids: total ω3 fatty acids in the fatty acid content of the extracted lipid is between about 0.05 and about 3.0, preferably less than about 0.50, the ratio of new ω6 fatty acids: new ω3 fatty acids in the fatty acid content of the extracted lipid is between about 0.03 and about 3.0, the triacylglycerol (TAG) content of the lipid is at least about 70%, the lipid is essentially free of cholesterol, and the lipid comprises tri-DHA TAG (TAG 66:18). Preferably, the lipid or oil is essentially free of SDA. EPA and ETA and/or is canola oil and/or has not been treated with a transesterification process after it was extracted from the plant or plant part. In a particular embodiment, the lipid or canola oil may subsequently be treated to convert the fatty acids in the oil to alkyl esters such as methyl or ethyl esters. Further treatment may be applied to enrich the lipid or oil for the DHA.

In a further preferred embodiment, the lipid or oil, preferably a seedoil, has the following features: in the total fatty acid content of the lipid or oil, the level of DHA is between about 7% and 20%, the level of palmitic acid is between about 2% and about 16%, the level of myristic acid is less than about 6%, the level of oleic acid is between about 1% and about 30%, the level of LA is between about 4% and about 35%. ALA is present, GLA is present, the level of SDA is between about 0.05% and about 7%, the level of ETA is less than about 6%, the level of EPA is between about 0.05% and about 10%, the level of DPA is between about 0.05% and about 8%.

In a further embodiment, the extracted lipid further comprises one or more sterols, preferably plant sterols.

In another embodiment, the extracted lipid is in the form of an oil, and comprises less than about 10 mg of sterols/g of oil, less than about 7 mg of sterols/g of oil, between about 1.5 mg and about 10 mg of sterols/g of oil, or between about 1.5 mg and about 7 mg of sterols/g of oil.

Examples of sterols which can be in the extracted lipid include, but are not necessarily limited to, one or more or all of campesterol/24-methylcholesterol. Δ5-stigmasterol, eburicol, p-sitosterol/24-ethylcholesterol, Δ5-avenasterol/isofucosterol, Δ7-stigmasterol/stigmast-7-en-3β-ol, and Δ7-avenasterol.

In an embodiment, the plant species is one listed in Table 26, such as canola, and the level of sterols are about the same as that listed in Table 26 for that particular plant species.

In an embodiment, the extracted lipid comprises less than about 0.5 mg of cholesterol/g of oil, less than about 0.25 mg of cholesterol/g of oil, between about 0 mg and about 0.5 mg of cholesterol/g of oil, or between about 0 mg and about 0.25 mg of cholesterol/g of oil, or which is essentially free of cholesterol.

In a further embodiment, the lipid is an oil, preferably oil from an oilseed. Examples of such oils include, but are not limited to, Brassica sp. oil such as canola oil. Gossypium hirsutum oil, Linum usitatissimum oil, Helianthus sp. oil, Carthamus tinctorius oil, Glycine max oil, Zea mays oil, Arabidopsis thaliana oil. Sorghum bicolor oil, Sorghum vulgare oil, Avena sativa oil, Trifolium sp. oil, Elaesis guineenis oil, Nicotiana benthamiana oil, Hordeum vulgare oil, Lupinus angustifolius oil, Oryza sativa oil, Oryza glaberrima oil, Camelina sativa oil, Crambe abyssinica oil, Miscanthus x giganteus oil, or Miscanthus sinensis oil.

Also provided is extracted plant lipid, preferably extracted canola seedoil, comprising fatty acids in an esterified form, the fatty acids comprising oleic acid, palmitic acid, ω6 fatty acids which comprise linoleic acid (LA), ω3 fatty acids which comprise α-linolenic acid (ALA), and docosahexaenoic acid (DHA), and optionally one or more of stearidonic acid (SDA), eicosapentaenoic acid (EPA), docosapentaenoic acid (DPA) and eicosatetraenoic acid (ETA), wherein lipid has the following features in the total fatty acid content of the lipid;
i) the level of DHA is about 3%, about 4%, about 5%, about 6% or about 7%,
ii) the level of palmitic acid is between about 2% and about 16%,
iii) the level of myristic acid is less than about 2%,
iv) the level of oleic acid is between about 30% and about 60%, preferably between about 45% and about 60%,
v) the level of LA is between about 4% and about 20%,
vi) the level of ALA is between about 2% and about 16%,
vii) the level of GLA is less than about 4%,
viii) the level of SDA is less than about 6%, or less than about 4%,
ix) the level of ETA is less than about 6%, or less than about 4%,
x) the level of ETrA less than about 1%,
xi) the level of EPA is less than about 10% and/or the level of EPA is 0.5-2.0 fold the level of DHA,
xii) the level of DPA is less than about 4%,
xiii) the level of total saturated fatty acids in the total fatty acid content of the extracted lipid is between about 4% and about 25%,
xiv) the level of total monounsaturated fatty acids in the total fatty acid content of the extracted lipid is between about 30% and about 70%,
xv) the level of total polyunsaturated fatty acids in the total fatty acid content of the extracted lipid is between about 15% and about 75%, preferably between about 15% and about 30%,
xvi) the level of new ω6 fatty acids in the total fatty acid content of the extracted lipid is between about 0.5% and about 10%,
xvii) the level of total ω3 fatty acids in the total fatty acid content of the extracted lipid is between about 10% and about 20%,
xviii) the level of new ω3 fatty acids in the total fatty acid content of the extracted lipid is between about 3% and about 20%,
xix) the ratio of total ω6 fatty acids: total ω3 fatty acids in the fatty acid content of the extracted lipid is between about 0.05 and about 3.0, preferably less than about 0.50,
xx) the ratio of new ω6 fatty acids: new (3 fatty acids in the fatty acid content of the extracted lipid is between about 0.03 and about 3.0,
xxi) the triacylglycerol (TAG) content of the lipid is at least about 70%, and
xxii) the lipid is essentially free of cholesterol. In an embodiment, the lipid comprises tri-DHA TAG (TAG 66:18). More preferably, the lipid is essentially free of SDA and ETA, and/or has not been treated with a transesterification process after it was extracted from the plant or plant part.

In another aspect, provided is extracted plant lipid, comprising fatty acids in an esterified form, the fatty acids comprising oleic acid, palmitic acid, ω6 fatty acids which comprise linoleic acid (LA), ω3 fatty acids which comprise α-linolenic acid (ALA) and docosahexaenoic acid (DHA), and one or more of stearidonic acid (SDA), eicosapentaenoic acid (EPA), docosapentaenoic acid (DPA) and eicosatetraenoic acid (ETA), wherein (i) the level of DHA in the total fatty acid content of the extracted lipid is between 7% and 20%, (ii) the level of palmitic acid in the total fatty acid content of the extracted lipid is between 2% and 16%, (iii) the level of myristic acid (C14:0) in the total fatty acid content of the extracted lipid is less than 6%, (iv) the level of oleic acid in the total fatty acid content of the extracted lipid is between 1% and 30% or between 30% and 60%, (v) the level of linoleic acid (LA) in the total fatty acid content of the extracted lipid is between 4% and 35%, (vi) the level of α-linolenic acid (ALA) in the total fatty acid content of the extracted lipid is between 4% and 40%. (vii) the level of eicosatrienoic acid (ETrA) in the total fatty acid content of the extracted lipid is less than 4%, (viii) the level of total saturated fatty acids in the total fatty acid content of the extracted lipid is between 4% and 25%, (ix) the ratio of total ω6 fatty acids: total ω3 fatty acids in the fatty acid content of the extracted lipid is between 1.0 and 3.0 or between 0.1 and 1, (x) the triacylglycerol (TAG) content of the lipid is at least 70%, and (xi) at least 70% of the DHA esterified in the form of TAG is in the sn-1 or sn-3 position of the TAG. In an embodiment, one or more or all of the following features
i) the level of palmitic acid in the total fatty acid content of the extracted lipid is between 2% and 15%,
ii) the level of myristic acid (C14:0) in the total fatty acid content of the extracted lipid is less than 1%,
iii) the level of oleic acid in the total fatty acid content of the extracted lipid is between about 3% and about 30%, between about 6% and about 30%, between 1% and about 20%, between about 45% and about 60%, or is about 30%,
iv) the level of linoleic acid (LA) in the total fatty acid content of the extracted lipid is between about 4% and about 20%, or between about 4% and 17%,
v) the level of α-linolenic acid (ALA) in the total fatty acid content of the extracted lipid is between about 7% and about 40%, between about 10% and about 35%, between about 20% and about 35%, or between about 4% and 16%.
vi) the level of γ-linolenic acid (GLA) in the total fatty acid content of the extracted lipid is less than 4%, less than about 3%, less than about 2%, less than about 1%, less than about 0.5%, between 0.05% and 7%, between 0.05% and 4%, or between 0.05% and about 3%, or between 0.05% and about 2%,
vii) the level of stearidonic acid (SDA) in the total fatty acid content of the extracted lipid is less than about 4%, less than about 3%, between about 0.05% and about 7%, between about 0.05% and about 4%, between about 0.05% and about 3%, or between 0.05% and about 2%,
viii) the level of eicosatetraenoic acid (ETA) in the total fatty acid content of the extracted lipid is less than about 4%, less than about 1%, less than about 0.5%, between about 0.05% and about 5%, between about 0.05% and about 4%, between about 0.05% and about 3%, or between about 0.05% and about 2%,
ix) the level of eicosatrienoic acid (ETrA) in the total fatty acid content of the extracted lipid is less than about 2%, less than about 1%, between 0.05% and 4%, between 0.05% and 3%, or between 0.05% and about 2%, or between 0.05% and about 1%,
x) the level of eicosapentaenoic acid (EPA) in the total fatty acid content of the extracted lipid is less than 4%, less than about 3%, less than about 2%, between 0.05% and 10%, between 0.05% and 5%, or between 0.05% and about 3%, or between 0.05% and about 2%,
xi) the level of docosapentaenoic acid (DPA) in the total fatty acid content of the extracted lipid is less than 4%, less than about 3%, less than about 2%, between 0.05% and 8%, between 0.05% and 5%, or between 0.05% and about 3%, or between 0.05% and about 2%.
xii) the level of DHA in the total fatty acid content of the extracted lipid is about 8%, about 9%, about 10%, about 11%, about 12%, about 13%, about 14%, about 15%, about 16%, about 17%, about 18%, between about 8% and 20%, between about 10% and 20%, between about 11% and 20%, between about 10% and about 16%, or between about 14% and 20%,
xiii) the lipid comprises ω6-docosapentaenoic acid (22:5^{Δ4,7,10,13,16}) in its fatty acid content,
xiv) the lipid is essentially free of (6-docosapentaenoic acid (22:5^{Δ4,7,10,13,16}) in its fatty acid content,
xv) the lipid is essentially free of SDA. EPA and ETA in its fatty acid content,
xvi) the level of total saturated fatty acids in the total fatty acid content of the extracted lipid is between about 4% and about 20%, or between about 6% and about 20%,
xvii) the level of total monounsaturated fatty acids in the total fatty acid content of the extracted lipid is between about 4% and about 35%, between about 8% and about 25%, or between 8% and about 22%,
xviii) the level of total polyunsaturated fatty acids in the total fatty acid content of the extracted lipid is between about 20% and about 75%, between about 50% and about 75%, or between about 60% and about 75%,
xix) the level of total ω6 fatty acids in the total fatty acid content of the extracted lipid is between about 35% and about 50%, between about 20% and about 35%, between about 6% and 20%, less than 20%, less than about 16%, less than about 10%, between about 1% and about 16%, between about 2% and about 10%, or between about 4% and about 10%,
xx) the level of new ω6 fatty acids in the total fatty acid content of the extracted lipid is less than about 10%, less than about 8%, less than about 6%, less than 4%, between about 1% and about 20%, between about 1% and about 10%, between about 0.5% and about 8%, or between about 0.5% and 4%,
xxi) the level of total ω3 fatty acids in the total fatty acid content of the extracted lipid is between 36% and about 65%, between 40% and about 60%, between about 20% and about 35%, between about 10% and about 20%, about 25%, about 30%, about 35% or about 40%,
xxii) the level of new ω3 fatty acids in the total fatty acid content of the extracted lipid is between 9% and about 33%, between about 10% and about 20%, between about 20% and about 30%, between about 12% and about 25%, about 13%, about 15%, about 17% or about 20%.
xxiii) the ratio of total ω6 fatty acids: total ω3 fatty acids in the fatty acid content of the extracted lipid is between about 0.1 and about 0.5, less than about 0.50, less than about 0.40, less than about 0.30, less than about 0.20, less than about 0.15, about 1.0, about 0.1 or about 0.2,
xxiv) the ratio of new ω6 fatty acids, new ω3 fatty acids in the fatty acid content of the extracted lipid is between about 1.0 and about 3.0, between about 0.1 and about 1, between about 0.1 and about 0.5, less than about 0.50, less than about 0.40, less than about 0.30, less than about 0.20, less than about 0.15, about 0.1, about 0.2 or about 1.0,
xxv) the fatty acid composition of the lipid is based on an efficiency of conversion of oleic acid to DHA of at least about 10%, at least about 15%, at least about 20%, between about 10% and about 50%, between about 10% and about 30%, or between about 10% and about 25%,
xxvi) the fatty acid composition of the lipid is based on an efficiency of conversion of LA to DHA of at least about 15%, at least about 20%, at least about 22%, at least about 25%, between about 15% and about 50%, between about 20% and about 40%, or between about 20% and about 30%,
xxvii) the fatty acid composition of the lipid is based on an efficiency of conversion of ALA to DHA of at least about 17%, at least about 22%, at least about 24%, between about 17% and about 55%, between about 22% and about 35%, or between about 24% and about 35%.
xxviii) the total fatty acid in the extracted lipid has less than 1% C20:1,
xxix) the triacylglycerol (TAG) content of the lipid is at least about 80%, at least about 90%, at least 95%, between about 70% and about 99%, or between about 90% and about 99%,
xxx) the lipid comprises diacylglycerol (DAG).
xxxi) the lipid comprises less than about 10%, less than about 5%, less than about 1%, or between about 0.001% and about 5%, free (non-esterified) fatty acids and/or phospholipid, or is essentially free thereof.
xxxii) at least 80%, of the DHA esterified in the form of TAG is in the sn-1 or sn-3 position of the TAG,
xxxiii) the most abundant DHA-containing TAG species in the lipid is DHA/18:3/18:3 (TAG 58:12), and
xxxiv) the lipid comprises tri-DHA TAG (TAG 66:18).

With specific regard to the above aspect, in an embodiment
i) the lipid is in the form of an oil, wherein the oil comprises one or more sterols such as one or more or all of campesterol, Δ5-stigmasterol, eburicol, β-sitosterol, Δ5-avenasterol, Δ7-stigmasterol and Δ7-avenasterol, and optionally the oil comprises less than 10 mg of sterols/g of oil and/or the oil is essentially free of cholesterol, and/or
ii) the lipid is in the form of an oil from an oilseed such as oilseed is a Brassica sp oilseed or canola seed.

In another aspect, the present invention provides a process for producing extracted plant lipid, comprising the steps of
i) obtaining a plant part comprising lipid, the lipid comprising fatty acids in an esterified form, the fatty acids comprising oleic acid, palmitic acid, ω6 fatty acids which comprise linoleic acid (LA), ω3 fatty acids which comprise α-linolenic acid (ALA), and docosahexaenoic acid (DHA), and optionally one or more of eicosapentaenoic acid (EPA), stearidonic acid (SDA), docosapentaenoic acid (DPA) and eicosatetraenoic acid (ETA), wherein the level of DHA in the total fatty acid content of extractable lipid in the plant part is about 7% to 20%, and
ii) extracting lipid from the plant part,
   wherein the level of DHA in the total fatty acid content of the extracted lipid is about 7% to 20%.

In a preferred embodiment, the extracted lipid has one or more of the features defined above.

In an embodiment, wherein the plant part is a seed, preferably an oilseed.

Examples of such seeds include, but are not limited to, Brassica sp., Gossypium hirsutum, Linum usitatissimum, Helianthus sp., Carthamus tinctorius, Glycine max, Zea mays, Arabidopsis thaliana, Sorghum bicolor, Sorghum vulgare, Avena sativa, Trifolium sp., Elaesis guineenis, Nicotiana benthamiana, Hordeum vulgare, Lupinus angustifolius, Oryza sativa, Oryza glaberrima, Camelina sativa, or Crambe abyssinica, preferably a Brassica napus. B. juncea or C. sativa seed.

In another embodiment, the seed comprises at least about 18 mg, at least about 22 mg, at least about 26 mg, between about 18 mg and about 100 mg, between about 22 mg and about 70 mg, or between about 24 mg and about 50 mg, of DHA per gram of seed.

In a further embodiment, the plant part comprises exogenous polynucleotides encoding one of the following sets of enzymes;
i) an ω3-desaturase, a Δ6-desaturase, a Δ5-desaturase, a Δ4-desaturase, a Δ6-elongase and a Δ5-elongase,
ii) a Δ15-desaturase, a Δ6-desaturase, a Δ5-desaturase, a Δ4-desaturase, a Δ6-elongase and a Δ5-elongase,
iii) a Δ12-desaturase, a Δ6-desaturase, a Δ5-desaturase, a Δ4-desaturase, a Δ6-elongase and an Δ5-elongase,
iv) a Δ12-desaturase, a ω3-desaturase or a Δ15-desaturase, a Δ6-desaturase, a Δ5-desaturase, a Δ4-desaturase, a Δ6-elongase and an Δ5-elongase,
v) an ω3-desaturase, a Δ8-desaturase, a Δ5-desaturase, a Δ4-desaturase, a Δ9-elongase and an Δ5-elongase,
vi) a Δ15-desaturase, a Δ8-desaturase, a Δ5-desaturase, a Δ4-desaturase, a Δ9-elongase and a Δ5-elongase,
vii) a Δ12-desaturase, a Δ8-desaturase, a Δ5-desaturase, a Δ4-desaturase, a Δ9-elongase and an Δ5-elongase, or
viii) a Δ12-desaturase, a ω3-desaturase or a Δ15-desaturase, a Δ8-desaturase, a Δ5-desaturase, a Δ4-desaturase, a Δ9-elongase and an Δ5-elongase, and wherein each polynucleotide is operably linked to one or more promoters that are capable of directing expression of said polynucleotides in a cell of the plant part.

In yet a further embodiment, the plant part has one or more or all of the following features
i) the Δ12-desaturase converts oleic acid to linoleic acid in one or more cells of the plant with an efficiency of at least about 60%, at least about 70%, at least about 80%, between about 60% and about 98%, between about 70% and about 95%, or between about 75% and about 90%,
ii) the ω3-desaturase converts ω6 fatty acids to ω3 fatty acids in one or more cells of the plant with an efficiency of at least about 65%, at least about 75%, at least about 85%, between about 65% and about 95%, between about 75% and about 95%, or between about 80% and about 95%.
iii) the Δ6-desaturase converts ALA to SDA in one or more cells of the plant with an efficiency of at least about 30%, at least about 40%, at least about 50%, at least about 60%, at least about 70%, between about 30% and about 70%, between about 35% and about 60%, or between about 50% and about 70%,
iv) the Δ6-desaturase converts linoleic acid to γ-linolenic acid in one or more cells of the plant with an efficiency of less than about 5%, less than about 2.5%, less than about 1%, between about 0.1% and about 5%, between about 0.5% and about 2.5%, or between about 0.5% and about 1%,
v) the Δ6-elongase converts SDA to ETA in one or more cells of the plant with an efficiency of at least about 60%, at least about 70%, at least about 75%, between about 60% and about 95%, between about 70% and about 88%, or between about 75% and about 85%,
vi) the Δ5-desaturase converts ETA to EPA in one or more cells of the plant with an efficiency of at least about 60%, at least about 70%, at least about 75%, at least about 80%, at least about 90%, between about 60% and about 99%, between about 70% and about 99%, or between about 75% and about 98%,
vii) the Δ5-elongase converts EPA to DPA in one or more cells of the plant with an efficiency of at least about 80%, at least about 85%, at least about 90%, between about 50% and about 95%, or between about 85% and about 95%,
viii) the Δ4-desaturase converts DPA to DHA in one or more cells of the plant with an efficiency of at least about 80%, at least about 90%, at least about 93%, between about 50% and about 95%, between about 80% and about 95%, or between about 85% and about 95%,
ix) the efficiency of conversion of oleic acid to DHA in one or more cells of the plant part is at least about 10%, at least about 15%, at least about 20%° s, between about 10% and about 50%, between about 10% and about 30%, or between about 10% and about 25%.
x) the efficiency of conversion of LA to DHA in one or more cells of the plant part is at least about 15%, at least about 20%, at least about 22%, at least about 25%, between about 15% and about 50%, between about 20% and about 40%, or between about 20% and about 30%,
xi) the efficiency of conversion of ALA to DHA in one or more cells of the plant part is at least about 17%, at least about 22%, at least about 24%, between about 17% and about 55%, between about 22% and about 35%, or between about 24% and about 35%,
xii) one or more cells of the plant part comprise at least about 15%, at least about 20%, between about 15% and about 30%, or between about 22.5% and about 27.5%, more ω3 fatty acids than corresponding cells lacking the exogenous polynucleotides,
xiii) the Δ6-desaturase preferentially desaturates α-linolenic acid (ALA) relative to linoleic acid (LA),
xiv) the Δ6-elongase also has Δ9-elongase activity,
xv) the Δ12-desaturase also has Δ15-desaturase activity,
xvi) the Δ6-desaturase also has Δ8-desaturase activity,
xvii) the Δ8-desaturase also has Δ6-desaturase activity or does not have Δ6-desaturase activity.
xviii) the Δ15-desaturase also has ω3-desaturase activity on GLA,
xix) the ω3-desaturase also has Δ15-desaturase activity on LA,
xx) the ω3-desaturase desaturates both LA and/or GLA,
xxi) the ω3-desaturase preferentially desaturates GLA relative to LA,
xxii) the level of DHA in the plant part is based on an efficiency of conversion of oleic acid to DHA in the plant part of at least about 10%, at least about 15%, at least about 20%, between about 10% and about 50%, between about 15% and about 30%, or between about 20% and about 25%.
xxiii) the level of DHA in the plant part is based on an efficiency of conversion of LA to DHA in the plant part of at least about 15%, at least about 20%, at least about 22%, between about 15% and about 60%, between about 20% and about 40%, or between about 22% and about 30%,
xxiv) the level of DHA in the plant part is based on an efficiency of conversion of ALA to DHA in the plant part of at least about 17%, at least about 22%, at least about 24%, between about 17% and about 65%, between about 22% and about 35%, or between about 24% and about 35%
xxx) one or more or all of the desaturases have greater activity on an acyl-CoA substrate than a corresponding acyl-PC substrate,
xxxi) the Δ6-desaturase has greater Δ6-desaturase activity on ALA than LA as fatty acid substrate,
xxxii) the Δ6-desaturase has greater Δ6-desaturase activity on ALA-CoA as fatty acid substrate than on ALA joined to the sn-2 position of PC as fatty acid substrate,
xxxiii) the Δ6-desaturase has at least about a 2-fold greater Δ6-desaturase activity, at least 3-fold greater activity, at least 4-fold greater activity, or at least 5-fold greater activity, on ALA as a substrate compared to LA,
xxxiv) the Δ6-desaturase has greater activity on ALA-CoA as fatty acid substrate than on ALA joined to the sn-2 position of PC as fatty acid substrate,
xxxv) the Δ6-desaturase has at least about a 5-fold greater Δ6-desaturase activity or at least 10-fold greater activity, on ALA-CoA as fatty acid substrate than on ALA joined to the sn-2 position of PC as fatty acid substrate,
xxxvi) the desaturase is a front-end desaturase,
xxxvii) the Δ6-desaturase has no detectable Δ5-desaturase activity on ETA.

In yet a further embodiment, the plant part has one or more or all of the following features
i) the Δ12-desaturase comprises amino acids having a sequence as provided in SEQ ID NO:10, a biologically active fragment thereof, or an amino acid sequence which is at least 50% identical to SEQ ID NO:10,
ii) the ω3-desaturase comprises amino acids having a sequence as provided in SEQ ID NO:12, a biologically active fragment thereof, or an amino acid sequence which is at least 50% identical to SEQ ID NO:12,
iii) the Δ6-desaturase comprises amino acids having a sequence as provided in SEQ ID NO:16, a biologically active fragment thereof, or an amino acid sequence which is at least 50% identical to SEQ ID NO:16,
iv) the Δ6-elongase comprises amino acids having a sequence as provided in SEQ ID NO:25, a biologically active fragment thereof such as SEQ ID NO:26, or an amino acid sequence which is at least 50% identical to SEQ ID NO:25 and/or SEQ ID NO:26.
v) the Δ5-desaturase comprises amino acids having a sequence as provided in SEQ ID NO:30, a biologically active fragment thereof, or an amino acid sequence which is at least 50% identical to SEQ ID NO:30.
vi) the Δ5-elongase comprises amino acids having a sequence as provided in SEQ ID NO:37, a biologically active fragment thereof, or an amino acid sequence which is at least 50% identical to SEQ ID NO:37,
vii) the Δ4-desaturase comprises amino acids having a sequence as provided in SEQ ID NO:41, a biologically active fragment thereof, or an amino acid sequence which is at least 50% identical to SEQ ID NO:41.

In an embodiment, the plant part further comprises an exogenous polynucleotide encoding a diacylglycerol acyltransferase (DGAT), monoacylglycerol acyltransferase (MGAT), glycerol-3-phosphate acyltransferase (GPAT), 1-acyl-glycerol-3-phosphate acyltransferase (LPAAT) preferably an LPAAT which can use a C22 polyunsaturated fatty acyl-CoA substrate, acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), phosphatidylcholine:diacylglycerol choline phosphotransferase (PDCT), acyl-CoA synthase (ACS), or a combination of two or more thereof.

In another embodiment, the plant part further comprises an introduced mutation or an exogenous polynucleotide which down regulates the production and/or activity of an endogenous enzyme in a cell of the plant part selected from FAE1. DGAT, MGAT, GPAT, LPAAT, LPCAT, PLA₂, PLC, PLD, CPT, PDAT, a thioesterase such as FATB, or a Δ12-desaturase, or a combination of two or more thereof.

In a further embodiment, at least one, or all, of the promoters are seed specific promoters. In an embodiment, at least one, or all, of the promoters have been obtained from oil biosynthesis or accumulation genes such as oleosin, or from seed storage protein genes such as conlinin.

In another embodiment, the promoter(s) directing expression of the exogenous polynucleotides encoding the Δ4-desaturase and the Δ5-elongase initiate expression of the polynucleotides in developing seed of the plant part before, or reach peak expression before, the promoter(s) directing expression of the exogenous polynucleotides encoding the Δ12-desaturase and the ω3-desaturase.

In a further embodiment, the exogenous polynucleotides are covalently linked in a DNA molecule, preferably a T-DNA molecule, integrated into the genome of cells of the plant part and preferably where the number of such DNA molecules integrated into the genome of the cells of the plant part is not more than one, two or three, or is two or three.

In yet another embodiment, the plant comprises at least two different, exogenous polynucleotides each encoding a Δ6-desaturase which have the same or different amino acid sequences.

In a further embodiment, the total oil content of the plant part comprising the exogenous polynucleotides is at least about 40%, or at least about 50%, or at least about 60%, or at least about 70%, or between about 50% and about 80% of the total oil content of a corresponding plant part lacking the exogenous polynucleotides. In these embodiments, the maximum oil content may be about 100% of the oil content of a corresponding wild-type plant part.

In another embodiment, the lipid is in the form of an oil, preferably a seedoil from an oilseed, and wherein at least about 90%, or about least 95%, at least about 98%, or between about 95% and about 98%, by weight of the lipid is triacylglycerols.

In a further embodiment, the process further comprises treating the lipid to increase the level of DHA as a percentage of the total fatty acid content. For example, the treatment is transesterification. For example, the lipid such as canola oil may be treated to convert the fatty acids in the oil to alkyl esters such as methyl or ethyl esters, which may then be fractionated to enrich the lipid or oil for the DHA.

Further, provided is a process for producing extracted plant lipid, comprising the steps of
i) obtaining a plant part, preferably canola seed, comprising lipid, the lipid comprising fatty acids in an esterified form, the fatty acids comprising oleic acid, palmitic acid, ω6 fatty acids which comprise linoleic acid (LA), ω3 fatty acids which comprise α-linolenic acid (ALA), and docosahexaenoic acid (DHA), and optionally one or more of eicosapentaenoic acid (EPA), stearidonic acid (SDA), docosapentaenoic acid (DPA) and eicosatetraenoic acid (ETA), wherein the level of DHA in the total fatty acid content of extractable lipid in the plant part is about 3%, about 4%, about 5%, about 6% or about 7%, and
ii) extracting lipid from the plant part, wherein the extracted lipid has the following features in the total fatty acid content of the lipid;
i) the level of DHA is about 3%, about 4%, about 5%, about 6% or about 7%,
ii) the level of palmitic acid is between about 2% and about 16%,
iii) the level of myristic acid is less than about 2%,
iv) the level of oleic acid is between about 30% and about 60%, preferably between about 45% and about 60%.
v) the level of LA is between about 4% and about 20%,
vi) the level of ALA is between about 2% and about 16%,
vii) the level of GLA is less than about 4%,
viii) the level of SDA is less than about 6%, or less than about 4%,
ix) the level of ETA is less than about 6%, or less than about 4%.
x) the level of ETrA less than about 1%,
xi) the level of EPA is less than about 10% and/or the level of EPA is 0.5-2.0 fold the level of DHA,
xii) the level of DPA is less than about 4%,
xiii) the level of total saturated fatty acids in the total fatty acid content of the extracted lipid is between about 4% and about 25%,
xiv) the level of total monounsaturated fatty acids in the total fatty acid content of the extracted lipid is between about 30% and about 70%,
xv) the level of total polyunsaturated fatty acids in the total fatty acid content of the extracted lipid is between about 15% and about 75%, preferably between about 15% and about 30%,
xvi) the level of new ω6 fatty acids in the total fatty acid content of the extracted lipid is between about 0.5% and about 10%,
xvii) the level of total ω3 fatty acids in the total fatty acid content of the extracted lipid is between about 10% and about 20%,
xviii) the level of new ω3 fatty acids in the total fatty acid content of the extracted lipid is between about 3% and about 20%,
xix) the ratio of total (ω6 fatty acids: total (3 fatty acids in the fatty acid content of the extracted lipid is between about 0.05 and about 3.0, preferably less than about 0.50,
xx) the ratio of new ω6 fatty acids: new ω3 fatty acids in the fatty acid content of the extracted lipid is between about 0.03 and about 3.0,
xxi) the triacylglycerol (TAG) content of the lipid is at least about 70%, and xxii) the lipid is essentially free of cholesterol. In an embodiment, the lipid comprises tri-DHA TAG (TAG 66:18). More preferably, the lipid is essentially free of SDA and ETA, and/or has not been treated with a transesterification process after it was extracted from the plant or plant part.

Also provided is a process for producing extracted plant lipid, comprising the steps of
i) obtaining a plant part comprising lipid, the lipid comprising fatty acids in an esterified form, the fatty acids comprising oleic acid, palmitic acid, ω6 fatty acids which comprise linoleic acid (LA), ω3 fatty acids which comprise α-linolenic acid (ALA) and docosahexaenoic acid (DHA), and one or more of stearidonic acid (SDA), eicosapentaenoic acid (EPA), docosapentaenoic acid (DPA) and eicosatetraenoic acid (ETA), wherein (i) the level of DHA in the total fatty acid content of the extracted lipid is between 7% and 20%, (ii) the level of palmitic acid in the total fatty acid content of the extracted lipid is between 2% and 16%, (iii) the level of myristic acid (C14:0) in the total fatty acid content of the extracted lipid is less than 6%, (iv) the level of oleic acid in the total fatty acid content of the extracted lipid is between 1% and 30% or between 30% and 60%, (v) the level of linoleic acid (LA) in the total fatty acid content of the extracted lipid is between 4% and 35%, (vi) the level of α-linolenic acid (ALA) in the total fatty acid content of the extracted lipid is between 4% and 40%, (vii) the level of eicosatrienoic acid (ETrA) in the total fatty acid content of the extracted lipid is less than 4%. (viii) the level of total saturated fatty acids in the total fatty acid content of the extracted lipid is between 4% and 25%, (ix) the ratio of total ω6 fatty acids: total ω3 fatty acids in the fatty acid content of the extracted lipid is between 1.0 and 3.0 or between 0.1 and 1, (x) the triacylglycerol (TAG) content of the lipid is at least 70%, and (xi) at least 70% of the DHA esterified in the form of TAG is in the sn-1 or sn-3 position of the TAG. %, and
ii) extracting lipid from the plant part,
   wherein the level of DHA in the total fatty acid content of the extracted lipid is about 7% to 20%.

Also provided is lipid, or oil comprising the lipid, produced using a process of the invention.

In another aspect, the present invention provides a process for producing ethyl esters of polyunsaturated fatty acids, the process comprising transesterifying triacylglycerols in extracted plant lipid, wherein the extracted plant lipid comprises fatty acids esterified in the form, the fatty acids comprising oleic acid, palmitic acid, ω6 fatty acids which comprise linoleic acid (LA), ω3 fatty acids which comprise α-linolenic acid (ALA), and docosahexaenoic acid (DHA), and optionally one or more of stearidonic acid (SDA), eicosapentaenoic acid (EPA), docosapentaenoic acid (DPA) and eicosatetraenoic acid (ETA), wherein the level of DHA in the total fatty acid content of the extracted lipid is about 7% to 20%, thereby producing the ethyl esters.

In a preferred embodiment, the extracted lipid has one or more of the features defined above.

In another aspect, the present invention provides a process for producing ethyl esters of polyunsaturated fatty acids, the process comprising transesterifying triacylglycerols in extracted plant lipid, wherein the extracted plant lipid comprises fatty acids esterified in the form of the triacylglycerols, the fatty acids comprising oleic acid, palmitic acid, ω6 fatty acids which comprise linoleic acid (LA), ω3 fatty acids which comprise α-linolenic acid (ALA) and docosahexaenoic acid (DHA), and one or more of stearidonic acid (SDA), eicosapentaenoic acid (EPA), docosapentaenoic acid (DPA) and eicosatetraenoic acid (ETA), wherein (i) the level of DHA in the total fatty acid content of the extracted lipid is about 3%, about 4%, about 5%, about 6% or between 7% and 20%, (ii) the level of palmitic acid in the total fatty acid content of the extracted lipid is between 2% and 16%, (iii) the level of myristic acid (C14:0) in the total fatty acid content of the extracted lipid is less than 6%, (iv) the level of oleic acid in the total fatty acid content of the extracted lipid is between 1% and 30% or between 30% and 60%, (v) the level of linoleic acid (LA) in the total fatty acid content of the extracted lipid is between 4% and 35%, (vi) the level of α-linolenic acid (ALA) in the total fatty acid content of the extracted lipid is between 4% and 40%, (vii) the level of eicosatrienoic acid (ETrA) in the total fatty acid content of the extracted lipid is less than 4%, (viii) the level of total saturated fatty acids in the total fatty acid content of the extracted lipid is between 4% and 25%. (ix) the ratio of total ω6 fatty acids: total ω3 fatty acids in the fatty acid content of the extracted lipid is between 1.0 and 3.0 or between 0.1 and 1, (x) the triacylglycerol (TAG) content of the lipid is at least 70%, and (xi) at least 70% of the DHA esterified in the form of TAG is in the sn-1 or sn-3 position of the TAG, thereby producing the ethyl esters. In an embodiment, the extracted plant lipid has one or more or all of the following features
i) the level of palmitic acid in the total fatty acid content of the extracted lipid is between 2% and 15%,
ii) the level of myristic acid (C14:0) in the total fatty acid content of the extracted lipid is less than 1%,
xxxv) the level of oleic acid in the total fatty acid content of the extracted lipid is between about 3% and about 30%, between about 6% and about 30%, between 1% and about 20%, between about 45% and about 60%, or is about 30%,
xxxvi) the level of linoleic acid (LA) in the total fatty acid content of the extracted lipid is between about 4% and about 20%, or between about 4% and 17%,
xxxvii) the level of α-linolenic acid (ALA) in the total fatty acid content of the extracted lipid is between about 7% and about 40%, between about 10% and about 35%, between about 20% and about 35%, or between about 4% and 16%,
xxxviii) the level of γ-linolenic acid (GLA) in the total fatty acid content of the extracted lipid is less than 4%, less than about 3%, less than about 2%, less than about 1%, less than about 0.5%, between 0.05% and 7%, between 0.05% and 4%, or between 0.05% and about 3%, or between 0.05% and about 2%.
xxxix) the level of stearidonic acid (SDA) in the total fatty acid content of the extracted lipid is less than about 4%, less than about 3%, between about 0.05% and about 7%, between about 0.05% and about 4%, between about 0.05% and about 3%, or between 0.05% and about 2%,
xl) the level of eicosatetraenoic acid (ETA) in the total fatty acid content of the extracted lipid is less than about 4%, less than about 1%, less than about 0.5%, between about 0.05% and about 5%, between about 0.05% and about 4%, between about 0.05% and about 3%, or between about 0.05% and about 2%,
xli) the level of eicosatrienoic acid (ETrA) in the total fatty acid content of the extracted lipid is less than about 2%, less than about 1%, between 0.05% and 4%, between 0.05% and 3%, or between 0.05% and about 2%, or between 0.05% and about 1%,
xlii) the level of eicosapentaenoic acid (EPA) in the total fatty acid content of the extracted lipid is less than 4%, less than about 3%, less than about 2%, between 0.05% and 10%, between 0.05% and 5%, or between 0.05% and about 3%, or between 0.05% and about 2%,
xliii) the level of docosapentaenoic acid (DPA) in the total fatty acid content of the extracted lipid is less than 4%, less than about 3%, less than about 2%, between 0.05% and 8%, between 0.05% and 5%, or between 0.05% and about 3%, or between 0.05% and about 2%,
xliv) the level of DHA in the total fatty acid content of the extracted lipid is about 8%, about 9%, about 10%, about 11%, about 12%, about 13%, about 14%, about 15%, about 16%, about 17%, about 18%, between about 8% and 20%, between about 10% and 20%, between about 11% and 20%, between about 10% and about 16%, or between about 14% and 20%,
xlv) the lipid comprises ω6-docosapentaenoic acid (22:5^{Δ4,7,10,13,16}) in its fatty acid content,
xlvi) the lipid is essentially free of ω6-docosapentaenoic acid (22:5^{Δ4,7,10,13,16}) in its fatty acid content,
xlvii) the lipid is essentially free of SDA. EPA and ETA in its fatty acid content,
xlviii) the level of total saturated fatty acids in the total fatty acid content of the extracted lipid is between about 4% and about 20%, or between about 6% and about 20%,
xlix) the level of total monounsaturated fatty acids in the total fatty acid content of the extracted lipid is between about 4% and about 35%, between about 8% and about 25%, or between 8% and about 22%,
l) the level of total polyunsaturated fatty acids in the total fatty acid content of the extracted lipid is between about 20% and about 75%, between about 50% and about 75%, or between about 60% and about 75%,
li) the level of total ω6 fatty acids in the total fatty acid content of the extracted lipid is between about 35% and about 50%, between about 20% and about 35%, between about 6% and 20%, less than 20%, less than about 16%, less than about 10%, between about 1% and about 16%, between about 2% and about 10%, or between about 4% and about 10%,
lii) the level of new ω6 fatty acids in the total fatty acid content of the extracted lipid is less than about 10%, less than about 8%, less than about 6%, less than 4%, between about 1% and about 20%, between about 1% and about 10%, between about 0.5% and about 8%, or between about 0.5% and 4%,
liii) the level of total ω3 fatty acids in the total fatty acid content of the extracted lipid is between 36% and about 65%, between 40% and about 60%, between about 20% and about 35%, between about 10% and about 20%, about 25%, about 30%, about 35% or about 40%,
liv) the level of new ω3 fatty acids in the total fatty acid content of the extracted lipid is between 9% and about 33%, between about 10% and about 20%, between about 20% and about 30%, between about 12% and about 25%, about 13%, about 15%, about 17% or about 20%,
lv) the ratio of total ω6 fatty acids: total ω3 fatty acids in the fatty acid content of the extracted lipid is between about 0.1 and about 0.5, less than about 0.50, less than about 0.40, less than about 0.30, less than about 0.20, less than about 0.15, about 1.0, about 0.1 or about 0.2,
lvi) the ratio of new ω6 fatty acids: new ω3 fatty acids in the fatty acid content of the extracted lipid is between about 1.0 and about 3.0, between about 0.1 and about 1, between about 0.1 and about 0.5, less than about 0.50, less than about 0.40, less than about 0.30, less than about 0.20, less than about 0.15, about 0.1, about 0.2 or about 1.0,
lvii) the fatty acid composition of the lipid is based on an efficiency of conversion of oleic acid to DHA of at least about 10%, at least about 15%, at least about 20%, between about 10% and about 50%, between about 10% and about 30%, or between about 10% and about 25%,
lviii) the fatty acid composition of the lipid is based on an efficiency of conversion of LA to DHA of at least about 15%, at least about 20%, at least about 22%, at least about 25%, between about 15% and about 50%, between about 20% and about 40%, or between about 20% and about 30%,
lix) the fatty acid composition of the lipid is based on an efficiency of conversion of ALA to DHA of at least about 17%, at least about 22%, at least about 24%, between about 17% and about 55%, between about 22% and about 35%, or between about 24% and about 35%,
lx) the total fatty acid in the extracted lipid has less than 1% C20:1,
lxi) the triacylglycerol (TAG) content of the lipid is at least about 80%, at least about 90%, at least 95%, between about 70% and about 99%, or between about 90% and about 99%,
lxii) the lipid comprises diacylglycerol (DAG),
lxiii) the lipid comprises less than about 10%, less than about 5%, less than about 1%, or between about 0.001% and about 5%, free (non-esterified) fatty acids and/or phospholipid, or is essentially free thereof,
lxiv) at least 80%, of the DHA esterified in the form of TAG is in the sn-1 or sn-3 position of the TAG,
lxv) the most abundant DHA-containing TAG species in the lipid is DHA/18:3/18:3 (TAG 58:12), and
lxvi) the lipid comprises tri-DHA TAG (TAG 66:18).

With specific regard to the above aspect, in an embodiment one or more or all of the following apply
i) the lipid is in the form of an oil, wherein the oil comprises one or more sterols such as one or more or all of campesterol, Δ5-stigmasterol, eburicol, β-sitosterol, Δ5-avenasterol, Δ7-stigmasterol and Δ7-avenasterol, and optionally the oil comprises less than 10 mg of sterols/g of oil and/or the oil is essentially free of cholesterol,
ii) the lipid is in the form of an oil from an oilseed such as oilseed is a Brassica sp oilseed or canola seed.
iii) the level of DHA in the total fatty acid content of the extracted plant lipid is about 3%, about 4%, about 5%, about 6%, or is between 7% and 20%.

In a further aspect, the present invention provides a chimeric genetic construct comprising in order a first gene, a second gene, a third gene, a fourth gene, a fifth gene and a sixth gene which are all covalently linked on a single DNA molecule.
wherein the first, second and third genes are joined together as a first gene cluster and the fourth, fifth and sixth genes are joined together as a second gene cluster,
wherein each gene comprises a promoter, a coding region and a transcription terminator and/or polyadenylation region such that each promoter is operably linked to the coding region and transcription terminator and/or polyadenylation region,
wherein each promoter is independently identical or different to the other promoters such that the DNA molecule comprises three, four, five or six different promoters,
wherein one or more or all of the promoters are heterologous with respect to the coding region to which it is operably linked,
wherein the direction of transcription of the first gene is away from the third gene and opposite to the direction of transcription of the third gene,
wherein the direction of transcription of the fourth gene is away from the sixth gene and opposite to the direction of transcription of the sixth gene,
wherein the direction of transcription of the second gene is the same as for the first gene or the third gene,
wherein the direction of transcription of the fifth gene is the same as for the fourth gene or the sixth gene,
wherein the transcription terminator and/or polyadenylation region of the second gene is spaced apart from the promoter of the first or third genes, whichever is closer, by a first spacer region of between about 0.2 and about 3.0 kilobases,
wherein the first gene cluster is spaced apart from the second gene cluster by a second spacer region of between about 1.0 and about 10.0 kilobases, and
wherein the transcription terminator and/or polyadenylation region of the fifth gene is spaced apart from the promoter of the fourth or sixth genes, whichever is closer, by a third spacer region of between about 0.2 and about 3.0 kilobases.

In an embodiment, the DNA molecule comprises a seventh gene which is spaced apart from the first gene cluster or the second gene cluster, whichever is closer, by a spacer region of between about 1.0 and about 10.0 kilobases.

In another embodiment, the DNA molecule comprises two or more different transcription terminator and/or polyadenylation regions.

In yet a further embodiment, at least one of the spacer regions comprises a matrix attachment region (MAR).

In a further embodiment, the DNA molecule comprises right and left border regions flanking the genes and is a T-DNA molecule.

In another embodiment, the genetic construct is in an Agrobacterium cell or is integrated into the genome of a plant cell.

In a preferred embodiment, at least one of the genes encodes a fatty acid desaturase or a fatty acid elongase.

In another embodiment, the genetic construct comprises genes encoding a set of enzymes as defined herein, and/or wherein one or more of the genes encode an enzyme as defined herein.

In a further aspect, the present invention provides an isolated and/or exogenous polynucleotide comprising:
i) a sequence of nucleotides selected from any one of SEQ ID NOs: 1 to 9, 11, 14, 18, 22, 23, 28, 34, 35, 39 or 45, and/or
ii) a sequence of nucleotides which are at least 95% identical or 99% identical to one or more of the sequences set forth in SEQ ID NOs: 1 to 9, 11, 14, 18, 22, 23, 28, 34, 35, 39 or 45.

In a particularly preferred embodiment, the isolated and/or exogenous polynucleotide comprises:
i) a sequence of nucleotides of SEQ ID NO: 2, and/or
ii) a sequence of nucleotides which are at least 95% identical or 99% identical to the sequence set forth in SEQ ID NO: 2.

In another aspect, the present invention provides a vector or genetic construct comprising the polynucleotide of the invention and/or the genetic construct of the invention.

In an embodiment, the sequence of nucleotides selected from any one of SEQ ID NOs: 11, 14, 18, 22, 23, 28, 34, 35, 39 or 45, or the sequence of nucleotides which is at least 95% identical or 99% identical to one or more of the sequences set forth in SEQ ID NOs: 11, 14, 18, 22, 23, 28, 34, 35, 39 or 45, is operably linked to a promoter.

In a further aspect, the present invention provides a host cell comprising exogenous polynucleotides encoding one of the following sets of enzymes;
i) an ω3-desaturase, a Δ6-desaturase, a Δ5-desaturase, a Δ4-desaturase, a Δ6-elongase and a Δ5-elongase,
ii) a Δ15-desaturase, a Δ6-desaturase, a Δ5-desaturase, a Δ4-desaturase, a Δ6-elongase and a Δ5-elongase,
iii) a Δ12-desaturase, a Δ6-desaturase, a Δ5-desaturase, a Δ4-desaturase, a Δ6-elongase and an Δ5-elongase,
iv) a Δ12-desaturase, a ω3-desaturase or a Δ15-desaturase, a Δ6-desaturase, a Δ5-desaturase, a Δ4-desaturase, a Δ6-elongase and an Δ5-elongase,
v) an ω3-desaturase, a Δ8-desaturase, a Δ5-desaturase, a Δ4-desaturase, a Δ9-elongase and an Δ5-elongase,
vi) a Δ15-desaturase, a Δ8-desaturase, a Δ5-desaturase, a Δ4-desaturase, a Δ9-elongase and a Δ5-elongase,
vii) a Δ12-desaturase, a Δ8-desaturase, a Δ5-desaturase, a Δ4-desaturase, a Δ9-elongase and an Δ5-elongase, or
viii) a Δ12-desaturase, a ω3-desaturase or a Δ15-desaturase, a Δ8-desaturase, a Δ5-desaturase, a Δ4-desaturase, a Δ9-elongase and an Δ5-elongase, and wherein each polynucleotide is operably linked to one or more promoters that are capable of directing expression of said polynucleotides in the cell.

In an embodiment, the cell comprises lipid as defined above, or wherein one or more or all of the desaturases or elongases have one or more of the features as defined above.

In another aspect, the present invention provides a host cell comprising
i) a first exogenous polynucleotide encoding a Δ12-desaturase which comprises amino acids having a sequence as provided in SEQ ID NO:10, a biologically active fragment thereof, or an amino acid sequence which is at least 50% identical to SEQ ID NO:10, and
ii) a second exogenous polynucleotide encoding a ω3-desaturase which comprises amino acids having a sequence as provided in SEQ ID NO:12, a biologically active fragment thereof, or an amino acid sequence which is at least 50% identical to SEQ ID NO:12,
wherein each polynucleotide is operably linked to one or more promoters that are capable of directing expression of said polynucleotides in the cell.

In a further aspect, the present invention provides a host cell comprising one or more of the polynucleotide of the invention, the genetic construct of the invention, or the vector or genetic construct of the invention.

In an embodiment, the cell is in a plant, in a plant part and/or is a mature plant seed cell.

In an embodiment, the plant or plant seed is an oilseed plant or an oilseed, respectively.

Also provided is a transgenic non-human organism comprising a cell of the invention. Preferably, the transgenic non-human organism is a transgenic plant, preferably an oilseed plant or Arabidopsis thaliana. In an embodiment, the plant is a Brassica plant, preferably B. napus or B. juncea, or a plant other than Arabidopsis thaliana.

In another aspect, the present invention provides an oilseed plant comprising
a) lipid in its seed, the lipid comprising fatty acids in an esterified form, and
b) exogenous polynucleotides encoding one of the following sets of enzymes;
   i) a Δ12-desaturase, a fungal ω3-desaturase and/or fungal Δ15-desaturase, a Δ6-desaturase, a Δ5-desaturase, a Δ4-desaturase, a Δ6-elongase and an Δ5-elongase, or
   ii) a Δ12-desaturase, a fungal ω3-desaturase and/or fungal Δ15-desaturase, a Δ8-desaturase, a Δ5-desaturase, a Δ4-desaturase, a Δ9-elongase and an Δ5-elongase,
wherein each polynucleotide is operably linked to one or more seed-specific promoters that are capable of directing expression of said polynucleotides in developing seed of the plant, wherein the fatty acids comprise oleic acid, palmitic acid, ω6 fatty acids which comprise linoleic acid (LA) and γ-linolenic acid (GLA), ω3 fatty acids which comprise α-linolenic acid (ALA), stearidonic acid (SDA), docosapentaenoic acid (DPA) and docosahexaenoic acid (DHA), and optionally eicosapentaenoic acid (EPA) and/or eicosatetraenoic acid (ETA), and wherein the level of DHA in the total fatty acid content of the lipid is about 7% to 20%.

Examples of oilseed plants include, but are not limited to, Brassica sp.,

Gossypium hirsutum, Linum usitatissimum, Helianthus sp., Carthamus tinctorius, Glycine max, Zea mays, Arabidopsis thaliana, Sorghum bicolor, Sorghum vulgare, Avena sativa, Trifolium sp., Elaesis guineenis. Nicotiana benthamiana, Hordeum vulgare, Lupinus angustifolius, Oryza sativa, Oryza glaberrima, Camelina sativa, or Crambe abyssinica. In an embodiment, the oilseed plant is a canola, Glycine max, Camelina sativa or Arabidopsis thaliana plant. In an alternate embodiment, the oilseed plant is other than A. thaliana.

In an embodiment, one or more of the desaturases is capable of using an acyl-CoA substrate. In a preferred embodiment, one or more of the Δ6-desaturase, Δ5-desaturase, Δ4-desaturase and Δ8-desaturase, if present, is capable of using an acyl-CoA substrate, preferably each of the i) Δ6-desaturase, Δ5-desaturase and Δ4-desaturase or ii) Δ5-desaturase, Δ4-desaturase and Δ8-desaturase is capable of using an acyl-CoA substrate. In an embodiment, a Δ12-desaturase and/or an ω3-desaturase is capable of using an acyl-CoA substrate. The acyl-CoA substrate is preferably an ALA-CoA, ETA-CoA, DPA-CoA, ETrA-CoA, LA-CoA, GLA-CoA, or ARA-CoA.

In an embodiment, mature, harvested seed of the plant has a DHA content of at least about 28 mg per gram seed, preferably at least about 32 mg per gram seed, at least about 36 mg per gram seed, at least about 40 mg per gram seed, more preferably at least about 44 mg per gram seed or at least about 48 mg per gram seed. The maximum DHA content may be about 80 to about 100 mg per gram seed, or about 80 mg or about 100 mg per gram seed.

In a further aspect, the present invention provides a Brassica napus, B. juncea or Camelina sativa plant which is capable of producing seed comprising DHA, wherein mature, harvested seed of the plant has a DHA content of at least about 28 mg per gram seed, preferably at least about 32 mg per gram seed, at least about 36 mg per gram seed, at least about 40 mg per gram seed, more preferably at least about 44 mg per gram seed or at least about 48 mg per gram seed. The maximum DHA content may be about 80 to about 100 mg per gram seed, or about 80 mg or about 100 mg per gram seed.

In another aspect, the present invention provides plant cell of a plant of the invention comprising the exogenous polynucleotides.

Also provided is a plant part, preferably a seed, which has one or more of the following features
i) is from a plant of the invention,
ii) comprises lipid as defined herein,
iii) can be used in a process of the invention,
iv) comprises a genetic construct of the invention, or
v) comprises a set of exogenous polynucleotides as defined herein.

In yet another aspect, the present invention provides mature, harvested Brassica napus, B. juncea or Camelina sativa seed comprising DHA and a moisture content of between about 4% and about 15% by weight, wherein the DHA content of the seed at least about 28 mg per gram seed, preferably at least about 32 mg per gram seed, at least about 36 mg per gram seed, at least about 40 mg per gram seed, more preferably at least about 44 mg per gram seed or at least about 48 mg per gram seed. The maximum DHA content may be about 80 to about 100 mg per gram seed, or about 80 mg or about 100 mg per gram seed.

In an embodiment, the cell of the invention, the transgenic organism of the invention, the oilseed plant of the invention, the Brassica napus, B. juncea or Camelina sativa plant of the invention, the plant part of the invention, or the seed of the invention, which can be used to produce extracted lipid comprising one or more or all of the features defined herein.

In yet a further aspect, the present invention provides a method of producing a cell of the invention, the method comprising
a) introducing into the cell, preferably a cell which is not capable of synthesising a LC-PUFA, the gene construct of the invention, the isolated and/or exogenous polynucleotide of the invention, the vector or genetic construct of the invention, one or more of the combinations of exogenous polynucleotides defined herein,
b) optionally, expressing the genes or polynucleotide(s) in the cell;
c) optionally, analysing the fatty acid composition of the cell, and
d) optionally, selecting a cell which express the genes or polynucleotide(s).

In an embodiment, the lipid in the cell has one or more of the features defined herein.

In another embodiment, the gene construct, the isolated and/or exogenous polynucleotide, the vector, the genetic construct or combinations of exogenous polynucleotides, become stably integrated into the genome of the cell.

In a further embodiment, the cell is a plant cell, and the method further comprises the step of regenerating a transformed plant from the cell of step a).

In another embodiment, the genes and/or exogenous polynucleotide(s) are expressed transiently in the cell.

Also provided is a cell produced using a method of the invention.

In another aspect, the present invention provides a method of producing seed, the method comprising,
a) growing a plant of the invention, or a plant which produces a part as defined herein, preferably in a field as part of a population of at least 1000 such plants or in an area of at least 1 hectare planted at a standard planting density.
b) harvesting seed from the plant or plants, and
c) optionally, extracting lipid from the seed, preferably to produce oil with a total DHA yield of at least 60 kg DHA/hectare.

In an embodiment, the plant, plant cell, plant part or seed of the invention has one or more of the following features
i) the oil is as defined herein,
ii) the plant part or seed is capable of being used in a process of the invention,
iii) the exogenous polynucleotides are comprised in a genetic construct of the invention,
iv) the exogenous polynucleotides comprise an exogenous polynucleotide of the invention,
v) the plant cell is a cell of the invention, and
vi) the seed was produced according to the method of the invention.

In another aspect, the present invention provides a method of producing one or more fatty acid desaturases and/or fatty acid elongases, or one or more fatty acid desaturases and one or more fatty acid elongases, the method comprising expressing in a cell or cell free expression system the gene construct of the invention, the isolated and/or exogenous polynucleotide of the invention, the vector or genetic construct of the invention, one or more of the combinations of exogenous polynucleotides defined herein, preferably in a developing oilseed in an oilseed plant in the field.

In a further aspect, the present invention provides lipid, or oil, produced by, or obtained from, using the process of the invention, the cell of the invention, the transgenic organism of the invention, the oilseed plant of the invention, the Brassica napus, B. juncea or Camelina sativa plant of the invention, the plant part of the invention, the seed of the invention, or the plant, plant cell, plant part or seed of the invention.

In an embodiment, the lipid or oil is obtained by extraction of oil from an oilseed. Examples of oil from oilseeds include, but are not limited to, canola oil (Brassica napus. Brassica rapa ssp.), mustard oil (Brassica juncea), other Brassica oil, sunflower oil (Helianthus annus), linseed oil (Linum usitatissimum), soybean oil (Glycine max), safflower oil (Carthamus tinctorius), corn oil (Zea mays), tobacco oil (Nicotiana tabacum), peanut oil (Arachis hypogaea), palm oil, cottonseed oil (Gossypium hirsutum), coconut oil (Cocos nucifera), avocado oil (Persea americana), olive oil (Olea europaea), cashew oil (Anacardium occidentale), macadamia oil (Macadamia intergrifolia), almond oil (Prunus amygdalus) or Arabidopsis seed oil (Arabidopsis thaliana).

In a further aspect, the present invention provides fatty acid produced by, or obtained from, using the process of the invention, the cell of the invention, the transgenic organism of the invention, the oilseed plant of the invention, the Brassica napus, B. juncea or Camelina sativa plant of the invention, the plant part of the invention, the seed of the invention, or the plant, plant cell, plant part or seed of the invention. Preferably the fatty acid is DHA. The fatty acid may be in a mixture of fatty acids having a fatty acid composition as described herein. In an embodiment, the fatty acid is non-esterified.

Also provided is seedmeal obtained from seed of the invention. Preferred seedmeal includes, but not necessarily limited to, Brassica napus, B. juncea, Cameina sativa or Glycine max seedmeal. In an embodiment, the seedmeal comprises an exogenous polynucleotide(s) and/or genentic constructs as defined herein.

In another aspect, the present invention provides a composition comprising one or more of a lipid or oil of the invention, the fatty acid of the invention, the genetic construct of the invention, the isolated and/or exogenous polynucleotide of the invention, the vector or genetic construct of the invention, the cell according of the invention, the transgenic organism of the invention, the oilseed plant of the invention, the Brassica napus, B. juncea or Camelina sativa plant of the invention, the plant part of the invention, the seed of the invention, the plant, plant cell, plant part or seed of the invention, or the seedmeal of the invention. In embodiments, the composition comprises a carrier suitable for pharmaceutical, food or agricultural use, a seed treatment compound, a fertiliser, another food or feed ingredient, or added protein or vitamins.

Also provided is feedstuffs, cosmetics or chemicals comprising one or more of the lipid or oil of the invention, the fatty acid of the invention, the genetic construct of the invention, the isolated and/or exogenous polynucleotide of the invention, the vector or genetic construct of the invention, the cell according of the invention, the transgenic organism of the invention, the oilseed plant of the invention, the Brassica napus, B. juncea or Camelina sativa plant of the invention, the plant part of the invention, the seed of the invention, the plant, plant cell, plant part or seed of the invention, the seedmeal of the invention, or the composition of the invention.

In another aspect, the present invention provides a method of producing a feedstuff, the method comprising mixing one or more of the lipid or oil of the invention, the fatty acid of the invention, the genetic construct of the invention, the isolated and/or exogenous polynucleotide of the invention, the vector or genetic construct of the invention, the cell according of the invention, the transgenic organism of the invention, the oilseed plant of the invention, the Brassica napus, B. juncea or Camelina sativa plant of the invention, the plant part of the invention, the seed of the invention, the plant, plant cell, plant part or seed of the invention, the seedmeal of the invention, or the composition of the invention, with at least one other food ingredient.

In another aspect, the present invention provides a method of treating or preventing a condition which would benefit from a PUFA, the method comprising administering to a subject one or more of the lipid or oil of the invention, the fatty acid of the invention, the genetic construct of the invention, the isolated and/or exogenous polynucleotide of the invention, the vector or genetic construct of the invention, the cell according of the invention, the transgenic organism of the invention, the oilseed plant of the invention, the Brassica napus, B. juncea or Camelina sativa plant of the invention, the plant part of the invention, the seed of the invention, the plant, plant cell, plant part or seed of the invention, the seedmeal of the invention, the composition of the invention, or the feedstuff of the invention.

Examples of conditions which would benefit from a PUFA include, but are not limited to, cardiac arrhythmia's, angioplasty, inflammation, asthma, psoriasis, osteoporosis, kidney stones, AIDS, multiple sclerosis, rheumatoid arthritis, Crohn's disease, schizophrenia, cancer, foetal alcohol syndrome, attention deficient hyperactivity disorder, cystic fibrosis, phenylketonuria, unipolar depression, aggressive hostility, adrenoleukodystophy, coronary heart disease, hypertension, diabetes, obesity, Alzheimer's disease, chronic obstructive pulmonary disease, ulcerative colitis, restenosis after angioplasty, eczema, high blood pressure, platelet aggregation, gastrointestinal bleeding, endometriosis, premenstrual syndrome, myalgic encephalomyelitis, chronic fatigue after viral infections or an ocular disease.

Also provided is the use of one or more of the lipid or oil of the invention, the fatty acid of the invention, the genetic construct of the invention, the isolated and/or exogenous polynucleotide of the invention, the vector or genetic construct of the invention, the cell according of the invention, the transgenic organism of the invention, the oilseed plant of the invention, the Brassica napus, B. juncea or Camelina sativa plant of the invention, the plant part of the invention, the seed of the invention, the plant, plant cell, plant part or seed of the invention, the seedmeal of the invention, the composition of the invention, or the feedstuff of the invention for the manufacture of a medicament for treating or preventing a condition which would benefit from a PUFA.

The production of the medicament may comprise mixing the oil of the invention with a pharmaceutically acceptable carrier, for treatment of a condition as described herein.

The method may comprise firstly purifying the oil and/or transesterification, and/or fractionation of the oil to increase the level of DHA. In a particular embodiment, the method comprises treating the lipid or oil such as canola oil to convert the fatty acids in the oil to alkyl esters such as methyl or ethyl esters. Further treatment such as fractionation or distillation may be applied to enrich the lipid or oil for the DHA. In a preferred embodiment, the medicament comprises ethyl esters of DHA. In an even more preferred embodiment, the level of ethyl esters of DHA in the medicament is between 30% and 50% The medicament may further comprise ethyl esters of EPA, such as between 30% and 50% of the total fatty acid content in the medicament. Such medicaments are suitable for administration to human or animal subjects for treatment of medical conditions as described herein.

In another aspect, the present invention provides a method of trading seed, comprising obtaining seed of the invention, and trading the obtained seed for pecuniary gain.

In an embodiment, obtaining the seed comprises cultivating plants of the invention and/or harvesting the seed from the plants.

In another embodiment, obtaining the seed further comprises placing the seed in a container and/or storing the seed.

In a further embodiment, obtaining the seed further comprises transporting the seed to a different location.

In yet another embodiment, the method further comprises transporting the seed to a different location after the seed is traded.

In a further embodiment, the trading is conducted using electronic means such as a computer.

In yet a further aspect, the present invention provides a process of producing bins of seed comprising:
a) swathing, windrowing and/or or reaping above-ground parts of plants comprising seed of the invention,
b) threshing and/or winnowing the parts of the plants to separate the seed from the remainder of the plant parts, and
c) sifting and/or sorting the seed separated in step b), and loading the sifted and/or sorted seed into bins, thereby producing bins of seed.

In an embodiment, where relevant, the lipid or oil, preferably seedoil, of, or useful for, the invention has fatty levels about those provided in a Table in the Examples section, such as seed 14 of Table 16.

Any embodiment herein shall be taken to apply mutatis mutandis to any other embodiment unless specifically stated otherwise.

The present invention is not to be limited in scope by the specific embodiments described herein, which are intended for the purpose of exemplification only.

Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Aerobic DHA biosynthesis pathways.

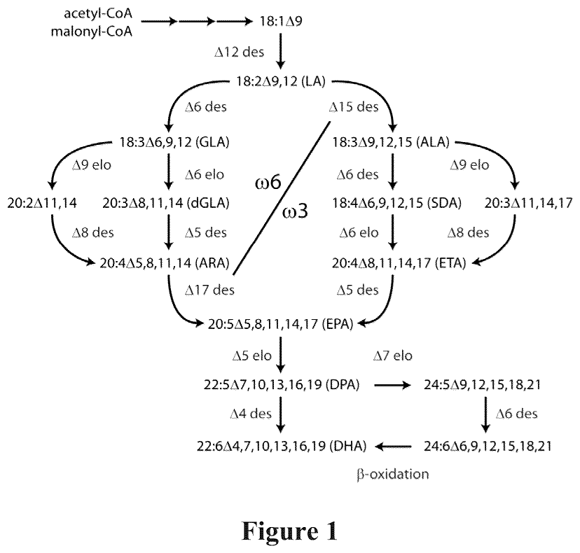

FIG. 2. Map of the T-DNA insertion region between the left and right borders of pJP3416-GA7. RB denotes right border; LB, left border; TER, transcription terminator/polyadenylation region: PRO, promoter; Coding regions are indicated above the arrows, promoters and terminators below the arrows. Micpu-Δ6D, Micromonas pusilla Δ6-desaturase; Pyrco-Δ6E, Pyramimonas cordata Δ6-elongase; Pavsa-Δ5D, Pavlova salina Δ5-desaturase; Picpa-ω3D, Pichia pastoris ω3-desaturase: Pavsa-Δ4D, P. salina Δ4-desaturase; Lackl-Δ12D, Lachancea kluyveri Δ12-desaturase; Pyrco-Δ5E, Pyramimonas cordata Δ5-elongase. NOS denotes the Agrobacterium tumefaciens nopaline synthase transcription terminator/polyadenylation region; FP1, Brassica napus truncated napin promoter; FAE1, Arabidopsis thaliana FAE1 promoter; Lectin, Glycine max lectin transcription terminator/polyadenylation region: Cnl1 and Cn12 denotes the Linum usitatissimum conlinin1 or conlinin2 promoter or terminator. MAR denotes the Rb7 matrix attachment region from Nicotiana tabacum.

FIG. 3. Map of the T-DNA insertion region between the left and right borders of pJP3404. Labels are as in FIG. 2.

FIG. 4. Map of the insertion region between the left and right borders of pJP3367. Labels are as in FIG. 2.

FIG. 5. DHA levels as a percentage of total fatty acids in seed lipid from multiple independent transgenic Arabidopsis thaliana seeds in both the T₂ and T₃ generations. The bracketed T₂ events were taken to T₃. Events from both the Columbia and fad2 mutant A. thaliana backgrounds are shown.

FIG. 6. Oil content (w/w) vs. DHA content, as a percentage of total fatty acid content of lipid from transgenic Arabidopsis thaliana seeds.

FIG. 7. Representative RT-PCR gel showing the low expression of the Δ6-desaturase gene relative to the other transgenes in the T-DNA of B. napus embryos transformed using pJP3416-GA7. Lanes from the left show RT-PCR products: 1, DNA size markers; lane 2, Δ12 desaturase: lane 3, ω3-desaturase: lane 4, Δ6-desaturase (low expression); lane 5, Δ6-elongase; lane 6. Δ5-desaturase: lane 7. Δ5-elongase; lane 8, Δ4-desaturase.

FIG. 8. Percentage of ALA plotted against percentage of oleic acid, each as a percentage of total fatty acids in lipid obtained from transgenic 35S:LEC2 Brassica napus somatic embryos.

FIG. 9. Positional distribution analysis by NMR on A) Tuna oil and, B) transgenic DHA Arabidopsis seed oil. The peaks labelled ‘DHA-alpha’ represent the amount of DHA present at the sn-1 and sn-3 positions of TAG (with no positional preference this would equal 660% of total DHA) whilst the peaks labelled ‘DHA-beta’ represent the amount of DHA present at the sn-2 position of TAG (with no preference this would equal 33% of DHA).

FIG. 10. LC-MS analysis of major DHA-containing triacylglycerol species in transgenic A. thaliana developing (grey) and mature (black) seeds. The number following the DHA denotes the total number of carbon atoms and total number of double bonds in the other two fatty acids. Therefore DHA/34:1 can also be designated TAG 56:7, etc.

FIG. 11. Map of the T-DNA insertion region between the left and right borders of pORE04+11ABGBEC_Cowpea_EPA_insert. Labels are as in FIG. 2; SSU, Arabidopsis thaliana rubisco small subunit promoter.

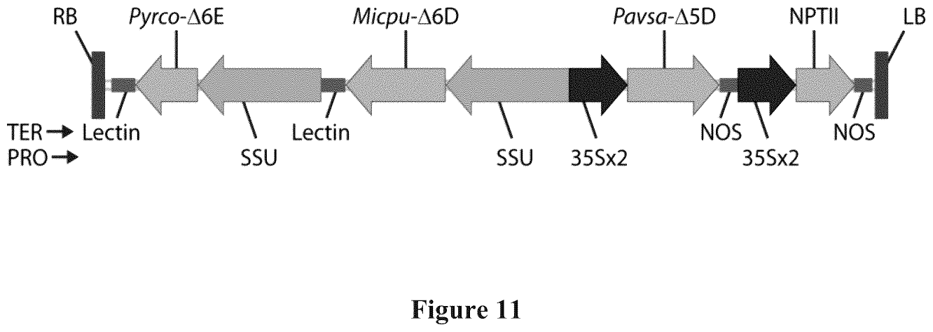

FIG. 12. Map of the binary vector pJP3364 showing the NotI restriction site into which the candidate Δ12-desaturases were cloned.

FIG. 13. BoxPlot generated using SigmaPlot showing the percentage of fatty acid 20:4ω6 (ARA) in seed lipid of Arabidopsis T2 seed populations transformed with pFN045-pFN050. The boundary of each box closest to zero indicates the 25th percentile, a line within each box marks the median, and the boundary of each box farthest from zero indicates the 75th percentile. Error bars shown above and below each box indicate the 90th and 10th percentiles.

FIG. 14. Average level of ARA as a percentage of the total fatty acid content in seed lipid of Arabidopsis T2 seed transformed with pFN045-pFN050.

FIG. 15. BoxPlot showing the percentage of fatty acid 20:20ω6 (EDA) in seed lipid of Arabidopsis T2 seed populations transformed with pFN045-pFN050. The BoxPlot represents values as described in FIG. 13.

FIG. 16. BoxPlot showing the percentage of ARA in seed lipid of Arabidopsis T4 seed populations transformed with pFN045-pFN050. The BoxPlot represents values as described in FIG. 13.

FIG. 17. Average level of ARA as a percentage of the total fatty acid content in seed lipid of Arabidopsis T4 seed populations transformed with pFN045-pFN050.

FIG. 18. BoxPlot showing the percentage of EDA in seed lipid of Arabidopsis T4 seed populations transformed with pFN045-pFN050. The BoxPlot represents values as described in FIG. 13.

FIG. 19. (A) Basic phytosterol structure with ring and side chain numbering. (B) Chemical structures of some of the phytosterols.

FIG. 20. Phylogenetic tree of known LPAATs.

FIG. 21. The various acyl exchange enzymes which transfer fatty acids between PC, CoA pools, and TAG pools. Adapted from Singh et al. (2005).

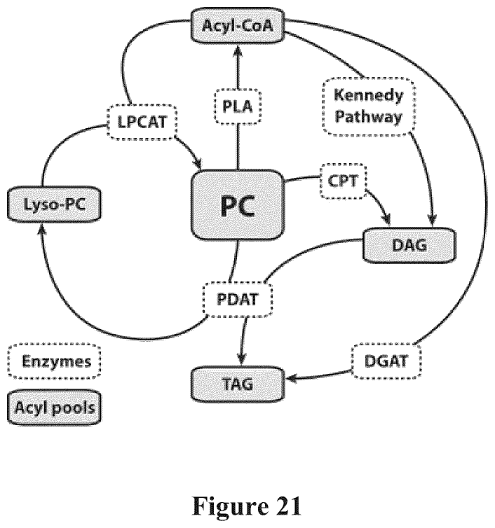

### KEY TO THE SEQUENCE LISTING

SEQ ID NO:1—pJP3416-GA7 nucleotide sequence.

SEQ ID NO:2—pGA7— mod_B nucleotide sequence.

SEQ ID NO:3—pGA7— mod_C nucleotide sequence.

SEQ ID NO:4—pGA7— mod_D nucleotide sequence.

SEQ ID NO:5—pGA7— mod_E nucleotide sequence.

SEQ ID NO:6—pGA7— mod_F nucleotide sequence.

SEQ ID NO:7—pGA7— mod_G nucleotide sequence.

SEQ ID NO:8—pORE04+11ABGBEC_Cowpea_EPA_insert nucleotide sequence.

SEQ ID NO:9—Codon-optimized open reading frame for expression of Lachancea kluyveri Δ12 desaturase in plants.

SEQ ID NO:10 —Lachancea kluyveri Δ12-desaturase.

SEQ ID NO:11—Codon-optimized open reading frame for expression of Pichia pastoris e3 desaturase in plants.

SEQ ID NO:12 —Pichia pastoris ω3 desaturase.

SEQ ID NO:13—Open reading frame encoding Micromonas pusilla Δ6-desaturase.

SEQ ID NO:14—Codon-optimized open reading frame for expression of Micromonas pusilla Δ6-desaturase in plants (version 1).

SEQ ID NO:15—Codon-optimized open reading frame for expression of Micromonas pusilla Δ6-desaturase in plants (version 2).

SEQ ID NO:16 —Micromonas pusilla Δ6-desaturase.

SEQ ID NO:17—Open reading frame encoding Ostreococcus lucimarinus Δ6-desaturase.

SEQ ID NO:18—Codon-optimized open reading frame for expression of Ostreococcus lucimarinus Δ6-desaturase in plants.

SEQ ID NO:19 —Ostreococcus lucimarinus Δ6-desaturase.

SEQ ID NO:20 —Ostreococcus tauri Δ6-desaturase.

SEQ ID NO:21—Open reading frame encoding Pyramimonas cordata Δ6-elongase.

SEQ ID NO:22—Codon-optimized open reading frame for expression of Pyramimonas cordata Δ6-elongase in plants (truncated at 3′ end and encoding functional elongase) (version 1).

SEQ ID NO:23—Codon-optimized open reading frame for expression of Pyramimonas cordata Δ6-elongase in plants (truncated at 3′ end and encoding functional elongase) (version 2).

SEQ ID NO:24—Codon-optimized open reading frame for expression of Pyramimonas cordata Δ6-elongase in plants (truncated at 3′ end and encoding functional elongase) (version 3).

SEQ ID NO:25 —Pyramimonas cordata Δ6-elongase.

SEQ ID NO:26—Truncated Pyramimonas cordata Δ6-elongase.

SEQ ID NO:27—Open reading frame encoding Pavlova salina Δ5-desaturase.

SEQ ID NO:28—Codon-optimized open reading frame for expression of Pavlova salina Δ5-desaturase in plants (version 1).

SEQ ID NO:29—Codon-optimized open reading frame for expression of Pavlova salina Δ5-desaturase in plants (version 2).

SEQ ID NO:30 —Pavlova salina Δ5-desaturase.

SEQ ID NO:31—Open reading frame encoding Pyramimonas cordata Δ5-desaturase.

SEQ ID NO:32 —Pyramimonas cordata Δ5-desaturase.

SEQ ID NO:33—Open reading frame encoding Pyramimonas cordata Δ5-elongase.

SEQ ID NO:34—Codon-optimized open reading frame for expression of Pyramimonas cordata Δ5-elongase in plants (version 1).

SEQ ID NO:35—Codon-optimized open reading frame for expression of Pyramimonas cordata Δ5-elongase in plants (version 2).

SEQ ID NO:36—Codon-optimized open reading frame for expression of Pyramimonas cordata Δ5-elongase in plants (version 3).

SEQ ID NO:37 —Pyramimonas cordata Δ5-elongase.

SEQ ID NO:38—Open reading frame encoding Pavlova salina Δ4-desaturase.

SEQ ID NO:39—Codon-optimized open reading frame for expression of Pavlova salina Δ4-desaturase in plants (version 1).

SEQ ID NO:40—Codon-optimized open reading frame for expression of Pavlova salina Δ4-desaturase in plants (version 2).

SEQ ID NO:41 —Pavlova salina Δ4-desaturase.

SEQ ID NO:42—Open reading frame encoding Isochrysis galbana Δ9-elongase.

SEQ ID NO:43 —Isochrysis galbana Δ9-elongase.

SEQ ID NO:44—Open reading frame encoding Emiliania huxleyi CCMP1516 Δ9-elongase.

SEQ ID NO:45—Codon-optimized open reading frame for expression of Emiliania huxleyi Δ9-elongase in plants.

SEQ ID NO:46 —Emiliania huxleyi CCMP1516 Δ9-elongase.

SEQ ID NO:47—Open reading frame encoding Pavlova pinguis Δ9-elongase.

SEQ ID NO:48 —Pavlova pinguis Δ9-elongase.

SEQ ID NO:49—Open reading frame encoding Pavlova salina Δ9-elongase.

SEQ ID NO:50 —Pavlova salina Δ9-elongase.

SEQ ID NO:51—Open reading frame encoding Pavlova salina Δ8-desaturase.

SEQ ID NO:52 —Pavlova salina Δ8-desaturase.

SEQ ID NO:53—P19 viral suppressor.

SEQ ID NO:54—V2 viral suppressor.

SEQ ID NO:55—P38 viral suppressor.

SEQ ID NO:56—Pe-P0 viral suppressor.

SEQ ID NO:57—RPV-P0 viral suppressor.

SEQ ID NO:58—Open reading frame encoding P19 viral suppressor.

SEQ ID NO:59—Open reading frame encoding V2 viral suppressor.

SEQ ID NO:60—Open reading frame encoding P38 viral suppressor.

SEQ ID NO:61—Open reading frame encoding Pe-P0 viral suppressor.

SEQ ID NO:62—Open reading frame encoding RPV-P0 viral suppressor.

SEQ ID NO: 63 —Arabidopsis thaliana LPAAT2.

SEQ ID NO: 64 —Limnanthes alba LPAAT.

SEQ ID NO: 65 —Saccharomyces cerevisiae LPAAT.

SEQ ID NO: 66 —Micromonas pusilla LPAAT.

SEQ ID NO: 67 —Mortierella alpina LPAAT.

SEQ ID NO: 68 —Braccisa napus LPAAT.

SEQ ID NO: 69 —Brassica napus LPAAT.

SEQ ID NO: 70 —Phytophthora infestans ω3 desaturase.

SEQ ID NO: 71 —Thalassiosira pseudonana ω3 desaturase.

SEQ ID NO: 72 —Pythium irregulare ω3 desaturase.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, fatty acid synthesis, transgenic plants, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984). J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), F. M. Ausubel et al. (editors), Current Protocols in Molecular Biology, Greene Pub. Associates and Wiley-Interscience (1988, including all updates until present), Ed Harlow and David Lane (editors), Antibodies: A Laboratory Manual, Cold Spring Harbour Laboratory, (1988), and J. E. Coligan et al. (editors), Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, more preferably +/−1% of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, the terms “extracted plant lipid” and “isolated plant lipid” refer to a lipid composition which has been extracted from, for example by crushing, a plant or part thereof such as seed. The extracted lipid can be a relatively crude composition obtained by, for example, crushing a plant seed, or a more purified composition where most, if not all, of one or more or each of the water, nucleic acids, proteins and carbohydrates derived from the plant material have been removed. Examples of purification methods are described below. In an embodiment, the extracted or isolated plant lipid comprises at least about 60%, at least about 70%, at least about 80%, at least about 90%, or at least about 95% (w/w) lipid by weight of the composition. The lipid may be solid or liquid at room temperature, when liquid it is considered to be an oil. In an embodiment, extracted lipid of the invention has not been blended with another lipid such as DHA not produced by another source (for example, DHA from fish oil). In an embodiment, following extraction the ratio of one or more or all of, oleic acid to DHA, palmitic acid to DHA, linoleic acid to DHA, and total ω6 fatty acids: total ω3 fatty acids, has not been significantly altered (for example, no greater than a 10% or 5% alteration) when compared to the ratio in the intact seed or cell. In an another embodiment, the extracted plant lipid has not been exposed to a procedure, such as hydrogenation or fractionation, which may alter the ratio of one or more or all of, oleic acid to DHA, palmitic acid to DHA, linoleic acid to DHA, and total ω6 fatty acids: total ω3 fatty acids, when compared to the ratio in the intact seed or cell. When the extracted plant lipid of the invention is comprised in an oil, the oil may further comprise non-fatty acid molecules such as sterols.

As used herein, the terms “extracted plant oil” and “isolated plant oil” refer to a substance or composition comprising extracted plant lipid or isolated plant lipid and which is a liquid at room temperature. The oil is obtained from a plant or part thereof such as seed. The extracted or isolated oil can be a relatively crude composition obtained by, for example, crushing a plant seed, or a more purified composition where most, if not all, of one or more or each of the water, nucleic acids, proteins and carbohydrates derived from the plant material have been removed. The composition may comprise other components which may be lipid or non-lipid. In an embodiment, the oil composition comprises at least about 60%, at least about 70%, at least about 80%, at least about 90%, or at least about 95% (w/w) extracted plant lipid. In an embodiment, extracted oil of the invention has not been blended with another oil such as DHA not produced by another source (for example, DHA from fish oil). In an embodiment, following extraction, the ratio of one or more or all of, oleic acid to DHA, palmitic acid to DHA, linoleic acid to DHA, and total ω6 fatty acids: total ω3 fatty acids, has not been significantly altered (for example, no greater than a 10% or 5% alteration) when compared to the ratio in the intact seed or cell. In an another embodiment, the extracted plant oil has not been exposed to a procedure, such as hydrogenation or fractionation, which may alter the ratio of one or more or all of, oleic acid to DHA, palmitic acid to DHA, linoleic acid to DHA, and total ω6 fatty acids: total ω3 fatty acids, when compared to the ratio in the intact seed or cell. Extracted plant oil of the invention may comprise non-fatty acid molecules such as sterols.

As used herein, an “oil” is a composition comprising predominantly lipid and which is a liquid at room temperature. For instance, oil of the invention preferably comprises at least 75%, at least 80%, at least 85% or at least 90% lipid by weight. Typically, a purified oil comprises at least 90% triacylglycerols (TAG) by weight of the lipid in the oil. Minor components of an oil such as diacylglycerols (DAG), free fatty acids (FFA), phospholipid and sterols may be present as described herein.

As used herein, the term “fatty acid” refers to a carboxylic acid (or organic acid), often with a long aliphatic tail, either saturated or unsaturated. Typically fatty acids have a carbon-carbon bonded chain of at least 8 carbon atoms in length, more preferably at least 12 carbons in length. Most naturally occurring fatty acids have an even number of carbon atoms because their biosynthesis involves acetate which has two carbon atoms. The fatly acids may be in a free state (non-esterified) or in an esterified form such as part of a triglyceride, diacylglyceride, monoacylglyceride, acyl-CoA (thio-ester) bound or other bound form. The fatty acid may be esterified as a phospholipid such as a phosphatidylcholine, phosphatidylethanolamine, phosphatidylserine, phosphatidylglycerol, phosphatidylinositol or diphosphatidylglycerol forms.

“Saturated fatly acids” do not contain any double bonds or other functional groups along the chain. The term “saturated” refers to hydrogen, in that all carbons (apart from the carboxylic acid [—COOH] group) contain as many hydrogens as possible. In other words, the omega (ω) end contains 3 hydrogens (CH₃—) and each carbon within the chain contains 2 hydrogens (—CH₂—).

“Unsaturated fatty acids” are of similar form to saturated fatty acids, except that one or more alkene functional groups exist along the chain, with each alkene substituting a singly-bonded “—CH₂—CH₂—” part of the chain with a doubly-bonded “—CH═CH—” portion (that is, a carbon double bonded to another carbon). The two next carbon atoms in the chain that are bound to either side of the double bond can occur in a cis or trans configuration.

As used herein, the term “monounsaturated fatty acid” refers to a fatty acid which comprises at least 12 carbon atoms in its carbon chain and only one alkene group (carbon-carbon double bond) in the chain. As used herein, the terms “polyunsaturated fatty acid” or “PUFA” refer to a fatty acid which comprises at least 12 carbon atoms in its carbon chain and at least two alkene groups (carbon-carbon double bonds).

As used herein, the terms “long-chain polyunsaturated fatty acid” and “LC-PUFA” refer to a fatty acid which comprises at least 20 carbon atoms in its carbon chain and at least two carbon-carbon double bonds, and hence include VLC-PUFAs.

As used herein, the terms “very long-chain polyunsaturated fatty acid” and “VLC-PUFA” refer to a fatty acid which comprises at least 22 carbon atoms in its carbon chain and at least three carbon-carbon double bonds. Ordinarily, the number of carbon atoms in the carbon chain of the fatty acids refers to an unbranched carbon chain. If the carbon chain is branched, the number of carbon atoms excludes those in sidegroups. In one embodiment, the long-chain polyunsaturated fatty acid is an ω3 fatty acid, that is, having a desaturation (carbon-carbon double bond) in the third carbon-carbon bond from the methyl end of the fatty acid. In another embodiment, the long-chain polyunsaturated fatty acid is an ω6 fatty acid, that is, having a desaturation (carbon-carbon double bond) in the sixth carbon-carbon bond from the methyl end of the fatty acid. In a further embodiment, the long-chain polyunsaturated fatty acid is selected from the group consisting of; arachidonic acid (ARA, 20:4Δ5,8,11,14; ω6), eicosatetraenoic acid (ETA, 20:4Δ8,11,14,17, ω3), eicosapentaenoic acid (EPA, 20:5Δ5,8,11,14,17; ω3), docosapentaenoic acid (DPA, 22:5Δ7,10,13,16,19, ω3), or docosahexaenoic acid (DHA, 22:6Δ4,7,10,13,16,19, ω3). The LC-PUFA may also be dihomo-γ-linoleic acid (DGLA) or eicosatrienoic acid (ETrA, 20:3Δ11,14,17, ω3). It would readily be apparent that the LC-PUFA that is produced according to the invention may be a mixture of any or all of the above and may include other LC-PUFA or derivatives of any of these LC-PUFA. In a preferred embodiment, the ω3 fatty acids are at least DHA, preferably. DPA and DHA, or EPA, DPA and DHA.

Furthermore, as used herein the terms “long-chain polyunsaturated fatty acid” and “very long-chain polyunsaturated fatty acid” refer to the fatty acid being in a free state (non-esterified) or in an esterified form such as part of a triglyceride, diacylglyceride, monoacylglyceride, acyl-CoA bound or other bound form. The fatty acid may be esterified as a phospholipid such as a phosphatidylcholine (PC), phosphatidylethanolamine, phosphatidylserine, phosphatidylglycerol, phosphatidylinositol or diphosphatidylglycerol forms. Thus, the LC-PUFA may be present as a mixture of forms in the lipid of a cell or a purified oil or lipid extracted from cells, tissues or organisms. In preferred embodiments, the invention provides oil comprising at least 75% or at least 85% triacylglycerols, with the remainder present as other forms of lipid such as those mentioned, with at least said triacylglycerols comprising the LC-PUFA. The oil may subsequently be further purified or treated, for example by hydrolysis with a strong base to release the free fatty acids, or by distillation or the like.

As used herein, “total ω6 fatty acids” or “total ω6 fatty acid content” or the like refers to the sum of all the ω6 fatty acids, esterified and non-esterified, in the extracted lipid, oil, recombinant cell, plant part or seed, as the context determines, expressed as a percentage of the total fatty acid content. These ω6 fatty acids include (if present) LA, GLA, DGLA, ARA, EDA and ω6-DPA, and exclude any ω3 fatty acids and monounsaturated fatty acids.

As used herein, “new ω6 fatty acids” or “new ω6 fatty acid content” or the like refers to the sum of all the ω6 fatty acids excluding LA, esterified and non-esterified, in the extracted lipid, oil, recombinant cell, plant part or seed, as the context determines, expressed as a percentage of the total fatty acid content. These new ω6 fatty acids are the fatty acids that are produced in the cells, plants, plant parts and seeds of the invention by the expression of the genetic constructs (exogenous polynucleotides) introduced into the cells, and include (if present) GLA, DGLA, ARA, EDA and ω6-DPA, but exclude LA and any ω3 fatty acids and monounsaturated fatty acids. Exemplary total ω6 fatty acid contents and new ω6 fatty acid contents are determined by conversion of fatty acids in a sample to FAME and analysis by GC, as described in Example 1.

As used herein, “total ω3 fatty acids” or “total ω3 fatty acid content” or the like refers to the sum of all the ω3 fatty acids, esterified and non-esterified, in the extracted lipid, oil, recombinant cell, plant part or seed, as the context determines, expressed as a percentage of the total fatty acid content. These ω3 fatty acids include (if present) ALA, SDA, ETrA, ETA, EPA, DPA and DHA, and exclude any 06 fatty acids and monounsaturated fatty acids.

As used herein, “new ω3 fatty acids” or “new ω3 fatty acid content” or the like refers to the sum of all the ω3 fatty acids excluding ALA, esterified and non-esterified, in the extracted lipid, oil, recombinant cell, plant part or seed, as the context determines, expressed as a percentage of the total fatty acid content. These new ω3 fatty acids are the fatty acids that are produced in the cells, plants, plant parts and seeds of the invention by the expression of the genetic constructs (exogenous polynucleotides) introduced into the cells, and include (if present) SDA, ETrA, ETA, EPA, DPA and DHA, but exclude ALA and any 06 fatty acids and monounsaturated fatty acids. Exemplary total ω3 fatty acid contents and new ω3 fatty acid contents are determined by conversion of fatty acids in a sample to FAME and analysis by GC, as described in Example 1.

The desaturase, elongase and acyl transferase proteins and genes encoding them that may be used in the invention are any of those known in the art or homologues or derivatives thereof. Examples of such genes and encoded protein sizes are listed in Table 1. The desaturase enzymes that have been shown to participate in LC-PUFA biosynthesis all belong to the group of so-called “front-end” desaturases.

As used herein, the term “front-end desaturase” refers to a member of a class of enzymes that introduce a double bond between the carboxyl group and a pre-existing unsaturated part of the acyl chain of lipids, which are characterized structurally by the presence of an N-terminal cytochrome b5 domain, along with a typical fatty acid desaturase domain that includes three highly conserved histidine boxes (Napier et al., 1997).

Activity of any of the elongases or desaturases for use in the invention may be tested by expressing a gene encoding the enzyme in a cell such as, for example, a yeast cell, a plant cell or preferably in somatic embryos or transgenic plants, and determining whether the cell, embryo or plant has an increased capacity to produce LC-PUFA compared to a comparable cell, embryo or plant in which the enzyme is not expressed.

In one embodiment one or more of the desaturases and/or elongases for use in the invention can purified from a microalga, i.e. is identical in amino acid sequence to a polypeptide which can be purified from a microalga.

Whilst certain enzymes are specifically described herein as “bifunctional”, the absence of such a term does not necessarily imply that a particular enzyme does not possess an activity other than that specifically defined.

### Desaturases

As used herein, the term “desaturase” refers to an enzyme which is capable of introducing a carbon-carbon double bond into the acyl group of a fatty acid substrate which is typically in an esterified form such as, for example, acyl-CoA esters. The acyl group may be esterified to a phospholipid such as phosphatidylcholine (PC), or to acyl carrier protein (ACP), or in a preferred embodiment to CoA. Desaturases generally may be categorized into three groups accordingly. In one embodiment, the desaturase is a front-end desaturase.

As used herein, a “Δ4-desaturase” refers to a protein which performs a desaturase reaction that introduces a carbon-carbon double bond at the 4^{th} carbon-carbon bond from the carboxyl end of a fatty acid substrate. The “Δ4-desaturase” is at least capable of converting DPA to DHA. The desaturation step to produce DHA from DPA is catalysed by a Δ4-desaturase in organisms other than mammals, and a gene encoding this enzyme has been isolated from the freshwater protist species Euglena gracilis and the marine species Thraustochytrium sp. (Qiu et al., 2001; Meyer et al., 2003). In one embodiment, the Δ4-desaturase comprises amino acids having a sequence as provided in SEQ ID NO:41, or a Thraustochytrium sp. Δ4-desaturase, a biologically active fragment thereof, or an amino acid sequence which is at least 80% identical to SEQ ID NO:41.

TABLE 1
Cloned genes involved in LC-PUFA biosynthesis

| | | | | Protein size | |
|---|---|---|---|---|---|
| Enzyme | Type of organism | Species | Accession Nos. | (aa's) | References |
| Δ4- | Protist | Euglena gracilis | AY278558 | 541 | Meyer et al., 2003 |
| desaturase | | | | | |
| | Algae | Pavlova lutherii | AY332747 | 445 | Tonon et al., 2003 |
| | | Isochrysis galbana | AAV33631 | 433 | Pereira et al., 2004b |
| | | Pavlova salina | AAY15136 | 447 | Zhou et al., 2007 |
| | Thraustochytrid | Thraustochytrium aureum | AAN75707 | 515 | N/A |
| | | | AAN75708 | | |
| | | | AAN75709 | | |
| | | | AAN75710 | | |
| | | Thraustochytrium sp. | AAM09688 | 519 | Qiu et al, 2001 |
| | | ATCC21685 | | | |
| Δ5- | Mammals | Homo sapiens | AF199596 | 444 | Cho et al., 1999b |
| desaturase | | | | | Leonard et al., 2000b |
| | Nematode | Caenorhabditis elegans | AF11440, | 447 | Michaelson et al., 1998b; |
| | | | NM_069350 | | Watts and Browse, 1999b |
| | Fungi | Mortierella alpina | AF067654 | 446 | Michaelson et al., 1998a; |
| | | | | | Knutzon et al., 1998 |
| | | Pythium irregulare | AF419297 | 456 | Hong et al., 2002a |
| | | Dictyostelium discoideum | AB022097 | 467 | Saito et al., 2000 |
| | | Saprolegnia diclina | | 470 | WO02081668 |
| | Diatom | Phaeodactylum tricornutum | AY082392 | 469 | Domergue et al., 2002 |
| | Algae | Thraustochytrium sp | AF489588 | 439 | Qiu et al., 2001 |
| | | Thraustochytrium aureum | | 439 | WO02081668 |
| | | Isochrysis galbana | | 442 | WO02081668 |
| | Moss | Marchantia polymorpha | AY583465 | 484 | Kajikawa et al., 2004 |
| Δ6-desaturase | Mammals | Homo sapiens | NM_013402 | 444 | Cho et al., 1999a; |
| | | | | | Leonard et al., 2000 |
| | | Mus musculus | NM_019699 | 444 | Cho et al., 1999a |
| | Nematode | Caenorhabditis elegans | Z70271 | 443 | Napier et al., 1998 |
| | Plants | Borago officinales | U79010 | 448 | Sayanova et al., 1997 |
| | | Echium | AY055117 | | Garcia-Maroto et al., 2002 |
| | | | AY055118 | | |
| | | Primula vialii | AY234127 | 453 | Sayanova et al., 2003 |
| | | Anemone leveillei | AF536525 | 446 | Whitney et al., 2003 |
| | Mosses | Ceratodon purpureus | AJ250735 | 520 | Sperling et al., 2000 |
| | | Marchantia polymorpha | AY583463 | 481 | Kajikawa et al., 2004 |
| | | Physcomitrella patens | CAA11033 | 525 | Girke et al., 1998 |
| | Fungi | Mortierella alpina | AF110510 | 457 | Huang et al., 1999; |
| | | | AB020032 | | Sakuradani et al., 1999 |
| | | Pythium irregulare | AF419296 | 459 | Hong et al., 2002a |
| | | Mucor circinelloides | AB052086 | 467 | NCBI* |
| | | Rhizopus sp. | AY320288 | 458 | Zhang et al., 2004 |
| | | Saprolegnia diclina | | 453 | WO02081668 |
| | Diatom | Phaeodactylum tricornutum | AY082393 | 477 | Domergue et al., 2002 |
| | Bacteria | Synechocystis | L11421 | 359 | Reddy et al., 1993 |
| | Algae | Thraustochytrium aureum | | 456 | WO02081668 |
| Bifunctional | Fish | Dania rerio | AF309556 | 444 | Hastings et al., 2001 |
| Δ5/Δ6- | | | | | |
| desaturase | | | | | |
| C20 Δ8- | Algae | Euglena gracilis | AF139720 | 419 | Wallis and Browse, 1999 |
| desaturase | | | | | |
| | Plants | Boraga officinales | AAG43277 | 446 | Sperling et al., 2001 |
| Δ6-elongase | Nematode | Caenorhabditis elegans | NM_069288 | 288 | Beaudoin et al., 2000 |
| | Mosses | Physcomitrella patens | AF428243 | 290 | Zank et al., 2002 |
| | | Marchantia polymorpha | AY583464 | 290 | Kajikawa et al., 2004 |
| | Fungi | Mortierella alpina | AF206662 | 318 | Parker-Barnes et al., 2000 |
| | Algae | Pavlova lutheri** | | 501 | WO 03078639 |
| | | Thraustochytrium | AX951565 | 271 | WO 03093482 |
| | | Thraustochytrium sp** | AX214454 | 271 | WO 0159128 |
| PUFA- | Mammals | Homo sapiens | AF231981 | 299 | Leonard et al., 2000; |
| elongase | | | | | Leonard et al., 2002 |
| | | Rattus norvegicus | AB071985 | 299 | Inagaki et al., 2002 |
| | | Rattus norvegicus** | AB071986 | 267 | Inagaki et al., 2002 |
| | | Mus musculus | AF170907 | 279 | Tvrdik et al., 2000 |
| | | Mus musculus | AF170908 | 292 | Tvrdik et al., 2000 |
| | Fish | Danio rerio | AF532782 | 291 (282) | Agaba et al., 2004 |
| | | Danio rerio** | NM_199532 | 266 | Lo et al., 2003 |
| | Worm | Caenorhabditis elegans | Z68749 | 309 | Abbott et al., 1998 |
| | | | | | Beaudoin et al., 2000 |
| | Algae | Thraustochytrium aureum** | AX464802 | 272 | WO 0208401-A2 |
| | | Pavlova lutheri** | | 320 | WO 03078639 |
| Δ9-elongase | Algae | Isochrysis galbana | AF390174 | 263 | Qi et al., 2002 |
| | | Euglena gracilis | | 258 | WO 08/128241 |
| Δ5-elongase | Algae | Ostreococcus tauri | AAV67798 | 300 | Meyer et al., 2004 |
| | | Pyramimonas cordata | | 268 | WO 2010/057246 |
| | | Pavlova sp. CCMP459 | AAV33630 | 277 | Pereira et at., 2004b |
| | | Pavlova salina | AAY15135 | 302 | Robert et al., 2009 |
| | Diatom | Thalassiosira pseudonana | AAV67800 | 358 | Meyer et al., 2004 |
| | Fish | Oncorhynchus mykiss | CAM55862 | 295 | WO 06/008099 |
| | Moss | Marchantia polymorpha | BAE71129 | 348 | Kajikawa et al., 2006 |

*http://www.ncbi.nlm.nih.gov/
**Function not proven/not demonstrated

As used herein, a “Δ5-desaturase” refers to a protein which performs a desaturase reaction that introduces a carbon-carbon double bond at the 5^{th} carbon-carbon bond from the carboxyl end of a fatty acid substrate. Examples of Δ5-desaturases are listed in Ruiz-Lopez et al. (2012) and Petrie et al. (2010a) and in Table 1 herein. In one embodiment, the Δ5-desaturase comprises amino acids having a sequence as provided in SEQ ID NO:30, a biologically active fragment thereof, or an amino acid sequence which is at least 80% identical to SEQ ID NO:30. In another embodiment, the Δ5-desaturase comprises amino acids having a sequence as provided in SEQ ID NO:32, a biologically active fragment thereof, or an amino acid sequence which is at least 53% identical to SEQ ID NO:32. In another embodiment, the Δ5-<desaturase is from Thraustochytrium sp or Emiliania huxleyi.

As used herein, a “Δ6-desaturase” refers to a protein which performs a desaturase reaction that introduces a carbon-carbon double bond at the 6^{th} carbon-carbon bond from the carboxyl end of a fatty acid substrate. Examples of Δ6-desaturases are listed in Ruiz-Lopez et al. (2012) and Petrie et al. (2010a) and in Table 1 herein. Preferred Δ6-desaturases are from Micromonas pusilla, Pythium irregulare or Ostreococcus taurii.

In an embodiment, the Δ6-desaturase is further characterised by having at least two, preferably all three and preferably in a plant cell, of the following: i) greater Δ6-desaturase activity on α-linolenic acid (ALA, 18:3Δ9,12,15, ω3) than linoleic acid (LA, 18:2Δ9,12, ω6) as fatty acid substrate; ii) greater Δ6-desaturase activity on ALA-CoA as fatty acid substrate than on ALA joined to the sn-2 position of PC as fatty acid substrate; and iii) Δ8-desaturase activity on ETrA. Examples of such Δ6-desaturases are provided in Table 2.

In an embodiment the Δ6-desaturase has greater activity on an ω3 substrate than the corresponding ω6 substrate and has activity on ALA to produce octadecatetraenoic acid (stearidonic acid, SDA, 18:4Δ6,9,12, 15, ω3) with an efficiency of at least 30%, more preferably at least 40%, or most preferably at least 50% when expressed from an exogenous polynucleotide in a recombinant cell such as a plant cell, or at least 35% when expressed in a yeast cell. In one embodiment, the Δ6-desaturase has greater activity, for example, at least about a 2-fold greater Δ6-desaturase activity, on ALA than LA as fatty acid substrate. In another embodiment, the Δ6-desaturase has greater activity, for example, at least about 5 fold greater Δ6-desaturase activity or at least 10-fold greater activity, on ALA-CoA as fatty acid substrate than on ALA joined to the sn-2 position of PC as fatty acid substrate. In a further embodiment, the Δ6-desaturase has activity on both fatty acid substrates ALA-CoA and on ALA joined to the sn-2 position of PC.

TABLE 2
Desaturases demonstrated to have activity on an acyl-CoA substrate

| | Type | | | Protein | |
|---|---|---|---|---|---|
| | of | | Accession | size | |
| Enzyme | organism | Species | Nos. | (aa's) | References |
| Δ6-desaturase | Algae | Mantoniella | CAQ30479 | 449 | Hoffmann et al., 2008 |
| | | squamata | | | |
| | | Ostreococcus | AAW70159 | 456 | Domergue et al., 2005 |
| | | tauri | | | |
| | | Micromonas | EEH58637 | | Petrie et al., 2010a |
| | | pusilla | | | (SEQ ID NO: 13) |
| Δ5-desaturase | Algae | Mantoniella | CAQ30478 | 482 | Hoffmann et al., 2008 |
| | | squamata | | | |
| | Plant | Anemone | N/A | | Sayanova et al., 2007 |
| | | leveillei | | | |
| ω3-desaturase | Fungi | Pythium | FW362186.1 | 359 | Xue et al., 2012; |
| | | aphanidermatum | | | WO2008/054565 |
| | Fungi | Phytophthora | FW362214.1 | 363 | Xue et al., 2012; |
| | (oomycete) | sojae | | | WO2008/054565 |
| | Fungi | Phytophthora | FW362213.1 | 361 | Xue et al., 2012; |
| | (oomycete) | ramorum | | | WO2008/054565 |

In one embodiment, the Δ6-desaturase has no detectable Δ5-desaturase activity on ETA. In another embodiment, the Δ6-desaturase comprises amino acids having a sequence as provided in SEQ ID NO: 16, SEQ ID NO: 19 or SEQ ID NO:20, a biologically active fragment thereof, or an amino acid sequence which is at least 77% identical to SEQ ID NO:16, SEQ ID NO:19 or SEQ ID NO:20. In another embodiment, the 06-desaturase comprises amino acids having a sequence as provided in SEQ ID NO; 19 or SEQ ID NO:20, a biologically active fragment thereof, or an amino acid sequence which is at least 67% identical to one or both of SEQ ID NO: 19 or SEQ ID NO:20. The Δ6-desaturase may also have Δ8-desaturase activity.

As used herein, a “Δ8-desaturase” refers to a protein which performs a desaturase reaction that introduces a carbon-carbon double bond at the 8^{th} carbon-carbon bond from the carboxyl end of a fatty acid substrate. The Δ8-desaturase is at least capable of converting ETrA to ETA. Examples of Δ8-desaturases are listed in Table 1. In one embodiment, the Δ8-desaturase comprises amino acids having a sequence as provided in SEQ ID NO:52, a biologically active fragment thereof, or an amino acid sequence which is at least 80% identical to SEQ ID NO:52.

As used herein, an “ω3-desaturase” refers to a protein which performs a desaturase reaction that introduces a carbon-carbon double bond at the 3rd carbon-carbon bond from the methyl end of a fatty acid substrate. A ω3-desaturase therefore may convert LA to ALA and GLA to SDA (all C18 fatty acids), or DGLA to ETA and/or ARA to EPA (C20 fatty acids). Some ω3-desaturases (group I) have activity only on C18 substrates, such as plant and cyanobacterial ω3-desaturases. Such ω3-desaturases are also Δ15-desaturases. Other ω3-desaturases have activity on C20 substrates with no activity (group II) or some activity (group III) on C18 substrates. Such ω3-desaturases are also Δ17-desaturases. Preferred ω3-desaturases are group III type which convert LA to ALA, GLA to SDA. DGLA to ETA and ARA to EPA, such as the Pichia pastoris ω3-desaturase (SEQ ID NO: 12). Examples of ω3-desaturases include those described by Pereira et al. (2004a) (Saprolegnia diclina ω3-desaturase, group II). Horiguchi et al. (1998). Berberich et al. (1998) and Spychalla et al. (1997) (C. elegans ω3-desaturase, group III). In a preferred embodiment, the ω3-desaturase is a fungal ω3-desaturase. As used herein, a “fungal ω3-desaturase” refers to an ω3-desaturase which is from a fungal source, including an oomycete source, or a variant thereof whose amino acid sequence is at least 95% identical thereto. Genes encoding numerous ω3-desaturases have been isolated from fungal sources such as, for example, from Phytophthora infestans (Accession No. CAJ30870, WO2005083053; SEQ ID NO: 70), Saprolegnia diclina (Accession No. AAR20444, Pereira et al., 2004a & U.S. Pat. No. 7,211,656), Pythium irregulare (WO2008022963, Group II; SEQ ID NO: 72), Mortierella alpina (Sakuradani et al., 2005: Accession No. BAD91495; WO2006019192), Thalassiosira pseudonana (Armbrust et al., 2004; Accession No. XP_002291057; WO2005012316, SEQ ID NO: 71), Lachancea kluyveri (also known as Saccharomyces kluyveri; Oura et al., 2004; Accession No. AB118663). Xue et al. (2012) describes ω3-desaturases from the oomycetes Pythium aphanidermatum, Phytophthora sojae, and Phytophthora ramorum which were able to efficiently convert ω6 fatty acid substrates to the corresponding ω3 fatty acids, with a preference for C20 substrates, i.e. they had stronger Δ17-desaturase activity than Δ15-desaturase activity. These enzymes lacked Δ12-desaturase activity, but could use fatty acids in both acyl-CoA and phospholipid fraction as substrates.

In a more preferred embodiment, the fungal ω3-desaturase is the Pichia pastoris (also known as Komagataella pastoris) ω3-desaturase/Δ15-desaturase (Zhang et al., 2008; Accession No. EF116884; SEQ ID NO: 12), or a polypeptide which is at least 95% identical thereto.

In an embodiment, the ω3-desaturase is at least capable of converting one of ARA to EPA, DGLA to ETA, GLA to SDA, both ARA to EPA and DGLA to ETA, both ARA to EPA and GLA to SDA, or all three of these.

In one embodiment, the ω3-desaturase has Δ17-desaturase activity on a C20 fatty acid which has at least three carbon-carbon double bonds, preferably ARA. In another embodiment, the ω3-desaturase has Δ15-desaturase activity on a C18 fatty acid which has three carbon-carbon double bonds, preferably GLA. Preferably, both activities are present.

As used herein, a “Δ12-desaturase” refers to a protein which performs a desaturase reaction that introduces a carbon-carbon double bond at the 12^{th} carbon-carbon bond from the carboxyl end of a fatty acid substrate. Δ12-desaturases typically convert either oleoyl-phosphatidylcholine or oleoyl-CoA to linoleoyl-phosphatidylcholine (18:1-PC) or linoleoyl-CoA (18:1-CoA), respectively. The subclass using the PC linked substrate are referred to as phospholipid-dependent Δ12-desaturases, the latter subclass as acyl-CoA dependent Δ12-desaturases. Plant and fungal Δ12-desaturases are generally of the former sub-class, whereas animal Δ12-desaturases are of the latter subclass, for example the Δ12-desaturases encoded by genes cloned from insects by Zhou et al. (2008). Many other Δ12-desaturase sequences can be easily identified by searching sequence databases.

As used herein, a “Δ15-desaturase” refers to a protein which performs a desaturase reaction that introduces a carbon-carbon double bond at the 15^{th} carbon-carbon bond from the carboxyl end of a fatty acid substrate. Numerous genes encoding Δ15-desaturases have been cloned from plant and fungal species. For example, U.S. Pat. No. 5,952,544 describes nucleic acids encoding plant Δ15-desaturases (FAD3). These enzymes comprise amino acid motifs that were characteristic of plant Δ15-desaturases. WO200114538 describes a gene encoding soybean FAD3. Many other Δ15-desaturase sequences can be easily identified by searching sequence databases.

As used herein, a “Δ17-desaturase” refers to a protein which performs a desaturase reaction that introduces a carbon-carbon double bond at the 17^{th} carbon-carbon bond from the carboxyl end of a fatty acid substrate. A Δ17-desaturase is also regarded as an ω3-desaturase if it acts on a C20 substrate to introduce a desaturation at the ω3 bond.

In a preferred embodiment, the Δ12-desaturase and/or Δ15-desaturase is a fungal Δ12-desaturase or fungal Δ15-desaturase. As used herein, a “fungal Δ12-desaturase” or “a fungal Δ15-desaturase” refers to a Δ12-desaturase or Δ15-desaturase which is from a fungal source, including an oomycete source, or a variant thereof whose amino acid sequence is at least 95% identical thereto. Genes encoding numerous desaturases have been isolated from fungal sources. U.S. Pat. No. 7,211,656 describes a Δ12 desaturase from Saprolegnia diclina. WO2009016202 describes fungal desaturases from Helobdella robusta, Laccaria bicolor, Lottia gigantea, Microcoleus chthonoplastes, Monosiga brevicollis, Mycosphaerella fijiensis, Mycospaerella graminicola, Naegleria gruben, Nectria haematococca, Nematostella vectensis, Phycomyces blakesleeanus. Trichoderma resii, Physcomitrella patens, Postia placenta, Selaginella moellendorffii and Microdochium nivale. WO2005/012316 describes a Δ12-desaturase from Thalassiosira pseudonana and other fungi. WO2003/099216 describes genes encoding fungal Δ12-desaturases and Δ15-desaturases isolated from Neurospora crassa, Aspergillus nidulans, Botrytis cinerea and Mortierella alpina. WO2007133425 describes fungal Δ15 desaturases isolated from: Saccharomyces kluyveri, Mortierella alpina, Aspergillus nidulans, Neurospora crassa, Fusarium graminearum, Fusarium moniliforme and Magnaporthe grisea. A preferred Δ12 desaturase is from Phytophthora sojae (Ruiz-Lopez et al., 2012).

A distinct subclass of fungal Δ12-desaturases, and of fungal Δ15-desaturases, are the bifunctional fungal Δ12/Δ15-desaturases. Genes encoding these have been cloned from Fusarium monoliforme (Accession No. DQ272516, Damude et al., 2006), Acanthamoeba castellanii (Accession No. EF017656, Sayanova et al., 2006), Perkinsus marinus (WO2007042510), Claviceps purpurea (Accession No. EF536898, Meesapyodsuk et al., 2007) and Coprinus cinereus (Accession No. AF269266, Zhang et al., 2007).

In another embodiment, the ω3-desaturase has at least some activity on, preferably greater activity on, an acyl-CoA substrate than a corresponding acyl-PC substrate. As used herein, a “corresponding acyl-PC substrate” refers to the fatty acid esterified at the sn-2 position of phosphatidylcholine (PC) where the fatty acid is the same fatty acid as in the acyl-CoA substrate. For example, the acyl-CoA substrate may be ARA-CoA and the corresponding acyl-PC substrate is sn-2 ARA-PC. In an embodiment, the activity is at least two-fold greater. Preferably, the ω3-desaturase has at least some activity on both an acyl-CoA substrate and its corresponding acyl-PC substrate and has activity on both C18 and C20 substrates. Examples of such ω3-desaturases are known amongst the cloned fungal desaturases listed above.

In a further embodiment, the ω3-desaturase comprises amino acids having a sequence as provided in SEQ ID NO:12, a biologically active fragment thereof, or an amino acid sequence which is at least 60% identical to SEQ ID NO:12, preferably at least 90% or at least 95% identical to SEQ ID NO:12.

In yet a further embodiment, a desaturase for use in the present invention has greater activity on an acyl-CoA substrate than a corresponding acyl-PC substrate. In another embodiment, a desaturase for use in the present invention has greater activity on an acyl-PC substrate than a corresponding acyl-CoA substrate, but has some activity on both substrates. As outlined above, a “corresponding acyl-PC substrate” refers to the fatty acid esterified at the sn-2 position of phosphatidylcholine (PC) where the fatty acid is the same fatty acid as in the acyl-CoA substrate. In an embodiment, the greater activity is at least two-fold greater. In an embodiment, the desaturase is a Δ5 or Δ6-desaturase, or an ω3-desaturase, examples of which are provided, but not limited to, those listed in Table 2. To test which substrate a desaturase acts on, namely an acyl-CoA or an acyl-PC substrate, assays can be carried out in yeast cells as described in Domergue et al. (2003) and (2005). Acyl-CoA substrate capability for a desaturase can also be inferred when an elongase, when expressed together with the desaturase, has an enzymatic conversion efficiency in plant cells of at least about 90% where the elongase catalyses the elongation of the product of the desaturase. On this basis, the Δ5-desaturase and Δ4-desaturases expressed from the GA7 construct (Examples 2 and 3) and variants thereof (Example 5) are capable of desaturating their respective acyl-CoA substrates, ETA-CoA and DPA-CoA.

### Elongases

Biochemical evidence suggests that the fatty acid elongation consists of 4 steps: condensation, reduction, dehydration and a second reduction. In the context of this invention, an “elongase” refers to the polypeptide that catalyses the condensing step in the presence of the other members of the elongation complex, under suitable physiological conditions. It has been shown that heterologous or homologous expression in a cell of only the condensing component (“elongase”) of the elongation protein complex is required for the elongation of the respective acyl chain. Thus, the introduced elongase is able to successfully recruit the reduction and dehydration activities from the transgenic host to carry out successful acyl elongations. The specificity of the elongation reaction with respect to chain length and the degree of desaturation of fatty acid substrates is thought to reside in the condensing component. This component is also thought to be rate limiting in the elongation reaction.

As used herein, a “Δ5-elongase” is at least capable of converting EPA to DPA. Examples of Δ5-elongases include those disclosed in WO2005/103253. In one embodiment, the Δ5-elongase has activity on EPA to produce DPA with an efficiency of at least 60%, more preferably at least 65%, more preferably at least 70% or most preferably at least 80% or 90%. In a further embodiment, the Δ5-elongase comprises an amino acid sequence as provided in SEQ ID NO:37, a biologically active fragment thereof, or an amino acid sequence which is at least 47% identical to SEQ ID NO:37. In a further embodiment, the Δ6-elongase is from Ostreococcus taurii or Ostreococcus lucimarinus (US2010/088776).

As used herein, a “Δ6-elongase” is at least capable of converting SDA to ETA. Examples of Δ6-elongases include those listed in Table 1. In one embodiment, the elongase comprises amino acids having a sequence as provided in SEQ ID NO:25, a biologically active fragment thereof (such as the fragment provided as SEQ ID NO:26), or an amino acid sequence which is at least 55% identical to one or both of SEQ ID NO:25 or SEQ ID NO:26. In an embodiment, the Δ6-elongase is from Physcomitrella patens (Zank et al., 2002; Accession No. AF428243) or Thalassiosira pseudonana (Ruiz-Lopez et al., 2012).

As used herein, a “Δ9-elongase” is at least capable of converting ALA to ETrA. Examples of Δ9-elongases include those listed in Table 1. In one embodiment, the Δ9-elongase comprises amino acids having a sequence as provided in SEQ ID NO:43, a biologically active fragment thereof, or an amino acid sequence which is at least 80% identical to SEQ ID NO:43. In another embodiment, the Δ9-elongase comprises amino acids having a sequence as provided in SEQ ID NO:46, a biologically active fragment thereof, or an amino acid sequence which is at least 81% identical to SEQ ID NO:46. In another embodiment, the Δ9-elongase comprises amino acids having a sequence as provided in SEQ ID NO:48, a biologically active fragment thereof, or an amino acid sequence which is at least 50% identical to SEQ ID NO:48. In another embodiment, the Δ9-elongase comprises amino acids having a sequence as provided in SEQ ID NO:50, a biologically active fragment thereof, or an amino acid sequence which is at least 50% identical to SEQ ID NO:50. In a further embodiment, the Δ9-elongase has greater activity on an ω6 substrate than the corresponding ω3 substrate, or the converse.

As used herein, the term “has greater activity on an ω6 substrate than the corresponding ω3 substrate” refers to the relative activity of the enzyme on substrates that differ by the action of an ω3 desaturase. Preferably, the ω6 substrate is LA and the ω3 substrate is ALA.

An elongase with Δ6-elongase and Δ9-elongase activity is at least capable of (i) converting SDA to ETA and (ii) converting ALA to ETrA and has greater Δ6-elongase activity than Δ9-elongase activity. In one embodiment, the elongase has an efficiency of conversion on SDA to produce ETA which is at least 50%, more preferably at least 60%, and/or an efficiency of conversion on ALA to produce ETrA which is at least 6% or more preferably at least 9%. In another embodiment, the elongase has at least about 6.5 fold greater Δ6-elongase activity than Δ9-elongase activity. In a further embodiment, the elongase has no detectable Δ5-elongase activity

### Other Enzymes

As used herein, the term “1-acyl-glycerol-3-phosphate acyltransferase” (LPAAT), also termed lysophosphatidic acid-acyltransferase or acylCoA-lysophosphatidate-acyltransferase, refers to a protein which acylates sn-1-acyl-glycerol-3-phosphate (sn-1 G-3-P) at the sn-2 position to form phosphatidic acid (PA). Thus, the term “1-acyl-glycerol-3-phosphate acyltransferase activity” refers to the acylation of (sn-1 G-3-P) at the sn-2 position to produce PA (EC 2.3.1.51). Preferred LPAATs are those that can use a polyunsaturated C22 acyl-CoA as substrate to transfer the polyunsaturated C22 acyl group to the sn-2 position of LPA, forming PA. Such LPAATs are exemplified in Example 13 and can be tested as described therein. In an embodiment, an LPAAT useful for the invention comprises amino acids having a sequence as provided in any one of SEQ ID NOs. 63 to 69, a biologically active fragment thereof, or an amino acid sequence which is at least 40% identical to any one or more of SEQ ID NOs: 63 to 69. In a preferred embodiment, an LPAAT useful for the invention comprises amino acids having a sequence as provided in any one of SEQ ID NOs: 64, 65 and 67, a biologically active fragment thereof, or an amino acid sequence which is at least 40% identical to any one or more of SEQ ID NOs: 64, 65 and 67.

As used herein, the term “diacylglycerol acyltransferase” (EC 2.3.1.20; DGAT) refers to a protein which transfers a fatty acyl group from acyl-CoA to a diacylglycerol substrate to produce a triacylglycerol. Thus, the term “diacylglycerol acyltransferase activity” refers to the transfer of acyl-CoA to diacylglycerol to produce triacylglycerol. There are three known types of DGAT referred to as DGAT1, DGAT2 and DGAT3 respectively. DGAT1 polypeptides typically have 10 transmembrane domains, DGAT2 typically have 2 transmembrane domains, whilst DGAT3 is typically soluble. Examples of DGAT1 polypeptides include polypeptides encoded by DGAT1 genes from Aspergillus fumigatus (Accession No. XP_755172), Arabidopsis thaliana (CAB44774), Ricinus communis (AAR11479), Vernicia fordii (ABC94472), Vernonia galamensis (ABV21945. ABV21946), Euonymus alatus (AAV31083), Caenorhabditis elegans (AAF82410), Rattus norvegicus (NP_445889), Homo sapiens (NP_036211), as well as variants and/or mutants thereof. Examples of DGAT2 polypeptides include polypeptides encoded by DGAT2 genes from Arabidopsis thaliana (Accession No. NP_566952), Ricinus communis (AAY16324), Vernicia fordii (ABC94474), Mortierella ramanniana (AAK84179). Homo sapiens (Q96PD7, Q58HT5), Bos taurus (Q70VD8), Mus musculus (AAK84175), Micromonas CCMP1545, as well as variants and/or mutants thereof. Examples of DGAT3 polypeptides include polypeptides encoded by DGAT3 genes from peanut (Arachis hypogaea, Saha, et al., 2006), as well as variants and/or mutants thereof.

### Polypeptides/Peptides

The term “recombinant” in the context of a polypeptide refers to the polypeptide when produced by a cell, or in a cell-free expression system, in an altered amount or at an altered rate, compared to its native state if it is produced naturally. In one embodiment the cell is a cell that does not naturally produce the polypeptide. However, the cell may be a cell which comprises a non-endogenous gene that causes an altered amount of the polypeptide to be produced. A recombinant polypeptide of the invention includes polypeptides in the cell, tissue, organ or organism, or cell-free expression system, in which it is produced i.e. a polypeptide which has not been purified or separated from other components of the transgenic (recombinant) cell in which it was produced, and polypeptides produced in such cells or cell-free systems which are subsequently purified away from at least some other components.

The terms “polypeptide” and “protein” are generally used interchangeably.

A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970; GCG program) with parameters of a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over their entire length. The polypeptide or class of polypeptides may have the same enzymatic activity as, or a different activity than, or lack the activity of, the reference polypeptide. Preferably, the polypeptide has an enzymatic activity of at least 10%, at least 50%, at least 75% or at least 90%, of the activity of the reference polypeptide.

As used herein a “biologically active” fragment is a portion of a polypeptide defined herein which maintains a defined activity of a full-length reference polypeptide, for example possessing desaturase and/or elongase activity or other enzyme activity. Biologically active fragments as used herein exclude the full-length polypeptide. Biologically active fragments can be any size portion as long as they maintain the defined activity. Preferably, the biologically active fragment maintains at least 10%, at least 50%, at least 75% or at least 90%, of the activity of the full length protein.

With regard to a defined polypeptide or enzyme, it will be appreciated that % identity figures higher than those provided herein will encompass preferred embodiments. Thus, where applicable, in light of the minimum % identity figures, it is preferred that the polypeptide/enzyme comprises an amino acid sequence which is at least 60%, more preferably at least 65%, more preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence variants/mutants of the polypeptides of the defined herein can be prepared by introducing appropriate nucleotide changes into a nucleic acid defined herein, or by in vitro synthesis of the desired polypeptide. Such variants/mutants include, for example, deletions, insertions or substitutions of residues within the amino acid sequence. A combination of deletion, insertion and substitution can be made to arrive at the final construct, provided that the final peptide product possesses the desired enzyme activity.

Mutant (altered) peptides can be prepared using any technique known in the art. For example, a polynucleotide defined herein can be subjected to in vitro mutagenesis or DNA shuffling techniques as broadly described by Harayama (1998). Products derived from mutated/altered DNA can readily be screened using techniques described herein to determine if they possess, for example, desaturase or elongase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites which are not conserved amongst naturally occurring desaturases or elongases. These sites are preferably substituted in a relatively conservative manner in order to maintain enzyme activity. Such conservative substitutions are shown in Table 3 under the heading of “exemplary substitutions”.

In a preferred embodiment a mutant/variant polypeptide has only, or not more than, one or two or three or four conservative amino acid changes w % ben compared to a naturally occurring polypeptide. Details of conservative amino acid changes are provided in Table 3. As the skilled person would be aware, such minor changes can reasonably be predicted not to alter the activity of the polypeptide when expressed in a recombinant cell.

Polypeptides can be produced in a variety of ways, including production and recovery of natural polypeptides or recombinant polypeptides according to methods known in the art. In one embodiment, a recombinant polypeptide is produced by culturing a cell capable of expressing the polypeptide under conditions effective to produce the polypeptide, such as a host cell defined herein. A more preferred cell to produce the polypeptide is a cell in a plant, especially in a seed in a plant.

### Polynucleotides

The invention also provides and/or uses polynucleotides which may be, for example, a gene, an isolated polynucleotide, a chimeric genetic construct such as a T-DNA molecule, or a chimeric DNA. It may be DNA or RNA of genomic or synthetic origin, double-stranded or single-stranded, and combined with carbohydrate, lipids, protein or other materials to perform a particular activity defined herein. The term “polynucleotide” is used interchangeably herein with the term “nucleic acid molecule”. By “isolated polynucleotide” we mean a polynucleotide which, if obtained from a natural source, has been separated from the polynucleotide sequences with which it is associated or linked in its native state, or a non-naturally occurring polynucleotide. Preferably, the isolated polynucleotide is at least 60% free, more preferably at least 75% free, and more preferably at least 90% free from other components with which it is naturally associated.

TABLE 3
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; vil; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In an embodiment, a polynucleotide of the invention is non-naturally occurring. Examples of non-naturally occurring polynucleotides include, but are not limited to, those that have been mutated (such as by using methods described herein), and polynucleotides where an open reading frame encoding a protein is operably linked to a promoter to which it is not naturally associated (such as in the constructs described herein).

As used herein, the term “gene” is to be taken in its broadest context and includes the deoxyribonucleotide sequences comprising the transcribed region and, if translated, the protein coding region, of a structural gene and including sequences located adjacent to the coding region on both the 5′ and 3′ ends for a distance of at least about 2 kb on either end and which are involved in expression of the gene. In this regard, the gene includes control signals such as promoters, enhancers, termination and/or polyadenylation signals that are naturally associated with a given gene, or heterologous control signals in which case the gene is referred to as a “chimeric gene”. The sequences which are located 5′ of the protein coding region and which are present on the mRNA are referred to as 5′ non-translated sequences. The sequences which are located 3′ or downstream of the protein coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA). Introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, a “chimeric DNA” or “chimeric genetic construct” refers to any DNA molecule that is not a native DNA molecule in its native location, also referred to herein as a “DNA construct”. Typically, a chimeric DNA or chimeric gene comprises regulatory and transcribed or protein coding sequences that are not found operably linked together in nature i.e. that are heterologous with respect to each other. Accordingly, a chimeric DNA or chimeric gene may comprise regulatory sequences and coding sequences that are derived from different sources, or regulatory sequences and coding sequences derived from the same source, but arranged in a manner different than that found in nature.

The term “endogenous” is used herein to refer to a substance that is normally present or produced in, for example, an unmodified plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA technology. The terms “foreign polynucleotide” or “exogenous polynucleotide” or “heterologous polynucleotide” and the like refer to any nucleic acid which is introduced into the genome of a cell by experimental manipulations. Foreign or exogenous genes may be genes that are inserted into a non-native organism, native genes introduced into a new location within the native host, or chimeric genes. A “transgene” is a gene that has been introduced into the genome by a transformation procedure. The terms “genetically modified”, “transgenic” and variations thereof include introducing genes into cells by transformation or transduction, mutating genes in cells and altering or modulating the regulation of a gene in a cell or organisms to which these acts have been done or their progeny. A “genomic region” as used herein refers to a position within the genome where a transgene, or group of transgenes (also referred to herein as a cluster), have been inserted into a cell, or an ancestor thereof. Such regions only comprise nucleotides that have been incorporated by the intervention of man such as by methods described herein.

The term “exogenous” in the context of a polynucleotide refers to the polynucleotide when present in a cell in an altered amount compared to its native state. In one embodiment, the cell is a cell that does not naturally comprise the polynucleotide. However, the cell may be a cell which comprises a non-endogenous polynucleotide resulting in an altered amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically such chimeric polynucleotides comprise at least an open reading frame encoding a polypeptide of the invention operably linked to a promoter suitable of driving transcription of the open reading frame in a cell of interest. As used herein, the term “different exogenous polynucleotides” or variations thereof means that the nucleotide sequence of each polynucleotide are different by at least one, preferably more, nucleotides. The polynucleotides encode RNAs which may or may not be translated to a protein within the cell. In an example, it is preferred that each polynucleotide encodes a protein with a different activity. In another example, each exogenous polynucleotide is less than 95%, less than 90%, or less than 80% identical to the other exogenous polynucleotides. Preferably, the exogenous polynucleotides encode functional proteins/enzymes. Furthermore, it is preferred that the different exogenous polynucleotides are non-overlapping in that each polynucleotide is a distinct region of the, for example, extrachromosomal transfer nucleic acid which does not overlap with another exogenous polynucleotide. At a minimum, each exogenous polynucleotide has a transcription start and stop site, as well as the designated promoter. An individual exogenous polynucleotide may or may not comprise introns.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

A polynucleotide of the present invention may selectively hybridise, under stringent conditions, to a polynucleotide that encodes a polypeptide of the present invention. As used herein, stringent conditions are those that (1) employ during hybridisation a denaturing agent such as formamide, for example, 50% (v/v) formamide with 0.1% (w/v) bovine serum albumin. 0.1% Ficoll, 0.1% polyvinylpyrrolidone, 50 mM sodium phosphate buffer at pH 6.5 with 750 mM NaCl, 75 mM sodium citrate at 42° C.; or (2) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS and/or (3) employ low ionic strength and high temperature for washing, for example, 0.015 M NaCl/0.0015 M sodium citrate/0.1% SDS at 50° C.

Polynucleotides of the invention may possess, when compared to naturally occurring molecules, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid as described above). It is thus apparent that polynucleotides of the invention can be either from a naturally occurring source or recombinant. Preferred polynucleotides are those which have coding regions that are codon-optimised for translation in plant cells, as is known in the art.

### Recombinant Vectors

One embodiment of the present invention includes a recombinant vector, which comprises at least one polynucleotide molecule defined herein, inserted into any vector capable of delivering the polynucleotide molecule into a host cell. Recombinant vectors include expression vectors. Recombinant vectors contain heterologous polynucleotide sequences, that is, polynucleotide sequences that are not naturally found adjacent to polynucleotide molecules defined herein that preferably are derived from a species other than the species from which the polynucleotide molecule(s) are derived. The vector can be either RNA or DNA and typically is a plasmid. Plasmid vectors typically include additional nucleic acid sequences that provide for easy selection, amplification, and transformation of the expression cassette in prokaryotic cells, e.g., pUC-derived vectors, pSK-derived vectors, pGEM-derived vectors, pSP-derived vectors, pBS-derived vectors, or preferably binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells. The recombinant vector may comprise more than one polynucleotide defined herein, for example three, four, five or six polynucleotides defined herein in combination, preferably a chimeric genetic construct of the invention, each polynucleotide being operably linked to expression control sequences that are operable in the cell of interest. More than one polynucleotide defined herein, for example 3, 4, 5 or 6 polynucleotides, are preferably covalently joined together in a single recombinant vector, preferably within a single T-DNA molecule, which may then be introduced as a single molecule into a cell to form a recombinant cell according to the invention, and preferably integrated into the genome of the recombinant cell, for example in a transgenic plant. Thereby, the polynucleotides which are so joined will be inherited together as a single genetic locus in progeny of the recombinant cell or plant. The recombinant vector or plant may comprise two or more such recombinant vectors, each containing multiple polynucleotides, for example wherein each recombinant vector comprises 3, 4, 5 or 6 polynucleotides.

“Operably linked” as used herein refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence, such as a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

When there are multiple promoters present, each promoter may independently be the same or different.

Recombinant molecules such as the chimeric DNAs or genetic constructs may also contain (a) one or more secretory signals which encode signal peptide sequences, to enable an expressed polypeptide defined herein to be secreted from the cell that produces the polypeptide or which provide for localisation of the expressed polypeptide, for example for retention of the polypeptide in the endoplasmic reticulum (ER) in the cell or transfer into a plastid, and/or (b) contain fusion sequences which lead to the expression of nucleic acid molecules as fusion proteins. Examples of suitable signal segments include any signal segment capable of directing the secretion or localisation of a polypeptide defined herein. Recombinant molecules may also include intervening and/or untranslated sequences surrounding and/or within the nucleic acid sequences of nucleic acid molecules defined herein.

To facilitate identification of transformants, the nucleic acid construct desirably comprises a selectable or screenable marker gene as, or in addition to, the foreign or exogenous polynucleotide. By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., 0-glucuronidase, luciferase, GFP or other enzyme activity not present in untransformed cells). The marker gene and the nucleotide sequence of interest do not have to be linked. The actual choice of a marker is not crucial as long as it is functional (i.e., selective) in combination with the cells of choice such as a plant cell.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418: a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP 275957, a gene encoding a 5-enolshikimate-3-phosphate synthase (EPSPS) conferring tolerance to N-phosphonomethylglycine as, for example, described by Hinchee et al. (1988), a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a green fluorescent protein gene (Niedz et al., 1995) or derivatives thereof; a luciferase (hic) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of the cell, such as the plant cell. Accordingly, the nucleic acid may comprise appropriate elements which allow the molecule to be incorporated into the genome, preferably the right and left border sequences of a T-DNA molecule, or the construct is placed in an appropriate vector which can be incorporated into a chromosome of the cell.

### Expression

As used herein, an expression vector is a DNA vector that is capable of transforming a host cell and of effecting expression of one or more specified polynucleotide molecule(s). Preferred expression vectors of the present invention can direct gene expression in yeast and/or plant cells. Expression vectors useful for the invention contain regulatory sequences such as transcription control sequences, translation control sequences, origins of replication, and other regulatory sequences that are compatible with the recombinant cell and that control the expression of polynucleotide molecules of the present invention. In particular, polynucleotides or vectors useful for the present invention include transcription control sequences. Transcription control sequences are sequences which control the initiation, elongation, and termination of transcription. Particularly important transcription control sequences are those which control transcription initiation, such as promoter and enhancer sequences. Suitable transcription control sequences include any transcription control sequence that can function in at least one of the recombinant cells of the present invention. The choice of the regulatory sequences used depends on the target organism such as a plant and/or target organ or tissue of interest. Such regulatory sequences may be obtained from any eukaryotic organism such as plants or plant viruses, or may be chemically synthesized. A variety of such transcription control sequences are known to those skilled in the art. Particularly preferred transcription control sequences are promoters active in directing transcription in plants, either constitutively or stage and/or tissue specific, depending on the use of the plant or parts thereof.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989: and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase promoter from wheat, the nuclear photosynthetic ST-LS1 promoter from potato, the serine/threonine kinase promoter and the glucoamylase (CHS) promoter from Arabidopsis thaliana. Also reported to be active in photosynthetically active tissues are ribulose-1,5-bisphosphate carboxylase promoters, and Cab promoters.

A variety of plant gene promoters that are regulated in response to environmental, hormonal, chemical, and/or developmental signals, also can be used for expression of genes in plant cells, including promoters regulated by (1) heat, (2) light (e.g., pea RbcS-3A promoter, maize RbcS promoter); (3) hormones, such as abscisic acid, (4) wounding (e.g., WunI); or (5) chemicals, such as methyl jasmonate, salicylic acid, steroid hormones, alcohol, Safeners (WO97/06269), or it may also be advantageous to employ (6) organ-specific promoters.

As used herein, the term “plant seed specific promoter” or variations thereof refer to a promoter that preferentially, when compared to other plant tissues, directs gene transcription in a developing seed of a plant. In an embodiment, the seed specific promoter is expressed at least 5-fold more strongly in the developing seed of the plant relative to the leaves and/or stems of the plant, and is preferably expressed more strongly in the embryo of the developing seed compared to other plant tissues. Preferably, the promoter only directs expression of a gene of interest in the developing seed, and/or expression of the gene of interest in other parts of the plant such as leaves is not detectable by Northern blot analysis and/or RT-PCR. Typically, the promoter drives expression of genes during growth and development of the seed, in particular during the phase of synthesis and accumulation of storage compounds in the seed. Such promoters may drive gene expression in the entire plant storage organ or only part thereof such as the seedcoat, or cotyledon(s), preferably in the embryos, in seeds of dicotyledonous plants or the endosperm or aleurone layer of a seeds of monocotyledonous plants.

Preferred promoters for seed-specific expression include i) promoters from genes encoding enzymes involved in fatty acid biosynthesis and accumulation in seeds, such as desaturases and elongases, ii) promoters from genes encoding seed storage proteins, and iii) promoters from genes encoding enzymes involved in carbohydrate biosynthesis and accumulation in seeds. Seed specific promoters which are suitable are the oilseed rape napin gene promoter (U.S. Pat. No. 5,608,152), the Vicia faba USP promoter (Baumlein et al., 1991), the Arabidopsis oleosin promoter (WO98/45461), the Phaseolus vulgaris phaseolin promoter (U.S. Pat. No. 5,504,200), the Brassica Bce4 promoter (WO91/13980) or the legumin LeB4 promoter from Viciafaba (Baumlein et al., 1992), and promoters which lead to the seed-specific expression in monocots such as maize, barley, wheat, rye, rice and the like. Notable promoters which are suitable are the barley lpt2 or lpt1 gene promoter (WO95/15389 and WO95/23230) or the promoters described in WO99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene, the rye secalin gene). Other promoters include those described by Broun et al. (1998), Potenza et al. (2004), US20070192902 and US20030159173. In an embodiment, the seed specific promoter is preferentially expressed in defined parts of the seed such as the embryo, cotyledon(s) or the endosperm. Examples of such specific promoters include, but are not limited to, the FP1 promoter (Ellerstrom et al., 1996), the pea legumin promoter (Perrin et al., 2000), the bean phytohemagglutnin promoter (Perrin et al., 2000), the conlinin 1 and conlinin 2 promoters for the genes encoding the flax 2S storage proteins (Cheng et al., 2010), the promoter of the FAE1 gene from Arabidopsis thaliana, the BnGLP promoter of the globulin-like protein gene of Brassica napus, the LPXR promoter of the peroxiredoxin gene from Linum usitatissimum.

The 5′ non-translated leader sequence can be derived from the promoter selected to express the heterologous gene sequence of the polynucleotide of the present invention, or preferably is heterologous with respect to the coding region of the enzyme to be produced, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. Nos. 5,362,865 and 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene or a flax conlinin gene are commonly used in this capacity. The 3′ transcribed, non-translated regions containing the polyadenylate signal of Agrobacterium tumor-inducing (Ti) plasmid genes are also suitable.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules defined herein include, but are not limited to, integration of the polynucleotide molecule into one or more host cell chromosomes, addition of stability sequences to mRNAs, substitutions or modifications of transcription control signals (e.g., promoters, operators, enhancers), substitutions or modifications of translational control signals (e.g., ribosome binding sites, Shine-Dalgarno sequences), modification of polynucleotide molecules to correspond to the codon usage of the host cell, and the deletion of sequences that destabilize transcripts.

### Recombinant Cells

The invention also provides a recombinant cell, preferably a recombinant plant cell, which is a host cell transformed with one or more recombinant molecules, such as the polynucleotides, chimeric genetic constructs or recombinant vectors defined herein. The recombinant cell may comprise any combination thereof, such as two or three recombinant vectors, or a recombinant vector and one or more additional polynucleotides or chimeric DNAs. Suitable cells of the invention include any cell that can be transformed with a polynucleotide, chimeric DNA or recombinant vector of the invention, such as for example, a molecule encoding a polypeptide or enzyme described herein. The cell is preferably a cell which is thereby capable of being used for producing LC-PUFA. The recombinant cell may be a cell in culture, a cell in vitro, or in an organism such as for example a plant, or in an organ such as for example a seed or a leaf. Preferably, the cell is in a plant or plant part, more preferably in the seed of a plant.

Host cells into which the polynucleotide(s) are introduced can be either untransformed cells or cells that are already transformed with at least one nucleic acid molecule. Such nucleic acid molecules may be related to LC-PUFA synthesis, or unrelated. Host cells of the present invention either can be endogenously (i.e., naturally) capable of producing proteins defined herein, in which case the recombinant cell derived therefrom has an enhanced capability of producing the polypeptides, or can be capable of producing such proteins only after being transformed with at least one polynucleotide of the invention. In an embodiment, a recombinant cell of the invention has a enhanced capacity to synthesize a long chain polyunsaturated fatty acid. As used herein, the term “cell with an enhanced capacity to synthesize a long chain polyunsaturated fatty acid” is a relative term where the recombinant cell of the invention is compared to the host cell lacking the polynucleotide(s) of the invention, with the recombinant cell producing more long chain polyunsaturated fatty acids, or a greater concentration of LC-PUFA such as DHA (relative to other fatty acids), than the native cell. The cell with an enhanced capacity to synthesize another product, such as for example another fatty acid, a lipid, a carbohydrate such as starch, an RNA molecule, a polypeptide, a pharmaceutical or other product has a corresponding meaning.

Host cells of the present invention can be any cell capable of producing at least one protein described herein, and include bacterial, fungal (including yeast), parasite, arthropod, animal and plant cells. The cells may be prokaryotic or eukaryotic. Preferred host cells are yeast and plant cells. In a preferred embodiment, the plant cell is a seed cell, in particular a cell in a cotyledon or endosperm of a seed. In one embodiment, the cell is an animal cell or an algal cell. The animal cell may be of any type of animal such as, for example, a non-human animal cell, a non-human vertebrate cell, a non-human mammalian cell, or cells of aquatic animals such as, fish or crustacea, invertebrates, insects, etc. The cells may be of an organism suitable for a fermentation process. As used herein, the term the “fermentation process” refers to any fermentation process or any process comprising a fermentation step. Examples of fermenting microorganisms include fungal organisms, such as yeast. As used herein, “yeast” includes Saccharomyces spp., Saccharomyces cerevisiae, Saccharomyces carlbergensis. Candida spp., Kluyveromyces spp., Pichia spp., Hansenula spp., Trichoderma spp., Lipomyces starkey, and Yarrowia lipolytica. Preferred yeast include strains of the Saccharomyces spp., and in particular. Saccharomyces cerevisiae.

### Transgenic Plants

The invention also provides a plant comprising a cell of the invention, such as a transgenic plant comprising one or more polynucleotides of the invention. The term “plant” as used herein as a noun refers to whole plants, but as used as an adjective refers to any substance which is present in, obtained from, derived from, or related to a plant, such as for example, plant organs (e.g. leaves, stems, roots, flowers), single cells (e.g. pollen), seeds, plant cells and the like. The term “plant part” refers to all plant parts that comprise the plant DNA, including vegetative structures such as, for example, leaves or stems, roots, floral organs or structures, pollen, seed, seed parts such as an embryo, endosperm, scutellum or seed coat, plant tissue such as, for example, vascular tissue, cells and progeny of the same, as long as the plant part synthesizes lipid according to the invention.

A “transgenic plant”, “genetically modified plant” or variations thereof refers to a plant that contains a gene construct (“transgene”) not found in a wild-type plant of the same species, variety or cultivar. Transgenic plants as defined in the context of the present invention include plants and their progeny which have been genetically modified using recombinant techniques to cause production of the lipid or at least one polypeptide defined herein in the desired plant or plant organ. Transgenic plant cells and transgenic plant parts have corresponding meanings. A “transgene” as referred to herein has the normal meaning in the art of biotechnology and includes a genetic sequence which has been produced or altered by recombinant DNA or RNA technology and which has been introduced into a cell of the invention, preferably a plant cell. The transgene may include genetic sequences derived from a plant cell which may be of the same species, variety or cultivar as the plant cell into which the transgene is introduced or of a different species, variety or cultivar, or from a cell other than a plant cell. Typically, the transgene has been introduced into the cell, such as a plant, by human manipulation such as, for example, by transformation but any method can be used as one of skill in the art recognizes.

The terms “seed” and “grain” are used interchangeably herein. “Grain” refers to mature grain such as harvested grain or grain which is still on a plant but ready for harvesting, but can also refer to grain after imbibition or germination, according to the context. Mature grain or seed commonly has a moisture content of less than about 18-20%. “Developing seed” as used herein refers to a seed prior to maturity, typically found in the reproductive structures of the plant after fertilisation or anthesis, but can also refer to such seeds prior to maturity which are isolated from a plant.

As used herein, the term “obtaining a plant part” or “obtaining a seed” refers to any means of obtaining a plant part or seed, respectively, including harvesting of the plant parts or seed from plants in the field or in containment such as a greenhouse or growth chamber, or by purchase or receipt from a supplier of the plant parts or seed. The seed may be suitable for planting i.e. able to germinate and produce progeny plants, or alternatively has been processed in such a way that it is no longer able to germinate, e.g. cracked, polished or milled seed which is useful for food or feed applications, or for extraction of lipid of the invention.

As used herein, the term “plant storage organ” refers to a part of a plant specialized to storage energy in the form of, for example, proteins, carbohydrates, fatty acids and/or oils. Examples of plant storage organs are seed, fruit, tuberous roots, and tubers. A preferred plant storage organ of the invention is seed.

As used herein, the term “phenotypically normal” refers to a genetically modified plant or plant organ, particularly a storage organ such as a seed, tuber or fruit of the invention not having a significantly reduced ability to grow and reproduce when compared to an unmodified plant or plant organ. In an embodiment, the genetically modified plant or plant organ which is phenotypically normal comprises an exogenous polynucleotide encoding a silencing suppressor operably linked to a plant storage organ specific promoter and has an ability to grow or reproduce which is essentially the same as an isogenic plant or organ not comprising said polynucleotide. Preferably, the biomass, growth rate, germination rate, storage organ size, seed size and/or the number of viable seeds produced is not less than 90% of that of a plant lacking said exogenous polynucleotide when grown under identical conditions. This term does not encompass features of the plant which may be different to the wild-type plant but which do not effect the usefulness of the plant for commercial purposes such as, for example, a ballerina phenotype of seedling leaves.

Plants provided by or contemplated for use in the practice of the present invention include both monocotyledons and dicotyledons. In preferred embodiments, the plants of the present invention are crop plants (for example, cereals and pulses, maize, wheat, potatoes, tapioca, rice, sorghum, millet, cassava, barley, or pea), or other legumes. The plants may be grown for production of edible roots, tubers, leaves, stems, flowers or fruit. The plants may be vegetables or ornamental plants. The plants of the invention may be: corn (Zea mays), canola (Brassica napus, Brassica rapa ssp.), mustard (Brassica juncea), flax (Linum usitatissimum), alfalfa (Medicago sativa), rice (Oryza sativa), rye (Secale cerale), sorghum (Sorghum bicolour, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Lopmoea batatus), cassava (Manihot esculenta), coffee (Cofea spp.), coconut (Cocos nucifera), pineapple (Anana comosus), citris tree (Citrus spp.), cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, or barley.

In a preferred embodiment, the plant is an angiosperm.

In an embodiment, the plant is an oilseed plant, preferably an oilseed crop plant. As used herein, an “oilseed plant” is a plant species used for the commercial production of oils from the seeds of the plant. The oilseed plant may be oil-seed rape (such as canola), maize, sunflower, soybean, sorghum, flax (linseed) or sugar beet. Furthermore, the oilseed plant may be other Brassicas, cotton, peanut, poppy, mustard, castor bean, sesame, safflower, or nut producing plants. The plant may produce high levels of oil in its fruit, such as olive, oil palm or coconut. Horticultural plants to which the present invention may be applied are lettuce, endive, or vegetable brassicas including cabbage, broccoli, or cauliflower. The present invention may be applied in tobacco, cucurbits, carrot, strawberry, tomato, or pepper.

In a further preferred embodiment, the non-transgenic plant used to produce a transgenic plant of the invention produces oil, especially in the seed, which has i) less than 20%, less than 10% or less than 5% 18:2 fatty acids and/or ii) less than 10% or less than 5% 18:3 fatty acids.

In a preferred embodiment, the transgenic plant is homozygous for each and every gene that has been introduced (transgene) so that its progeny do not segregate for the desired phenotype. The transgenic plant may also be heterozygous for the introduced transgene(s), preferably uniformly heterozygous for the transgene, such as for example in F1 progeny which have been grown from hybrid seed. Such plants may provide advantages such as hybrid vigour, well known in the art.

Where relevant, the transgenic plants may also comprise additional transgenes encoding enzymes involved in the production of LC-PUFAs such as, but not limited to, a Δ6-desaturase, a Δ9-elongase, a Δ8-desaturase, a Δ6-elongase, a Δ5-desaturase, an ω3-desaturase, a Δ4-desaturase, a Δ5-elongase, diacylglycerol acyltransferase. LPAAT, a Δ17-desaturase, a Δ15-desaturase and/or a Δ12 desaturase. Examples of such enzymes with one of more of these activities are known in the art and include those described herein. In specific examples, the transgenic plant at least comprises exogenous polynucleotides encoding:
a) a Δ4-desaturase, a Δ5-desaturase, a Δ6-desaturase, a Δ5-elongase and a Δ6-elongase,
b) a Δ4-desaturase, a Δ5-desaturase, a Δ8-desaturase, a Δ5-elongase and a Δ9-elongase,
c) a Δ4-desaturase, a Δ5-desaturase, a Δ6-desaturase, a Δ5-elongase, a Δ6-elongase, and a Δ15-desaturase,
d) a Δ4-desaturase, a Δ5-desaturase, a Δ8-desaturase, a Δ5-elongase, a Δ9-elongase, and a Δ15-desaturase,
e) a Δ4-desaturase, a Δ5-desaturase, a Δ6-desaturase, a Δ5-elongase, a Δ6-elongase, and a Δ17-desaturase, or
f) a Δ4-desaturase, a Δ5-desaturase, a Δ8-desaturase, a Δ5-elongase, a Δ9-elongase, and a Δ17-desaturase.

In an embodiment, the exogenous polynucleotides encode set of polypeptides which are a Pythium irregulare Δ6-desaturase, a Thraustochyprid Δ5-desaturase or an Emiliana huxleyi Δ5-desaturase, a Physcomitrella patens Δ6-elongase, a Thraustochytrid Δ5-elongase or an Ostreocccus taurii Δ5-elongase, a Phytophthora infestans ω3-desaturase or a Pythium irregulare ω3-desaturase, and a Thraustochytrid Δ4-desaturase.

In an embodiment, plants of the invention are grown in the field, preferably as a population of at least 1,000 or 1,000,000 plants that are essentially the same, or in an area of at least 1 hectare. Planting densities differ according to the plant species, plant variety, climate, soil conditions, fertiliser rates and other factors as known in the art. For example, canola is typically grown at a planting density of 1.2-1.5 million plants per hectare. Plants are harvested as is known in the art, which may comprise swathing, windrowing and/or reaping of plants, followed by threshing and/or winnowing of the plant material to separate the seed from the remainder of the plant parts often in the form of chaff. Alternatively, seed may be harvested from plants in the field in a single process, namely combining.

### Transformation of Plants

Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

As used herein, the terms “stably transforming”, “stably transformed” and variations thereof refer to the integration of the exogenous nucleic acid molecules into the genome of the cell such that they are transferred to progeny cells during cell division without the need for positively selecting for their presence. Stable transformants, or progeny thereof, can be selected by any means known in the art such as Southern blots on chromosomal DNA or in situ hybridization of genomic DNA.

Agrobacterium-mediated transfer is a widely applicable system for introducing genes into plant cells because DNA can be introduced into cells in whole plant tissues or plant organs or explants in tissue culture, for either transient expression or for stable integration of the DNA in the plant cell genome. The use of Agrobacterium-mediated plant integrating vectors to introduce DNA into plant cells is well known in the art (see, for example, U.S. Pat. Nos. 5,177,010, 5,104,310, 5,004,863 or U.S. Pat. No. 5,159,135) including floral dipping methods using Agrobacterium or other bacteria that can transfer DNA into plant cells. The region of DNA to be transferred is defined by the border sequences, and the intervening DNA (T-DNA) is usually inserted into the plant genome. Further, the integration of the T-DNA is a relatively precise process resulting in few rearrangements. In those plant varieties where Agrobacterium-mediated transformation is efficient, it is the method of choice because of the facile and defined nature of the gene transfer. Preferred Agrobacterium transformation vectors are capable of replication in E. coli as well as Agrobacterium, allowing for convenient manipulations as described (Klee et al., In: Plant DNA Infectious Agents, Hohn and Schell, eds., Springer-Verlag, New York, pp. 179-203 (1985).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required.

In another alternative embodiment, plastids can be stably transformed. Methods disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO99/05265).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

A transgenic plant formed using Agrobacterium or other transformation methods typically contains a single genetic locus on one chromosome. Such transgenic plants can be referred to as being hemizygous for the added gene(s). More preferred is a transgenic plant that is homozygous for the added gene(s); i.e., a transgenic plant that contains two added genes, one gene at the same locus on each chromosome of a chromosome pair. A homozygous transgenic plant can be obtained by self-fertilising a hemizygous transgenic plant, germinating some of the seed produced and analyzing the resulting plants for the gene of interest.

It is also to be understood that two different transgenic plants that contain two independently segregating exogenous genes or loci can also be crossed (mated) to produce offspring that contain both sets of genes or loci. Selfing of appropriate F1 progeny can produce plants that are homozygous for both exogenous genes or loci. Back-crossing to a parental plant and out-crossing with a non-transgenic plant are also contemplated, as is vegetative propagation. Descriptions of other breeding methods that are commonly used for different traits and crops can be found in Fehr. In: Breeding Methods for Cultivar Development, Wilcox J. ed., American Society of Agronomy. Madison Wis. (1987).

### Enhancing Exogenous RNA Levels and Stabilized Expression

### Silencing Suppressors

In an embodiment, a cell, plant or plant part of the invention comprises an exogenous polynucleotide encoding a silencing suppressor protein.

Post-transcriptional gene silencing (PTGS) is a nucleotide sequence-specific defense mechanism that can target both cellular and viral mRNAs for degradation PTGS occurs in plants or fungi stably or transiently transformed with foreign (heterologous) or endogenous DNA and results in the reduced accumulation of RNA molecules with sequence similarity to the introduced nucleic acid.

It has widely been considered that co-expression of a silencing suppressor with a transgene of interest will increase the levels of RNA present in the cell transcribed from the transgene. Whilst this has proven true for cells in vitro, significant side-effects have been observed in many whole plant co-expression studies. More specifically, as described in Mallory et al. (2002), Chapman et al. (2004). Chen et al. (2004), Dunoyer et al. (2004), Zhang et al. (2006). Lewsey et al. (2007) and Meng et al. (2008) plants expressing silencing suppressors, generally under constitutive promoters, are often phenotypically abnormal to the extent that they are not useful for commercial production.

Recently, it has been found that RNA molecule levels can be increased, and/or RNA molecule levels stabilized over numerous generations, by limiting the expression of the silencing suppressor to a seed of a plant or part thereof (WO2010/057246). As used herein, a “silencing suppressor protein” or SSP is any polypeptide that can be expressed in a plant cell that enhances the level of expression product from a different transgene in the plant cell, particularly over repeated generations from the initially transformed plant. In an embodiment, the SSP is a viral silencing suppressor or mutant thereof. A large number of viral silencing suppressors are known in the art and include, but are not limited to P19, V2, P38, Pe-Po and RPV-P0. In an embodiment, the viral silencing suppressor comprises amino acids having a sequence as provided in any one of SEQ ID NOs 53 to 57, a biologically active fragment thereof, or an amino acid sequence which is at least 50% identical to any one or more of SEQ ID NOs 53 to 57 and which has activity as a silencing suppressor.

As used herein, the terms “stabilising expression”, “stably expressed”, “stabilised expression” and variations thereof refer to level of the RNA molecule being essentially the same or higher in progeny plants over repeated generations, for example at least three, at least five or at least 10 generations, when compared to isogenic plants lacking the exogenous polynucleotide encoding the silencing suppressor. However, this term(s) does not exclude the possibility that over repeated generations there is some loss of levels of the RNA molecule when compared to a previous generation, for example not less than a 10% loss per generation.

The suppressor can be selected from any source e.g. plant, viral, mammal etc. See WO2010/057246 for a list of viruses from which the suppressor can be obtained and the protein (eg B2, P14 etc) or coding region designation for the suppressor from each particular virus. Multiple copies of a suppressor may be used. Different suppressors may be used together (e.g., in tandem).

### RNA Molecules

Essentially any RNA molecule which is desirable to be expressed in a plant seed can be co-expressed with the silencing suppressor. The encoded polypeptides may be involved in metabolism of oil, starch, carbohydrates, nutrients, etc., or may be responsible for the synthesis of proteins, peptides, fatty acids, lipids, waxes, oils, starches, sugars, carbohydrates, flavors, odors, toxins, carotenoids, hormones, polymers, flavonoids, storage proteins, phenolic acids, alkaloids, lignins, tannins, celluloses, glycoproteins, glycolipids, etc, preferably the biosynthesis or assembly of TAG.

In a particular example, the plants produced increased levels of enzymes for oil production in plants such as Brassicas, for example canola or sunflower, safflower, flax, cotton, soya bean. Camelina or maize.

### Levels of LC-PUFA Produced

The levels of the LC-PUFA or combination of LC-PUFAs that are produced in the recombinant cell or plant part such as seed are of importance. The levels may be expressed as a composition (in percent) of the total fatty acid that is a particular LC-PUFA or group of related LC-PUFA, for example the ω3 LC-PUFA or the 06 LC-PUFA, or the VLC-PUFA, or other which may be determined by methods known in the art. The level may also be expressed as a LC-PUFA content, such as for example the percentage of LC-PUFA in the dry weight of material comprising the recombinant cells, for example the percentage of the weight of seed that is LC-PUFA. It will be appreciated that the LC-PUFA that is produced in an oilseed may be considerably higher in terms of LC-PUFA content than in a vegetable or a grain that is not grown for oil production, yet both may have similar LC-PUFA compositions, and both may be used as sources of LC-PUFA for human or animal consumption.

The levels of LC-PUFA may be determined by any of the methods known in the art. In a preferred method, total lipid is extracted from the cells, tissues or organisms and the fatty acid converted to methyl esters before analysis by gas chromatography (GC). Such techniques are described in Example 1. The peak position in the chromatogram may be used to identify each particular fatty acid, and the area under each peak integrated to determine the amount. As used herein, unless stated to the contrary, the percentage of particular fatty acid in a sample is determined as the area under the peak for that fatty acid as a percentage of the total area for fatty acids in the chromatogram. This corresponds essentially to a weight percentage (w/w). The identity of fatty acids may be confirmed by GC-MS. Total lipid may be separated by techniques known in the art to purify fractions such as the TAG fraction. For example, thin-layer chromatography (TLC) may be performed at an analytical scale to separate TAG from other lipid fractions such as DAG, acyl-CoAs or phospholipid in order to determine the fatty acid composition specifically of TAG.

In one embodiment, the sum total of ARA, EPA, DPA and DHA in the fatty acids in the extracted lipid is between about 7% and about 25% of the total fatty acids in the cell. In a further embodiment, the total fatty acid in the cell has less than 1% C20:1. In preferred embodiments, the extractable TAG in the cell comprises the fatty acids at the levels referred to herein. Each possible combination of the features defining the lipid as described herein is also encompassed.

The level of production of LC-PUFA in the recombinant cell, plant or plant part such as seed may also be expressed as a conversion percentage of a specific substrate fatty acid to one or more product fatty acids, which is also referred to herein as a “conversion efficiency” or “enzymatic efficiency”. This parameter is based on the fatty acid composition in the lipid extracted from the cell, plant, plant part or seed, i.e., the amount of the LC-PUFA formed (including other LC-PUFA derived therefrom) as a percentage of one or more substrate fatty acids (including all other fatty acids derived therefrom). The general formula for a conversion percentage is: 100×(the sum of percentages of the product LC-PUFA and all products derived therefrom)/(the sum of the percentages of the substrate fatty acid and all products derived therefrom). With regard to DHA, for example, this may be expressed as the ratio of the level of DHA (as a percentage in the total fatty acid content in the lipid) to the level of a substrate fatty acid (e.g. OA, LA, ALA. SDA, ETA or EPA) and all products other than DHA derived from the substrate. The conversion percentage or efficiency of conversion can be expressed for a single enzymatic step in a pathway, or for part or the whole of a pathway.

Specific conversion efficiencies are calculated herein according to the formulae.
1. OA to DHA=100×(% DHA)/(sum % for OA, LA, GLA, DGLA, ARA, EDA, ALA, SDA, ETrA, ETA, EPA, DPA and DHA).
2. LA to DHA=100×(% DHA)/(sum % for LA, GLA, DGLA, ARA, EDA, ALA, SDA, ETrA, ETA, EPA, DPA and DHA).
3. ALA to DHA=100×(% DHA)/(sum % for ALA, SDA, ETrA, ETA, EPA, DPA and DHA).
4. EPA to DHA=100×(% DHA)/(sum % for EPA. DPA and DHA).
5. DPA to DHA (Δ4-desaturase efficiency)=100×(% DHA)/(sum % for DPA and DHA).
6. Δ12-desaturase efficiency=100×(sum % for LA, GLA, DGLA, ARA, EDA, ALA, SDA, ETrA, ETA, EPA, DPA and DHA)/(sum % for OA, LA, GLA, DGLA, ARA, EDA, ALA, SDA, ETrA, ETA, EPA, DPA and DHA).
7. ω3-desaturase efficiency=100×(sum % for ALA, SDA, ETrA, ETA, EPA, DPA and DHA)/(sum % for LA, GLA, DGLA, ARA, EDA, ALA, SDA, ETrA, ETA, EPA, DPA and DHA).
8. OA to ALA=100×(sum % for ALA, SDA, ETrA, ETA, EPA, DPA and DHA)/(sum % for OA, LA, GLA, DGLA, ARA, EDA, ALA, SDA, ETrA, ETA, EPA, DPA and DHA).
9. Δ6-desaturase efficiency (on ω3 substrate ALA)=100×(sum % for SDA, ETA, EPA, DPA and DHA)/(% ALA, SDA, ETrA, ETA, EPA, DPA and DHA).
10. Δ6-elongase efficiency (on ω3 substrate SDA)=100×(sum % for ETA, EPA, DPA and DHA)/(sum % for SDA, ETA, EPA, DPA and DHA).
11. Δ5-desaturase efficiency (on ω3 substrate ETA)=100×(sum % for EPA, DPA and DHA)/(sum % for ETA, EPA, DPA and DHA).
12. Δ5-elongase efficiency (on (3 substrate EPA)=100×(sum % for DPA and DHA)/(sum % for EPA, DPA and DHA).

The fatty acid composition of the lipid, preferably seedoil, of the invention, is also characterised by the ratio of ω6 fatty acids:ω3 fatty acids in the total fatty acid content, for either total ω6 fatty acids:total ω3 fatty acids or for new ω6 fatty acids:new ω3 fatty acids. The terms total ω6 fatty acids, total ω3 fatty acids, new ω6 fatty acids and new ω3 fatty acids have the meanings as defined herein. The ratios are calculated from the fatty acid composition in the lipid extracted from the cell, plant, plant part or seed, in the manner as exemplified herein. It is desirable to have a greater level of ω3 than ω6 fatty acids in the lipid, and therefore an ω6:ω3 ratio of less than 1.0 is preferred. A ratio of 0.0 indicates a complete absence of the defined ω6 fatty acids: a ratio of 0.03 was achieved as described in Example 6. Such low ratios can be achieved through the combined use of a Δ6-desaturase which has an ω3 substrate preference together with an ω3-desaturase, particularly a fungal ω3-desaturase such as the Pichia pastoris ω3-desaturase as exemplified herein.

The yield of LC-PUFA per weight of seed may also be calculated based on the total oil content in the seed and the % DHA in the oil. For example, if the oil content of canola seed is about 40% (w/w) and about 12% of the total fatty acid content of the oil is DHA, the DHA content of the seed is about 4.8% or about 48 mg per gram of seed. As described in Example 2, the DHA content of Arabidopsis seed having about 9% DHA, which has a lower oil content than canola, was about 25 mg/g seed. At a DHA content of about 7%, canola seed or Camelina sativa seed has a DHA content of about 28 mg per gram of seed. The present invention therefore provides Brassica napus. B. juncea and Camelina sativa plants, and seed obtained therefrom, comprising at least about 28 mg DHA per gram seed. The seed has a moisture content as is standard for harvested mature seed after drying down (4-15% moisture). The invention also provides a process for obtaining oil, comprising obtaining the seed and extracting the oil from the seed, and uses of the oil and methods of obtaining the seed comprising harvesting the seeds from the plants according to the invention.

The amount of DHA produced per hectare can also be calculated if the seed yield per hectare is known or can be estimated. For example, canola in Australia typically yields about 2.5 tonnes seed per hectare, which at 40% oil content yields about 1000 kg of oil. At 12% DHA in the total oil, this provides about 120 kg of DHA per hectare. If the oil content is reduced by 50%, this still provides about 60 kg DHA/ha.

Evidence to date suggests that some desaturases expressed heterologously in yeast or plants have relatively low activity in combination with some elongases. This may be alleviated by providing a desaturase with the capacity of to use an acyl-CoA form of the fatty acid as a substrate in LC-PUFA synthesis, and this is thought to be advantageous in recombinant cells particularly in plant cells. A particularly advantageous combination for efficient DHA synthesis is a fungal ω3-desaturase, for example such as the Pichia pastoris ω3-desaturase (SEQ ID NO: 12), with a Δ6-desaturase which has a preference for ω3 acyl substrates such as, for example, the Micromonas pusilla Δ6-desaturase (SEQ ID NO: 13), or variants thereof which have at least 95% amino acid sequence identity.

As used herein, the term “essentially free” means that the composition (for example lipid or oil) comprises little (for example, less than about 0.5%, less than about 0.25%, less than about 0.1%, or less than about 0.01%) or none of the defined component. In an embodiment, “essentially free” means that the component is undetectable using a routine analytical technique, for example a specific fatty acid (such as (6-docosapentaenoic acid) cannot be detected using gas chromatography as outlined in Example 1.

### Production of Oils

Techniques that are routinely practiced in the art can be used to extract, process, and analyze the oils produced by cells, plants, seeds, etc of the instant invention. Typically, plant seeds are cooked, pressed, and extracted to produce crude oil, which is then degummed, refined, bleached, and deodorized. Generally, techniques for crushing seed are known in the art. For example, oilseeds can be tempered by spraying them with water to raise the moisture content to, e.g., 8.5%, and flaked using a smooth roller with a gap setting of 0.23 to 0.27 mm. Depending on the type of seed, water may not be added prior to crushing. Application of heat deactivates enzymes, facilitates further cell rupturing, coalesces the oil droplets, and agglomerates protein particles, all of which facilitate the extraction process.

In an embodiment, the majority of the seed oil is released by passage through a screw press. Cakes expelled from the screw press are then solvent extracted, e.g., with hexane, using a heat traced column. Alternatively, crude oil produced by the pressing operation can be passed through a settling tank with a slotted wire drainage top to remove the solids that are expressed with the oil during the pressing operation. The clarified oil can be passed through a plate and frame filter to remove any remaining fine solid particles. If desired, the oil recovered from the extraction process can be combined with the clarified oil to produce a blended crude oil.

Once the solvent is stripped from the crude oil, the pressed and extracted portions are combined and subjected to normal oil processing procedures. As used herein, the term “purified” when used in connection with lipid or oil of the invention typically means that that the extracted lipid or oil has been subjected to one or more processing steps of increase the purity of the lipid/oil component. For example, a purification step may comprise one or more or all of the group consisting of: degumming, deodorising, decolourising, drying and/or fractionating the extracted oil. However, as used herein, the term “purified” does not include a transesterification process or other process which alters the fatty acid composition of the lipid or oil of the invention so as to increase the DHA content as a percentage of the total fatty acid content. Expressed in other words, the fatty acid composition of the purified lipid or oil is essentially the same as that of the unpurified lipid or oil.

### Degumming

Degumming is an early step in the refining of oils and its primary purpose is the removal of most of the phospholipids from the oil, which may be present as approximately 1-2% of the total extracted lipid. Addition of ˜2% of water, typically containing phosphoric acid, at 70-80° C. to the crude oil results in the separation of most of the phospholipids accompanied by trace metals and pigments. The insoluble material that is removed is mainly a mixture of phospholipids and triacylglycerols and is also known as lecithin. Degumming can be performed by addition of concentrated phosphoric acid to the crude seedoil to convert non-hydratable phosphatides to a hydratable form, and to chelate minor metals that are present. Gum is separated from the seedoil by centrifugation.

### Alkali Refining

Alkali refining is one of the refining processes for treating crude oil, sometimes also referred to as neutralization. It usually follows degumming and precedes bleaching. Following degumming, the seedoil can treated by the addition of a sufficient amount of an alkali solution to titrate all of the fatty acids and phosphoric acids, and removing the soaps thus formed. Suitable alkaline materials include sodium hydroxide, potassium hydroxide, sodium carbonate, lithium hydroxide, calcium hydroxide, calcium carbonate and ammonium hydroxide. This process is typically carried out at room temperature and removes the free fatty acid fraction. Soap is removed by centrifugation or by extraction into a solvent for the soap, and the neutralised oil is washed with water. If required, any excess alkali in the oil may be neutralized with a suitable acid such as hydrochloric acid or sulphuric acid.

### Bleaching

Bleaching is a refining process in which oils are heated at 90-120° C. for 10-30 minutes in the presence of a bleaching earth (0.2-2.0%) and in the absence of oxygen by operating with nitrogen or steam or in a vacuum. This step in oil processing is designed to remove unwanted pigments (carotenoids, chlorophyll, gossypol etc), and the process also removes oxidation products, trace metals, sulphur compounds and traces of soap.

### Deodorization

Deodorization is a treatment of oils and fats at a high temperature (200-260° C.) and low pressure (0.1-1 mm Hg). This is typically achieved by introducing steam into the seedoil at a rate of about 0.1 ml/minute/100 ml of seedoil. After about 30 minutes of sparging, the seedoil is allowed to cool under vacuum. The seedoil is typically transferred to a glass container and flushed with argon before being stored under refrigeration. This treatment improves the colour of the seedoil and removes a majority of the volatile substances or odorous compounds including any remaining free fatty acids, monoacylglycerols and oxidation products.

### Winterisation

Winterization is a process sometimes used in commercial production of oils for the separation of oils and fats into solid (stearin) and liquid (olein) fractions by crystallization at sub-ambient temperatures. It was applied originally to cottonseed oil to produce a solid-free product. It is typically used to decrease the saturated fatty acid content of oils.

### Transesterification

Transesterification is a process that exchanges the fatty acids within and between TAGs or transfers the fatty acids to another alcohol to form an ester, initially by releasing fatty acids from the TAGs either as free fatty acids or as fatty acid esters, usually fatty acid methyl esters or ethyl esters. When combined with a fractionation process, transesterification can be used to modify the fatty acid composition of lipids (Marangoni et al., 1995). Transesterification can use either chemical (e.g. strong acid or base catalysed) or enzymatic means, the latter using lipases which may be position-specific (sn-1/3 or sn-2 specific) for the fatty acid on the TAG, or having a preference for some fatty acids over others (Speranza et al, 2012). The fatty acid fractionation to increase the concentration of LC-PUFA in an oil can be achieved by any of the methods known in the art, such as, for example, freezing crystallization, complex formation using urea, molecular distillation, supercritical fluid extraction and silver ion complexing. Complex formation with urea is a preferred method for its simplicity and efficiency in reducing the level of saturated and monounsaturated fatty acids in the oil (Gamez et al., 2003). Initially, the TAGs of the oil are split into their constituent fatty acids, often in the form of fatty acid esters, by hydrolysis under either acid or base catalysed reaction conditions, whereby one mol of TAG is reacted with at least 3 mol of alcohol (e.g. ethanol for ethyl esters or methanol for methyl esters) with excess alcohol used to enable separation of the formed alkyl esters and the glycerol that is also formed, or by lipases. These free fatty acids or fatty acid esters, which are usually unaltered in fatty acid composition by the treatment, may then be mixed with an ethanolic solution of urea for complex formation. The saturated and monounsaturated fatty acids easily complex with urea and crystallize out on cooling and may subsequently be removed by filtration. The non-urea complexed fraction is thereby enriched with LC-PUFA.

### Feedstuffs

The present invention includes compositions which can be used as feedstuffs. For purposes of the present invention, “feedstuffs” include any food or preparation for human or animal consumption which when taken into the body (a) serve to nourish or build up tissues or supply energy; and/or (b) maintain, restore or support adequate nutritional status or metabolic function. Feedstuffs of the invention include nutritional compositions for babies and/or young children such as, for example, infant formula, and seedmeal of the invention.

Feedstuffs of the invention comprise, for example, a cell of the invention, a plant of the invention, the plant part of the invention, the seed of the invention, an extract of the invention, the product of the method of the invention, the product of the fermentation process of the invention, or a composition along with a suitable carrier(s). The term “carrier” is used in its broadest sense to encompass any component which may or may not have nutritional value. As the skilled addressee will appreciate, the carrier must be suitable for use (or used in a sufficiently low concentration) in a feedstuff such that it does not have deleterious effect on an organism which consumes the feedstuff.

The feedstuff of the present invention comprises an oil, fatty acid ester, or fatty acid produced directly or indirectly by use of the methods, cells or plants disclosed herein. The composition may either be in a solid or liquid form. Additionally, the composition may include edible macronutrients, protein, carbohydrate, vitamins, and/or minerals in amounts desired for a particular use. The amounts of these ingredients will vary depending on whether the composition is intended for use with normal individuals or for use with individuals having specialized needs, such as individuals suffering from metabolic disorders and the like.

Examples of suitable carriers with nutritional value include, but are not limited to, macronutrients such as edible fats, carbohydrates and proteins. Examples of such edible fats include, but are not limited to, coconut oil, borage oil, fungal oil, black current oil, soy oil, and mono- and diglycerides. Examples of such carbohydrates include (but are not limited to): glucose, edible lactose, and hydrolyzed starch. Additionally, examples of proteins which may be utilized in the nutritional composition of the invention include (but are not limited to) soy proteins, electrodialysed whey, electrodialysed skim milk, milk whey, or the hydrolysates of these proteins.

With respect to vitamins and minerals, the following may be added to the feedstuff compositions of the present invention: calcium, phosphorus, potassium, sodium, chloride, magnesium, manganese, iron, copper, zinc, selenium, iodine, and Vitamins A, E, D, C, and the B complex. Other such vitamins and minerals may also be added.

The components utilized in the feedstuff compositions of the present invention can be of semi-purified or purified origin. By semi-purified or purified is meant a material which has been prepared by purification of a natural material or by de noo synthesis.

A feedstuff composition of the present invention may also be added to food even when supplementation of the diet is not required. For example, the composition may be added to food of any type, including (but not limited to): margarine, modified butter, cheeses, milk, yogurt, chocolate, candy, snacks, salad oils, cooking oils, cooking fats, meats, fish and beverages.

The genus Saccharomyces spp is used in both brewing of beer and wine making and also as an agent in baking, particularly bread. Other yeasts such as oleaginous yeast including, for example, Yarrowia spp, are also useful in LC-PUFA production. Yeasts may be used as an additive in animal feed, such as in aquaculture. It will be apparent that genetically engineered yeast strains can be provided which are adapted to synthesise LC-PUFA as described herein. These yeast strains, or LC-PUFA produced therein, can then be used in food stuffs and in wine and beer making to provide products which have enhanced fatty acid content.

Additionally, fatty acids produced in accordance with the present invention or host cells transformed to contain and express the subject genes may also be used as animal food supplements to alter an animal's tissue, egg or milk fatty acid composition to one more desirable for human or animal consumption. Examples of such animals include sheep, cattle, horses, poultry such as chickens and the like.

Furthermore, feedstuffs of the invention can be used in aquaculture to increase the levels of fatty acids in fish or crustaceans such as, for example, prawns for human or animal consumption. Preferred fish are salmon.

Preferred feedstuffs of the invention are the plants, seed and other plant parts such as leaves and stems which may be used directly as food or feed for humans or other animals. For example, animals may graze directly on such plants grown in the field or be fed more measured amounts in controlled feeding. The invention includes the use of such plants and plant parts as feed for increasing the LC-PUFA levels in humans and other animals.

### Compositions

The present invention also encompasses compositions, particularly pharmaceutical compositions, comprising one or more of the fatty acids and/or resulting oils produced using the methods of the invention.

A pharmaceutical composition may comprise one or more of the fatty acids and/or oils, in combination with a standard, well-known, non-toxic pharmaceutically-acceptable carrier, adjuvant or vehicle such as phosphate-buffered saline, water, ethanol, polyols, vegetable oils, a wetting agent or an emulsion such as a water/oil emulsion. The composition may be in either a liquid or solid form. For example, the composition may be in the form of a tablet, capsule, ingestible liquid or powder, injectible, or topical ointment or cream. Proper fluidity can be maintained, for example, by the maintenance of the required particle size in the case of dispersions and by the use of surfactants. It may also be desirable to include isotonic agents, for example, sugars, sodium chloride, and the like. Besides such inert diluents, the composition can also include adjuvants, such as wetting agents, emulsifying and suspending agents, sweetening agents, flavoring agents and perfuming agents.

Suspensions, in addition to the active compounds, may comprise suspending agents such as ethoxylated isostearyl alcohols, polyoxyethylene sorbitol and sorbitan esters, microcrystalline cellulose, aluminum metahydroxide, bentonite, agar-agar, and tragacanth or mixtures of these substances.

Solid dosage forms such as tablets and capsules can be prepared using techniques well known in the art. For example, fatty acids produced in accordance with the present invention can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant fatty acid(s).

For intravenous administration, the fatty acids produced in accordance with the present invention or derivatives thereof may be incorporated into commercial formulations.

A typical dosage of a particular fatty acid is from 0.1 mg to 20 g, taken from one to five times per day (up to 100 g daily) and is preferably in the range of from about 10 mg to about 1, 2, 5, or 10 g daily (taken in one or multiple doses). As known in the art, a minimum of about 300 mg/day of fatty acid, especially LC-PUFA, is desirable. However, it will be appreciated that any amount of fatty acid will be beneficial to the subject.

Possible routes of administration of the pharmaceutical compositions of the present invention include, for example, enteral (e.g., oral and rectal) and parenteral. For example, a liquid preparation may be administered orally or rectally. Additionally, a homogenous mixture can be completely dispersed in water, admixed under sterile conditions with physiologically acceptable diluents, preservatives, buffers or propellants to form a spray or inhalant.

The dosage of the composition to be administered to the patient may be determined by one of ordinary skill in the art and depends upon various factors such as weight of the patient, age of the patient, overall health of the patient, past history of the patient, immune status of the patient, etc.

Additionally, the compositions of the present invention may be utilized for cosmetic purposes. It may be added to pre-existing cosmetic compositions such that a mixture is formed or a fatty acid produced according to the subject invention may be used as the sole “active” ingredient in a cosmetic composition.

### EXAMPLES

### Example 1. Materials and Methods

### Expression of Genes in Plant Cells in a Transient Expression System

Exogenous genetic constructs were expressed in plant cells in a transient expression system essentially as described by Voinnet et al. (2003) and Wood et al. (2009). Plasmids containing a coding region to be expressed from a strong constitutive promoter such as the CaMV 35S promoter were introduced into Agrobacterium tumefaciens strain AGL1. A chimeric gene 35S:p19 for expression of the p19 viral silencing suppressor was separately introduced into AGL1, as described in WO 2010/057246. The recombinant Agrobacterium cells were grown at 28° C. in LB broth supplemented with 50 mg/L kanamycin and 50 mg/L rifampicin to stationary phase. The bacteria were then pelleted by centrifugation at 5000 g for 15 min at room temperature before being resuspended to OD600=1.0 in an infiltration buffer containing 10 mM MES pH 5.7, 10 mM MgCl₂ and 100 μM acetosyringone. The cells were then incubated at 28° C. with shaking for 3 hours before equal volumes of Agrobacterium cultures containing 35S:p19 and the test chimeric construct(s) of interest were mixed prior to infiltration into leaf tissue. The plants were typically grown for a further five days after infiltration before leaf discs were taken and freeze-dried for GC analysis of the fatty acids.

Fatty acid methyl esters (FAME) of total leaf lipids in freeze-dried samples were produced by incubating the samples in methanol/HCl/dichloromethane (10/1/1 v/v) solution for 2 hours at 80° C. together with a known amount of hexadecanoic acid as an internal standard. FAMEs were extracted in hexane/DCM, concentrated to a small volume in hexane and injected into a GC. The amount of individual and total fatty acids present in the lipid fractions were quantified on the basis of the known amount of internal standard.

### Gas Chromatography (GC) Analysis of Fatty Acids

FAME were analysed by gas chromatography using an Agilent Technologies 7890A GC (Palo Alto, California. USA) equipped with a 30 m SGE-BPX70 column (70% cyanopropyl polysilphenylene-siloxane, 0.25 mm inner diameter, 0.25 mm film thickness), an FID, a split/splitless injector and an Agilent Technologies 7693 Series auto sampler and injector. Helium was used as the carrier gas. Samples were injected in split mode (50:1 ratio) at an oven temperature of 150° C. After injection, the oven temperature was held at 150° C. for 1 min then raised to 210° C. at 3° C. min⁻¹, again raised to 240° C. at 50° C. min⁻¹ and finally holding for 1.4 min at 240° C. Peaks were quantified with Agilent Technologies ChemStation software (Rev B.04.03 (16), Palo Alto, California. USA) based on the response of the known amount of the external standard GLC-411 (Nucheck) and C17:0-ME internal standard.

Liquid Chromatography-Mass Spectrometry (LC-MS) analysis of lipids Total lipids were extracted from freeze-dried developing seeds, twelve days after flowering (daf), and mature seeds after adding a known amount of tri-C17:0-TAG as an internal quantitation standard. The extracted lipids were dissolved into 1 mL of 10 mM butylated hydroxytoluene in butanol:methanol (1:1 v/v) per 5 mg dry material and analysed using an Agilent 1200 series LC and 6410b electrospray ionisation triple quadrupole LC-MS. Lipids were chromatographically separated using an Ascentis Express RP-Amide column (50 mm×2.1 mm, 2.7 μm, Supelco) operating a binary gradient with a flow rate of 0.2 mL/min. The mobile phases were: A. 10 mM ammonium formate in H₂O:methanol:tetrahydrofuran (50:20:30 v/v/v); B. 10 mM ammonium formate in H₂O:methanol:tetrahydrofuran (5:20:75, v/v/v). Multiple reaction monitoring (MRM) lists were based on the following major fatty acids: 16:0, 18:0, 18:1, 18:2, 18:3, 18:4, 20:1, 20:2, 20:3, 20:4, 20:5, 22:4, 22:5, 22:6 using a collision energy of 30 V and fragmentor of 60 V. Individual MRM TAG was identified based on ammoniated precursor ion and product ion from neutral loss of 22:6. TAG was quantified using a 10 μM tristearin external standard.

### Determination of Seed Fatty Acid Profile and Oil Content

Where seed oil content was to be determined, seeds were dried in a desiccator for 24 h and approximately 4 mg of seed was transferred to a 2 ml glass vial containing Teflon-lined screw cap. 0.05 mg triheptadecanoin dissolved in 0.1 ml toluene was added to the vial as internal standard.

Seed FAME were prepared by adding 0.7 ml of IN methanolic HCl (Supelco) to the vial containing seed material, vortexed briefly and incubated at 80° C. for 2 h. After cooling to room temperature, 0.3 ml of 0.9% NaCl (w/v) and 0.1 ml hexane was added to the vial and mixed well for 10 min in Heidolph Vibramax 110. The FAME was collected into 0.3 ml glass insert and analysed by GC with a flame ionization detector (FID) as mentioned earlier.

The peak area of individual FAME were first corrected on the basis of the peak area responses of known amount of the same FAMEs present in a commercial standard GLC-411 (NU-CHEK PREP, INC., USA). GLC-411 contains equal amounts of 31 fatty acids (% by wt), ranging from C8:0 to C22:6. In case of fatty acids, which were not present in the standard, the inventors took the peak area responses of the most similar FAME. For example, peak area response of FAMEs of 16:1d9 was used for 16:1d7 and FAME response of C22:6 was used for C22:5. The corrected areas were used to calculate the mass of each FAME in the sample by comparison to the internal standard mass. Oil is stored mainly in the form of TAG and its weight was calculated based on FAME weight. Total moles of glycerol was determined by calculating moles of each FAMES and dividing total moles of FAMEs by three. TAG was calculated as the sum of glycerol and fatty acyl moieties using a relation: % oil by weight=100×((41×total mol FAME/3)+(total g FAME−(15×total mol FAME)))/g seed, where 41 and 15 are molecular weights of glycerol moiety and methyl group, respectively.

### Analysis of the Sterol Content of Oil Samples

Samples of approximately 10 mg of oil together with an added aliquot of C24:0 monol as an internal standard were saponified using 4 mL 5% KOH in 80% MeOH and heating for 2 h at 80° C. in a Teflon-lined screw-capped glass tube. After the reaction mixture was cooled, 2 mL of Milli-Q water were added and the sterols were extracted into 2 mL of hexane:dichloromethane (4:1 v/v) by shaking and vortexing. The mixture was centrifuged and the sterol extract was removed and washed with 2 mL of Milli-Q water. The sterol extract was then removed after shaking and centrifugation. The extract was evaporated using a stream of nitrogen gas and the sterols silylated using 200 mL of BSTFA and heating for 2 h at 80° C.

For GC/GC-MS analysis of the sterols, sterol-OTMSi derivatives were dried under a stream of nitrogen gas on a heat block at 40° C., and then re-dissolved in chloroform or hexane immediately prior to GC/GC-MS analysis. The sterol-OTMS derivatives were analysed by gas chromatography (GC) using an Agilent Technologies 6890A GC (Palo Alto, California, USA) fitted with an Supelco Equity™-1 fused silica capillary column (15 m×0.1 mm i.d., 0.1 μm film thickness), an FID, a split/splitless injector and an Agilent Technologies 7683B Series auto sampler and injector. Helium was the carrier gas. Samples were injected in splitless mode at an oven temperature of 120′C. After injection, the oven temperature was raised to 270° C. at 10° C. min⁻¹ and finally to 300° C. at 5° C. min⁻¹. Peaks were quantified with Agilent Technologies ChemStation software (Palo Alto, California. USA). GC results are subject to an error of ±5% of individual component areas.

GC-mass spectrometric (GC-MS) analyses were performed on a Finnigan Thermoquest GCQ GC-MS and a Finnigan Thermo Electron Corporation GC-MS; both systems were fitted with an on-column injector and Thermoquest Xcalibur software (Austin, Texas, USA). Each GC was fitted with a capillary column of similar polarity to that described above. Individual components were identified using mass spectral data and by comparing retention time data with those obtained for authentic and laboratory standards. A full procedural blank analysis was performed concurrent to the sample batch.

RT-PCR Conditions

Reverse transcription-PCR (RT-PCR) amplification was typically carried out using the Superscript III One-Step RT-PCR system (Invitrogen) in a volume of 25 μL using 10 pmol of the forward primer and 30 pmol of the reverse primer, MgSO₄ to a final concentration of 2.5 mM, 400 ng of total RNA with buffer and nucleotide components according to the manufacturer's instructions. Typical temperature regimes were: 1 cycle of 45° C. for 30 minutes for the reverse transcription to occur: then 1 cycle of 94° C. for 2 minutes followed by 40 cycles of 94° C. for 30 seconds, 52° C. for 30 seconds, 70° C. for 1 minute; then 1 cycle of 72° C. for 2 minutes before cooling the reaction mixtures to 5° C.

Production of B. napus Somatic Embryos by Induction with 35S-LEC2

B. napus (cv. Oscar) seeds were sterilized using chlorine gas as described by (Attila Kereszt et al., 2007). Sterilized seeds were germinated on ½ strength MS media (Murashige and Skoog, 1962) with 0.8% agar adjusted to pH 5.8, and grown at 24° C. under fluorescent lighting (50 μE/m²s) with a 18/6 h (light/dark) photoperiod for 6-7 days. Cotyledonary petioles with 2-4 mm stalk length were isolated aseptically from these seedlings and used as explants. Cultures of the transformed A. tumefaciens strain AGL1, one harbouring a seed specific binary vector and a second with a 35S-LEC2 construct were inoculated from single colonies from fresh plates and grown in 10 mL of LB medium with appropriate antibiotics and grown overnight at 28° C. with agitation at 150 rpm. The bacterial cells were collected by centrifugation at 4000 rpm for 5 minutes, washed with MS media containing 2% sucrose and re-suspended in 10 mL of the same medium and grown with antibiotics for selection as appropriate for 4 hours after the addition of acetosyringone to 100 μM. Two hours before addition to the plant tissues, spermidine was added to a final concentration of 1.5 mM and the final density of the bacteria adjusted to OD 600 nm=0.4 with fresh medium. The two bacterial cultures, one carrying the seed specific construct and other carrying 35S-AtLEC2, were mixed in 1:1 to 1:1.5 ratios.

Freshly-isolated B. napus cotyledonary petioles were infected with 20 mL A. tumefaciens cultures for 6 minutes. The cotyledonary petioles were blotted on sterile filter paper to remove excess A. tumefaciens and then transferred to co-cultivation media (MS media with 1 mg/L TDZ, 0.1 mg/L NAA, 100 μM acetosyringone supplemented with L-cysteine (50 mg/L), ascorbic acid (15 mg/L) and MES (250 mg/l)). The plates were sealed with micro-pore tape and incubated in the dark at 24° C. for 48 hrs. The co-cultivated explants were transferred to pre-selection media (MS containing 1 mg/L TDZ, 0.1 mg/L NAA, 3 mg/L AgNO₃, 250 mg/L cefotaxime and 50 mg/L timentin) and cultured for 4-5 days at 24° C. with a 16 h/8 h photoperiod. The explants were then transferred to selection media (MS containing 1 mg/L TDZ, 0.1 mg/L NAA, 3 mg/L AgNO₃, 250 mg/L cefotaxime and 50 mg/L timentin) according to the selectable marker gene on the seed specific vector and cultured for 2-3 weeks at 24° C. with a 16 h/8 h photoperiod. Explants with green embryogenic callus were transferred to hormone free MS media (MS with 3 mg/L AgNO₃, 250 mg/L cefotaxime, 50 mg/L timentin and the selection agent) and cultured for another 2-3 weeks. Torpedo or cotyledonary stage embryos isolated from surviving explants on the selection medium were analysed for fatty acid composition in their total lipid using GC.

### Example 2. Stable Expression of Transgenic DHA Pathways in Arabidopsis thaliana Seeds

### Binary Vector Construction

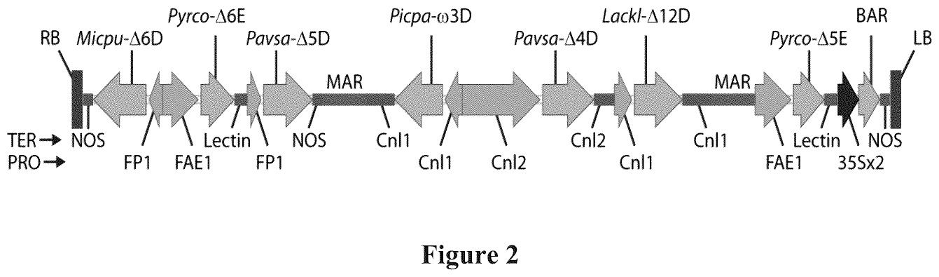

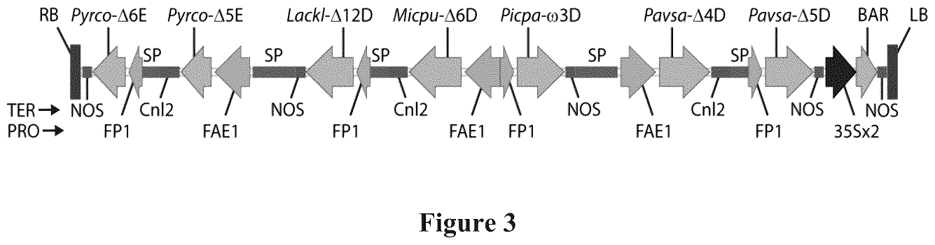

The binary vectors pJP3416-GA7 and pJP3404 each contained seven heterologous fatty acid biosynthesis genes, encoding 5 desaturases and 2 elongases, and a plant selectable marker between the left and right border repeats of the T-DNA present in each vector (FIGS. 2 and 3). SEQ ID NO:1 provides the nucleotide sequence of the T-DNA region of pJP3416-GA7 from the right to left border sequences. Both genetic constructs contained plant codon-optimised genes encoding a Lachancea kluyveri Δ12-desaturase (comprising nucleotides 14143-16648 of SEQ ID NO:1), a Pichia pastoris ω3-desaturase (comprising nucleotides 7654-10156 of SEQ ID NO:1), a Micromonas pusilla Δ6-desaturase (comprising nucleotides 226-2309 of SEQ ID NO:1), Pavlova salina Δ5- and Δ4-desaturases (comprising nucleotides 4524-6485 and 10157-14142 of SEQ ID NO:1, respectively) and Pyramimonas cordata Δ6- and Δ5-elongases (comprising nucleotides 2310-4523 and 17825-19967 of SEQ ID NO: 1, respectively). The specific regions of the T-DNA (Orientation: right to left border sequences) region of the binary vector pJP3416-GA7 with respect to SEQ ID NO:1 are as follows:

Nucleotides 1-163: Right border: 480-226, Agrobacterium tumefaciens nopaline synthase terminator (TER_NOS); 1883-489, Micromonas pusilla Δ6-desaturase; 2309-1952, Brassica napus truncated napin promoter (PRO_FP1); 2310-3243, Arabidopsis thaliana FAE1 promoter (PRO_FAE1); 3312-4181, Pyramimonas cordata Δ6-elongase; 4190-4523, Glycine max lectin terminator (TER_Lectin); 4524-4881, PRO_FP1; 4950-6230: Pavlova salina Δ5-desaturase: 6231-6485: TER_NOS; 7653-6486, Nicotiana tabacum Rb7 matrix attachment region (MAR); 8387-7654, Linum usitatissimum conlinin1 terminator (TER_Cnl1); 9638-8388, Pichia pastoris ω3-desaturase; 10156-9707, Linum usitatissimum conlinin1 promoter (PRO_Cnl1); 10157-12189, Linum usitatissimum conlinin1 promoter: 12258-13604. Pavlova salina Δ4-desaturase; 13605-14142, Linum usitatissimum conlinin2 terminator: 14143-14592, PRO_Cnl1; 14661-15914, Lachancea kluyveri Δ12-desaturase: 15915-16648, TER_Cnl1: 17816-16649, MAR; 17825-18758, PRO_FAE1; 18827-19633, Pyramimonas cordata Δ5-elongase: 19634-19967, TER_Lectin: 19990-20527, Cauliflower mosaic virus 35S promoter with duplicated enhancer region; 20537-21088, Streptomyces viridochromogenes phosphinothricin-N-acetyltransferase: 21097-21349, TER_NOS: 21367-21527, Left border.

The seven coding regions in the constructs were each under the control of a seed specific promoter-three different promoters were used, namely the truncated Brassica napus napin promoter (pBnFP1), the Arabidopsis thaliana FAE1 promoter (pAtFAE1) and the Linum usitatissimum conlinin 1 promoter (pLuCnl1). The seven fatty acid biosynthesis genes together coded for an entire DHA synthesis pathway that was designed to convert 18:1^{Δ9} (oleic acid) through to 22:6^{Δ4,7,10,13,16,19} (DHA). Both binary vectors contained a BAR plant selectable marker coding region operably linked to a Cauliflower Mosaic Virus (CaMV) 35S promoter with duplicated enhancer region and A. tumefaciens nos3′ polyadenylation region-transcription terminator. The plant selectable marker was situated adjacent to the left border of the T-DNA region, therefore distally located on the T-DNA with respect to the orientation of T-DNA transfer into the plant cells. This increased the likelihood that partial transfer of the T-DNA, which would be likely to not include the selectable marker gene, would not be selected. pJP3416-GA7 and pJP3404 each contained an RiA4 origin of replication from Agrobacterium rhizogenes (Hamilton, 1997).

pJP3416-GA7 was generated by synthesising the DNA region corresponding to nucleotides 226-19975 of SEQ ID NO:1 (GA7 region) and inserting this region into the recipient binary vector pJP3416 at the PspOMI site. Each fatty acid biosynthetic gene on GA7 included a Tobacco Mosaic Virus 5′ untranslated region (5′UTR) sequence which was operably linked to each coding region, between the promoter and the translation initiation ATG, to maximise translation efficiency of the mRNAs produced from the genes. The GA7 construct also included two Nicotiana tabacum Rb7 matrix attachment region (MAR) sequences, as described by Hall et al. (1991). MAR sequences, sometimes termed nuclear attachment regions, are known to bind specifically to the nuclear matrix in vitro and may mediate binding of chromatin to the nuclear matrix in vivo. MARs are thought to function to reduce transgene silencing. In pJP3416-GA7 the MARs were also inserted and positioned within the T-DNA region in order to act as DNA spacers to insulate transgenic expression cassettes. The pJP3416 vector prior to insertion of the GA7 region contained only the plant selectable marker cassette between the borders.

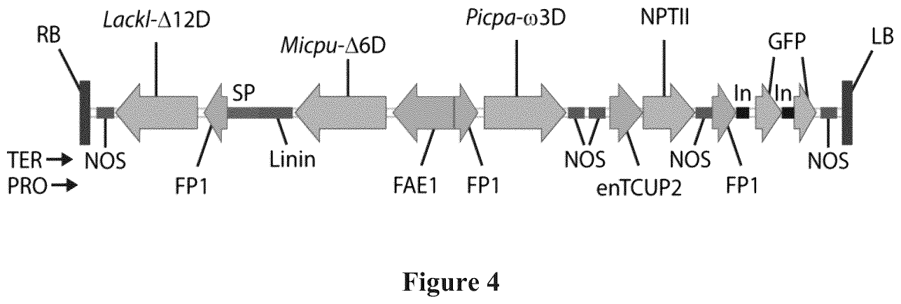

The genetic construct pJP3404 was made by sequential restriction enzyme-based insertions in which gene cassettes were added to the binary vector, pJP3367, which comprised genes for production of SDA in seeds. This construct contained genes encoding the L. kluyveri Δ12-desaturase and P. pastoris ω3-desaturase, both expressed by the B. napus truncated napin promoter (FP1), and the M. pusilla Δ6-desaturase expressed by the A. thaliana FAE1 promoter (FIG. 4). First, the A. thaliana FAD2 intron was flanked by EcoRI sites and cloned into the pJP3367 MfeI site to generate pJP3395. A fragment containing the P. cordata Δ6- and Δ5-elongase cassettes driven by the FAE1 and FP1 promoters, respectively, was cloned into the KasI site of pJP3395 to generate pJP3398. pJP3399 was then generated by replacing the RK2 origin of replication in pJP3398 with a RiA4 origin of replication. The final binary vector, pJP3404, was generated by cloning a Sb/I-flanked fragment containing the P. salina Δ5- and Δ4-desaturase cassettes driven by the FP1 and FAE1 promoters, respectively, into the Sb/I site of pJP3399.

A. thaliana Transformation and Analysis of Fatty Acid Composition

The chimeric vectors were introduced into A. tumefaciens strain AGL1 and cells from cultures of the transformed Agrobacterium used to treat A. thaliana (ecotypes Columbia and a fad2 mutant) plants using the floral dip method for transformation (Clough and Bent, 1998). After maturation, the T₁ seeds from the treated plants were harvested and plated onto MS plates containing PPT for selection of plants containing the BAR selectable marker gene. Surviving, healthy Ti seedlings were transferred to soil. After growth of the plants to maturity and allowing for self-fertilisation, T₂ seeds from these plants were harvested and the fatty acid composition of their seed lipid analysed by GC analysis as described in Example 1.

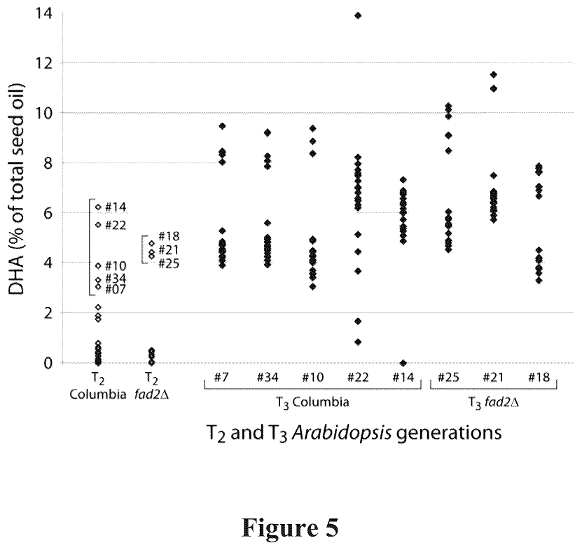

The data for the DHA level in the seed lipids are shown in FIG. 5 (lanes labelled T₂) for 13 transformants using pJP3416-GA7 into the Columbia genetic background, and for six transformants using the fad2 mutant. The pJP3416-GA7 construct resulted in the production of slightly higher levels of DHA, as a percentage of total fatty acid content, on average than the pJP3404 construct. Table 4 shows the fatty acid composition of total seed lipid from the T₂ lines with the highest DHA levels. The calculated conversion efficiencies for each enzymatic step in the production of DHA from oleic acid in the same seeds are shown in Table 5. Conversion efficiencies were calculated as (% products×100)/(% remaining substrate+% products), thereby expressed as a percentage.

The highest observed level of DHA produced in the pJP3416-GA7 T₂ transformed lines was 6.2%, additionally with 0.5% EPA and 0.2% DPA (line #14). These T₂ seeds were still segregating for the transgene i.e. were not yet uniformly homozygous. Compiled data from the total seed lipid profiles from independent transgenic seed (Table 4) are shown in Table 6. The level of ω3 fatty acids produced as a result of the transgenes in these seeds (total new ω3 fatty acids, excluding the level of ALA which was produced endogenously in the Columbia background) was 10.7% while the level of ω6 fatty acids (total new ω6 fatty acids but excluding 18:2^{Δ9,12}) was 1.5%. This represents an extremely favourable ration of new ω3 fatty acids:new ω6 fatty acids, namely 7.3:1.

T₂ seeds of selected lines transformed with pJP3416-GA7, namely for lines designated 7, 10, 14, 22 and 34 in the Columbia background and for lines designated 18, 21 and 25 in the fad2 mutant background, were plated onto MS media containing PPT for selection of transgenic seedlings in vitro. Twenty PPT-resistant seedlings for each line were transferred to soil and grown to maturity after self-fertilisation. These plants were highly likely to be homozygous for the selectable marker gene, and therefore for at least one T-DNA insertion in the genome of the plants. T₃ seed from these plants were harvested and analysed for fatty acid composition in their seedoil by GC. The data are shown in Table 7. This analysis revealed that the pJP3416-GA7 construct generated higher levels of the ω3 LC-PUFA DHA in T₃ seeds of the homozygous plants than in the segregating T₂ seed. Up to about 13.9% DHA was observed in the T₃ pJP3416-GA7 transformed line designated 22.2 in the Columbia background, increased from about 5.5% in the hemizygous T₂ seed, with a sum level of about 24.3% of new ω3 fatty acids as a percentage of the total fatty acids in the seed lipid content. New ω6 fatty acids were at a level of 1.1% of total fatty acids, representing a very favourable ratio of new ω3 fatty acids:new ω6 fatty acids, namely about 22:1. Similarly, transformants in the fad2 mutant background yielded 20.6% as a sum of new ω3 fatty acids, including 11.5% DHA, as a percentage of the total fatty acids in the seed lipid content.

TABLE 4
Fatty acid composition of total seed lipid from independent transgenic T₂ Arabidopsis seeds with DHA levels at
the higher end of the observed range. ‘Col’ refers to the Columbia ecotype and ‘FAD2’ to the fad2 mutant
ecotype. ‘GA7’ refers to transformation with the T-DNA of the pJP3416-GA7 vector, pJP3404 with the T-DNA
of the pJP3404 vector. 20:1n-9 and 20:1n-11 fatty acids were not resolved in the GC analysis. “Other minor”
fatty acids include 14:0, 16:1n7, 16:1n9, 16:1n13t, 16:2n6, 16:3n3, i18:0, 18:1n5, 20:1n5, 22:0, 22:1n7, 22:1n11/n13, 24:0, 24:1n9.

| | pJP3404_Col_#1 | pJP3404_FAD2_#31 | GA7_Col_#7 | GA7_Col_#34 | GA7_Col_#2 | GA7_Col_#10 |
|---|---|---|---|---|---|---|
| 16:0 | 9.6 | 7.8 | 8.7 | 8.2 | 8.7 | 8.6 |
| 18:0 | 2.9 | 3.9 | 3.7 | 3.9 | 3.6 | 3.3 |
| 18:1d11 | 2.2 | 1.8 | 2.0 | 1.9 | 2.0 | 2.3 |
| 20:0 | 1.6 | 2.3 | 2.0 | 2.0 | 2.1 | 1.6 |
| 20:1d13 | 2.2 | 1.8 | 1.6 | 1.5 | 1.7 | 1.6 |
| 20:1d9/d11 | 13.0 | 15.9 | 16.1 | 16.1 | 16.3 | 15.0 |
| 22:1d13 | 1.1 | 1.2 | 1.1 | 1.1 | 1.3 | 1.0 |
| Other minor | 1.9 | 1.5 | 1.5 | 1.4 | 1.5 | 1.3 |
| 18:1d9 | 10.8 | 14.0 | 10.6 | 10.6 | 10.1 | 11.1 |
| 18:2ω6 | 28.9 | 28.3 | 16.4 | 16.1 | 18.2 | 13.7 |
| 18:3ω3 | 16.6 | 14.9 | 29.6 | 29.6 | 27.5 | 32.4 |
| 18:3ω6 | 0.7 | 0.5 | 0.1 | 0.1 | 0.1 | 0.1 |
| 20:2ω6 | 1.6 | 1.5 | 1.1 | 1.2 | 1.3 | 1.0 |
| 20:3ω6 | 0.0 | 0.0 | 0.0 | 0.0 | 0.0 | 0.0 |
| 20:4ω6 | 0.0 | 0.0 | 0.0 | 0.0 | 0.0 | 0.0 |
| 22:4ω6 | 1.6 | 0.6 | 0.3 | 0.3 | 0.3 | 0.4 |
| 22:5ω6 | 0.1 | 0.1 | 0.0 | 0.0 | 0.0 | 0.0 |
| 18:4ω3 | 1.0 | 0.5 | 1.2 | 1.1 | 1.1 | 1.5 |
| 20:3ω3 | 0.0 | 0.0 | 0.0 | 0.6 | 0.0 | 0.0 |
| 20:4ω3 | 0.4 | 0.6 | 0.6 | 0.7 | 0.5 | 0.8 |
| 20:5ω3 | 0.2 | 0.2 | 0.3 | 0.3 | 0.3 | 0.3 |
| 22:5ω3 | 0.0 | 0.2 | 0.2 | 0.2 | 0.2 | 0.2 |
| 22:6ω3 | 3.6 | 2.4 | 3.0 | 3.1 | 3.3 | 3.9 |

| | GA7_Col_#22 | GA7_Col_#14 | GA7_FAD2_#25 | GA7_FAD2_#21 | GA7_FAD2_#18 |
|---|---|---|---|---|---|
| 16:0 | 8.3 | 9.7 | 7.2 | 8.5 | 7.5 |
| 18:0 | 3.4 | 3.6 | 3.2 | 3.9 | 3.0 |
| 18:1d11 | 2.3 | 2.7 | 1.9 | 2.0 | 1.8 |
| 20:0 | 1.6 | 1.8 | 1.6 | 2.2 | 1.5 |
| 20:1d13 | 1.5 | 1.7 | 1.5 | 1.7 | 1.4 |
| 20:1d9/d11 | 13.9 | 13.5 | 18.3 | 15.9 | 17.0 |
| 22:1d13 | 1.0 | 1.0 | 1.0 | 1.3 | 1.2 |
| Other minor | 1.6 | 1.7 | 1.6 | 1.4 | 1.6 |
| 18:1d9 | 10.0 | 7.7 | 26.0 | 8.2 | 20.9 |
| 18:2ω6 | 13.7 | 11.4 | 6.6 | 16.6 | 4.3 |
| 18:3ω3 | 30.4 | 32.8 | 21.9 | 27.7 | 30.1 |
| 18:3ω6 | 0.2 | 0.1 | 0.1 | 0.2 | 0.1 |
| 20:2ω6 | 1.0 | 1.0 | 0.4 | 1.4 | 0.4 |
| 20:3ω6 | 0.0 | 0.0 | 0.0 | 0.0 | 0.0 |
| 20:4ω6 | 0.0 | 0.0 | 0.0 | 0.0 | 0.0 |
| 22:4ω6 | 0.5 | 0.4 | 0.5 | 0.4 | 0.4 |
| 22:5ω6 | 0.0 | 0.0 | 0.0 | 0.0 | 0.0 |
| 18:4ω3 | 2.7 | 2.7 | 1.9 | 1.8 | 1.7 |
| 20:3ω3 | 0.6 | 0.7 | 0.0 | 0.8 | 0.6 |
| 20:4ω3 | 0.8 | 0.4 | 1.0 | 0.8 | 0.8 |
| 20:5ω3 | 0.7 | 0.5 | 0.6 | 0.4 | 0.5 |
| 22:5ω3 | 0.2 | 0.2 | 0.3 | 0.3 | 0.3 |
| 22:6ω3 | 5.5 | 6.2 | 4.3 | 4.4 | 4.8 |

TABLE 5
Conversion efficiencies of the individual enzymatic steps for production of DHA from oleic
acid, observed in total seed lipid from independent transgenic seed as for Table 4.

| | | pJP3404_Col_#1 | pJP3404_FAD2_#31 | GA7_Col_#7 | GA7_Col_#34 | GA7_Col_#2 | GA7_Col_#10 |
|---|---|---|---|---|---|---|---|
| | d12-des | 69.6% | 62.5% | 66.4% | 66.6% | 66.7% | 67.5% |
| | d15-des | 39.8% | 37.8% | 66.1% | 66.8% | 62.3% | 72.1% |
| Omega-6 | d6-des | 4.5% | 2.5% | 0.7% | 0.7% | 0.7% | 0.9% |
| | (d9-elo) | 3.1% | 3.1% | 2.2% | 2.3% | 2.4% | 1.8% |
| | d6-elo | 71.4% | 56.9% | 83.3% | 83.4% | 83.0% | 84.7% |
| | d5-des | 100.0% | 100.0% | 100.0% | 100.0% | 100.0% | 100.0% |
| | d5-elo | 100.0% | 97.8% | 100.0% | 100.0% | 100.0% | 100.0% |
| | d4-des | 6.2% | 13.0% | 0.0% | 0.0% | 0.0% | 0.0% |
| Omega-3 | d6-des | 23.9% | 21.0% | 15.2% | 15.4% | 16.4% | 17.1% |
| | (d9-elo) | 0.0% | 0.0% | 0.0% | 1.8% | 0.0% | 0.0% |
| | d6-elo | 80.6% | 86.6% | 77.7% | 79.6% | 79.4% | 77.5% |
| | d5-des | 93.7% | 92.1% | 91.7% | 91.4% | 91.5% | 92.6% |
| | d5-elo | 93.7% | 92.1% | 91.7% | 91.4% | 91.5% | 92.6% |
| | d4-des | 100.0% | 90.6% | 94.8% | 94.0% | 95.3% | 94.4% |

| | | GA7_Col_#22 | GA7_Col_#14 | GA7_FAD2_#25 | GA7_FAD2_#21 | GA7_FAD2_#18 |
|---|---|---|---|---|---|---|
| | d12-des | 70.2% | 72.7% | 45.9% | 69.5% | 53.7% |
| | d15-des | 72.7% | 77.2% | 79.7% | 66.0% | 88.1% |
| Omega-6 | d6-des | 1.3% | 1.0% | 1.6% | 1.1% | 1.1% |
| | (d9-elo) | 1.8% | 1.7% | 1.2% | 2.7% | 0.9% |
| | d6-elo | 70.3% | 74.5% | 85.5% | 66.1% | 88.0% |
| | d5-des | 100.0% | 100.0% | 100.0% | 100.0% | 100.0% |
| | d5-elo | 100.0% | 100.0% | 100.0% | 100.0% | 100.0% |
| | d4-des | 0.0% | 0.0% | 0.0% | 0.0% | 0.0% |
| Omega-3 | d6-des | 24.7% | 23.6% | 27.1% | 21.9% | 21.0% |
| | (d9-elo) | 2.0% | 2.2% | 0.0% | 2.6% | 2.1% |
| | d6-elo | 72.7% | 73.0% | 76.7% | 77.4% | 79.2% |
| | d5-des | 89.6% | 92.4% | 88.0% | 91.8% | 91.0% |
| | d5-elo | 89.6% | 92.4% | 88.0% | 91.8% | 91.0% |
| | d4-des | 95.8% | 96.9% | 93.1% | 92.9% | 94.2% |

TABLE 6
Compiled data from the total seed lipid profiles from independent transgenic seed
shown in Table 2. Calculations do not include the ‘minor fatty acids’ in Table 4.

| Parameter | pJP3404_Col_#1 | pJP3404_FAD2_#31 | GA7_Col_#7 | GA7_Col_#34 | GA7_Col_#2 | GA7_Col_#10 |
|---|---|---|---|---|---|---|
| total w3 (% of total FA) | 21.8 | 18.8 | 34.9 | 35.6 | 32.9 | 39.1 |
| total w6 (% of total FA) | 32.9 | 31.0 | 17.9 | 17.7 | 19.9 | 15.2 |
| w3/w6 ratio | 0.66 | 0.61 | 1.95 | 2.01 | 1.65 | 2.57 |
| w6/w3 ratio | 1.51 | 1.65 | 0.51 | 0.50 | 0.60 | 0.39 |
| total novel w3 (% of total FA) | 5.2 | 3.9 | 5.3 | 6.0 | 5.4 | 6.7 |
| total novel w6 (% of total FA) | 4.0 | 2.7 | 1.5 | 1.6 | 1.7 | 1.5 |
| novel w3/w6 ratio | 1.30 | 1.44 | 3.53 | 3.75 | 3.18 | 4.47 |
| novel w6/w3 ratio | 0.77 | 0.69 | 0.28 | 0.27 | 0.31 | 0.22 |
| OA to EPA efficiency | 4.8% | 3.5% | 4.3% | 4.4% | 4.7% | 5.4% |
| OA to DHA efficiency | 4.5% | 3.0% | 3.7% | 3.8% | 4.1% | 4.8% |
| LA to EPA efficiency | 6.9% | 5.6% | 6.6% | 6.8% | 7.2% | 8.1% |
| LA to DHA efficiency | 6.6% | 4.8% | 5.7% | 5.8% | 6.3% | 7.2% |
| ALA to EPA efficiency | 17.4% | 14.9% | 10.0% | 10.1% | 11.6% | 11.3% |
| ALA to DHA efficiency | 16.5% | 12.8% | 8.6% | 8.7% | 10.0% | 10.0% |
| total saturates | 14.1 | 14.0 | 14.4 | 14.1 | 14.4 | 13.5 |
| total monounsaturates | 29.3 | 34.7 | 31.4 | 31.2 | 31.4 | 31.0 |
| total polyunsaturates | 54.7 | 49.8 | 52.8 | 53.3 | 52.8 | 54.3 |
| total C20 | 17.4 | 20 | 19.7 | 20.4 | 20.1 | 18.7 |
| total C22 | 6.4 | 4.5 | 4.6 | 4.7 | 5.1 | 5.5 |
| C20/C22 ratio | 2.72 | 4.44 | 4.28 | 4.34 | 3.94 | 3.40 |

| Parameter | GA7_Col_#22 | GA7_Col_#14 | GA7_FAD2_#25 | GA7_FAD2_#21 | GA7_FAD2_#18 |
|---|---|---|---|---|---|
| total w3 (% of total FA) | 40.9 | 43.5 | 30.0 | 36.2 | 38.8 |
| total w6 (% of total FA) | 15.4 | 12.9 | 7.6 | 18.6 | 5.2 |
| w3/w6 ratio | 2.66 | 3.37 | 3.95 | 1.95 | 7.46 |
| w6/w3 ratio | 0.38 | 0.30 | 0.25 | 0.51 | 0.13 |
| total novel w3 (% of total FA) | 10.5 | 10.7 | 8.1 | 8.5 | 8.7 |
| total novel w6 (% of total FA) | 1.7 | 1.5 | 1.0 | 2.0 | 0.9 |
| novel w3/w6 ratio | 6.18 | 7.13 | 8.10 | 4.25 | 9.67 |
| novel w6/w3 ratio | 0.16 | 0.14 | 0.12 | 0.24 | 0.10 |
| OA to EPA efficiency | 7.9% | 8.8% | 6.3% | 6.4% | 6.7% |
| OA to DHA efficiency | 6.8% | 7.9% | 5.2% | 5.5% | 5.8% |
| LA to EPA efficiency | 11.4% | 12.2% | 13.8% | 9.3% | 12.7% |
| LA to DHA efficiency | 9.8% | 11.0% | 11.4% | 8.0% | 10.9% |
| ALA to EPA efficiency | 15.6% | 15.9% | 17.3% | 14.1% | 14.4% |
| ALA to DHA efficiency | 13.4% | 14.3% | 14.3% | 12.2% | 12.4% |
| total saturates | 13.3 | 15.1 | 12.0 | 14.6 | 12.0 |
| total monounsaturates | 28.7 | 26.6 | 48.7 | 29.1 | 42.3 |
| total polyunsaturates | 56.3 | 56.4 | 37.6 | 54.8 | 44.0 |
| total C20 | 18.5 | 17.8 | 21.8 | 21 | 20.7 |
| total C22 | 7.2 | 7.8 | 6.1 | 6.4 | 6.7 |
| C20/C22 ratio | 2.57 | 2.28 | 3.57 | 3.28 | 3.09 |

TABLE 7
Fatty acid composition of total seed lipid from independent transgenic
T₃ and T₄ Arabidopsis progeny seeds obtained from plant lines as in Table
3. The error shown in the T₄ generation denotes the SD of n = 10.

| | GA7_Col_7.2 | GA7_Col_34.2 | GA7_Col_10.13 | GA7_Col_22.2 | GA7_Col_14.19 |
|---|---|---|---|---|---|
| 16:0 | 9.8 | 9.0 | 9.5 | 11.2 | 10.4 |
| 18:0 | 4.0 | 3.8 | 4.2 | 3.4 | 3.5 |
| 18:1n7 | 2.0 | 1.9 | 2.2 | 2.9 | 2.5 |
| 20:0 | 2.2 | 1.9 | 1.7 | 1.4 | 2.3 |
| 20:1d13 | 1.4 | 1.3 | 1.2 | 1.6 | 2.5 |
| 20:1d9/11 | 13.6 | 14.7 | 12.4 | 9.5 | 13.0 |
| 22:1d13 | 1.2 | 1.2 | 0.8 | 0.6 | 1.6 |
| Other minor | 1.8 | 1.5 | 1.5 | 2.1 | 2.6 |
| 18:1d9 | 5.5 | 6.7 | 6.8 | 4.6 | 6.9 |
| 18:2ω6 | 7.5 | 7.9 | 7.4 | 5.6 | 14.8 |
| 18:3ω3 | 33.7 | 33.7 | 36.1 | 31.5 | 26.1 |
| 18:3ω6 | 0.2 | 0.2 | 0.2 | 0.4 | 0.1 |
| 20:2ω6 | 1.0 | 1.0 | 0.7 | 0.7 | 1.4 |
| 20:3ω6 | 0 | 0 | 0 | 0 | 0 |
| 20:4ω6 | 0 | 0 | 0 | 0 | 0 |
| 22:4ω6 | 0 | 0 | 0 | 0 | 0 |
| 22:5ω6 | 0 | 0 | 0 | 0 | 0 |
| 18:4ω3 | 3.1 | 2.6 | 3.0 | 5.3 | 3.3 |
| 20:3ω3 | 1.4 | 1.3 | 1.2 | 1.3 | 1.2 |
| 20:4ω3 | 0.7 | 0.6 | 0.6 | 0.9 | 0.2 |
| 20:5ω3 | 0.9 | 0.9 | 0.7 | 1.9 | 0.8 |
| 22:5ω3 | 0.7 | 0.6 | 0.6 | 1.0 | 0.4 |
| 22:6ω3 | 9.5 | 9.2 | 9.4 | 13.9 | 6.6 |

| | GA7_FAD2- | GA7_FAD2- | GA7_FAD2- | T₄ Col_22.2 | T₄ Col_22.2 |
|---|---|---|---|---|---|
| | 25.10 | 21.2 | 18.14 | (mean ± SD) | best line |
| 16:0 | 8.1 | 10.7 | 7.7 | 10.6 ± 0.9 | 12.2 |
| 18:0 | 3.5 | 3.8 | 3.3 | 3.5 ± 0.4 | 3.6 |
| 18:1n7 | 1.7 | 2.2 | 1.6 | 2.3 ± 0.2 | 2.6 |
| 20:0 | 1.8 | 2.0 | 1.9 | 1.9 ± 0.3 | 2.0 |
| 20:1d13 | 1.2 | 1.4 | 1.3 | 1.6 ± 0.2 | 1.9 |
| 20:1d9/11 | 15.7 | 12.4 | 18.4 | 11.7 ± 1.7 | 9.5 |
| 22:1d13 | 1.0 | 1.1 | 1.5 | 0.9 ± 0.1 | 0.8 |
| Other minor | 1.7 | 1.9 | 1.6 | 1.9 ± 0.1 | 2.3 |
| 18:1d9 | 11.3 | 4.2 | 11.5 | 4.6 ± 1.0 | 3.3 |
| 18:2ω6 | 5.8 | 8.9 | 5.6 | 5.3 ± 0.9 | 4.3 |
| 18:3ω3 | 28.3 | 28.9 | 30.8 | 31.0 ± 1.1 | 29.5 |
| 18:3ω6 | 0.3 | 0.6 | 0.1 | 0.4 ± 0.1 | 0.4 |
| 20:2ω6 | 0.6 | 1.2 | 0.6 | 0.9 ± 0.1 | 0.9 |
| 20:3ω6 | 0 | 0 | 0 | | |
| 20:4ω6 | 0 | 0 | 0 | | |
| 22:4ω6 | 0 | 0 | 0 | 0.1 ± 0.0 | 0.1 |
| 22:5ω6 | 0 | 0 | 0 | | |
| 18:4ω3 | 3.7 | 5.2 | 2.6 | 4.8 ± 0.9 | 5.5 |
| 20:3ω3 | 1.1 | 1.3 | 1.3 | 1.5 ± 0.2 | 1.7 |
| 20:4ω3 | 1.7 | 0.9 | 0.9 | 0.8 ± 0.2 | 0.8 |
| 20:5ω3 | 1.2 | 1.0 | 0.8 | 1.5 ± 0.3 | 1.8 |
| 22:5ω3 | 0.8 | 0.6 | 0.5 | 1.1 ± 0.2 | 1.5 |
| 22:6ω3 | 10.3 | 11.5 | 7.9 | 13.3 ± 1.6 | 15.1 |

Enzymatic conversion efficiencies for each enzyme step in the pathway for production of DHA from oleic acid are shown in Table 8 for the T₃ seeds with the higher DHA levels. The Δ12-desaturase conversion efficiency in seeds of line 22.2 was 81.6% and the ω3-desaturase efficiency was 89.1%, both of them remarkably high and indicating that these fungal (yeast) enzymes were able to function well in developing seeds. The activities of the other exogenous enzymes in the DHA pathway were similarly high for ω3 substrates with the Δ6-desaturase acting at 42.2% efficiency, Δ6-elongase at 76.8%, Δ5-desaturase at 95.0%, Δ5-elongase at 88.7% and Δ4-desaturase at 93.3% efficiency. The Δ6-desaturase activity on the ω6 substrate LA was much lower, with the Δ6-desaturase acting at only 0.7% conversion efficiency on LA. GLA was present at a level of only 0.4% and was the only new (ω6 product aside from 20:2ω6 detected in the T₃ seeds with the highest DHA content. Compiled data from the total seed lipid profiles from independent transgenic seed (Table 7) are shown in Table 9. This data for the line with the greatest DHA level included a total ω6 FA (including LA) to total ω3 FA (including ALA) ratio of 0.10. The new ω6 FA (excluding LA) to new ω3 FA (excluding ALA) ratio in the lipid of this line was 0.05. Total polyunsaturated fatty acid levels were more than 50% in these lines, and greater than 60% in at least 4 of the lines. Overall conversion efficiencies were calculated to be: OA to EPA=21.8%, OA to DHA=18.0%, LA to EPA=26.9%, LA to DHA=22.2%, ALA to EPA=30.1%, ALA to DHA=24.9%.

TABLE 8
Conversion efficiencies of the individual enzymatic steps for the production of DHA from oleic
acid, observed in total seed lipid from transgenic T₃ Arabidopsis seeds as in Table 7.

| | | GA7_Col_7.2 | GA7_Col_34.2 | GA7_Col_10.13 | GA7_Col_22.2 | GA7_Col_14.19 |
|---|---|---|---|---|---|---|
| | d12-des | 75.4% | 73.1% | 75.7% | 81.6% | 73.4% |
| | d15-des | 85.3% | 84.4% | 86.2% | 89.1% | 70.2% |
| Omega-6 | d6-des | 0.3% | 0.3% | 0.3% | 0.7% | 0.3% |
| | (d9-elo) | 1.7% | 1.7% | 1.2% | 1.2% | 2.6% |
| | d6-elo | | | | | |
| | d5-des | | | | | |
| | d5-elo | | | | | |
| | d4-des | | | | | |
| Omega-3 | d6-des | 30.7% | 29.3% | 28.2% | 42.2% | 30.2% |
| | (d9-elo) | 2.7% | 2.7% | 2.3% | 2.4% | 3.0% |
| | d6-elo | 79.0% | 81.1% | 79.0% | 76.8% | 70.9% |
| | d5-des | 94.0% | 94.6% | 94.5% | 95.0% | 97.9% |
| | d5-elo | 91.9% | 91.7% | 93.6% | 88.7% | 89.5% |
| | d4-des | 93.2% | 93.7% | 94.4% | 93.3% | 93.7% |

| | | GA7_FAD2- | GA7_FAD2- | GA7_FAD2- | T₄ Col_22.2 | T₄ Col_22.2 |
|---|---|---|---|---|---|---|
| | | 25.10 | 21.2 | 18.14 | (mean) | best line |
| | d12-des | 66.6% | 78.5% | 63.1% | 67.6% | 82.7% |
| | d15-des | 87.5% | 82.2% | 87.6% | 81.0% | 90.9% |
| Omega-6 | d6-des | 0.6% | 1.0% | 0.2% | 1.3% | 0.7% |
| | (d9-elo) | 1.1% | 2.0% | 1.3% | 1.6% | 1.5% |
| | d6-elo | | | | | |
| | d5-des | | | | | |
| | d5-elo | | | | | |
| | d4-des | | | | | |
| Omega-3 | d6-des | 38.5% | 40.0% | 29.2% | 41.0% | 45.7% |
| | (d9-elo) | 2.3% | 2.7% | 2.9% | 2.8% | 3.1% |
| | d6-elo | 79.2% | 73.2% | 79.1% | 77.5% | 77.7% |
| | d5-des | 87.8% | 93.3% | 91.1% | 95.0% | 95.8% |
| | d5-elo | 89.9% | 92.2% | 91.6% | 90.8% | 90.2% |
| | d4-des | 92.5% | 95.0% | 93.9% | 92.2% | 90.9% |

TABLE 9
Compiled data from the total seed lipid profiles from independent transgenic seed
shown in Table 5. Calculations do not include the ‘minor fatty acids’ in Table 7.

| Parameter | GA7-Col_7.2 | GA7-Col_34.2 | GA7-Col_10.13 | GA7-Col_22.2 | GA7-Col_14.19 |
|---|---|---|---|---|---|
| total w3 (% of total FA) | 50.0 | 48.9 | 51.6 | 55.8 | 38.6 |
| total w6 (% of total FA) | 8.7 | 9.1 | 8.3 | 6.7 | 16.3 |
| w3/w6 ratio | 5.75 | 5.37 | 6.22 | 8.33 | 2.37 |
| w6/w3 ratio | 0.17 | 0.19 | 0.16 | 0.12 | 0.42 |
| total novel w3 (% of total FA) | 16.3 | 15.2 | 15.5 | 24.3 | 12.5 |
| total novel w6 (% of total FA) | 1.2 | 1.2 | 0.9 | 1.1 | 1.5 |
| novel w3/w6 ratio | 13.58 | 12.67 | 17.22 | 22.09 | 8.33 |
| novel w6/w3 ratio | 0.07 | 0.08 | 0.06 | 0.05 | 0.12 |
| OA to EPA efficiency | 14.1% | 13.3% | 13.4% | 21.8% | 10.2% |
| OA to DHA efficiency | 12.0% | 11.4% | 11.8% | 18.0% | 8.6% |
| LA to EPA efficiency | 18.9% | 18.4% | 17.9% | 26.9% | 14.2% |
| LA to DHA efficiency | 16.2% | 15.9% | 15.7% | 22.2% | 12.0% |
| ALA to EPA efficiency | 22.2% | 21.9% | 20.7% | 30.1% | 20.2% |
| ALA to DHA efficiency | 19.0% | 18.8% | 18.2% | 24.9% | 17.1% |
| total saturates | 16.0 | 14.7 | 15.4 | 16.0 | 16.2 |
| total monounsaturates | 23.7 | 25.8 | 23.4 | 19.2 | 26.5 |
| total polyunsaturates | 58.7 | 58.0 | 59.9 | 62.5 | 54.9 |
| total C20 | 19 | 19.8 | 16.8 | 15.9 | 19.1 |
| total C22 | 11.4 | 11 | 10.8 | 15.5 | 8.6 |
| C20/C22 ratio | 1.67 | 1.80 | 1.56 | 1.03 | 2.22 |

| | GA7-FAD2- | GA7-FAD2- | GA7-FAD2- | T₄ Col_22.2 | T₄ Col_22.2 |
|---|---|---|---|---|---|
| Parameter | 25.10 | 21.2 | 18.14 | (mean ± SD) | best line |
| total w3 (% of total FA) | 47.1 | 49.4 | 44.8 | 54.0 | 55.9 |
| total w6 (% of total FA) | 6.7 | 10.7 | 6.3 | 6.7 | 5.7 |
| w3/w6 ratio | 7.03 | 4.62 | 7.11 | 8.06 | 9.81 |
| w6/w3 ratio | 0.14 | 0.22 | 0.14 | 0.12 | 0.10 |
| total novel w3 (% of total FA) | 18.8 | 20.5 | 14.0 | 23.0 | 26.4 |
| total novel w6 (% of total FA) | 0.9 | 1.8 | 0.7 | 1.4 | 1.4 |
| novel w3/w6 ratio | 20.89 | 11.39 | 20.00 | 16.43 | 18.86 |
| novel w6/w3 ratio | 0.05 | 0.09 | 0.05 | 0.06 | 0.05 |
| OA to EPA efficiency | 15.0% | 16.8% | 11.2% | 20.4% | 24.5% |
| OA to DHA efficiency | 12.6% | 14.8% | 9.6% | 17.1% | 20.1% |
| LA to EPA efficiency | 22.9% | 21.8% | 18.0% | 26.2% | 29.9% |
| LA to DHA efficiency | 19.1% | 19.1% | 15.5% | 21.9% | 24.5% |
| ALA to EPA efficiency | 26.1% | 26.5% | 20.5% | 29.4% | 32.9% |
| ALA to DHA efficiency | 21.9% | 23.3% | 17.6% | 24.6% | 27.0% |
| total saturates | 13.4 | 16.5 | 12.9 | 16.0 | 17.8 |
| total monounsaturates | 30.9 | 21.3 | 34.3 | 21.1 | 18.1 |
| total polyunsaturates | 53.8 | 60.1 | 51.1 | 60.7 | 61.6 |
| total C20 | 21.5 | 18.2 | 23.3 | 18 | 16.6 |
| total C22 | 12.1 | 13.2 | 9.9 | 15.4 | 17.5 |
| C20/C22 ratio | 1.78 | 1.38 | 2.35 | 1.17 | 0.95 |

T₃ seeds from the pJP3416-GA7 line 22.2 in the Columbia background, which were progeny from T₂ line 22, were sown directly to soil and the fatty acid composition of mature seed from the resultant T₃ plants analysed by GC. The average DHA level of these seeds was 13.3%±1.6 (n=10) as a percentage of total fatty acids in the seed lipid. As shown in Table 6 (right hand column), the line with the highest level of DHA contained 15.1% DHA in the total fatty acids of the seed lipid. The enzymatic conversion efficiencies are shown in Table 8 for each step in the production of DHA from oleic acid.

The total ω6 FA (including LA) to ω3 FA (including ALA) ratio in the line with the highest DHA level was 0.102. The new ω6 FA (excluding LA) to new ω3 FA (excluding ALA) ratio in the line with the highest DHA level was 0.053. The level of total saturated fatty acids was about 17.8% and the level of monounsaturated fatty acids was about 18.1%. The level of total ω6-fatty acids was about 5.7% and the level of ω3-fatty acids was about 55.9%. Overall conversion efficiencies were calculated to be: OA to EPA=24.5%, OA to DHA=20.1%, LA to EPA=29.9%, LA to DHA=24.5%, ALA to EPA=32.9%, ALA to DHA=27.0%. Total omega-3 fatty acids were found to accumulate to 55.9% of total fatty acids whereas omega-6 fatty acids were 5.7% of the total profile.

Southern blot hybridisation analysis was performed. The results showed that the high-accumulating DHA lines were either single- or double-copy for the T-DNA from the pJP3416-GA7 construct with the exception of transgenic line Columbia #22, which had three T-DNA insertions in the genome of the Arabidopsis plant. The T₅ generation seed was also analysed and found to have up to 13.6% DHA in the total seed lipids. The GA7 construct was found to be stable across multiple generations in terms of DHA production capability.

Determination of Oil Content in Transgenic A. thaliana DHA Lines

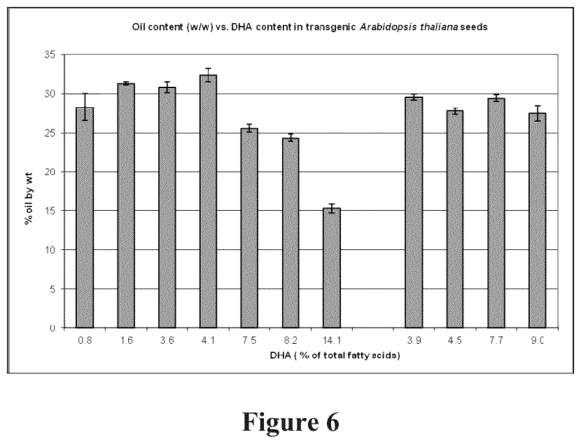

The oil content of transgenic A. thaliana seeds with various levels of DHA was determined by GC as described in Example 1. The data are shown in FIG. 6, graphing the oil content (% oil by weight of seed) against the DHA content (as a percentage of total fatty acids). Up to 26.5 mg of DHA per gram of seed was observed (Table 10). The oil content of the transgenic Arabidopsis seeds was found to be negatively correlated with DHA content. The amount of DHA per weight of seed was greater in the transformed seeds with a DHA level of about 9% relative to the seeds with about 14% DHA. Whether this would be true for seeds other than Arabidopsis has not been determined.

TABLE 10
Proportion and amount of DHA in
GA7-transformed Arabidopsis seeds.

| | DHA | Oil content | DHA content |
|---|---|---|---|
| | content | (% oil per | per weight |
| | (% of TFA) | g seeds) | (mg/g seed) |
| GA7/col 22.2-1 | 14.2 | 14.89 | 70.2 |
| GA7/col 22.2-2 | 14.3 | 15.02 | 20.5 |
| GA7/col 22.2-3 | 14.0 | 15.92 | 21.2 |
| GA7/col 10.15-1 | 8.7 | 30.23 | 25.06 |
| GA7/col 10.15-2 | 8.6 | 31.25 | 25.77 |
| GA7/col 10.15-3 | 8.8 | 31.70 | 26.49 |

### Example 3. Stable Expression of a Transgenic DHA Pathway in Camelina sativa Seeds

The binary vector pJP3416-GA7 as described above was introduced into A. tumefaciens strain AGL1 and cells from a culture of the transformed Agrobacterium used to treat C. sativa flowering plants using a floral dip method for transformation (Lu and Kang, 2008). After growth and maturation of the plants, the T₁ seeds from the treated plants were harvested, sown onto soil and the resultant plants treated by spraying with the herbicide BASTA to select for plants which were transgenic for, and expressing, the bar selectable marker gene present on the T-DNA of pJP3416-GA7. Surviving T₁ plants which were tolerant to the herbicide were grown to maturity after allowing them to self-fertilise, and the resultant T₂ seed harvested. Five transgenic plants were obtained, only three of which contained the entire T-DNA.

Lipid was extracted from a pool of approximately twenty seeds from each of the three plants that contained the entire T-DNA. Two of the pooled samples contained very low, barely detectable levels of DHA, but the third pool contained about 4.7% DHA (Table 12). Therefore, lipid was extracted from 10 individual T₂ seeds from this plant and the fatty acid composition analysed by GC. The fatty acid composition data of the individual seeds for this transformed line is also shown in Table 11. Compiled data from the total seed lipid profiles (Table 11) are shown in Table 12.

TABLE 11
Fatty acid composition of total seed lipids from transgenic T₂ Camelina sativa
seeds transformed with the T-DNA from pJP3416-GA7. The fatty acid composition
is shown for a pooled seed batch (FD5.46) and for 10 single seeds ranked
(left to right) from highest to lowest DHA.

| | FD5.46 | | | | | | | | | | |
|---|---|---|---|---|---|---|---|---|---|---|---|
| Fatty acid | pooled | # 2 | # 4 | # 8 | # 7 | # 9 | # 1 | # 3 | # 5 | # 6 | # 10 |
| 14:0 | 0 | 0.2 | 0.2 | 0.1 | 0.2 | 0.2 | 0.2 | 0.2 | 0.1 | 0.2 | 0.2 |
| 16:0 | 11.6 | 12.1 | 12.3 | 12.1 | 13.2 | 12.3 | 12.8 | 11.9 | 11.4 | 11.5 | 11.7 |
| 16:1 | 0.2 | 0.0 | 0.1 | 0.1 | 0.0 | 0.2 | 0.0 | 0.2 | 0.2 | 0.2 | 0.2 |
| 16:3 | 0.3 | 0 | 0.0 | 0.0 | 0.0 | 0.0 | 0.0 | 0.0 | 0.0 | 0.0 | 0.0 |
| 18:0 | 3.7 | 3.3 | 3.2 | 3.2 | 3.0 | 3.1 | 3.2 | 3.3 | 3.1 | 3.2 | 3.2 |
| 18:1 | 10.8 | 8.0 | 8.0 | 8.6 | 8.5 | 9.4 | 11.0 | 10.2 | 8.3 | 9.4 | 8.6 |
| 18:1d11 | 1.7 | 1.3 | 1.4 | 1.4 | 1.7 | 1.4 | 1.5 | 1.3 | 1.3 | 1.3 | 1.3 |
| 18:2 | 24.7 | 18.2 | 19.5 | 19.2 | 18.5 | 20.1 | 23.8 | 32.2 | 30.3 | 29.8 | 31.6 |
| 18:3ω3 | 27.4 | 26.7 | 26.6 | 27.3 | 28.9 | 28.2 | 27.4 | 28.3 | 29.2 | 29.5 | 28.2 |
| 18:3ω6 | 0.2 | 1.4 | 0.3 | 0.3 | 0.4 | 0.2 | 0.5 | 0.0 | 0.5 | 0.4 | 0.6 |
| 20:0 | 1.6 | 1.4 | 1.3 | 1.4 | 1.2 | 1.4 | 1.4 | 1.8 | 2.1 | 1.9 | 2.0 |
| 18:4ω3 | 2.2 | 6.8 | 6.4 | 5.7 | 7.2 | 5.7 | 4.1 | 0.0 | 0.0 | 0.0 | 0.0 |
| 20:1d11 | 5.3 | 4.4 | 4.6 | 4.8 | 3.3 | 4.1 | 3.5 | 4.4 | 6.1 | 5.8 | 5.5 |
| 20:1iso | 0.4 | 0.3 | 0.3 | 0.3 | 0.3 | 0.3 | 0.0 | 0.5 | 0.6 | 0.5 | 0.5 |
| 20:2ω6 | 0.8 | 0.8 | 0.9 | 0.8 | 0.6 | 0.8 | 0.7 | 1.3 | 1.5 | 1.4 | 1.4 |
| 20:3ω3 | 0.6 | 0.8 | 0.8 | 0.8 | 0.7 | 0.8 | 0.7 | 0.6 | 0.7 | 0.7 | 0.6 |
| 22:0 | 0.4 | 0.5 | 0.5 | 0.5 | 0.4 | 0.5 | 0.5 | 0.6 | 0.6 | 0.6 | 0.6 |
| 20:4ω3 | 0.2 | 0.3 | 0.3 | 0.3 | 0.4 | 0.4 | 0.5 | 0.0 | 0.0 | 0.0 | 0.0 |
| 22:1 | 1.1 | 1.1 | 1.2 | 1.1 | 0.5 | 0.9 | 0.8 | 1.6 | 2.2 | 1.9 | 2.0 |
| 20:5ω3 | 0.7 | 1.3 | 1.6 | 1.5 | 1.6 | 1.1 | 1.7 | 0.0 | 0.0 | 0.0 | 0.1 |
| 22:2ω6 | 0.1 | 0.0 | 0.0 | 0.0 | 0.0 | 0.0 | 0.0 | 0.2 | 0.3 | 0.2 | 0.2 |
| 22:4ω6 + | 0.3 | 0.2 | 0.3 | 0.3 | 0.0 | 0.3 | 0.0 | 0.4 | 0.6 | 0.5 | 0.5 |
| 22:3ω3 | | | | | | | | | | | |
| 24:0 | 0.3 | 0.3 | 0.3 | 0.3 | 0.0 | 0.3 | 0.0 | 0.4 | 0.4 | 0.4 | 0.4 |
| 24:1 | 0.3 | 0.4 | 0.4 | 0.3 | 0.0 | 0.3 | 0.0 | 0.5 | 0.6 | 0.5 | 0.5 |
| 22:5ω3 | 0.3 | 1.1 | 1.2 | 1.1 | 1.1 | 0.9 | 0.8 | 0.0 | 0.0 | 0.0 | 0.0 |
| 22:6ω3 | 4.7 | 9.0 | 8.5 | 8.3 | 8.3 | 7.1 | 4.9 | 0.0 | 0.0 | 0.0 | 0.0 |

TABLE 12
Compiled data from the total seed lipid profiles from transgenic seed shown
in Table 11. Calculations do not include the ‘minor fatty acids’ in Table 11.

| | FD5.46 | | | | | | | | | | |
|---|---|---|---|---|---|---|---|---|---|---|---|
| Parameter | pooled | # 2 | # 4 | # 8 | # 7 | # 9 | # 1 | # 3 | # 5 | # 6 | # 10 |
| total w3 (% of total FA) | 36.1 | 46 | 45.4 | 45 | 48.2 | 44.2 | 40.1 | 28.9 | 29.9 | 30.2 | 28.9 |
| total w6 (% of total FA) | 25.8 | 20.4 | 20.7 | 20.3 | 19.5 | 21.1 | 25 | 33.7 | 32.6 | 31.8 | 33.8 |
| w3/w6 ratio | 1.40 | 2.25 | 2.19 | 2.22 | 2.47 | 2.09 | 1.60 | 0.86 | 0.92 | 0.95 | 0.86 |
| w6/w3 ratio | 0.71 | 0.44 | 0.46 | 0.45 | 0.40 | 0.48 | 0.62 | 1.17 | 1.09 | 1.05 | 1.17 |
| total novel w3 (% of total FA) | 8.1 | 18.5 | 18 | 16.9 | 18.6 | 15.2 | 12 | 0 | 0 | 0 | 0.1 |
| total novel w6 (% of total FA) | 1.1 | 2.2 | 1.2 | 1.1 | 1 | 1 | 1.2 | 1.5 | 2.3 | 2 | 2.2 |
| novel w3/w6 ratio | 7.36 | 8.41 | 15.00 | 15.36 | 18.60 | 15.20 | 10.00 | | | | 0.05 |
| novel w6/w3 ratio | 0.14 | 0.12 | 0.07 | 0.07 | 0.05 | 0.07 | 0.10 | | | | 22.00 |
| OA to EPA efficiency | 8.2% | 15.6% | 15.5% | 15.1% | 15.1% | 12.8% | 10.5% | 0.0% | 0.0% | 0.0% | 0.1% |
| OA to DHA efficiency | 6.7% | 12.3% | 11.6% | 11.5% | 11.4% | 10.0% | 7.0% | 0.0% | 0.0% | 0.0% | 0.0% |
| LA to EPA efficiency | 9.2% | 17.2% | 17.1% | 16.7% | 16.2% | 13.9% | 11.4% | 0.0% | 0.0% | 0.0% | 0.2% |
| LA to DHA efficiency | 7.6% | 13.6% | 12.9% | 12.7% | 12.3% | 10.9% | 7.5% | 0.0% | 0.0% | 0.0% | 0.0% |
| ALA to EPA efficiency | 15.8% | 24.8% | 24.9% | 24.2% | 22.8% | 20.6% | 18.5% | 0.0% | 0.0% | 0.0% | 0.3% |
| ALA to DHA efficiency | 13.0% | 19.6% | 18.7% | 18.4% | 17.2% | 16.1% | 12.2% | 0.0% | 0.0% | 0.0% | 0.0% |
| total saturates | 17.6 | 17.8 | 17.8 | 17.6 | 18 | 17.8 | 18.1 | 18.2 | 17.7 | 17.8 | 18.1 |
| total monounsaturates | 19.8 | 15.5 | 16 | 16.6 | 14.3 | 16.6 | 16.8 | 18.7 | 19.3 | 19.6 | 18.6 |
| total polyunsaturates | 62.5 | 66.6 | 66.4 | 65.6 | 67.7 | 65.6 | 65.1 | 63 | 63.1 | 62.5 | 63.2 |
| total C20 | 9.6 | 9.3 | 9.8 | 9.9 | 8.1 | 8.9 | 8.5 | 8.6 | 11 | 10.3 | 10.1 |
| total C22 | 5.4 | 10.3 | 10 | 9.7 | 9.4 | 8.3 | 5.7 | 0.6 | 0.9 | 0.7 | 0.7 |
| C20/C22 ratio | 1.78 | 0.90 | 0.98 | 1.02 | 0.86 | 1.07 | 1.49 | 14.33 | 12.22 | 14.71 | 14.43 |

DHA was present in six of the 10 individual seeds. The four other seeds did not have DHA and were presumed to be null segregants which did not have the T-DNA, based on homozygosity of the T-DNA insertion in the parental plant. Extracted lipid from the single seed with the highest level of DHA had 9.0% DHA while the sum of the percentages for EPA, DPA and DHA was 11.4%. The sum of the percentages for the new ω3 fatty acids produced in this seed as a result of the transformation (SDA, ETrA, ETA, EPA, DPA, DHA) was 19.3% whilst the corresponding sum for the new ω6 fatty acids (GLA, EDA, DGLA, ARA and any ω6 elongation products) was 2.2%—only GLA and EDA were detected as new ω6 fatty acids. The total ω6 FA (including LA) to ω3 FA (including ALA) ratio was found to be 0.44. The new ω6 FA (excluding LA) to new ω3 FA (excluding ALA) ratio in the seed with the highest DHA level was 0.12. The level of total saturated fatty acids was about 17.8% and the level of monounsaturated fatty acids was about 15.5%. The level of total ω6-fatty acids was about 20.4% and the level of ω3-fatty acids was about 46%. Overall conversion efficiencies were calculated to be: OA to EPA=15.6%, OA to DHA=12.3%, LA to EPA=17.2%, LA to DHA=13.6%, ALA to EPA=24.8%, ALA to DHA=19.6%.

Homozygous seed from this line was obtained in the T4 generation. Up to 10.3% DHA was produced in event FD5-46-18-110 with an average of 7.3% DHA observed across the entire T4 generation.

Homozygous seed was planted out across several glasshouses to generate a total of over 600 individual plants. Oil is being extracted from the seed using a variety of methods including soxhlet, acetone and hexane extractions.

Since the number of independently transformed lines of C. sativa obtained as described above was low, further experiments to transform C. sativa with pJP3416-GA7 are performed. The inventors predict that DHA levels of greater than 10% as a percentage of total fatty acids in seed oil will be achieved in further transformed lines, and plants which are homozygous for the T-DNA to 20% DHA. Twenty C. sativa GA7_modH events were generated and seed is being analysed for DHA content. Three GA7_modB events were generated and analysis of the T1 seed from event CMD17.1 revealed a pooled seed DHA content of 9.8%. The highest single seed DHA value was found to be 13.5%.

### Example 4. Stable Expression of Transgenic DHA Pathways in Brassica nanus Seeds

B. napus Transformation and Analysis of Fatty Acid Composition Using Single Vector

The binary vector pJP3416-GA7 was used to generate transformed Brassica napus plants and seeds from the plants. The vector pJP3416-GA7 as described above was introduced into Agrobacterium tumefaciens strain AGL1 via standard electroporation procedures. Cultures of the transgenic Agrobacterium cells were grown overnight at 28° C. in LB medium with agitation at 150 rpm. The bacterial cells were collected by centrifugation at 4000 rpm for 5 minutes, washed with Winans AB medium (Winans, 1988) and re-suspended in 10 mL of Winans AB medium (pH 5.2) and growth continued overnight in the presence of kanamycin (50 mg/L), rifampicin (25 mg/L) and 100 μM acetosyringone. Two hours before infection of the Brassica cells, spermidine (120 mg/L) was added and the final density of the bacteria adjusted to an OD 600 nm of 0.3-0.4 with fresh AB media. Freshly isolated cotyledonary petioles from 8-day old Brassica napus seedlings grown on ½ MS (Murashige and Skoog, 1962) or hypocotyl segments preconditioned by 3-4 days on MS media with 1 mg/L thidiazuron (TDZ) and 0.1 mg/L α-naphthaleneacetic acid (NAA) were infected with 10 mL Agrobacterium cultures for 5 minutes. The explants infected with Agrobacterium were then blotted on sterile filter paper to remove the excess Agrobacterium and transferred to co-cultivation media (MS media with 1 mg/L TDZ, 0.1 mg/L NAA and 100 μM acetosyringone) supplemented with or without different antioxidants (L-cysteine 50 mg/L and ascorbic 15 mg/L). All the plates were sealed with parafilm and incubated in the dark at 23-24° C. for 48 hrs.

The treated explants were then washed with sterile distilled water containing 500 mg/L cefotaxime and 50 mg/L timentin for 10 minutes, rinsed in sterile distilled water for 10 minutes, blotted dry on sterile filter paper, transferred to pre-selection media (MS containing 1 mg/L TDZ, 0.1 mg/L NAA, 20 mg/L adenine sulphate (ADS), 1.5 mg/L AgNO₃, 250 mg/L cefotaxime and 50 mg/L timentin) and cultured for five days at 24° C. with a 16 h/8 h photoperiod. They were then transferred to selection media (MS containing 1 mg/L TDZ, 0.1 mg/L NAA, 20 mg/L ADS, 1.5 mg/L AgNO₃, 250 mg/L cefotaxime and 50 mg/L timentin) with 1.5 mg/L glufosinate ammonium as the agent for selection of transformed cells, and cultured for 4 weeks at 24° C. with 16 h/8 h photoperiod with a biweekly subculture on to the same media. Explants with green callus were transferred to shoot initiation media (MS containing 1 mg/L kinetin, 20 mg/L ADS, 1.5 mg/L AgNO₃, 250 mg/L cefotaxime, 50 mg/L timentin and 1.5 mg/L glufosinate ammonium) and cultured for another 2-3 weeks. Shoots emerging from the resistant explants were transferred to shoot elongation media (MS media with 0.1 mg/L gibberelic acid, 20 mg/L ADS, 1.5 mg/L AgNO₃, 250 mg/L cefotaxime and 1.5 mg/L glufosinate ammonium) and cultured for another two weeks. Healthy shoots 2-3 cm long were selected and transferred to rooting media (½ MS containing 1 mg/L NAA, 20 mg/L ADS, 1.5 mg/L AgNO₃ and 250 mg/L cefotaxime) and cultured for 2-3 weeks. Well established shoots with roots were transferred to pots containing seedling raising mix and grown in a growth cabinet for two weeks and subsequently transferred to a glasshouse. Approximately 40 (To) plants transformed with the GA7 construct were obtained by this method.

Plants were grown to maturity after being allowed to self-fertilise. Seeds obtained from transformed plants were analysed for fatty acid composition in their seedoil as described in Example 1. Data for a transformed line with the highest DHA level are shown in Table 13. DHA levels on average were significantly lower in the seedoil of the B. napus seeds transformed with the T-DNA from pJP3416-GA7 than in A. thaliana seeds (Example 2) or Camelina seeds (Example 3) transformed with the same construct. The highest level of DHA in approximately 40 lines was found to be 1.52% with the majority of the transgenic lines having detectable DHA. It was noted that there was a substantial accumulation of ALA, about 35% of the total fatty acids, in these seeds which was not being converted efficiently to SDA or following products in the pathway.

Fatty acid profile analysis of single B. napus seeds from a T₁ event, CT125-2, was performed to better determine the amount of DHA produced in transgenic seeds. Seeds were found to contain between 0% (null seeds) and 8.5% DHA (Table 13).

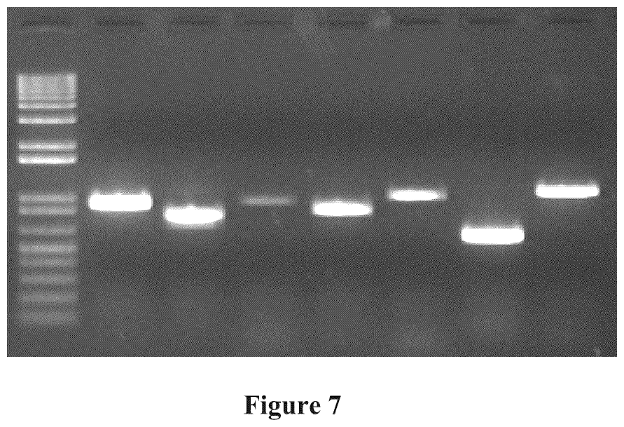

Some of the seeds from the plant line CT116 as well as other transgenic lines showing DHA production were sown to produce progeny plants. RT-PCR was performed on total RNA isolated from developing embryos from these plants in order to determine why the GA7 construct performed poorly for DHA production relative to transgenic A. thaliana and C. sativa having the same construct, and poorly relative to the combination of the genes on pJP3115 and pJP3116 (below). RT-PCR was performed on total RNA using a one-step RT-PCR kit (Invitrogen) and gene-specific primers targeting each transgene. This confirmed that each of the genes in the GA7 construct was expressed well in the B. napus transformants except for the Δ6-desaturase which was poorly expressed in the majority of transformed seeds. The other genes from this construct functioned well in both B. napus and A. thaliana seeds, for example the Δ12- and Δ15-desaturases which functioned to produce increased levels of LA and ALA in the seeds whilst decreasing oleic acid levels. A representative RT-PCR gel is shown in FIG. 7 which clearly shows the low expression of the Δ6-desaturase relative to the other transgenes from pJP3416-GA7.

Transgenic plants and seed which are homozygous for the transgenes are generated by planting out progeny from the lines with the highest DHA.

TABLE 13
Fatty acid composition as a percentage of total fatty acids
in seedoil from independent T₁ Brassica napus seed transformed
with pJP3416-GA7, lines CT116-11 and CT-125-2 compared to wild-
type (untransformed) control. 22:6ω3 is DHA. Data from single
CT125-2 B. napus seeds is denoted by ‘SS’.

| | | CT116- | CT125- | CT125-2 | CT125-2 | CT125-2 |
|---|---|---|---|---|---|---|
| | Control | 11 | 2 | #2 SS | #3 SS | #10 SS |
| 14:0 | 0.1 | 0.2 | 0.1 | 0.1 | 0.1 | 0.1 |
| 16:0 | 4.3 | 7.2 | 5.2 | 6.5 | 4.7 | 7.7 |
| 16:1 | 0.2 | 0.5 | 0.4 | 0.3 | 0.3 | 0.8 |
| 16:3 | 0.2 | 0.2 | 0.2 | 0.1 | 0.2 | 0.2 |
| 18:0 | 2.1 | 2.2 | 2.4 | 2.3 | 2.3 | 2.8 |
| 18:1d9 | 59.1 | 27.0 | 38.1 | 34.0 | 19.3 | 14.8 |
| 18:1d11 | 3.7 | 6.6 | 4.2 | 4.4 | 4.3 | 9.6 |
| 18:2 | 19.7 | 14.1 | 16.6 | 13.9 | 10.2 | 10.2 |
| 18:3ω3 | 8.3 | 35.2 | 27.7 | 34.1 | 49.5 | 37.9 |
| 20:0 | 0.6 | 0.5 | 0.6 | 0.4 | 0.3 | 0.7 |
| 18:4ω3 | 0.0 | 0.9 | 0.3 | 0.5 | 0.6 | 2.6 |
| 20:1d11 | 1.2 | 1.1 | 1.0 | 1.0 | 0.8 | 0.6 |
| 20:1is0 | | 0.2 | | 0.1 | | 0.2 |
| 20:2ω6 | 0.1 | 0.1 | 0.1 | 0.1 | 0.1 | 0.1 |
| 20:3ω3 | | 1.3 | 0.7 | 0.8 | 1.6 | 0.9 |
| 22:0 | 0.3 | 0.4 | 0.3 | 0.1 | 0.1 | 0.4 |
| 20:4ω3 | | 0.1 | 0.3 | 0.4 | 0.6 | 0.5 |
| 22:1 | | | | | | |
| 20:5ω3 | | | | | 0.1 | 0.3 |
| 22:3ω3 | | | | | 0.1 | |
| 24:0 | 0.2 | 0.4 | 0.3 | 0.1 | 0.1 | 0.3 |
| 24:1 | 0.1 | 0.3 | 0.1 | 0.1 | 0.2 | 0.1 |
| 22:5ω3 | | 0.1 | 0.1 | 0.1 | 0.1 | 0.5 |
| 22:6ω3 | | 1.52 | 1.2 | 1.3 | 2.7 | 8.5 |

B. napus Transformation and Analysis of Fatty Acid Composition Using Two Vectors

In another experiment in B. napus and as an alternative format for introducing the transgenes, the binary vectors pJP3115 and pJP3116 as described in WO 2010/057246 were used to separately generate transformed B. napus plants and transformed seeds were obtained from the plants. The T-DNA on pJP3115 comprised chimeric genes encoding the Crepis palestina Δ12-desaturase, Micromonas pusilla Δ6-desaturase. Pyramimonas cordata Δ6-elongase and Pavlova salina Δ5-desaturase and the T-DNA on pJP3116 contained chimeric genes encoding Perilla frutescens Δ15-desaturase, Pyramimonas cordata Δ5-elongase and Pavlova salina Δ4-desaturase. The two T-DNAs, when present together and expressed in developing seeds, formed a 7-gene pathway for producing DHA from endogenously produced oleic acid. These vectors were introduced into Agrobacterium tumefaciens strain AGL1 via standard electroporation procedures and the transformed cells used independently to transform B. napus using the method as described above to generate stably transformed T₀ plants, 29 pJP3115 and 19 pJP3116 transformants were obtained and these plants were grown to maturity and seeds obtained after self-fertilisation were analysed for fatty acid composition in their seedoil. Transformation with the T-DNA from pJP3115 was expected to result in EPA production from endogenously produced ALA whilst transformation with the T-DNA from pJP3116 was expected to result in increased ALA production from LA. Several plants were identified which displayed these phenotypes. The majority of events displayed a decreased OA/increased LA phenotype due to Δ12 desaturation with a low level of EPA production. Up to 2.6% EPA was observed in pJP31115 transgenic pooled seed. Similarly, the majority of pJP3116 events were found to have an elevated ALA phenotype due to Δ15-desaturase activity. Up to 18.5% ALA was found in pooled seed transformed with the T-DNA from pJP3116.

T₁ plants from the lines with the highest levels of EPA and ALA were crossed and the progeny seed (F1) from 24 recovered events analysed for DHA content. DHA was found in 17 of these events with up to 1.9% DHA found in pooled seed from these events. Single-seed analysis was performed to determine the range of DHA production the data are shown in Table 14. A large range of DHA levels were observed in the crossed progeny, probably due to the hemizygous nature of the T-DNAs in the parental plants, so that some seeds did not receive both T-DNAs. Up to 6.7% DHA was observed in total seed lipid.

TABLE 14
Fatty acid composition as a percentage of total fatty acids in seedoil from B. napus F1 single seeds
that were from a cross of plants transgenic for the T-DNA from pJP3115 with plants transgenic for
the T-DNA from pJP3116. B1, B2 and B4 designate events. 0.0 = not detectable by the GC method.

| | B1.1 | B1.2 | B1.3 | B1.4-g | B1.5-g | B2.1 | B2.2 | B2.3g | B2.4g | B2.5g | B3.1 | B3.2 |
|---|---|---|---|---|---|---|---|---|---|---|---|---|
| 14:0 | 0.1 | 0.1 | 0.1 | 0.2 | 0.2 | 0.1 | 0.1 | 0.2 | 0.2 | 0.1 | 0.1 | 0.1 |
| 16:0 | 6.6 | 6.4 | 4.5 | 12.3 | 7.9 | 5.1 | 5.0 | 10.1 | 8.5 | 6.8 | 5.3 | 7.2 |
| 16:1 | 0.4 | 0.5 | 0.2 | 1.0 | 0.6 | 0.4 | 0.4 | 0.6 | 1.1 | 0.5 | 0.5 | 0.6 |
| 16:3 | 0.1 | 0.1 | 0.1 | 0.1 | 0.1 | 0.1 | 0.1 | 0.1 | 0.2 | 0.1 | 0.1 | 0.2 |
| 18:0 | 2.3 | 2.6 | 2.2 | 1.6 | 2.9 | 2.9 | 3.4 | 2.2 | 1.8 | 2.9 | 3.4 | 2.4 |
| 18:1 | 34.1 | 39.3 | 46.9 | 14.9 | 20.7 | 41.6 | 46.3 | 14.4 | 23.4 | 38.3 | 43.6 | 32.0 |
| 18:1d11 | 4.6 | 5.8 | 2.7 | 6.8 | 6.2 | 3.8 | 4.9 | 5.9 | 8.7 | 4.5 | 5.5 | 5.1 |
| 18:2 | 33.6 | 30.7 | 30.4 | 29.2 | 34.4 | 31.7 | 27.7 | 33.2 | 23.9 | 33.3 | 27.9 | 33.4 |
| 18:3ω6 | 0.2 | 0.3 | 0.1 | 0.4 | 0.4 | 0.2 | 0.2 | 0.7 | 0.1 | 0.2 | 0.2 | 0.3 |
| 18:3ω3 | 10.3 | 7.1 | 7.7 | 18.7 | 14.9 | 8.2 | 5.9 | 14.8 | 28.1 | 6.3 | 7.3 | 10.0 |
| 20:0 | 0.6 | 0.7 | 0.6 | 0.5 | 0.7 | 0.8 | 0.9 | 0.6 | 0.4 | 0.7 | 0.9 | 0.7 |
| 18:4ω3 | 0.2 | 0.1 | 0.1 | 0.8 | 0.5 | 0.2 | 0.2 | 0.8 | 0.0 | 0.2 | 0.2 | 0.2 |
| 20:1d11 | 1.0 | 1.1 | 1.1 | 0.7 | 0.8 | 1.1 | 1.1 | 0.5 | 0.9 | 1.1 | 1.1 | 0.9 |
| 20:1iso | 0.1 | 0.1 | 0.0 | 0.1 | 0.1 | 0.1 | 0.1 | 0.1 | 0.3 | 0.1 | 0.1 | 0.1 |
| 20:2ω6 | 0.4 | 0.3 | 0.2 | 0.5 | 0.5 | 0.4 | 0.3 | 0.4 | 0.5 | 0.5 | 0.3 | 0.5 |
| 20:3ω6 | 0.0 | 0.0 | 0.0 | 0.0 | 0.0 | 0.0 | 0.0 | 0.0 | 0.0 | 0.0 | 0.0 | 0.0 |
| 20:4ω6 | 0.0 | 0.0 | 0.0 | 0.0 | 0.0 | 0.0 | 0.0 | 0.1 | 0.0 | 0.0 | 0.0 | 0.0 |
| 20:3ω3 | 1.8 | 1.6 | 1.1 | 2.8 | 2.1 | 1.1 | 1.0 | 2.7 | 0.7 | 1.4 | 0.9 | 1.6 |
| 22:0 | 0.3 | 0.4 | 0.3 | 0.3 | 0.4 | 0.4 | 0.5 | 0.3 | 0.3 | 0.4 | 0.5 | 0.4 |
| 20:4ω3 | 0.3 | 0.2 | 0.2 | 0.4 | 0.4 | 0.1 | 0.1 | 0.5 | 0.0 | 0.2 | 0.1 | 0.2 |
| 22:1 | 0.0 | 0.0 | 0.0 | 0.0 | 0.0 | 0.0 | 0.0 | 0.0 | 0.0 | 0.0 | 0.0 | 0.0 |
| 20:5ω3 | 0.0 | 0.0 | 0.0 | 0.1 | 0.1 | 0.0 | 0.0 | 0.2 | 0.0 | 0.0 | 0.0 | 0.0 |
| 22:2ω6 | 0.0 | 0.0 | 0.0 | 0.0 | 0.0 | 0.0 | 0.0 | 0.0 | 0.0 | 0.0 | 0.0 | 0.0 |
| 22:4ω6 | 0.1 | 0.2 | 0.1 | 0.2 | 0.2 | 0.1 | 0.1 | 0.4 | 0.2 | 0.2 | 0.1 | 0.2 |
| 24:0 | 0.3 | 0.4 | 0.2 | 0.2 | 0.3 | 0.3 | 0.3 | 0.3 | 0.4 | 0.4 | 0.4 | 0.3 |
| 22:5ω6 | 0.1 | 0.2 | 0.1 | 0.2 | 0.3 | 0.1 | 0.1 | 0.5 | 0.0 | 0.2 | 0.1 | 0.2 |
| 24:1 | 0.2 | 0.2 | 0.2 | 0.3 | 0.3 | 0.2 | 0.2 | 0.3 | 0.3 | 0.2 | 0.2 | 0.2 |
| 22:5ω3 | 0.7 | 0.7 | 0.3 | 2.1 | 1.6 | 0.3 | 0.4 | 3.2 | 0.0 | 0.5 | 0.4 | 1.2 |
| 22:6ω3 | 1.4 | 1.0 | 0.5 | 5.5 | 3.9 | 0.8 | 0.7 | 6.7 | 0.0 | 1.1 | 0.8 | 2.0 |

TABLE 15
Compiled data from the total seed lipid profiles from transgenic seed shown
in Table 14. Calculations do not include the ‘minor fatty acids’ in Table 14.

| Parameter | B1.1 | B1.2 | B1.3 | B1.4-g | B1.5-g | B2.1 | B2.2 | B2.3g | B2.4g | B2.5g | B3.1 |
|---|---|---|---|---|---|---|---|---|---|---|---|
| total w3 (% of total FA) | 4.6 | 3.9 | 2.3 | 12.1 | 9 | 2.7 | 2.6 | 14.8 | 0.8 | 3.6 | 2.6 |
| total w6 (% of total FA) | 44.5 | 38.5 | 38.5 | 48.8 | 50.3 | 40.5 | 34.1 | 49.4 | 52.7 | 40.5 | 35.7 |
| w3/w6 ratio | 0.10 | 0.10 | 0.06 | 0.25 | 0.18 | 0.07 | 0.08 | 0.30 | 0.02 | 0.09 | 0.07 |
| w6/w3 ratio | 9.67 | 9.87 | 16.74 | 4.03 | 5.59 | 15.00 | 13.12 | 3.34 | 65.88 | 11.25 | 13.73 |
| total novel w3 (% of total FA) | 2.6 | 2 | 1.1 | 8.9 | 6.5 | 1.4 | 1.4 | 11.4 | 0 | 2 | 1.5 |
| total novel w6 (% of total FA) | 10.5 | 7.5 | 7.9 | 19.1 | 15.4 | 8.4 | 6.1 | 15.8 | 28.3 | 6.7 | 7.5 |
| novel w3/w6 ratio | 0.25 | 0.27 | 0.14 | 0.47 | 0.42 | 0.17 | 0.23 | 0.72 | 0.00 | 0.30 | 0.20 |
| novel w6/w3 ratio | 4.04 | 3.75 | 7.18 | 2.15 | 2.37 | 6.00 | 4.36 | 1.39 | | 3.35 | 5.00 |
| OA to EPA efficiency | 2.5% | 2.1% | 0.9% | 10.1% | 6.9% | 1.3% | 1.3% | 12.8% | | 1.9% | 1.4% |
| OA to DHA efficiency | 1.7% | 1.2% | 0.6% | 7.2% | 4.8% | 0.9% | 0.8% | 8.5% | | 1.3% | 1.0% |
| LA to EPA efficiency | 4.3% | 4.0% | 2.0% | 12.6% | 9.4% | 2.5% | 3.0% | 15.7% | | 3.6% | 3.1% |
| LA to DHA efficiency | 2.9% | 2.4% | 1.2% | 9.0% | 6.6% | 1.9% | 1.9% | 10.4% | | 2.5% | 2.1% |
| ALA to EPA efficiency | 47.7% | 44.7% | 36.4% | 68.1% | 65.9% | 44.0% | 45.8% | 72.1% | | 47.1% | 50.0% |
| ALA to DHA efficiency | 31.8% | 26.3% | 22.7% | 48.7% | 45.9% | 32.0% | 29.2% | 47.9% | | 32.4% | 33.3% |
| total saturates | 10.2 | 10.6 | 7.9 | 15.1 | 12.4 | 9.6 | 10.2 | 13.7 | 11.6 | 11.3 | 10.6 |
| total monounsaturates | 40.4 | 47 | 51.1 | 23.8 | 28.7 | 47.2 | 53 | 21.8 | 34.7 | 44.7 | 51 |
| total polyunsaturates | 49.2 | 42.5 | 40.9 | 61 | 59.4 | 43.3 | 36.8 | 64.3 | 53.7 | 44.2 | 38.4 |
| total C20 | 4.2 | 4 | 3.2 | 5.1 | 4.7 | 3.6 | 3.5 | 5.1 | 2.8 | 4 | 3.4 |
| total C22 | 2.6 | 2.5 | 1.3 | 8.3 | 6.4 | 1.7 | 1.8 | 11.1 | 0.5 | 2.4 | 1.9 |
| C20/C22 ratio | 1.62 | 1.60 | 2.46 | 0.61 | 0.73 | 2.12 | 1.94 | 0.46 | 5.60 | 1.67 | 1.79 |

Compiled data from the total lipid profiles (Table 14) are shown in Table 15. From the data in Table 15, the total ω6 FA (including LA) to ω3 FA (including ALA) ratio in the seed with the highest level of DHA was 3.34. The new ω6 FA (excluding LA) to new ω3 FA (excluding ALA) ratio was 1.39. The level of total saturated fatty acids was about 13.7% and the level of monounsaturated fatty acids was about 21.8%. The level of total 06-fatty acids was about 46.4% and the level of ω3-fatty acids was about 14.8%. Overall conversion efficiencies were calculated to be: OA to EPA=12.8%, OA to DHA=8.5%, LA to EPA=15.7%, LA to DHA=10.4%. ALA to EPA=72.1%, ALA to DHA=47.9%. The reduced efficiency of the ω6 fatty acids to ω3 fatty acids conversion observed in this experiment with the combination of the pJP3115 and pJP3116 was thought to be due to a lower efficiency of the plant Δ15-desaturase compared to the fungal Δ15/ω3 desaturase (Examples 2 and 3) when combined with the genes for conversion of ALA to DHA.

Progeny from DHA-containing lines which are homozygous for all of the introduced transgenes are generated for analysis.

### Example 5. Modifications to T-DNAs Encoding DHA Pathways in Plant Seeds

In order to improve the DHA production level in B. napus beyond the levels described in Example 4, the binary vectors pJP3416-GA7-modA, pJP3416-GA7-modB, pJP3416-GA7-modC, pJP3416-GA7-modD, pJP3416-GA7-modE and pJP3416-GA7-modF were constructed as follows. These binary vectors were variants of the pJP3416-GA7 construct described in Example 2 and were designed to further increase the synthesis of DHA in plant seeds, particularly by improving Δ6-desaturase and Δ6-elongase functions. SDA had been observed to accumulate in some seed transformed with the GA7 construct due to a relatively low elongation efficiency compared to the Δ5-elongase, so amongst other modifications, the two elongase gene positions were switched in the T-DNA.

The two elongase coding sequences in pJP3416-GA7 were switched in their positions on the T-DNA to yield pJP3416-GA7-modA by first cloning a new P. cordata Δ6-elongase cassette between the SbfI sites of pJP3416-GA7 to replace the P. cordata Δ5-elongase cassette. This construct was further modified by exchanging the FP1 promoter driving the M. pusilla Δ6-desaturase with a conlinin Cnl2 promoter (pLuCnl2) to yield pJP3416-GA7-modB. This modification was made in an attempt to increase the Δ6-desaturase expression and thereby enzyme efficiency. It was thought that the Cn12 promoter might yield higher expression of the transgene in B. napus than the truncated napin promoter. pJP3416-GA7-modC was produced by adding a second M. pusilla Δ6-desaturase cassette with slightly different codon usage (SEQ ID NO:15) and driven by the FP1 promoter, which was inserted at the PmeI site just inside the right border of pJP3416-GA7-modB. The second Δ6-desaturase cassette was added to both pJP3416-GA7-modB and pJP3416-GA7-modF in order to increase the Δ6-desaturase expression level and extend the time period during seed development for expression of Δ6-desaturase by the use of multiple promoters. Different codon usages were used in the two nucleotide sequences to result in the translation of the same protein sequence without risking co-suppression from similar coding regions within the same T-DNA. pJP3416-GA7-modD and pJP3416-GA7-modE were similar variants in which a third MAR sequence, corresponding to nucleotides 16649-17816 of SEQ ID NO: 1, was added to pJP3416-GA7 and pJP3416-GA7-modB, respectively, at the PmeI site. pJP3416-GA7-modF was produced by adding a second M. pusilla Δ6-desaturase cassette containing the native Δ6-desaturase nucleotide sequence and driven by the FP1 promoter at the PmeI site at the right border of pJP3416-GA7-modB. pJP3416-GA7-modG was made by first replacing the M. pusilla Δ6-desaturase cassette with a Cnl2:P. cordata Δ5-elongase cassette by restriction cloning at the AscI-PacI sites. pJP3416-GA7-modG was then made by replacing the original FAE1:P. cordata Δ5-elongase cassette with a FAE1:M. pusilla Δ6-desaturase cassette by restriction cloning at the SbfI sites. The nucleotide sequences of the T-DNAs from each of these genetic constructs are shown as: pJP3416-GA7-modB (SEQ ID NO:2), pJP3416-GA7-modC (SEQ ID NO:3), pJP3416-GA7-modD (SEQ ID NO:4), pJP3416-GA7-modE (SEQ ID NO:5), pJP3416-GA7-modF (SEQ ID NO:6) and pJP3416-GA7-modG (SEQ ID NO:7).

The binary vectors pJP3416-GA7-modB, pJP3416-GA7-modC, pJP3416-GA7-modD, pJP3416-GA7-modE, pJP3416-GA7-modF and pJP3416-GA7-modG are used to generate transformed Brassica somatic embryos and Brassica napus, Camelina sativa and Arabidopsis thaliana plants and progeny seeds. Data for pJP3416-GA7-modB are shown in the next Example.

Eight transgenic pJP3416-GA7-modB A. thaliana events and 15 transgenic pJP3416-GA7-modG A. thaliana events were generated. Between 3.4% and 7.2% DHA in pooled pJP3416-GA7-modB seed was observed and between 0.6 and 4.1% DHA in pooled T₂ pJP3416-GA7-modG seed was observed. Several of the highest pJP3416-GA7-modB events were sown out on selectable media and surviving seedlings taken to the next generation. Seed is being analysed for DHA content. Since the pooled T₁ seeds represented populations that were segregating for the transgenes and included any null segregants, it is expected that the homozygous seeds from progeny plants will have increased levels of DHA, up to 20% of the total fatty acid content in the seed oil. The other modified constructs were used to transform A. thaliana. Although only a small number of transformed lines were obtained, none yielded higher levels of DHA than the modB construct.

The pJP3416-GA7-modB construct was also used to generate transformed B. napus plants of cultivar Oscar and in a breeding line designated NX005. Ten independent transformed plants (TO) were obtained so far for the Oscar transformation, and 20 independent lines for NX005. Seed (T1 seed) was harvested from these transgenic lines. Pools of seed were tested for levels of DHA in the seed oil, and two lines which showed the highest levels were selected, these were designated lines CT132.5 (in cultivar Oscar) and CT133.15 (in NX005). Twenty seeds from CT132.5 and 11 seeds from CT133.15 were imbibed and, after two days, oil was extracted from a half cotyledon from each of the individual seeds. The other half cotyledons with embryonic axes were kept and cultured on media to maintain the specific progeny lines. The fatty acid composition in the oil was determined; the data is shown in Table 16 for CT132.5. The DHA level in ten of the 20 seeds analysed was in the range of 7-20% of the total fatty acid content as determined by the GC analysis. Other seeds had less than 7% DHA and may have contained a partial (incomplete) copy of the T-DNA from pJP3416-GA7-modB. The transgenic line appeared to contain multiple transgene insertions that were genetically unlinked. The seeds of transgenic line CT133.15 exhibited DHA levels in the range 0-5%. Seeds with no DHA were likely to be null segregants. These data confirmed that the modB construct performed well for DHA production in canola seed.

The pJP3416-GA7-modB and pJP3416-GA7-modF constructs were also used to generate transformed Camelina sativa plants. At least 24 independent transformed plants (T0) were obtained and examined in more detail by progeny analysis. Seed (T1 seed) was harvested from these transgenic lines. Pools of seed were tested for levels of DHA in the seed oil, and 6 lines which showed the highest levels of DHA (between 6% and 9%) were selected. The DHA levels in 20 T1 seeds from each line were analysed-most seeds exhibited DHA levels in the range of 6-14% of the total fatty acid content as determined by the GC analysis. The fatty acid composition in the oil was determined, the data is shown in Table 17 for several transgenic seeds. These data confirmed that the modB and modF constructs both performed well for DHA production in Camelina seed.

TABLE 16
Fatty acid profiles of half cotyledons of germinating T1 transgenic B. napus seeds containing the
modB construct. Up to 18.1% DHA was observed with numerous samples containing greater than 10% DHA.

| Seed | 14:0 | 16:0 | 16:1d3? | 16:1 | 16:3 | 18:0 | 18:1 | 18:1d11 | 18:2 | 18:3n6 | 18:3n3 | 20:0 | 18:4n3 | C20:1d11 | 20:1d13 |
|---|---|---|---|---|---|---|---|---|---|---|---|---|---|---|---|
| 1 | 0.1 | 4.2 | 0.1 | 0.1 | 0.2 | 1.8 | 29.9 | 2.5 | 9.9 | 0.1 | 38.4 | 0.5 | 0.8 | 1.0 | 0.0 |
| 2 | 0.1 | 4.7 | 0.1 | 0.1 | 0.2 | 4.0 | 23.0 | 2.3 | 7.4 | 0.3 | 29.3 | 1.0 | 4.3 | 1.1 | 0.0 |
| 3 | 0.1 | 3.7 | 0.2 | 0.1 | 0.2 | 1.8 | 55.1 | 1.9 | 4.7 | 0.2 | 15.2 | 0.8 | 1.8 | 1.4 | 0.0 |
| 4 | 0.1 | 4.6 | 0.2 | 0.2 | 0.2 | 2.9 | 22.1 | 1.8 | 6.6 | 0.4 | 26.5 | 1.0 | 7.2 | 1.0 | 0.0 |
| 5 | 0.1 | 4.0 | 0.1 | 0.1 | 0.2 | 1.7 | 27.4 | 2.1 | 8.1 | 0.3 | 26.4 | 0.6 | 2.8 | 1.0 | 0.0 |
| 6 | 0.1 | 3.5 | 0.1 | 0.1 | 0.2 | 1.6 | 59.8 | 2.0 | 4.3 | 0.1 | 18.5 | 0.6 | 0.5 | 1.3 | 0.0 |
| 7 | 0.1 | 6.0 | 0.3 | 0.3 | 0.3 | 1.7 | 16.6 | 2.6 | 23.9 | 1.0 | 23.2 | 0.6 | 5.4 | 0.8 | 0.0 |
| 8 | 0.1 | 4.9 | 0.1 | 0.1 | 0.2 | 2.7 | 12.9 | 1.4 | 11.7 | 0.3 | 34.3 | 0.9 | 5.0 | 0.9 | 0.0 |
| 9 | 0.1 | 3.9 | 0.1 | 0.1 | 0.1 | 2.4 | 41.6 | 1.7 | 21.5 | 0.0 | 23.4 | 0.7 | 0.0 | 1.2 | 0.0 |
| 10 | 0.1 | 3.7 | 0.2 | 0.1 | 0.1 | 2.1 | 30.9 | 1.7 | 19.2 | 0.4 | 23.6 | 0.7 | 2.1 | 1.1 | 0.0 |
| 11 | 0.1 | 5.7 | 0.4 | 0.3 | 0.2 | 3.8 | 41.2 | 2.4 | 26.7 | 2.1 | 7.2 | 1.3 | 0.3 | 1.2 | 0.0 |
| 12 | 0.1 | 4.6 | 0.0 | 0.1 | 0.2 | 2.4 | 25.5 | 1.7 | 16.1 | 0.3 | 28.9 | 0.8 | 3.9 | 1.1 | 0.0 |
| 13 | 0.1 | 4.3 | 0.1 | 0.1 | 0.1 | 4.2 | 19.4 | 1.6 | 9.2 | 0.1 | 45.5 | 1.0 | 0.2 | 1.1 | 0.0 |
| 14 | 0.1 | 6.3 | 0.2 | 0.2 | 0.2 | 4.0 | 10.5 | 2.3 | 8.4 | 0.3 | 31.1 | 1.3 | 3.9 | 0.8 | 0.0 |
| 15 | 0.1 | 5.1 | 0.1 | 0.2 | 0.2 | 3.3 | 16.8 | 2.4 | 11.2 | 0.3 | 28.8 | 1.0 | 4.5 | 0.9 | 0.0 |
| 16 | 0.1 | 4.4 | 0.1 | 0.1 | 0.2 | 4.0 | 16.2 | 1.5 | 11.6 | 0.2 | 33.5 | 0.9 | 2.8 | 1.1 | 0.0 |
| 17 | 0.2 | 7.2 | 0.2 | 0.2 | 0.2 | 4.9 | 15.0 | 2.1 | 8.9 | 0.3 | 25.9 | 1.4 | 5.1 | 0.9 | 0.0 |
| 18 | 0.1 | 4.0 | 0.1 | 0.1 | 0.2 | 2.3 | 64.8 | 1.2 | 7.2 | 0.1 | 12.5 | 1.0 | 3.5 | 1.5 | 0.0 |
| 19 | 0.1 | 3.9 | 0.1 | 0.1 | 0.2 | 4.6 | 36.9 | 1.7 | 7.1 | 0.2 | 28.6 | 1.2 | 1.8 | 1.2 | 0.0 |
| 20 | 0.1 | 4.8 | 0.1 | 0.1 | 0.2 | 6.0 | 18.5 | 1.2 | 12.8 | 0.2 | 34.8 | 1.4 | 2.4 | 1.1 | 0.0 |

| Seed | C20:2n6 | C20:3n3 | C22:0 | 20:4n3 | 20:5n3 | 22:3n3 | C24:0 | C24:1 | 22:5n3 | C22:6n3 |
|---|---|---|---|---|---|---|---|---|---|---|
| 1 | 0.1 | 2.1 | 0.3 | 2.8 | 0.3 | 0.1 | 0.2 | 0.2 | 0.5 | 3.9 |
| 2 | 0.1 | 1.9 | 0.4 | 6.9 | 1.0 | 0.0 | 0.3 | 0.1 | 1.7 | 9.5 |
| 3 | 0.1 | 0.3 | 0.5 | 11.3 | 0.0 | 0.0 | 0.3 | 0.2 | 0.0 | 0.0 |
| 4 | 0.1 | 0.8 | 0.5 | 11.2 | 1.9 | 0.0 | 0.2 | 0.2 | 1.7 | 8.7 |
| 5 | 0.1 | 1.5 | 0.3 | 7.6 | 1.5 | 0.0 | 0.1 | 0.1 | 1.8 | 12.2 |
| 6 | 0.0 | 0.7 | 0.3 | 6.0 | 0.0 | 0.0 | 0.2 | 0.1 | 0.0 | 0.0 |
| 7 | 0.2 | 0.6 | 0.4 | 2.6 | 1.1 | 0.0 | 0.3 | 0.3 | 1.7 | 9.9 |
| 8 | 0.2 | 2.4 | 0.5 | 4.1 | 1.3 | 0.0 | 0.2 | 0.2 | 1.8 | 13.8 |
| 9 | 0.1 | 2.2 | 0.4 | 0.0 | 0.0 | 0.1 | 0.3 | 0.2 | 0.0 | 0.0 |
| 10 | 0.1 | 1.5 | 0.4 | 3.6 | 0.6 | 0.0 | 0.2 | 0.1 | 0.7 | 6.9 |
| 11 | 0.2 | 0.3 | 0.8 | 4.8 | 0.0 | 0.0 | 0.6 | 0.3 | 0.0 | 0.0 |
| 12 | 0.1 | 1.9 | 0.4 | 3.9 | 0.6 | 0.0 | 0.2 | 0.0 | 1.1 | 6.2 |
| 13 | 0.1 | 5.2 | 0.4 | 2.6 | 0.3 | 0.2 | 0.2 | 0.1 | 0.4 | 3.4 |
| 14 | 0.1 | 2.3 | 0.6 | 4.6 | 1.8 | 0.1 | 0.3 | 0.2 | 2.5 | 18.1 |
| 15 | 0.1 | 2.1 | 0.6 | 3.2 | 1.5 | 0.1 | 0.3 | 0.1 | 1.8 | 15.1 |
| 16 | 0.2 | 3.7 | 0.4 | 4.6 | 0.7 | 0.1 | 0.3 | 0.1 | 1.3 | 12.1 |
| 17 | 0.0 | 1.6 | 0.8 | 4.9 | 2.1 | 0.0 | 0.6 | 0.3 | 2.2 | 15.0 |
| 18 | 0.1 | 0.0 | 0.7 | 0.0 | 0.0 | 0.0 | 0.5 | 0.2 | 0.0 | 0.0 |
| 19 | 0.1 | 1.4 | 0.5 | 4.3 | 0.4 | 0.0 | 0.4 | 0.1 | 0.8 | 4.3 |
| 20 | 0.1 | 3.4 | 0.6 | 3.2 | 0.4 | 0.1 | 0.3 | 0.1 | 0.7 | 7.6 |

TABLE 17
Fatty acid profiles of T1 transgenic C. sativa seeds containing the modB or modF constructs

| | C14:0 | C16:0 | C16:1 | C18:0 | C18:1 | C18:1d11 | C18:2 | C18:3n6 | C18:3n3 | C20:0 |
|---|---|---|---|---|---|---|---|---|---|---|
| 123-8 | 0.1 | 7.3 | 0.0 | 5.2 | 7.9 | 1.0 | 7.7 | 0.7 | 29.9 | 2.3 |
| 123-12 | 0.1 | 8.3 | 0.0 | 5.3 | 7.2 | 1.2 | 8.7 | 0.9 | 27.2 | 2.5 |
| 5-8 | 0.1 | 8.3 | 0.1 | 3.5 | 9.4 | 1.3 | 8.1 | 1.1 | 29.0 | 1.0 |
| 5-9 | 0.1 | 8.1 | 0.0 | 3.5 | 9.4 | 1.2 | 8.4 | 1.2 | 29.2 | 1.0 |
| 17-10 | 0.1 | 8.7 | 0.1 | 4.1 | 8.4 | 1.3 | 5.5 | 1.2 | 26.1 | 1.6 |
| 17-26 | 0.1 | 8.8 | 0.1 | 5.5 | 5.0 | 1.3 | 7.6 | 0.9 | 27.8 | 2.7 |

| | 18:4n3 | C20:1d11 | 20:1d13 | C20:2n6 | C20:3n6 | C20:4n6 | C20:3n3 | C22:0 | 20:4n3 |
|---|---|---|---|---|---|---|---|---|---|
| 123-8 | 6.0 | 7.1 | 0.4 | 0.7 | 0.0 | 0.0 | 0.9 | 0.4 | 1.3 |
| 123-12 | 5.7 | 6.9 | 0.5 | 0.7 | 0.0 | 0.1 | 0.9 | 0.5 | 1.5 |
| 5-8 | 9.3 | 7.9 | 0.4 | 0.6 | 0.0 | 0.0 | 0.8 | 0.2 | 0.4 |
| 5-9 | 9.0 | 8.1 | 0.3 | 0.6 | 0.0 | 0.0 | 0.8 | 0.2 | 0.5 |
| 17-10 | 11.8 | 7.2 | 0.3 | 0.0 | 0.4 | 0.03 | 0.8 | 0.3 | 0.4 |
| 17-26 | 10.1 | 6.2 | 0.3 | 0.0 | 0.7 | 0.03 | 1.1 | 0.6 | 0.5 |

| | C22:1 | 20:5n3 | C22:2n6 | 22:3n3 | C24:0 | C24:1 | 22:5n3 | C22:6n3 |
|---|---|---|---|---|---|---|---|---|
| 123-8 | 1.0 | 4.6 | 0.0 | 0.1 | 0.2 | 0.3 | 1.5 | 13.3 |
| 123-12 | 1.2 | 5.0 | 0.0 | 0.1 | 0.2 | 0.4 | 1.5 | 13.2 |
| 5-8 | 0.8 | 3.4 | 0.0 | 0.1 | 0.2 | 0.4 | 0.9 | 12.6 |
| 5-9 | 0.8 | 3.5 | 0.0 | 0.1 | 0.1 | 0.3 | 0.9 | 12.6 |
| 17-10 | 0.7 | 5.5 | 0.0 | 0.0 | 0.2 | 0.3 | 1.3 | 13.5 |
| 17-26 | 1.0 | 4.7 | 0.1 | 0.1 | 0.3 | 0.4 | 1.0 | 13.1 |

The inventors considered that, in general, the efficiency of rate-limiting enzyme activities in the DHA pathway can be greater in multicopy T-DNA transformants compared to single-copy T-DNA transformants, or can be increased by inserting into the T-DNA multiple genes encoding the enzyme which might be limiting in the pathway. Evidence for the possible importance of multi-copy transformants was seen in the Arabidopsis seeds transformed with the GA7 construct (Example 2), where the highest yielding DHA event had three T-DNAs inserted into the host genome. The multiple genes can be identical, or preferably are different variants that encode the same polypeptide, or are under the control of different promoters which have overlapping expression patterns. For example, increased expression could be achieved by expression of multiple Δ6-desaturase coding regions, even where the same protein is produced. In pJP3416-GA7-modF and pJP3416-GA7-modC, for instance, two versions of the M. pusilla Δ6-desaturase were present and expressed by different promoters. The coding sequences had different codon usage and therefore different nucleotide sequences, to reduce potential silencing or co-suppression effects but resulting in the production of the same protein.

### Example 6. Activity of Seed-Specific Constructs in Somatic Embryos

In order to establish a rapid assay system which was predictive of expression of genetic constructs in seeds under the control of seed-specific promoters, a somatic embryo system was set up for Brassica napus. This used a vector to express the LEC2 transcription factor which is involved in initiation of somatic embryogenesis. As a demonstration, the binary vectors 35S:LEC2 and pJP107 (Petrie et al., 2010a and b) were introduced into Agrobacterium tumefaciens strain AGL1 via standard electroporation and the Agrobacterium transformants used to co-transform Brassica napus by co-cultivation. The T-DNA region of pJP107 contained genes encoding the Isochrysis galbana Δ9-elongase, P. salina Δ8-desaturase and P. salina Δ5-desaturase with each gene expressed by a seed-specific promoter. A control transformation used the 35S:LEC2 vector alone. 35S:LEC2 expression resulted in the generation of somatic embryos in tissue culture directly from the transformed B. napus callus tissue as described in Example 1.

Fatty acid analysis showed that the seed-specific genes on the T-DNA of the construct pJP107 were expressed in the transgenic somatic embryos in the presence of the co-transformed LEC2 gene and functioned to produce ARA (20:4^{Δ5,8,11,14}) from LA and EPA (20:5^{Δ5,8,11,14,17}) from ALA. The data for three co-transformed somatic embryos are shown in Table 18 and the fatty acid composition of each compared to the fatty acid composition of seed oil from Brassica napus seed which was transgenic for, and expressing, the T-DNA of pJP107 (Petrie et al., 2010a and b). Similar total percentages of ARA and the intermediate fatty acids EDA (20:2ω6) and DGLA (20:3ω6), as well as conversion efficiencies, were observed in somatic embryo tissue when compared with stably-transformed seed profiles. Similar results were observed in the fatty acid compositions of the stable T₂ transgenic seed and somatic embryos: ω6 fatty acids were at a level of 26.6% and 25.6% (on average), respectively, whilst ARA levels were found to be 9.7% and 10.6% (on average), respectively.

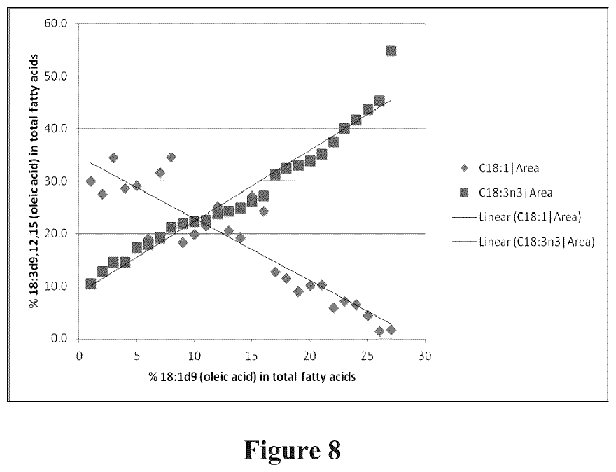

When 35S:LEC2 alone was introduced and the somatic embryos analysed in a time-course, the fatty acid profile was found to change to a more embryo-like profile with 18:3^{Δ9,12,15} decreasing and 18:1^{Δ9} increasing in an inversely correlated manner (FIG. 8). These results indicated that the somatic embryos were indeed becoming seed-like in character and the genes on the T-DNA from pJP107 were expressed. This demonstrated that the somatic embryo system allowed a rapid characterisation of transgenic seed-specific constructs in B. napus without requiring the full process of producing a transgenic plant and, from that, mature seed.

TABLE 18
Fatty acid composition of lipid obtained from Brassica napus
somatic embryos generated by co-transforming pJP107 with
35S:LEC2, compared to the control untransformed (WT) and
T₂ seeds transformed with pJP107. Individual enzymatic
conversion efficiencies are shown in brackets after the
relevant enzymatic steps. D9-Elo is Δ9-elongase.
D8-Des is Δ8-desaturase and D5-Des is Δ5-desaturase.

| | | T2 | | | |
|---|---|---|---|---|---|
| | | pJP107 | | | |
| | | trans- | | | |
| | | genic | | | |
| | WT | seed | LEC2:#45 | LEC2:#57 | LEC2:#58 |
| 18:1^{Δ9} | 57.2 | 45.7 | 3.8 | 2.5 | 1.9 |
| 18:2^{Δ9,} ¹² | 19.1 | 8.7 | 10 | 10.6 | 10 |
| 18:3^{Δ9,} ^{12, 15} | 10.2 | 4.1 | 22.5 | 27.5 | 24.2 |
| 20:2^{Δ11,} ¹⁴ | | 7.1 ± 1.9 | 5.2 | 3.7 | 4.6 |
| | | (67% | (61.8% | (56.7% | (61.8% |
| | | D9-elo) | D9-elo) | D9-elo) | D9-elo) |
| 20:3^{Δ8,} ^{11, 14} | | 1.1 ± 0.2 | 0.4 | 0.2 | 0.4 |
| | | (60% | (67% | (73% | (73% |
| | | D8-des) | D8-des) | D8-des) | D8-des) |
| 20:4^{Δ5,} ^{8, 11, 14} | | 9.7 ± 0.9 | 10.6 | 10 | 11.2 |
| | | (90% | (98% | (96% | (97% |
| | | D5-des) | D5-des) | D5-des) | D5-des) |
| 20:3^{Δ11,} ^{14, 17} | | 4.0 ± 0.8 | 9.9 | 5.5 | 7.3 |
| 20:4^{Δ8,} ^{11, 14, 17} | | 0.3 ± 0.1 | 0.4 | 0.3 | 0.4 |
| 20:5^{Δ5,} ^{8, 11, 14, 17} | | 2.4 ± 0.2 | 7.6 | 6.4 | 7.9 |
| Total new | | 24.6 | 34.1 | 26.1 | 31.8 |

Using the same system to generate somatic embryos, Brassica napus cells were transformed separately with pJP3416-GA7-modB and pJP3416-GA7-modD. 42 embryos were obtained, 18 for modB and 24 for modD. Total lipid was extracted from the embryos and analysed for fatty acid composition. The embryos contained between 0% and up to 16.9% DHA (Table 19). The results with 0% DHA was presumed to be due to integration of only a partial T-DNA or an insertion into a transcriptionally silent region of the genome. The total ω3 FA (including ALA) to total ω6 FA (including LA) ratio was found to be 2.3 for embryo #270 and 11.96 for embryo #284. The total ω6 FA (including LA) to total ω3 FA (including ALA) ratio was 0.08 for #284. The new ω6 FA (excluding LA) to new ω3 FA (excluding ALA) ratio was 0.03 for #284. Overall conversion efficiencies were calculated to be: (for embryos #270, #284) OA to EPA=14.0%, 29.8%: OA to DHA=9.7%, 24.2%; LA to EPA=15.4%, 30.7%: LA to DHA=10.7%, 25.0%: ALA to EPA=22.1%, 33.3%; ALA to DHA=15.3%, 27.0%. These efficiencies were similar, or greater than in the case of #284, to those observed for the T₃ pJP3416-GA7 Arabidopsis lines which indicated that the pJP3416-GA7-modB vector was capable of functioning well in B. napus cells. The SDA level was below 3.0%, indicating that the Δ6-elongase was performing even better than the GA7 construct. The individual enzyme efficiencies achieved in #284 were: Δ12-desaturase, 97.4%: ω3-desaturase, 92.3%; Δ6-desaturase, 38.2%; Δ6-elongase, 88.2%: Δ5-desaturase, 98.8%: Δ5-elongase, 94.1%; and Δ4-desaturase, 86.3%. Total saturates were 21.2%, total monounsaturates were 10.2%, total polyunsaturates were 68.6%.

The inventors believe this was the highest level of DHA achieved in B. napus cells to date, except for further data described below. This also demonstrated that the modification in pJP3416-GA7-modB relative to pJP3416-GA7 was effective in increasing the level of expression of the Δ6-desaturase gene. The binary vectors pJP3416-GA7, pJP3416-GA7-modA, pJP3416-GA7-modC, pJP3416-GA7-modD, pJP3416-GA7-modE and pJP3416-GA7-modF as described above are co-transformed with 35S:LEC2 to generate transformed B. napus somatic embryos. Up to 7.0% DHA was observed in modD embryos, 9.9% in modE embryos, 8.3% in modF embryos and 3.6% in a small number of modG embryos.

TABLE 19
Fatty acid composition of oil from
Brassica napus somatic embryos #270 and #284
generated by co-transforming the seed-
specific DHA acid construct pJP3416-
GA7-modB with 35S:LEC2,
and #286 and #289 (pJP3416-GA7-modD).

| | #270 | #284 | #286 | #289 |
|---|---|---|---|---|
| 14:0 | 0.3 | 0.2 | 0.7 | 0.2 |
| 16:0 | 14.0 | 15.7 | 17.2 | 16.6 |
| 16:1d9 | 0.7 | 0.4 | 0.8 | 0.8 |
| 16:3 | 0.5 | 0.6 | 1.1 | 1.3 |
| 18:0 | 2.6 | 2.4 | 2.5 | 2.5 |
| 18:1d9 | 6.6 | 1.8 | 1.5 | 1.1 |
| 18:1d11 | 6.3 | 6.8 | 6.5 | 6.7 |
| 18:2 | 18.9 | 4.5 | 10.0 | 9.8 |
| 18:3ω6 | 0.7 | 0.8 | 0.3 | 0.3 |
| 18:3ω3 | 33.0 | 37.2 | 42.0 | 41.5 |
| 20:0 | 0.9 | 0.9 | 0.8 | 0.8 |
| 18:4ω3 | 1.9 | 2.8 | 3.6 | 4.5 |
| 20:1d11 | 0.2 | 0.1 | 0.1 | 0.1 |
| 20:2ω6 | 0.1 | 0.1 | 0.1 | 0.2 |
| 20:3ω3 | 0.5 | 0.0 | 0.5 | 0.6 |
| 22:0 | 0.8 | 1.5 | 0.6 | 0.7 |
| 20:4ω3 | 0.2 | 0.9 | 0.7 | 0.7 |
| 20:5ω3 | 0.7 | 0.2 | 0.3 | 0.3 |
| 22:2ω6 | 0.0 | 1.2 | 0.0 | 0.0 |
| 22:3ω3 | 0.0 | 0.1 | 0.0 | 0.1 |
| 24:0 | 0.8 | 1.0 | 1.0 | 1.0 |
| 24:1 | 0.8 | 1.0 | 0.7 | 0.9 |
| 22:5ω3 | 2.4 | 2.7 | 3.2 | 3.0 |
| 22:6ω3 | 7.0 | 16.9 | 6.1 | 6.4 |

### Example 7. Analysis of TAG from Transgenic A. thaliana Seeds Producing DHA

The positional distribution of DHA on the TAG from the transformed A. thaliana seed was determined by NMR. Total lipid was extracted from approximately 200 mg of seed by first crushing them under hexane before transferring the crushed seed to a glass tube containing 10 mL hexane. The tube was warmed at approximately 55° C. in a water bath and then vortexed and centrifuged. The hexane solution was removed and the procedure repeated with a further 4×10 mL. The extracts were combined, concentrated by rotary evaporation and the TAG in the extracted lipid purified away from polar lipids by passage through a short silica column using 20 mL of 7% diethyl ether in hexane. Acyl group positional distributions on the purified TAG were determined quantitatively as previously described (Petrie et al., 2010a and b).

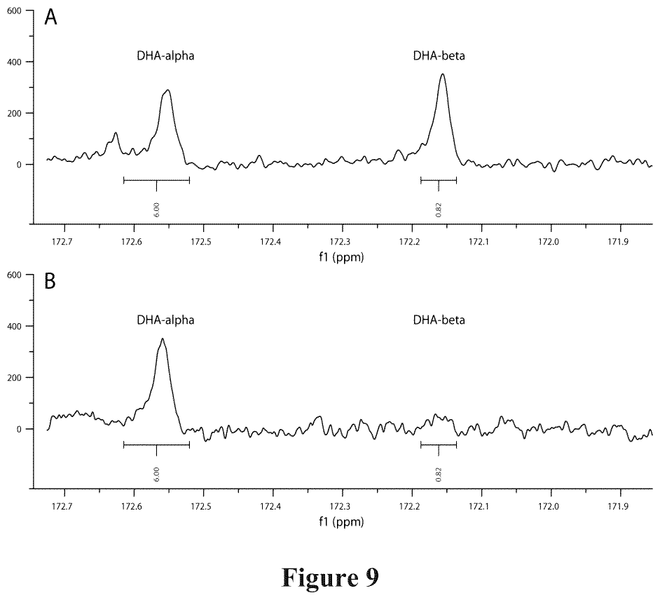

The analysis showed that the majority of the DHA in the total seed oil was located at the sn-1/3 positions of TAG with little found at the sn-2 position (FIG. 9). This was in contrast to TAG from ARA producing seeds which demonstrated that 50% of the ARA (20:4^{Δ5,8,11,14}) was located at the sn-2 position of transgenic canola oil whereas only 33% would be expected in a random distribution (Petrie et al., 2012).

Positional distribution of DHA in the TAG from the B. napus seeds transformed with pJP3416-GA7 or with the combination of pJP3115 and pJP3116 is determined by essentially the same method.

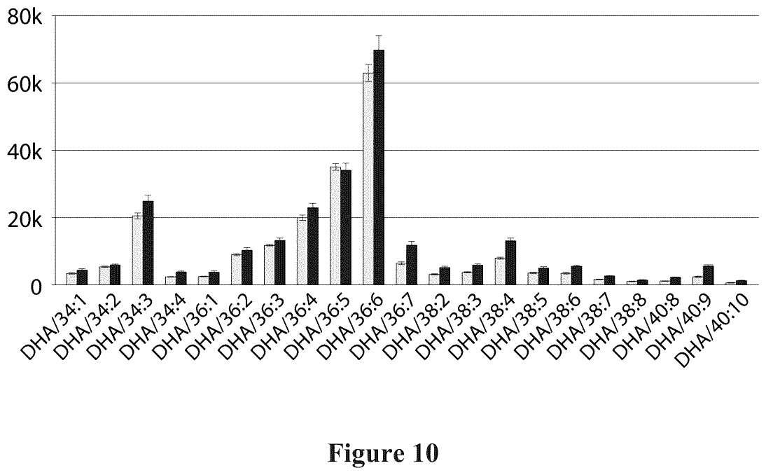

The total lipid from transgenic A. thaliana seeds was also analysed by triple quadrupole LC-MS to determine the major DHA-containing triacylglycerol (TAG) species (FIG. 10). The most abundant DHA-containing TAG species was found to be DHA-18:3-18:3 (TAG 58:12, nomenclature not descriptive of positional distribution) with the second-most abundant being DHA-18:3-18:2 (TAG 58:11). Tri-DHA TAG (TAG 66:18) was observed in total seed oil, albeit at low but detectable levels. Other major DHA-containing TAG species included DHA-34:3 (TAG 56:9), DHA-36:3 (TAG 58:9), DHA-36:4 (TAG 58:10), DHA-36:7 (TAG 58:13) and DHA-38:4 (TAG 60:10). The identities of the two major DHA-containing TAG were further confirmed by Q-TOF MS/MS.

### Example 8. Predicting DHA Production in B. napus Seeds

Efficient production of DHA in Arabidopsis seeds at a 15% level using the GA7 genetic construct was demonstrated in Example 2. The same construct in Brassica napus seeds produced only about 1.5% DHA in many (but not all) of the transformants, primarily due to the poor expression of the Δ6-desaturase gene of GA7 in this species (Example 4). Based on the realisation that modifications to the GA7 construct would overcome the Δ6-desaturase gene expression problem (see Example 5, as demonstrated in Example 6), calculations were performed to determine the likely fatty acid profile of B. napus transgenic seeds expressing the genes from a variant of pJP3416-GA7, where each transgene-encoded enzyme was performing as efficiently as was observed in A. thaliana with the GA7 construct. The predicted fatty acid compositions for three calculations (#1, #2, #3) are shown in Table 20. This was based on a wild-type (non-transformed) fatty acid composition for B. napus that included 59% oleic acid, 20% LA and 8% ALA. The three predicted partial fatty acid profiles shown in the lower half of the table were based on the conversion efficiencies for each enzymatic step shown in the upper half of the table. In prediction #2, a combination of Δ12-desaturation at 75% efficiency, Δ15-desaturation at 75%, Δ6-desaturation at 35%, Δ6-elongation at 80%, Δ5-desaturation at 90%. Δ5-elongation at 90% and Δ4-desaturation at 90% would result in the production of approximately 10% DHA in a typical canola transgenic seed. These efficiencies were all lower or about equal to the individual efficiencies seen in Arabidopsis, so prediction #2 represented a conservative estimate. The conversion efficiencies listed in #3 were approximations based on the efficient conversions seen in A. thaliana transformed with pJP3416-GA7. DHA was predicted to be produced at about 15% of the total fatty acid content in seedoil produced in B. napus seed, a result that mirrored the most efficient production levels observed in A. thaliana. Insertion of multiple T-DNAs in the homozygous state is expected to raise the DHA level to 20% in B. napus.

TABLE 20
Predicted fatty acid composition for selected fatty
acids as a percentage of total fatty acid content in
seedoil from Brassica napus transformed with a DHA
pathway construct, based on observed enzymatic
efficiencies in transgenic Arabidopsis. The enzymes are
listed in order in the pathway for producing DHA
from oleic acid. des = desaturase, elo = elongase.
Predicted fatty acid compositions #1, #2 and #3
are based on the efficiencies in the upper half of the Table.

| Enzyme | #1 | #2 | #3 | |
|---|---|---|---|---|
| d12-des | 70% | 75% | 80% | |
| d15-des | 70% | 75% | 80% | |
| d6-des (ω3) | 30% | 35% | 40% | |
| d6-elo | 80% | 80% | 90% | |
| d5-des | 80% | 90% | 90% | |
| d5-elo | 80% | 90% | 90% | |
| d4-des | 80% | 90% | 90% | |
| Fatty acid | WT | #1 | #2 | #3 |
| 18:1d9 | 59% | 26% | 22% | 18% |
| 18:2ω6 | 20% | 19% | 17% | 14% |
| 18:3ω6 | | 1% | 2% | 3% |
| 18:3ω3 | 8% | 30% | 32% | 34% |
| 18:4ω3 | | 3% | 3% | 2% |
| 20:4ω3 | | 2% | 1% | 2% |
| 20:5ω3 | | 2% | 1% | 2% |
| 22:5ω3 | | 1% | 1% | 2% |
| 22:6ω3 | | 5% | 10% | 15% |

### Example 9. Stable Expression of a Transgenic EPA Pathway in Plant Leaf

### Binary Vector Construction

A binary vector, pORE04+11ABGBEC_Cowpea_EPA_insert (SEQ ID NO:8), was designed for introduction of a T-DNA into plants for the synthesis of EPA in leaf tissues. It contained chimeric genes encoding the enzymes: M. pusilla Δ6-desaturase (SEQ ID NO:16), P. cordata Δ6-elongase (SEQ ID NO:25) and P. salina Δ5-desaturase (SEQ ID NO:30), each under the control of the CaMV 35S and A. thaliana rubisco small subunit (SSU) promoters (FIG. 9). The binary vector was constructed by synthesising the region 199-10878 of SEQ ID 2 and cloning this into the recipient binary vector pORE04 (Coutu et al., 1997) at the BsiWI and KasI sites. The three fatty acid biosynthesis genes coded for the enzymes required to convert ALA, 18:3^{Δ9,12,15} to EPA, 20:5^{Δ5,8,11,14,17}.

Transient Expression of EPA Construct in N. benthamiana Leaf Cells

To test that the construct was correct and would express the genes efficiently in leaf tissues, the chimeric vector pORE04+11ABGBEC_Cowpea_EPA_insert was introduced into A. tumefaciens strain AGL1. The chimeric vector 35S:p19 was also introduced into A. tumefaciens strain AGL1 as described in Example 1. Cells from cultures of these infiltrated into leaf tissue of Nicotiana benthamiana plants in a 24° C. growth room. Several direct comparisons were infiltrated with the samples being compared located on either side of the same leaf. Experiments were performed in triplicate. Following infiltration, the plants were grown for a further five days before leaf discs were taken for fatty acid profile analysis by GC as described in Example 1. GC analysis revealed that the EPA vector was functioning to produce EPA in Nicotiana benthamiana leaf (Table 21) with the highest level of EPA found to be 10.7% of total leaf lipids.

Nicotiana tabacum Stable Transformation

The chimeric vector pORE04+11ABGBEC_Cowpea_EPA_insert was used to stably transform Nicotiana tabacum. The vector was introduced into A. tumefaciens strain AGL1 via standard electroporation procedure. The transformed cells were grown on solid LB media supplemented with kanamycin (50 mg/L) and rifampicin (25 mg/L) and incubated at 28° C. for two days. A single colony was used to initiate fresh culture. Following 48 h vigorous culture, the cells were collected by centrifugation at 2,000×g and the supernatant was removed. The cells were resuspended in fresh solution containing 50% LB and 50% MS medium at the density of OD₆₀₀=0.5.

TABLE 21
Fatly acid composition of total leaf lipid from transgenic
Nicotiana benthamiana (transient) and Nicationa tabacum
(stable primary transformant)
events with the highest EPA levels from each experiment.

| | | N. benthamiana | N. tabacum |
|---|---|---|---|
| | 14:0 | 0.1 | 0.1 |
| | 16:0 | 18.5 | 17.8 |
| | 16:1w13t | 2.2 | 3.8 |
| | 16:1d9 | 0.1 | 0 |
| | 16:3 | 6.2 | 5.7 |
| | 18:0 | 3.4 | 3.2 |
| | 18:1d11 | 0.3 | 0.3 |
| | 20:0 | 0.5 | 0.5 |
| | 22:0 | 0.2 | 0.3 |
| | 24:0 | 0.1 | 0.4 |
| | 18:1 | 2.9 | 1.6 |
| | 18:2ω6 | 12.6 | 14.5 |
| Omega-6 | 18:3ω6 | 2.3 | 2.9 |
| | 20:2ω6 | 0.0 | 0.0 |
| | 20:3ω6 | 0.1 | 0.0 |
| | 20:4ω6 | 0.3 | 0.7 |
| Omega-3 | 18:3ω3 | 37.1 | 32.4 |
| | 18:4ω3 | 1.6 | 1.9 |
| | 20:3ω3 | 0.1 | 0.3 |
| | 20:4ω3 | 0.3 | 1.1 |
| | 20:5ω3 | 10.7 | 12.1 |
| | 22:5ω3 | 0.3 | 0.4 |

Leaf samples of N. tabacum cultivar ω38 grown in vitro were excised and cut into square sections around 0.5-1 cm² in size with a sharp scalpel while immersed in the A. tumefaciens solution. The wounded N. tabacum leaf pieces submerged in A. tumefaciens were allowed to stand at room temperature for 10 minutes prior to being blotted dry on a sterile filter paper and transferred onto MS plates without supplement. Following a co-cultivation period of two days at 24° C., the explants were washed three times with sterile, liquid MS medium, then blotted dry with sterile filter paper and placed on the selective MS agar supplemented with 1.0 mg/L benzylaminopurine (BAP), 0.25 mg/L indoleacetic acid (IAA), 50 mg/L kanamycin and 250 mg/L cefotaxime. The plates were incubated at 24° C. for two weeks to allow for shoot development from the transformed N. tabacum leaf pieces.

To establish rooted transgenic plants in vitro, healthy green shoots were cut off and transferred into 200 mL tissue culture pots containing MS agar medium supplemented with 25 μg/L IAA, 50 mg/L kanamycin and 250 mg/L cefotaxime. Transgenic shoots were transferred to soil after rooting and grown to maturity in the glasshouse. Sufficiently large leaf discs were taken from 21 mature transgenic plants from and analysed for fatty acid profile as described in Example 1. All transgenic samples were found to contain EPA (Table 21) with the highest level of EPA in a hemizygous primary transformant found to be 12.1% of total leaf lipids. he leaf samples also contained a small amount (<0.5%) of DPA in their lipid, which resulted from elongation of the EPA by a low level of Δ5-elongation activity of the Δ6-elongase. The total ω3 FA (including ALA) to ω6 FA (including LA) ratio was found to be 2.7. Overall conversion efficiencies were calculated to be: OA to EPA=18.4%, LA to EPA=18.9%, ALA to EPA=25.9%. The production of 12.1% EPA is notable especially since the events were hemizygous primary transformants. The ALA to EPA efficiency in particular is close to that observed in stable seed transformants. It is worth noting that the construct did not contain a Δ12 or Δ15-desaturase to increase the conversion of OA and LA to ALA. Increased efficiencies would be expected with addition of these activities.

Seed from hemizygous transformants is being harvested and sown out to generate homozygous plants.

Seed set in the top EPA lines appeared normal and seed from lines #10 and #17 germinated well to establish the T₂ generation. The ratio of EPA to null (no EPA) lines indicated that event #28 was single-locus and the T₃ generation of this line was therefore also established. Fatty acid profile analysis of the T₃ population indicated that the transgenes were homozygous with no null events found and a stable amount of EPA. The average amount of EPA in the total leaf lipids in the entire T₃ population was found to be 9.4%0.3 (Table 22).

TABLE 22
Representative fatty acid profiles of total leaf lipids from wildtype (WT) and independent
transgenic or transiently-transformed lines (EPA). Species are Nicotiana benthamiana (transient
transformation), N. tabacum (a stably transformed T₃ population), Vigna unguiculata (stably
transformed T₁ event). The errors denote standard deviation of multiple samples. Apparent
conversion efficiencies shown at the bottom describe the ω3 pathway and are calculated
as the sum of product FAs/sum of substrate + product FAs.

| | | N. benthamiana | | N. tabacum | | V. unguiculata | |
|---|---|---|---|---|---|---|---|
| | | WT | EPA | WT | EPA | WT | EPA |
| | 16:0 | 17.7 ± 0.1 | 18.7 ± 0.2 | 15.0 ± 0.6 | 16.5 ± 0.5 | 18.0 | 18.2 ± 0.2 |
| | 16:1ω13t | 3.2 ± 0.1 | 2.2 ± 0 | 3.5 ± 0.1 | 3.0 ± 0.3 | 3.8 | 2.0 ± 0.9 |
| | 16:3 | 6.8 ± 0.1 | 6.2 ± 0.1 | 5.2 ± 0.5 | 5.4 ± 0.3 | — | — |
| | 18:0 | 3.1 ± 0 | 3.5 ± 0.3 | 2.2 ± 0.2 | 2.6 ± 0.1 | 1.8 | 4.5 ± 0.4 |
| | Minor | 1.4 ± 0 | 1.4 ± 0.1 | 3.1 ± 0.4 | 2.5 ± 0.3 | 2.3 | 2.5 ± 0.4 |
| | OA | 1.7 ± 0.1 | 2.7 ± 0.2 | 1.6 ± 0.3 | 2.1 ± 0.3 | 2.0 | 4.3 ± 1.3 |
| | LA | 12.5 ± 0.4 | 12.7 ± 0.2 | 17.0 ± 1.1 | 18.0 ± 0.9 | 13.4 | 18.2 ± 3.0 |
| | ALA | 53.3 ± 0.2 | 37.2 ± 0.2 | 52.2 ± 1.9 | 34.0 ± 0.6 | 58.6 | 38.2 ± 0 |
| Omega-6 | GLA | — | 2.3 ± 0.1 | — | 2.3 ± 0.3 | — | 0.6 ± 0.2 |
| | 20:2ω6 | 0.1 ± 0 | — | 0.1 ± 0 | 0.1 ± 0 | — | 0.1 ± 0 |
| | DGLA | 0.1 ± 0 | 0.1 ± 0 | — | — | — | — |
| | ARA | — | 0.3 ± 0 | — | 0.7 ± 0.1 | — | 0.2 ± 0 |
| Omega-3 | SDA | — | 1.5 ± 0.1 | — | 1.6 ± 0.1 | — | 1.5 ± 0 |
| | 20:3ω3 | 0.1 ± 0 | 0.1 ± 0 | 0.1 ± 0 | 0.3 ± 0 | 0.1 ± 0 | 1.5 ± 0.1 |
| | ETA | — | 0.4 ± 0 | — | 1.1 ± 0.1 | — | 0.3 ± 0.2 |
| | EPA | — | 10.2 ± 0.5 | — | 9.4 ± 0.3 | — | 7.1 ± 0.2 |
| | DPA | — | 0.3 ± 0.1 | — | 0.4 ± 0 | — | 0.8 ± 0.1 |
| Omega-3 | Δ6-des | | 25% | | 27% | | 20% |
| conversion | Δ6-elo | | 88% | | 87% | | 85% |
| | Δ5-des | | 97% | | 90% | | 96% |
| | Δ5-elo | | 3% | | 4% | | 10% |

Leaf samples of homozygous T₃ N. tabacum plants were subjected to further biochemical analysis. Total lipids were extracted from freeze-dried leaf material and fractionated by thin-layer chromatography (TLC). EPA was found to be present in N. tabacum TAG at up to 30.1% as well as in the polar lipids at 6.3% (Table 23). It was interesting to note that the EPA produced by the transgenic pathway was present in all of the lipid fractions assessed including TAG, MGDG, DGDG, SQDG, PG, PC, PE, P1 and PS. All lipid pools contained low levels of novel intermediate or ω6 LC-PUFA fatty acids with the TAG ratio of novel ω3 to 06 fatty acids being 10:1.

### Stable Transformation of Cowpea

The chimeric vector pORE04+11ABGBEC-Cowpea-EPA-insert was transformed into cowpea (Vigna unguiculata) as follows. Mature dry seeds are the preferred starting material although seeds harvested from immature pods at maximum fresh weight of seeds can also be used. Dry seeds are threshed by hand to avoid cracking of seed coats and thus reduce contamination with microorganisms.

Dry seeds or immature pods are submerged in 70% ethanol for 2 min and then treated for 30 min in 20% commercial bleach (8.4 g/L sodium hypochlorite final concentration). The seeds are then washed several times with sterile water. Immature seeds are removed aseptically from pods while mature seeds are imbibed overnight. Two different explants can be used for multiple shoot production, ie the embryonic axis and the cotyledon itself, preferably the cotyledon with the bisected embryonic axis attached. The shoot and root tips are removed from the axis before wounding at the cotyledonary node, i.e, the point of attachment of the axis to the cotyledon. From an initial comparison of 19 cultivars and lines, it is now clear that most lines of cowpea can be transformed, the only caveat being that different tissue culture conditions need to be optimised for each line.

TABLE 23
Analysis of young and mature (young | mature) leaf lipid fractions triacylglycerol (TAG), total polar lipid (PL), monogalactosyldiacylglycerol
(MGDG), digalactosyldiacylglycerol (DGDG), sulfoquinovosyldiacylglycerol (SQDG), phosphatidylglycerol (PG), phosphatidylcholine (PC),
phosphatidylethanolamine (PE), phosphatidylinositol (PI) and phosphatidylserine (PS) from transgenic Nicotiana tabacum leaf samples.
The errors denote standard deviation of multiple samples. Up to 30% EPA was observed in leaf TAG with EPA also distributed throughout
the polar lipids. Differences between yound and mature leaf profiles were also observed for several fatty acids.

| | | | | Chloroplastidic | | | Extra-chloroplastidic | | | | |
|---|---|---|---|---|---|---|---|---|---|---|---|
| | | TAG | PL | MGDG | DGDG | SQDG | PG | PC | PE | PI | PS |
| | 16:0 | 9.8\|18.3 | 17.8\|23.8 | 3.1\|3.2 | 18.0\|16.8 | 48.3\|50.0 | 21.0\|26.4 | 22.9\|30.0 | 24.0\|30.5 | 38.7\|43.3 | 31.9\|36.2 |
| | 16:1ω13t | 0\|0 | 3.4\|3.1 | 0\|0 | 0\|0 | 0\|0 | 34.0\|32.0 | 0\|0 | 0\|0 | 0\|0 | 1.0\|1.4 |
| | 16:3 | 0.2\|0.9 | 5.6\|6.4 | 14.8\|19.4 | 1.2\|1.8 | 0.4\|1.2 | 0\|0 | 0\|0 | 0\|0 | 0\|0 | 0\|0 |
| | 18:0 | 7.3\|3.7 | 2.9\|3.9 | 1.1\|1.2 | 3.5\|3.5 | 5.4\|7.1 | 4.7\|6.9 | 6.6\|9.1 | 11.0\|11.4 | 9.4\|9.3 | 20.2\|19.4 |
| | Minor | 2.5\|2.9 | 1.4\|2.4 | 1.0\|0.4 | 0.8\|1.0 | 1.9\|2.1 | 1.0\|1.5 | 1.4\|1.6 | 4.9\|4.1 | 6.5\|7.7 | 2.5\|3.7 |
| | OA | 5.5\|0.8 | 2.8\|1.1 | 0.8\|0.3 | 1.8\|1.0 | 2.7\|1.3 | 5.3\|4.9 | 8.1\|2.9 | 2.5\|1.1 | 2.5\|0.8 | 4.9\|2.3 |
| | LA | 27.7\|13.7 | 17.3\|12.3 | 8.0\|6.8 | 9.2\|10.5 | 11.7\|8.9 | 17.1\|13.2 | 39.2\|25.2 | 37.9\|28.5 | 22.0\|13.4 | 24.4\|17.1 |
| | ALA | 9.6\|17.2 | 39.0\|34.4 | 60.3\|51.9 | 61.2\|58.6 | 23.7\|21.5 | 15.7\|14.1 | 7.3\|18.2 | 5.5\|10.5 | 7.6\|10.0 | 4.8\|10.5 |
| Omega-6 | GLA | 2.5\|3.0 | 1.5\|2.1 | 2.1\|3.0 | 1.1\|1.8 | 1.4\|1.9 | 0.2\|0 | 1.8\|2.5 | 1.7\|2.7 | 0.8\|0.9 | 1.1\|1.3 |
| | 20:2ω6 | 0\|0 | 0.1\|1.1 | 0\|0 | 0\|0 | 0\|0 | 0\|0 | 0\|0 | 0.5\|0 | 0\|0 | 0\|0 |
| | DGLA | 0\|0 | 0\|0 | 0\|0 | 0\|0 | 0\|0 | 0\|0 | 0\|0 | 0\|0 | 0\|0 | 0\|0 |
| | ARA | 0.6\|0.9 | 0.1\|0.2 | 0.2\|0.4 | 0\|0 | 0\|0 | 0\|0 | 0.3\|0.3 | 0.4\|0.4 | 0.4\|0.6 | 0\|0.2 |
| Omega-3 | SDA | 4.0\|7.6 | 1.6\|2.0 | 1.7\|2.0 | 0.6\|0.7 | 1.2\|1.2 | 0\|0 | 2.1\|3.6 | 1.3\|2.0 | 0.8\|0.8 | 0.9\|1.6 |
| | 20:3ω3 | 0.2\|0.3 | 0.1\|0.2 | 0\|0 | 0.2\|0.3 | 0\|0 | 0\|0.1 | 0.2\|0 | 0.3\|0.4 | 0\|0 | 0\|0 |
| | ETA | 0.9\|0.2 | 0.2\|0.3 | 0\|0.2 | 0\|0.3 | 0\|0 | 0\|0 | 0.2\|0 | 0.4\|0.2 | 0.1\|0.2 | 0\|0 |
| | EPA | 28.8\|30.1 | 6.1\|6.3 | 6.9\|11.2 | 2.3\|3.6 | 3.4\|4.6 | 1.0\|0.8 | 9.7\|6.4 | 9.1\|7.8 | 11.2\|12.8 | 8.4\|6.2 |
| | DPA | 0.4\|0.5 | 0 \|0 | 0\|0.1 | 0\|0 | 0\|0 | 0\|0 | 0.3\|0.4 | 0.5\|0.4 | 0\|0.2 | 0\|0.1 |

The selectable marker genes, bar or Np/I can be used for transformation. The Agrobacterium tumefaciens strain AGL1 is the preferred strain for cowpea transformation. Agrobacterium containing the pORE04+11ABGBEC-Cowpea-EPA-insert vector is cultured overnight at 28° C. on a shaker at 180 rpm and the suspension is centrifuged at 8000 g for 10 min and re-suspended in Medium 1 (MS-basic medium diluted one in ten and containing 30 g/l sucrose, 20 mM 2-MES, adjusted to pH 5.6 prior to autoclaving, supplemented with filter sterilized MS-vitamins, 100 mg/l myo-inositol, 1.7 mg/l BAP, 0.25 mg/l GA₃, 0.2 mM acetosyringone, 250 mg/l Na-thiosulphate, 150 mg/l dithiothreitol and 0.4 g/l L-cysteine). The explants are submerged without shaking in the bacterial suspension for one hour following wounding in the meristematic regions with a scalpel. The treated explants are then blotted on sterile filter paper and transferred to solidified Medium 2 (Medium 1 containing 0.8% agar) overlayed with filter paper. After four days of co-cultivation, explants are transferred to Medium 3 (full strength MS medium, supplemented with 100 mg/l myo-inositol, 150 mg/l timentin, 30 g/L sucrose, 3 mM MES, 1.7 mg/L BAP, 5 mg/L PPT or 25-50 mg/L geneticin or 150 mg/L kanamycin, 0.8 g/L agar and adjusted to pH 5.6) for shoot initiation and selection of transformed shoots. After two weeks the first shoots are visible. The cotyledons are removed from the cotyledonary node region and cultures are transferred to fresh Medium 3. Cultures are transferred to fresh Medium 3 every two weeks following removal of dead and dying tissue. The first four subcultures are on kanamycin selection followed by alternating with geneticin and kanamycin. After six sub-cultures, the surviving green shoots are transferred to Medium 4 (Medium 3 without BAP but supplemented with 0.5 mg/l GA3, 50 mg/l asparagine, 0.1 mg/l 3-indoleacetic acid (IAA), 150 mg/l timentin, and either PPT (10 mg/l), geneticin (50 mg/L) or kanamycin (150 mg/L), for shoot elongation. The shoots are sub-cultured every two weeks until single shoots are more than 1 cm long. These larger shoots are transferred from petri dishes to culture jars (80 mm height) for further growth under selection.

The majority of the regenerated shoots can be rooted in vitro, and the rooted plants are transferred to soil and allowed to establish in a high humidity chamber for 14-21 days before transfer to ambient greenhouse conditions.

To enhance gene transfer to cowpea, co-culture media is supplemented with thiol compounds. The addition of L-cysteine, dithiothreitol, and sodium thiosulfate reduces browning of wounded tissue.

Large numbers of cowpea explants can be processed in a simplified protocol. In brief, the protocol consists of the following steps: imbibition of sterilized mature seeds overnight in water, explants are derived by longitudinally bisecting the seed as a result of which, the split embryonic axis (with shoot and root apices removed) is still attached to the cotyledon, infection with Agrobacterium strain AGL1 aided by local wounding in the meristematic regions, co-culture on medium containing thiol compounds over 4 days at 25° C. in light, shoot initiation and elongation on medium containing selective agents, shoots are rooted in vitro and transferred to greenhouse conditions for flowering and seed setting, PCR or enzyme analysis of putative transgenic plants, and screening of next generation progeny by PCR or enzyme activity.

The progeny of transgenic T₀ plants are normal in phenotype. The transgenes are transmitted to the progeny and homozygous T₂ plants are identified by screening their T₃ progeny for enzyme activity or by PCR.

Using this transformation system about 10 transgenic plants are produced per 1000 explants, which is similar to the transformation frequency for other legumes. Depending on the cultivar or line to be transformed, this protocol requires 5-8 months from explant preparation to harvested T1 seeds.

The transformation system is used to introduce the pORE04+11ABGBEC-Cowpea-EPA-insert binary vector into regenerated, transformed cowpea plants.

Modifications to the pORE04+11ABGBEC-Cowpea-EPA-insert binary vector are made in which genes encoding a Δ5-elongase and Δ4-desaturase are added, to provide a genetic construct which confers the ability to further convert the produced EPA to DHA. The construct is transformed into plants for production of DHA in vegetative tissues.

EPA was found to be present in the small number of events surviving chemical selection. The highest line contained 7.1%±0.2 EPA in the total leaf lipids. The rate of transformation was lower than usually experienced for cowpea with only six lines confirmed transgenic. It is, as yet, unknown what caused this effect although it is interesting to note that a larger than usual proportion of transgenic events contained incomplete T-DNA regions. It is possible that the large construct size contributed to the reduced efficiency. The apparent conversion efficiencies of each of the three transgenic enzymes were also calculated (Table 22). Results were broadly similar in all three species with good conversion to EPA after initial Δ6-desaturation of the native ALA. Some Δ5-elongation of EPA to DPA was noted despite the absence of a specific Δ5-elongase. The P. cordata Δ6-elongase has previously been shown to have a low level of Δ9-elongase activity (i.e. 18:3^{Δ9,12,15} to 20:3^{Δ11,14,17} conversion) although no Δ5-elongase activity was detected in a yeast assay.

### Example 10. Testing Variations of Δ12-Desaturase Genes

### Binary Vector Construction

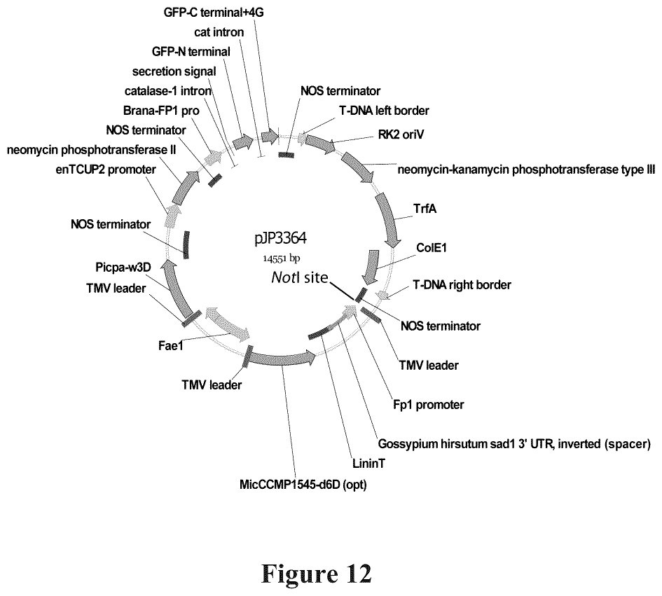

In an attempt to test and compare a series of chimeric Δ12-desaturase genes, several binary vectors were made which were used to transform A. thaliana and B. napus. The binary vectors pJP3365, pJP3366, pJP3367, pJP3368 and pJP3369 each contained genes that encoded the P. pastoris ω3-desaturase (SEQ ID NO:12) and M pusilla Δ6-desaturase (SEQ ID NO:16) enzymes, and one of a series of Δ12-desaturases. The Δ12-desaturases were from Cryptococcus neoformans (Accession No. XP_570226 in pJP3365), a version of the Cryptococcus neoformans Δ12-desaturase which contained a L151M mutation in an attempt to increase gene activity (in pJP3366), Lachancea kluyveri (SEQ ID NO:10 in pJP3367), Synechocystis PCC6803 (Accession No. BAA18169 in pJP3368) and Crepis palaestina (Accession No. CAA76157, Lee et al., 1998, in pJP3369). The Crepis desaturase was the only plant desaturase in the series; the others were fungal enzymes. The vectors were made by inserting a plant codon-optimised protein coding region, except for the Crepis palestina Δ12-desaturase which was wildtype, for each Δ12-desaturase into the Noll site of the vector pJP3364 (see FIG. 12), in the orientation operably linked to the FP1 promoter to provide for seed-specific expression of each desaturase. The vector pJP3364 already contained the chimeric genes encoding the P. pastoris ω3-desaturase and M pusilla Δ6-desaturase, each under the control of seed-specific promoters (FIG. 12). The combination of the three fatty acid biosynthesis enzymes, namely Δ12-desaturase, ω3-desaturase and Δ6-desaturase, was designed to assemble a pathway to convert oleic acid (18:10) to SDA (18:4^{Δ6,9,12,15}). Assays were therefore carried out to measure the level of SDA production in transformed seeds.

A. thaliana and B. napus Transformation and Analysis

The chimeric binary vectors were introduced into A. tumefaciens strain AGL1 and cells from cultures of the transformed Agrobacterium used to transform fad2 mutant A. thaliana plants using the floral dip method for transformation (Clough and Bent, 1998). After maturation, the T₁ seeds from the treated plants were harvested and plated on MS plates containing kanamycin for selection of plantlets having the NptII selectable marker gene present on the T-DNA of each chimeric vector. Surviving T₁ seedlings were transferred to soil. After allowing the plants to self-fertilise and growing them to maturity, the T₂ seeds from these plants were harvested and the fatty acid composition of seed lipids analysed by GC.

The chimeric vector pJP3367 was also used to transform B. napus by the method described in Example 4 to generate 12 transgenic events. SDA was found to range from 0.6% to 2.2% in pooled seed of the plants, and nine individual seeds from the transgenic plant with the highest SDA transgenic plant were analysed for fatty acid composition. Fatty acid composition data from such analysis is shown in Table 24.

The data showed that the Δ12-desaturase activity expressed from each of the T-DNAs in both A. thaliana and B. napus were unexpectedly low, providing an enzyme conversion efficiency of about 20% rather than the 70-80% seen with the same expression cassette in the GA7 construct (Examples 2 and 3). The reason for this relatively poor expression of the Δ12-desaturase genes from these vectors is unclear but could be related to the position of the genes in the construct as a whole.

In contrast, RT-PCR expression analysis demonstrated that the P. pastoris ω3-desaturase and M. pusilla Δ6-desaturase genes on the T-DNAs were relatively well expressed in the transformed seed. Table 24 includes the Δ6-desaturase conversion efficiencies in the transformed seeds, which ranged from about 11% to about 25% in the one B. napus transformed line. This was considerably higher than the Δ6-desaturase conversion efficiency of about 7% seen in the B. napus seeds transformed with the GA7 construct (Example 4).

TABLE 24
Fatty acid composition as a percentage of total fatty acids in seed oil from single seeds from a
T₁ Brassica napus plant transformed with the T-DNA from pJP3367. SDA (18:4ω3) is shown in bold.

| Sample | CT110-3#1 | CT110-3#2 | CT110-3#3 | CT110-3#4 | CT110-3#5 | CT110-3#6 | CT110-3#7 | CT110-3#8 | CT110-3#9 |
|---|---|---|---|---|---|---|---|---|---|
| C4:0 | 0.1 | 0.1 | 0.1 | 0.1 | 0.1 | 0.1 | 0.1 | 0.1 | 0.1 |
| C6:0 | 4.3 | 4.2 | 4.1 | 4.5 | 3.8 | 4.3 | 4.0 | 5.0 | 4.7 |
| 16:1d7 | 0.1 | 0.1 | 0.1 | 0.1 | 0.0 | 0.1 | 0.1 | 0.1 | 0.1 |
| C16:1d9 | 0.2 | 0.2 | 0.2 | 0.2 | 0.2 | 0.2 | 0.2 | 0.3 | 0.3 |
| 16:3 | 0.2 | 0.2 | 0.2 | 0.2 | 0.2 | 0.2 | 0.2 | 0.2 | 0.2 |
| C18:0 | 1.9 | 1.9 | 1.3 | 1.8 | 2.1 | 1.8 | 2.4 | 3.1 | 2.2 |
| C18:1 | 58.1 | 59.4 | 55.5 | 59.1 | 62.1 | 56.0 | 57.2 | 52.0 | 53.2 |
| C18:1d11 | 3.5 | 3.6 | 3.0 | 3.2 | 2.9 | 3.6 | 3.2 | 4.4 | 3.5 |
| C18:2 | 18.4 | 17.1 | 19.2 | 17.3 | 17.4 | 18.7 | 19.0 | 20.3 | 20.2 |
| C18:3ω6 | 0.3 | 0.2 | 0.3 | 0.2 | 0.2 | 0.2 | 0.2 | 0.2 | 0.3 |
| C18:3ω3 | 8.2 | 9.0 | 11.1 | 8.6 | 7.5 | 10.2 | 9.8 | 9.3 | 9.8 |
| C20:0 | 0.5 | 0.5 | 0.4 | 0.5 | 0.6 | 0.5 | 0.6 | 0.7 | 0.6 |
| 18:4ω3 | 2.4 | 2.0 | 2.8 | 2.5 | 1.4 | 2.6 | 1.3 | 2.4 | 3.2 |
| C20:1d11 | 1.1 | 1.1 | 1.2 | 1.2 | 1.2 | 1.1 | 1.2 | 1.1 | 1.1 |
| 20:liso | 0.03 | 0.03 | 0.03 | 0.03 | 0.01 | 0.03 | 0.02 | 0.03 | 0.02 |
| C20:2ω6 | 0.1 | 0.1 | 0.1 | 0.1 | 0.1 | 0.1 | 0.1 | 0.1 | 0.1 |
| C22:0 | 0.2 | 0.2 | 0.2 | 0.2 | 0.2 | 0.2 | 0.2 | 0.3 | 0.2 |
| C24:0 | 0.1 | 0.1 | 0.1 | 0.2 | 0.1 | 0.1 | 0.2 | 0.3 | 0.2 |
| C24:1 | 0.1 | 0.1 | 0.1 | 0.1 | 0.1 | 0.1 | 0.1 | 0.1 | 0.1 |
| Δ6-des % | 22.9 | 17.9 | 20.3 | 22.8 | 15.8 | 20.2 | 11.7 | 20.9 | 24.9 |

Therefore, to take advantage of the higher Δ6-desaturase conversion efficiencies conferred by the T-DNA from pJP3367, B. napus plants transformed with this T-DNA were crossed to plants transformed with the T-DNA from pJP3416-GA7 (Example 4) to produce progeny plants and seeds carrying both T-DNAs. The fatty acid composition of oil extracted from F1 seeds is analysed by GC for DHA content and other fatty acid contents. Increased DHA levels are observed as a consequence of increased expression of the Δ6-desaturase. Plants which are homozygous for both T-DNAs are produced and should produce higher levels of DHA.

### Example 11. Increasing Accumulation of Fatty Acids by Using Silencing Suppressor Proteins

### Binary Vector Construction

WO 2010/057246 describes the use of silencing suppressor proteins (SSP) to increase transgene expression in the seeds of plants. To demonstrate that the use of such proteins could enhance and stabilise the production of LC-PUFA in oilseeds over several generations, several SSP were selected for testing, namely V2 (Accession No. GU178820.1). p19 (Accession No. AJ288943.1), p38 (Accession No. DQ286869.1) and P0^{PE} (Accession No. L04573.1). p¹⁹ is a suppressor protein from Tomato Bushy Stunt Virus (TBSV) which binds to 21 nucleotide long siRNAs before they guide Argonaute-guided cleavage of homologous RNA (Voinnet et al., 2003). V2, a suppressor protein from Tomato Yellow Leaf Curl Virus (TYLCV), binds to the plant protein SGS3 (Glick et al., 2008), a protein thought to be required for the production of double stranded RNA intermediates from ssRNA substrates (Beclin et al., 2002), or binds to dsRNA structures that have a 5′ overhangs (Fukunaga et al., 2009). p38 is a suppressor protein from Turnip Crinkle Virus (TCV) which interferes with plant silencing mechanisms by binding to Dicer and Argonaute proteins (Azevedo et al., 2010). P0 proteins such as P0^{PE} and RPV-P0, from poleroviruses, target Argonaut proteins for enhanced degradation (Baumberger et al., 2007: Bortolamiol et al., 2007, Fusaro et al., 2012). Genetic constructs were therefore prepared for expression of these SSP in plant seed in combination with a set of fatty acid biosynthesis genes for production of ARA (20:4^{Δ5,8,11,14}) from LA (18:1^{Δ9,12}), as follows.

The fatty acid biosynthesis genes encoding the Isochrysis galbana Δ9-elongase and the Pavlova salina Δ8- and Δ5-desaturases and the bacterial selection marker were obtained on a single DNA fragment from pJP3010 by digestion with PmeI and AwII giving rise to a 9560 bp fragment. The Δ9-elongase coding region on this fragment was joined to an A. thaliana FAE1 promoter (pAtFAE1) and a conlinin transcription termination/polyadenylation region (LuCnl2-3′). The desaturase coding regions were each joined to a truncated napin FP1 promoter (pBnFP1) and a nos3′ transcription termination/polyadenylation region. The three fatty acid biosynthesis genes on this fragment were oriented and spaced in the same manner as in pJP107 (Petrie et al., 2012) and encoded the same proteins as pJP107. The DNA fragment also comprised a pFP1:GFiP:nos3′ gene from pCW141 (see WO2010/057246) which encoded a green fluorescent protein (GFP). This screenable marker gene was used as a visual seed-specific marker, allowing simple and non-destructive identification and thereby selection of transgenic seed comprising and expressing the gene.

The PmeI-AvrII fragment was inserted into the PmeI-AvrII site of each of a series of five vectors, each containing a different SSP gene (WO2010/057246), resulting in the genetic constructs designated pFN045, pFN046, pFN047, pFN048 and pFN049. These comprise the genes encoding the SSPs P0^{PE}, p38, p19, 35S:V2 and V2, respectively. Each of the SSP genes was under the control of the FP1 promoter and ocs3′ transcription termination/polyadenylation region except in the construct pFN048 where the V2 coding region was under the control of the constitutive CaMV 35S promoter. The SSP gene in each case was within the T-DNA region of the constructs, adjacent to the right border (RB) of the T-DNA. A sixth construct, pFN050 which lacked any SSP coding sequence, was made by digesting pFN045 with AhdI and NheI followed by recircularisation with DNA ligase to delete the FP1:POPE gene. Each of the six constructs comprised an NptII selectable marker gene within the T-DNA and adjacent to the left border of the T-DNA. All of the constructs had an RK2 origin of replication for maintenance of the plasmids in Agrobacterium.

Transformation of A. thaliana with ARA Expression Vectors in Combination with SSPs

To transform the genotype MC49 of Arabidopsis, which is a fad2:fae1 double mutant with high linoleic acid levels in its seed lipid, plants were treated by the floral dip method (Clough and Bent, 1998) with A. tumefaciens strain GV3101 separately transformed with each of the six constructs pFN045-pFN050. The treated plants were grown to maturity and T₁ seeds harvested from them were plated on MS media containing kanamycin to select for transformed T₁ plants. Screening for GFP expression in the seed was also used as a visual marker for transformed T₁ seeds. The seedlings which survived on MS/Kan plates or which were obtained from GFP-positive seeds were transferred to soil and grown to maturity for T₂ seeds. The numbers of transformed plants obtained were 5, 14, 32, 8, 23 and 24 for the transformations with pFN045, pFN046, pFN047, pFN048, pFN049 and pFN050, respectively. It was discovered at this stage that the gene encoding p38 in pFN046 was not functional and therefore plants transformed with the vector pFN046 were considered as additional controls i.e. essentially the same as for pFN050.

About 100 pooled T₂ seeds were taken from each transformed plant for fatty acid composition determination of seed lipid by FAME preparation and GC analysis. Six T₂ seedlings from each transgenic line were also grown to produce T₃ seeds.

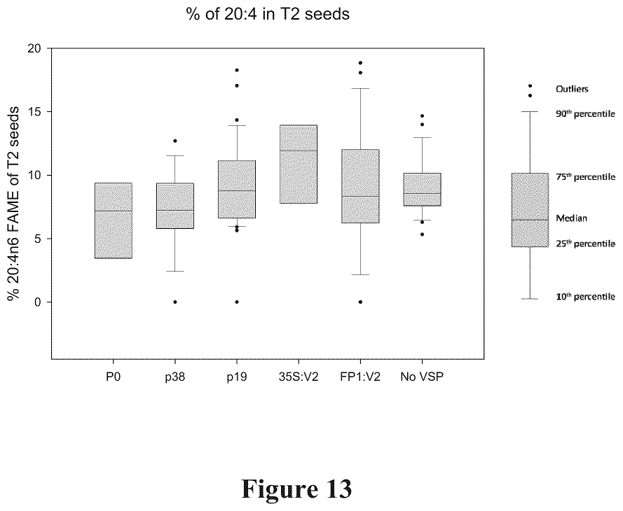

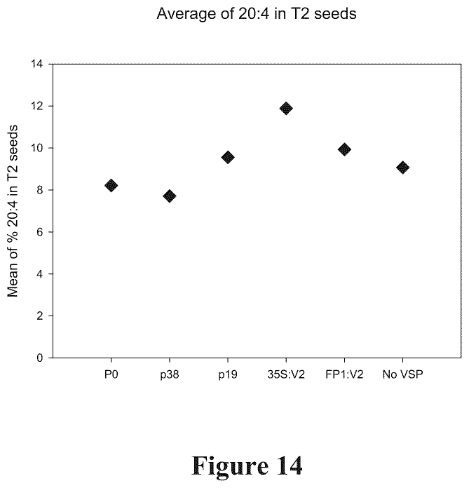

The fatty acid composition in total lipid extracted from the T₂ seeds was determined using GC. The analysis showed a range of levels of ARA and the intermediates EDA (20:2ω6) and DGLA (20:3ω6) in the T₂ populations. The data for ARA is shown in FIGS. 13 and 14.

FIG. 13 shows a box-plot analysis of the ARA level in the lipid of the populations of the T₂ seeds. It was evident that the median (50^{th} percentile) level of ARA in the populations of seeds which contained the FP1:p19 and 35S:V2 genes in addition to the ARA biosynthetic genes was significantly higher than in seeds containing the defective FP1:p38 gene or the control T-DNA from pFP050 which did not contain an SSP gene. The average ARA levels for seeds transformed with genes encoding p19 and V2 were greater than for seeds transformed with the p38 gene or those without an SSP (FIG. 14). One FP1:p19 and two FPL:V2 lines exhibited about 19%, 20% and 23% ARA, respectively. These were outliers and therefore not included in the calculations for the box-plot analysis. Fewer plants transformed with the T-DNAs comprising the genes FP1:P0^{PE} and 35S:V2 survived compared to the other constructs; it is thought that these genes could be detrimental to plant health in the MC49 background.

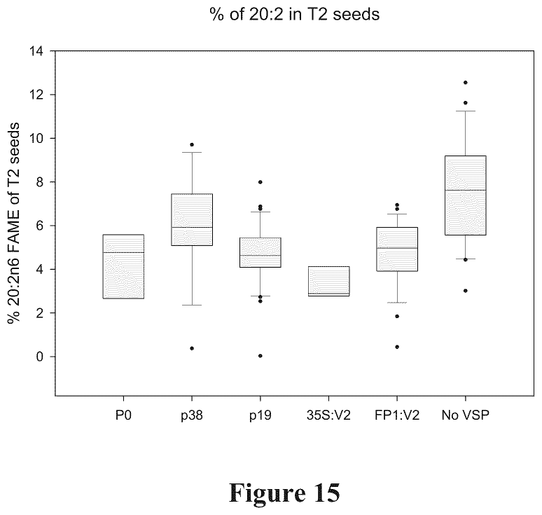

Not only were the ARA levels significantly different among the constructs, the levels in seed lipid of the first intermediate of the pathway from LA to ARA, namely EDA (20:2ω6), was observed to be lower in lines expressing either V2 or p19 than in seeds lacking an SSP or containing the p38 construct (FIG. 15). In T₃ seeds, one population containing the construct expressing p19 exhibited 38% ARA as a percentage of total fatty acids in the seed lipid.

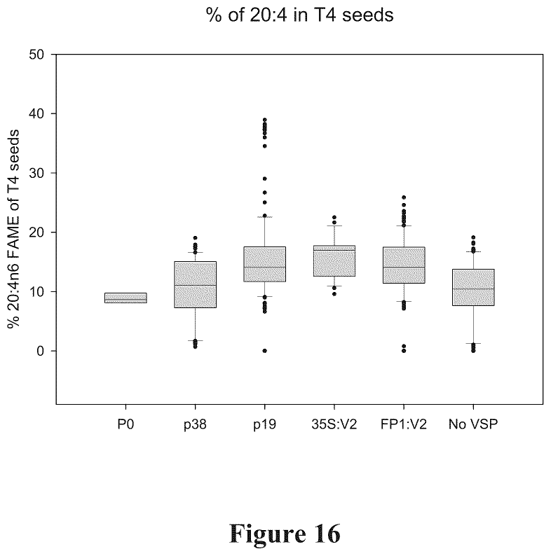

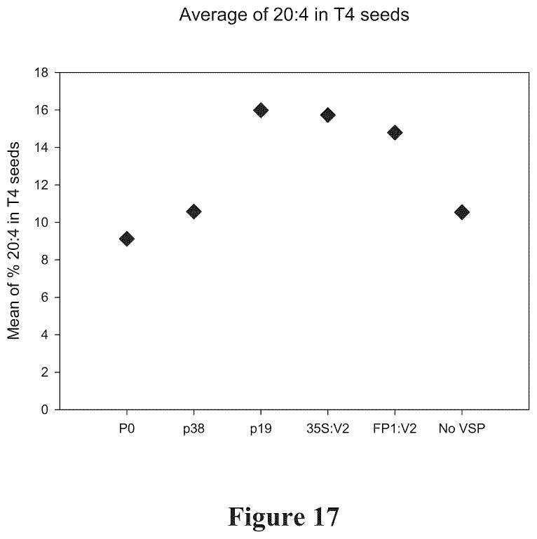

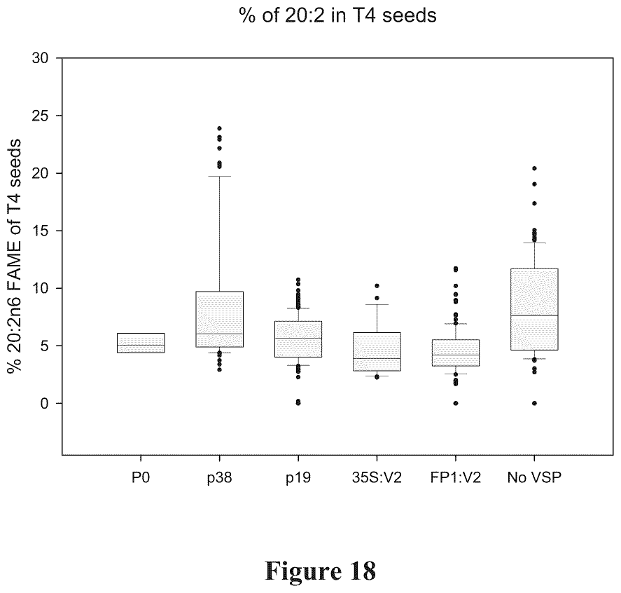

A range of transgenic T₃ lines were progressed to the T₄ generation. The levels of ARA in the T₄ seeds expressing V2 were either the same as compared to the previous generation, or indeed exhibited increased levels compared to their T3 parents (FIG. 16). The lines expressing p19 showed more varied ARA levels. The ARA level was decreased in some lines while in others it was the same or increased compared to the T3 parents. In contrast, the lines containing the defective p38 gene or lacking an SSP generally showed a decline in the level of ARA and an increase in the levels of intermediates (FIG. 18). In some of these lines, ARA was reduced to about 1% and levels of EDA had increased to about 20%. The mean levels of ARA in T₄ seeds were higher for lines expressing p19 and V2 compared to lines expressing p38 or lacking an SSP (FIG. 17).

This experiment showed that the expression of an SSP in seeds of a transgenic plant along with additional genes for a LC-PUFA biosynthetic pathway not only increased the level of production of the desired fatty acid in the first generation of progeny, but also stabilised the level of the fatty acid production in later generations such as the third or fourth generation of progeny. The increased fatty acid production was accompanied by decreased levels of intermediate fatty acids in the biosynthetic pathway. The SSP's p19 and V2 expressed from seed-specific promoters were preferred. The construct designed to express the p38 SSP was defective and no useful data were obtained with this construct. The V2 SSP and its homologs from other viruses are thought to be particularly preferred because they allow maximal expression of the biosynthetic pathway genes and the simultaneous silencing of other genes in the same cells in the developing seed.

### Example 12. Assaying Sterol Content and Composition in Oils

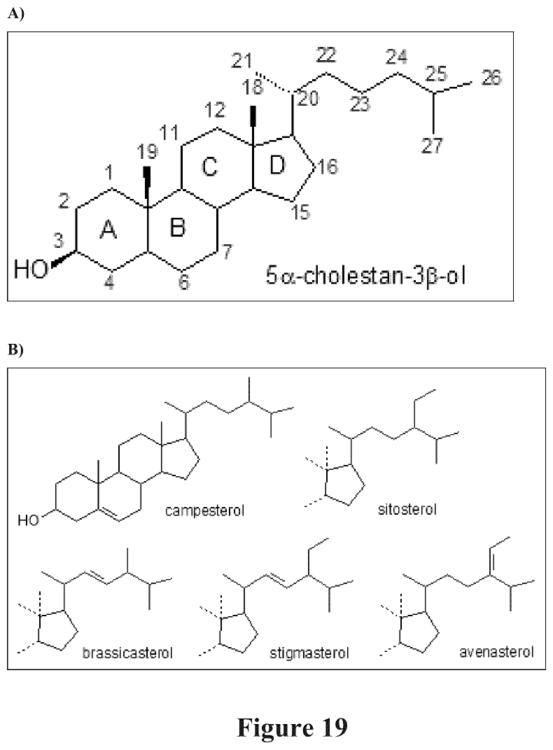

The phytosterols from 12 vegetable oil samples purchased from commercial sources in Australia were characterised by GC and GC-MS analysis as O-trimethylsilyl ether (OTMSi-ether) derivatives as described in Example 1. Sterols were identified by retention data, interpretation of mass spectra and comparison with literature and laboratory standard mass spectral data. The sterols were quantified by use of a 5β(H)-Cholan-24-ol internal standard. The basic phytosterol structure and the chemical structures of some of the identified sterols are shown in FIG. 19 and Table 25.

The vegetable oils analysed were from: sesame (Sesamum indicum), olive (Olea europaea), sunflower (Helianthus annus), castor (Ricinus communis), canola (Brassica napus), safflower (Carthamus tinctorius), peanut (Arachis hypogaea), flax (Linum usitatissimum) and soybean (Glycine max). In decreasing relative abundance, across all of the oil samples, the major phytosterols were: β-sitosterol (range 28-55% of total sterol content), Δ5-avenasterol (isofucosterol) (3-24%), campesterol (2-33%), Δ5-stigmasterol (0.7-18%). Δ7-stigmasterol (1-18%) and Δ7-avenasterol (0.1-5%). Several other minor sterols were identified, these were: cholesterol, brassicasterol, chalinasterol, campestanol and eburicol. Four C29:2 and two C30:2 sterols were also detected, but further research is required to complete identification of these minor components. In addition, several other unidentified sterols were present in some of the oils but due to their very low abundance, the mass spectra were not intense enough to enable identification of their structures.

The sterol contents expressed as mg/g of oil in decreasing amount were: canola oil (6.8 mg/g), sesame oil (5.8 mg/g), flax oil (4.8-5.2 mg/g), sunflower oil (3.7-4.1 mg/g), peanut oil (3.2 mg/g), safflower oil (3.0 mg/g), soybean oil (3.0 mg/g), olive oil (2.4 mg/g), castor oil (1.9 mg/g). The % sterol compositions and total sterol content are presented in Table 26.

TABLE 25
IUPAC/systematic names of identified sterols.

| Sterol | | |
|---|---|---|
| No. | Common name(s) | IUPAC/Systematic name |
| 1 | cholesterol | cholest-5-en-3β-ol |
| 2 | brassicasterol | 24-methylcholesta- |
| | | 5,22E-dien-3β-ol |
| 3 | chalinasterol/24- | 24-methylcholesta- |
| | methylene cholesterol | 5,24(28)E-dien-3β-ol |
| 4 | campesterol/ | 24-methylcholest-5-en- |
| | 24-methylcholesterol | 3β-ol |
| 5 | campestanol/24- | 24-methylcholestan- |
| | methylcholestanol | 3β-ol |
| 7 | Δ5-stigmasterol | 24-ethylcholesta-5,22E- |
| | | dien-3β-ol |
| 9 | ergost-7-en-3β-ol | 24-methylcholest-7-en- |
| | | 3β-ol |
| 11 | eburicol | 4,4,14-trimthylergosta- |
| | | 8,24(28)-dien-3β-ol |
| 12 | β-sitosterol/24- | 24-ethylcholest-5-en- |
| | ethylcholesterol | 3β-ol |
| 13 | D5-avenasterol/ | 24-ethylcholesta- |
| | isofucosterol | 5,24(28)Z-dien-3β-ol |
| 19 | D7-stigmasterol/ | 24-ethylcholest-7-en- |
| | stigmast-7-en-3b-ol | 3β-ol |
| 20 | D7-avenasterol | 24-ethylcholesta |
| | | 7,24(28)-dien-3β-ol |

Among all the seed oil samples, the major phytosterol was generally f3-sitosterol (range 30-57% of total sterol content). There was a wide range amongst the oils in the proportions of the other major sterols: campesterol (2-17%), Δ5-stigmasterol (0.7-18%), Δ5-avenasterol (4-23%), Δ7-stigmasterol (1-18%). Oils from different species had a different sterol profile with some having quite distinctive profiles. In the case of canola oil, it had the highest proportion of campesterol (33.6%), while the other species samples generally had lower levels, e.g. up to 17% in peanut oil. Safflower oil had a relatively high proportion of Δ7-stigmasterol (18%), while this sterol was usually low in the other species oils, up to 9% in sunflower oil. Because they were distinctive for each species, sterol profiles can therefore be used to help in the identification of specific vegetable or plant oils and to check their genuineness or adulteration with other oils.

TABLE 26
Sterol content and composition of assayed plant oils.

| Sterol | Sterol common | | | Sun- | Sun- | | | Saf- | Saf- | | Flax | Flax | Soy- |
|---|---|---|---|---|---|---|---|---|---|---|---|---|---|
| number* | name | Sesame | Olive | flower | flower | Castor | Canola | flower | flower | Peanut | (linseed) | (linseed) | bean |
| | | | | | cold- | | | | cold- | | | | |
| | | | | | pressed | | | | pressed | | | | |
| 1 | cholesterol | 0.2 | 0.8 | 0.2 | 0.0 | 0.1 | 0.3 | 0.2 | 0.1 | 0.2 | 0.4 | 0.4 | 0.2 |
| 2 | brassicasterol | 0.1 | 0.0 | 0.0 | 0.0 | 0.3 | 0.1 | 0.0 | 0.0 | 0.0 | 0.2 | 0.2 | 0.0 |
| 3 | chalinasterol/24- | 1.5 | 0.1 | 0.3 | 0.1 | 1.1 | 2.4 | 0.2 | 0.1 | 0.9 | 1.5 | 1.4 | 0.8 |
| | methylene | | | | | | | | | | | | |
| | cholesterol | | | | | | | | | | | | |
| 4 | campesterol/24- | 16.2 | 2.4 | 7.4 | 7.9 | 8.4 | 33.6 | 12.1 | 8.5 | 17.4 | 15.7 | 14.4 | 16.9 |
| | methylcholesterol | | | | | | | | | | | | |
| 5 | campestanol/24- | 0.7 | 0.3 | 0.3 | 0.1 | 0.9 | 0.2 | 0.8 | 0.8 | 0.3 | 0.2 | 0.2 | 0.7 |
| | methylcholestanol | | | | | | | | | | | | |
| 6 | C29:2* | 0.0 | 0.0 | 0.1 | 0.2 | 0.0 | 0.1 | 0.5 | 0.5 | 0.0 | 1.2 | 1.3 | 0.1 |
| 7 | Δ5-stigmasterol | 6.4 | 1.2 | 7.4 | 7.2 | 18.6 | 0.7 | 7.0 | 4.6 | 6.9 | 5.1 | 5.8 | 17.6 |
| 8 | unknown | 0.5 | 1.3 | 0.7 | 0.6 | 0.8 | 0.7 | 0.7 | 1.3 | 0.4 | 0.7 | 0.6 | 1.3 |
| 9 | ergost-7-en-3β-ol | 0.1 | 0.1 | 1.9 | 1.8 | 0.2 | 0.4 | 2.7 | 4.0 | 1.4 | 1.4 | 1.4 | 1.0 |
| 10 | unknown | 0.0 | 1.3 | 0.9 | 0.8 | 1.2 | 0.9 | 1.8 | 0.7 | 1.2 | 0.7 | 0.5 | 0.7 |
| 11 | eburicol | 1.6 | 1.8 | 4.1 | 4.4 | 1.5 | 1.0 | 1.9 | 2.9 | 1.2 | 3.5 | 3.3 | 0.9 |
| 12 | β-sitosterol/24- | 55.3 | 45.6 | 43.9 | 43.6 | 37.7 | 50.8 | 40.2 | 35.1 | 57.2 | 29.9 | 28.4 | 40.2 |
| | ethylcholesterol | | | | | | | | | | | | |
| 13 | Δ5-avenasterol/ | 8.6 | 16.9 | 7.2 | 4.1 | 19.3 | 4.4 | 7.3 | 6.3 | 5.3 | 23.0 | 24.2 | 3.3 |
| | isofucosterol | | | | | | | | | | | | |
| 14 | triterpenoid | 0.0 | 2.4 | 0.9 | 1.1 | 0.0 | 0.0 | 1.6 | 1.9 | 0.0 | 0.0 | 0.0 | 0.9 |
| | alcohol | | | | | | | | | | | | |
| 15 | triterpenoid | 0.0 | 0.0 | 0.7 | 0.6 | 0.0 | 0.0 | 2.8 | 1.8 | 0.0 | 0.0 | 0.3 | 0.0 |
| | alcohol | | | | | | | | | | | | |
| 16 | C29:2* | 0.0 | 0.5 | 0.7 | 0.7 | 1.5 | 1.2 | 2.8 | 1.9 | 2.0 | 1.0 | 0.7 | 0.5 |
| 17 | C29:2* | 1.0 | 0.9 | 2.3 | 2.4 | 0.6 | 0.4 | 1.3 | 1.9 | 0.9 | 1.0 | 1.0 | 1.0 |
| 18 | C30:2* | 0.0 | 0.0 | 0.0 | 0.0 | 1.9 | 0.0 | 0.0 | 0.0 | 0.0 | 0.0 | 0.0 | 0.0 |
| 19 | Δ7-stigmasterol/ | 2.2 | 7.1 | 9.3 | 10.9 | 2.3 | 0.9 | 10.5 | 18.3 | 1.1 | 7.9 | 8.7 | 5.6 |
| | stigmast-7-en-3β- | | | | | | | | | | | | |
| | ol | | | | | | | | | | | | |
| 20 | Δ7-avenasterol | 1.3 | 0.1 | 4.0 | 3.6 | 0.6 | 0.2 | 2.0 | 4.7 | 0.7 | 0.4 | 0.4 | 0.6 |
| 21 | unknown | 0.7 | 7.1 | 0.9 | 0.8 | 0.0 | 0.4 | 0.3 | 0.4 | 0.0 | 3.0 | 3.6 | 0.0 |
| 22 | unknown | 0.3 | 0.0 | 0.3 | 0.9 | 0.0 | 0.0 | 1.2 | 1.3 | 0.2 | 0.1 | 0.0 | 0.3 |
| 23 | unknown | 0.2 | 0.2 | 0.3 | 0.3 | 0.2 | 0.1 | 0.3 | 0.2 | 0.2 | 0.1 | 0.2 | 0.5 |
| 24 | unknown | 0.0 | 3.1 | 0.9 | 1.3 | 0.6 | 0.4 | 0.2 | 0.4 | 0.7 | 1.7 | 1.9 | 0.8 |
| 25 | unknown | 0.9 | 0.4 | 0.3 | 0.5 | 0.3 | 0.1 | 0.5 | 0.7 | 0.3 | 0.1 | 0.1 | 0.6 |
| 26 | C30:2 | 2.2 | 6.0 | 4.6 | 5.7 | 1.4 | 0.6 | 1.0 | 1.2 | 1.2 | 1.2 | 1.1 | 5.2 |
| 27 | unknown | 0.0 | 0.4 | 0.4 | 0.3 | 0.3 | 0.2 | 0.1 | 0.2 | 0.3 | 0.1 | 0.0 | 0.3 |
| | Sum | 100.0 | 100.0 | 100.0 | 100.0 | 100.0 | 100.0 | 100.0 | 100.0 | 100.0 | 100.0 | 100.0 | 100.0 |
| | Total sterol | 5.8 | 2.4 | 4.1 | 3.7 | 1.9 | 6.8 | 3.2 | 3.0 | 3.2 | 4.8 | 5.2 | 3.0 |
| | (mg/g oil) | | | | | | | | | | | | |

C29:2* and and C30:2* denotes a C29 sterol with two double bonds and a C30 sterol with two double bonds, respectively

Two samples each of sunflower and safflower were compared, in each case one was produced by cold pressing of seeds and unrefined, while the other was not cold-pressed and refined. Although some differences were observed, the two sources of oils had similar sterol compositions and total sterol contents, suggesting that processing and refining had little effect on these two parameters. The sterol content among the samples varied three-fold and ranged from 1.9 mg/g to 6.8 mg/g. Canola oil had the highest and castor oil the lowest sterol content.

### Example 13. Increasing Accumulation of DHA at the Sn-2 TAG Position

The present inventors considered that DHA accumulation at the sn-2 position in TAG could be increased by co-expressing an 1-acyl-glycerol-3-phosphate acyltransferase (LPAAT) together with the DHA biosynthesis pathway such as conferred by the GA7 construct or its variants. Preferred LPAATs are those which can act on polyunsaturated C22 fatty acyl-CoA as substrate, resulting in increased insertion of the polyunsaturated C22 chain at the sn-2 position of LPA to form PA, relative to the endogenous LPAAT. Cytoplasmic LPAAT enzymes often display varied substrate preferences, particularly where the species synthesises and accumulates unusual fatty acids in TAG. A LPAAT2 from Limnanthes douglasii was shown to use erucoyl-CoA (C22:1-CoA) as a substrate for PA synthesis, in contrast to an LPAAT1 from the same species that could not utilise the C22 substrate (Brown et al., 2002).

Known LPAATs were considered and a number were selected for testing, including some which were not expected to increase DHA incorporation at the sn-2 position, as controls. The known LPAATs included; Arabidopsis thaliana LPAAT2: (SEQ ID NO: 63, Accession No. ABG48392, Kim et al., 2005), Limnanthes alba LPAAT (SEQ ID NO: 64, Accession No. AAC49185, Lassner et al., 1995), Saccharomyces cerevisiae Slclp (SEQ ID NO: 65, Accession No. NP_010231, Zou et al., 1997), Mortierella alpina LPAAT1 (SEQ ID NO: 67, Accession No. AED33305; U.S. Pat. No. 7,879,591) and Brassica napus LPAATs (SEQ ID NO: 68 and SEQ ID NO:69, Accession Nos ADC97479 and ADC97478 respectively). These were chosen to cover three groups of LPAAT enzymes: 1) control plant seed LPAATs with typically low activity on unusual long-chain polyunsaturated fatty acids (including the Arabidopsis and Brassica LPAATs), 2. LPAATs that had previously been demonstrated to act on C22 fatty acids by using C22 acyl-CoA as substrate, in this case erucic acid C22:1 (including the Limnanthes and Saccharomyces LPAATs), 3. LPAATs which the inventors considered likely to be able to utilise long-chain polyunsaturated fatty acids such as EPA and DHA as substrates (including the Mortierella LPAAT).

The Arabidopsis LPAAT2 (also designated LPAT2) is an endoplasmic reticulum-localised enzyme shown to have activity on C16 and C18 substrates, however activity on C20 or C22 substrates was not tested (Kim et al., 2005). Limnanthes alba LPAAT2 was demonstrated to insert a C22:1 acyl chain into the sn-2 position of PA, although the ability to use DHA as a substrate was not tested (Lassner et al., 1995). The selected S. cerevisiae LPAAT Slclp was shown to have activity using 22:1-CoA in addition to 18:1-CoA as substrates, indicating a broad substrate specificity with respect to chain length (Zou et al., 1997). Again, DHA-CoA and other LC-PUFAs were not tested as substrates. The Mortierella LPAAT had previously been shown to have activity on EPA and DHA fatty acid substrates in transgenic Yarrowia lipolytica (U.S. Pat. No. 7,879,591).

Additional LPAATs were identified by the inventors. Micromonas pusilla is a microalga that produces and accumulates DHA in its oil, although the positional distribution of the DHA on TAG in this species has not been confirmed. The Micromonas pusilla LPAAT (SEQ ID NO: 66, Accession No. XP_002501997) was identified by searching the Micromonas pusilla genomic sequence using the Arabidopsis LPAAT2 as a BLAST query sequence. Several candidate sequences emerged and the sequence XP_002501997 was synthesised for testing as a likely LPAAT enzyme with activity on C22 LC-PUFA. The Ricinus communis LPAAT was annotated as a putative LPAAT in the castor genome sequence (Chan et al., 2010). Four candidate LPAATs from the castor genome were synthesised and tested in crude leaf lysates of infiltrated N. benthamiana leaf tissue. The candidate sequence described here showed LPAAT activity.

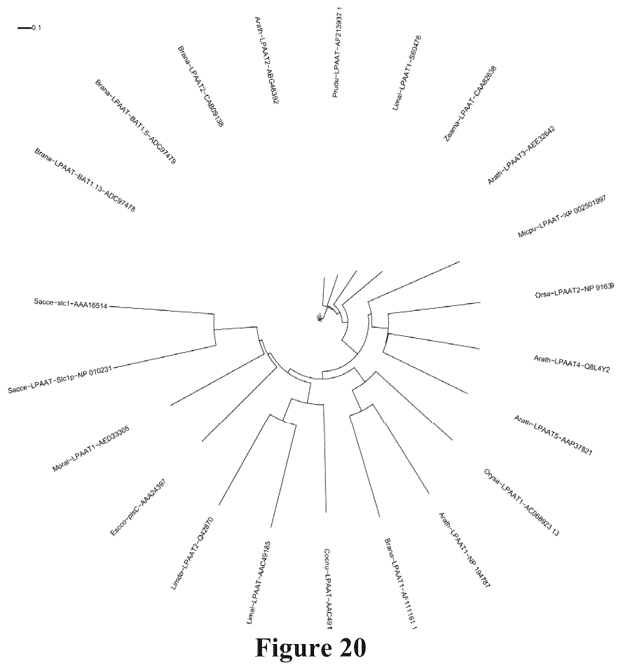

A number of candidate LPAATs were aligned with known LPAATs on a phylogenetic tree (FIG. 20). It was noted that the putative Micromonas LPAAT did not cluster with the putative C22 LPAATs but was a divergent sequence.

As an initial test of various LPAATs for their ability to use DHA-CoA as substrate, chimeric genetic constructs are made for constitutive expression of exogenous LPAATs in N. benthamiana leaves, each under the control of the 35S promoter, as follows: 35S:Arath-LPAAT2 (Arabidopsis ER LPAAT): 35S:Ricco-LPAAT2; 35S:Limal-LPAAT (Limnanthes alba LPAAT); 35S:Sacce-Slclp (S. cerevisiae LPAAT); 35S:Micpu-LPAAT (Micromonas pusilla LPAAT); 35S:Moral-LPAAT1 (Mortierella alpina LPAAT). A 35S:p19 construct lacking an exogenous LPAAT is used as a control in the experiment. Each of these constructs is introduced via Agrobacterium into N. benthamiana leaves as described in Example 1, and 5 days after infiltration, the treated leaf zones are excised and ground to make leaf lysates. Each lysate includes the exogenous LPAAT as well as the endogenous enzymes for synthesizing LPA. In vitro reactions are set up by separately adding ¹⁴C-labelled-OA, -LA or -ALA (C18 substrates), -ARA (a C20 substrate) and -DHA (C22) to the lysates, in triplicate. Reactions are incubated at 25° C., and the level of incorporation of the ¹⁴C labelled fatty acids into PA determined by TLC. The ability of each LPAAT to use DHA relative to ARA and the C18 fatty acids is calculated. The meadowfoam, Mortierella and Saccharomyces LPAATs were found to have activity on DHA substrate, with radiolabelled PA appearing for these but not the other LPAATs. All LPAATs were confirmed active by a similar oleic acid feed.

To test LPAAT activity in seeds, several of the protein coding sequences or LPAATs are inserted into a binary vector under the control of a conlinin (pLuCnl1) promoter. The resultant genetic constructs containing the chimeric genes, Cnl1:Arath-LPAAT (negative control), Cnl1:Limal-LPAAT, Cnl:Sacce-Slclp, and Cnl1:Moral-LPAAT, respectively, are then used transform B. napus and A. thaliana plants to generate stable transformants expressing the LPAATs in a seed-specific manner. The transformed plants having the Cnl1:LPAAT constructs are crossed with plants expressing the GA7 construct or its variants (Example 5) which produce DHA in the seed to result in increased incorporation of DHA at the sn-2 position of TAG. The constructs are also used to transform B. napus, (C. sativa and A. thaliana plants that already contain the GA7 construct and variants thereof (Examples 2 to 5) to generate progeny carrying both the parental and LPAAT genetic constructs. Increased incorporation of DHA at the sn-2 position of TAG is expected relative to the incorporation in plants lacking the LPAAT encoding transgenes. Oil content is also improved in the seeds, particularly for seeds producing higher levels of DHA, counteracting the trend seen in Arabidopsis seed as described in Example 2.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from U.S. 61/660,392 filed 15 Jun. 2012, U.S. 61/663,344 filed 22 Jun. 2012. U.S. 61/697,676 filed 6 Sep. 2012 and U.S. 61/782,680 filed 14 Mar. 2013, the entire contents of each of which are incorporated herein by reference.

All publications discussed and/or referenced herein are incorporated herein in their entirety.

This application incorporates herein by reference U.S. 61/660,392 filed 15 Jun. 2012, U.S. 61/663,344 filed 22 Jun. 2012 and U.S. 61/697,676 filed 6 Sep. 2012.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abbadi et al. (2004) Plant Cell 16: 2734-2748.
Abbott et al. (1998) Science 282:2012-2018.
Abdullah et al. (1986) Biotech. 4:1087.
Agaba et al. (2004) Marine Biotechnol. (NY) 6:251-261.
Alvarez et al. (2000) Theor Appl Genet 100:319-327.
Armbrust et al. (2004) Science 306:79-86.
Attila Kereszt et al. (2007) Nature Protocols 2:948-952.
Baumberger et al. (2007) Cuff. Biol. 17:1609-1614.
Baumlein et al. (1991) Mol. Gen. Genet. 225:459-467.
Baumlein et al. (1992) Plant J. 2:233-239.
Beaudoin et al. (2000) Proc. Natl. Acad. Sci. U.S.A. 97:6421-6426.
Beclin et al. (2002) Curr. Biol. 12:684-688.
Berberich. et al. (1998) Plant Mol. Biol. 36:297-306.
Bortolamiol et al. (2007) Cuff. Biol. 17:1615-1621.
Broun et al. (1998) Plant J. 13:201-210.
Brown et al. (2002) Biochem J. 364:795-805.
Chapman et al. (2004) Gen. Dev. 18:1179-1186.
Chen et al. (2004) The Plant Cell 16:1302-1313.
Cheng et al. (19%) Plant Cell Rep. 15:653-657.
Cheng et al. (2010) Transgenic Res 19: 221-229.
Chikwamba et al. (2003) Proc. Natl. Acad. Sci. U.S.A. 100:11127-11132.
Cho et al. (1999a) J. Biol. Chem. 274:471-477.
Cho et al. (1999b) J. Biol. Chem. 274:37335-37339.
Clough and Bent (1998) Plant J. 16:735-43.
Coutu et al. (2007) Transgenic Res. 16: 771-781.
Damude et al. (2006). Proc Natl Acad Sci USA 103: 9446-9451.
Denic and Weissman (2007) Cell 130:663-677.
Domergue et al (2002) Eur. J. Biochem. 269:4105-4113.
Domergue et al. (2002) Eur. J. Biochem. 269:4105-4113.
Domergue et al. (2003) J. Biol. Chem. 278: 35115-35126.
Domergue et al. (2005) Biochem. J. 1389: 483-490.
Dunover et al. (2004) The Plant Cell 16:1235-1250.
Ellerstrom et a. (1996) Plant Mol. Biol. 32:1019-1027.
Fujimura et al. (1985) Plant Tissue Culture Lett. 2:74.
Fukunaga (2009) EMBO J. 28:545-55.
Gamez et al. (2003) Food Res International 36: 721-727.
Garcia-Maroto et al. (2002) Lipids 37:417426.
Girke et al. (1998) Plant J. 15:39-48.
Glick et al. (2008) Proc. Natl. Acad. Sci U.S.A. 105-157-161.
Grant et al. (1995) Plant Cell Rep. 15:254-258.
Hall et al. (1991) Proc. Natl. Acad. Sci. USA 88:9320-9324
Hamilton and Baulcombe (1999) Science 286:950-952.
Hamilton et al. (1997) Gene 200:107-16.
Harayama (1998). Trends Biotechnol. 16: 76-82.
Hastings et al. (2001) Proc. Natl. Acad. Sci. U.S.A. 98:14304-14309.
Hinchee et al. (1988) Biotechnology 6:915-922.
Hoffmann et al. (2008) J Biol. Chem. 283:22352-22362.
Hong et al. (2002a) Lipids 37:863-868.
Horiguchi et al. (1998) Plant Cell Physiol. 39:540-544.
Horvath et a. (2000) Proc. Natl. Acad. Sci. U.S.A. 97:1914-1919.
Huang et al. (1999) Lipids 34:649-659.
Inagaki et al. (2002) Biosci. Biotechnol. Biochem. 66:613-621.
Johansen and Carrington (2001) Plant Physiol. 126-930-938.
Kajikawa et al. (2004) Plant Mol. Biol. 54:335-52.
Kajikawa et al. (2006) FEBS Lett 580:149-154.
Kim et al. (2005) Plant Cell. 2005 1073-89.
Knutzon et al. (1998) J. Biol Chem. 273:29360-6.
Koziel et al. (1996) Plant Mol. Biol. 32:393-405.
Lassner (1995) Plant Physiol. 109:1389-94.
Leonard et al. (2000) Biochem. J. 347:719-724.
Leonard et al. (2000b) Biochem. J. 350:765-770.
Leonard et al. (2002) Lipids 37:733-740.
Lewsey et al. (2007) Plant J. 50:240-252.
Lo et al. (2003) Genome Res. 13:455-466.
Lu and Kang (2008) Plant Cell Rep. 27:273-8.
Mallory et al. (2002) Nat. Biotech. 20:622-625.
Marangoni et al. (1995) Trends in Food Sci. Technol. 6: 329-335.
Meesapyodsuk et al. (2007) J Biol Chem 282: 20191-20199.
Meng et al. (2008) J. Gen. Virol. 89:2349-2358.
Meyer et al. (2003) Biochem. 42:9779-9788.
Meyer et al. (2004) Lipid Res 45:1899-1909.
Michaelson et al. (1998a) J. Biol. Chem. 273:19055-19059.
Michaelson et al. (1998b) FEBS Lett. 439:215-218.
Murashige and Skoog (1962) Physiologia Plantarum 15:473497.
Napier et al. (1998) Biochem. J. 330:611-614.
Needleman and Wunsch (1970) J. Mol. Biol. 48:443-453.
Niedz et al. (1995) Plant Cell Reports 14:403.
Ow et al. (1986) Science 234:856-859.
Parker-Barnes et al. (2000) Proc. Natl. Acad. Sci. USA 97:8284-8289.
Pereira et al. (2004a) Biochem. J. 378:665-671.
Pereira et al. (2004b) Biochem. J. 384:357-366.
Perrin et al. (2000) Mol Breed 6:345-352.
Petrie et al. (2010a) Metab. Eng. 12:233-240.
Petrie et al. (2010b) Plant Methods 11:6:8.
Petrie et al. (2012) Transgenic Res. 21:139-147.
Potenza et al. (2004) In Vitro Cell Dev Biol—Plant 40:1-22.
Prasher et al (1985) Biochem. Biophys. Res. Commun. 127:31-36.
Qi et al. (2002) FEBS Lett. 510:159-165.
Qi et al. (2004) Nat. Biotech. 22: 739-745.
Qiu et al. (2001) J. Biol. Chem. 276:31561-31566.
Reddy and Thomas (1996) Nat. Biotech. 14:639-642.
Reddy et al. (1993) Plant Mol. Biol. 22:293-300.
Robert et al. (2005) Func. Plant Biol. 32:473-479.
Robert et al. (2009) Marine Biotech 11:410418.
Ruiz-Lopez et al. (2012) Transgenic Res. 21:139-147.
Saha et al. (2006) Plant Physiol. 141:1533-1543.
Saito et al. (2000) Eur. J. Biochem. 267:1813-1818.
Sakuradani et al. (1999) Gene 238:445-453.
Sato et al. (2004) Crop Sci. 44: 646-652.
Sakuradani et al. (2005) Appl. Microbiol. Biotechnol. 66:648-654.
Sayanova et al. (2006) J Biol Chem 281: 36533-36541.
Sayanova et al. (1997) Proc. Natl. Acad. Sci. U.S.A. 94:4211-4216.
Savanova et al. (2003) FEBS Lett. 542:100-104.
Savanova et al. (2006) Planta 224:1269-1277.
Sayanova et al. (2007) Plant Physiol 144:455-467.
Singh et al. (2005) Curr. Opin, in Plant Biol. 8:197-203.
Speranza et al. (2012) Process Biochemistry (In Press).
Sperling et al. (2000) Eur. J. Biochem. 267:3801-3811.
Sperling et al. (2001) Arch. Biochm. Biophys. 388:293-8.
Sprecher et al. (1995) J. Lipid Res. 36:2471-2477.
Spychalla et al. (1997) Proc. Natl. Acad. Sci. U.S.A. 94:1142-1147.
Stalker et al. (1998) J. Biol. Chem. 263:6310-6314.
Thillet et al. (1988) J. Biol. Chem 263:12500-12508.
Tonon et al. (2003) FEBS Lett. 553:440-444.
Toriyama et al. (1986) Theor. Appl. Genet. 205:34.
Trautwein (2001) European J. Lipid Sci, and Tech. 103:45-55.
Tvrdik (2000) J. Cell Biol. 149:707-718.
Venegas-Caleron et al. (2010) Prog. Lipid Res. 49:108-119.
Voinnet et al. (2003) Plant J. 33:949-956.
Wallis and Browse (1999) Arch. Biochem. Biophys. 365:307-316.
Watts and Browse (1999b) Arch. Biochem. Biophys. 362:175-182.
Weiss et al. (2003) Int. J. Med. Microbiol. 293:95:106.
Whitney et al. (2003) Planta 217:983-992.
Winans (1988) J. Bacteriol. 170:4047-54.
Wood (2009) Plant Biotechnol J. 7:914-24.
Wu et al. (2005) Nat. Biotech. 23:1013-1017.
Yang et al. (2003) Planta 216:597-603.
Zank et al. (2002) Plant J. 31:255-268.
Zank et al. (2005) WO 2005/012316
Zhang et al. (2004) FEBS Lett. 556:81-85.
Zhang et al. (2006) 20:3255-3268.
Zhang et al. (2007a) FEBS Letters 581: 315-319.
Zhang et al. (2008) Yeast 25: 21-27.
Zhou et al. (2007) Phytochem. 68:785-796.
Zhou et al. (2008) Insect Mol Biol 17: 667-676.
Zou et al. (1997) Plant Cell. 9:909-23.

1. Extracted plant lipid, comprising fatty acids in an esterified form, the fatty acids comprising oleic acid, palmitic acid, ω6 fatty acids which comprise linoleic acid (LA), ω3 fatty acids which comprise α-linolenic acid (ALA) and docosahexaenoic acid (DHA), and optionally one or more of stearidonic acid (SDA), eicosapentaenoic acid (EPA), docosapentaenoic acid (DPA) and eicosatetraenoic acid (ETA), wherein the level of DHA in the total fatty acid content of the extracted lipid is about 7% to 20%.

2. The lipid of claim 1 which has one or more or all of the following features
i) the level of palmitic acid in the total fatty acid content of the extracted lipid is between about 2% and 18%, or between about 2% and 16%, or between about 2% and 15%,
ii) the level of myristic acid (C14:0) in the total fatty acid content of the extracted lipid is less than 6%, or less than 3% or less than 2%, or less than 1%,
iii) the level of oleic acid in the total fatty acid content of the extracted lipid is between about 1% and about 30%, between about 3% and about 30%, or between about 6% and about 30%, or between 1% and about 20%, or between about 30% and about 60%, or about 45% to about 60%, or is about 30%,
iv) the level of linoleic acid (LA) in the total fatty acid content of the extracted lipid is between about 4% and about 35%, or between about 4% and about 20%, or between about 4% and 17%,
v) the level of α-linolenic acid (ALA) in the total fatty acid content of the extracted lipid is between about 4% and about 40%, between about 7% and about 40%, or between about 10% and about 35%, or between about 20% and about 35%, or between about 4% and 16%, or between about 2% and 16%,
vi) the level of γ-linolenic acid (GLA) in the total fatty acid content of the extracted lipid is less than 4%, less than about 3%, less than about 2%, less than about 1%, less than about 0.5%, between 0.05% and 7%, between 0.05% and 4%, or between 0.05% and about 3%, or between 0.05% and about 2%,
vii) the level of stearidonic acid (SDA) in the total fatty acid content of the extracted lipid is less than about 7%, less than about 6%, less than about 4%, less than about 3%, between about 0.05% and about 7%, between about 0.05% and about 6%, between about 0.05% and about 4%, between about 0.05% and about 3%, or between 0.05% and about 2%,
viii) the level of eicosatetraenoic acid (ETA) in the total fatty acid content of the extracted lipid is less than about 6%, less than about 5%, less than about 4%, less than about 1%, less than about 0.5%, between about 0.05% and about 6%, between about 0.05% and about 5%, between about 0.05% and about 4%, between about 0.05% and about 3%, or between about 0.05% and about 2%,
ix) the level of eicosatrienoic acid (ETrA) in the total fatty acid content of the extracted lipid is less than 4%, less than about 2%, less than about 1%, between 0.05% and 4%, between 0.05% and 3%, or between 0.05% and about 2%, or between 0.05% and about 1%,
x) the level of eicosapentaenoic acid (EPA) in the total fatty acid content of the extracted lipid is less than 4%, less than about 3%, less than about 2%, between 0.05% and 10%, between 0.05% and 5%, or between 0.05% and about 3%, or between 0.05% and about 2%,
xi) the level of docosapentaenoic acid (DPA) in the total fatty acid content of the extracted lipid is less than 4%, less than about 3%, less than about 2%, between 0.05% and 8%, between 0.05% and 5%, or between 0.05% and about 3%, or between 0.05% and about 2%,
xii) the level of DHA in the total fatty acid content of the extracted lipid is about 8%, about 9%, about 10%, about 11%, about 12%, about 13%, about 14%, about 15%, about 16%, about 17%, about 18%, between about 8% and 20%, between about 10% and 20%, between about 11% and 20%, between about 10% and about 16%, or between about 14% and 20%,
xiii) the lipid comprises ω6-docosapentaenoic acid (22:5^{Δ4,7,1,13,16}) in its fatty acid content,
xiv) the lipid is essentially free of ω6-docosapentaenoic acid (22:5^{Δ4,7,10,13,16}) in its fatty acid content,
xv) the lipid is essentially free of SDA, EPA and ETA in its fatty acid content,
xvi) the level of total saturated fatty acids in the total fatty acid content of the extracted lipid is between about 4% and about 25%, or between about 4% and about 20%, between about 6% and about 20%, between about 4% and about 60%, between about 30% and about 60%, or between about 45% and about 60%,
xvii) the level of total monounsaturated fatty acids in the total fatty acid content of the extracted lipid is between about 4% and about 35%, or between about 8% and about 25%, or between 8% and about 22%,
xviii) the level of total polyunsaturated fatty acids in the total fatty acid content of the extracted lipid is between about 20% and about 75%, or between about 50% and about 75%, or between about 60% and about 75%,
xix) the level of total ω6 fatty acids in the total fatty acid content of the extracted lipid is between about 35% and about 50%, between about 20% and about 35%, between about 6% and 20%, less than 20%, less than about 16%, less than about 10%, between about 1% and about 16%, between about 2% and about 10%, or between about 4% and about 10%,
xx) the level of new (6 fatty acids in the total fatty acid content of the extracted lipid is less than about 10%, less than about 8%, less than about 6%, less than 4%, between about 1% and about 20%, between about 1% and about 10%, between about 0.5% and about 8%, or between about 0.5% and 4%,
xxi) the level of total ω3 fatty acids in the total fatty acid content of the extracted lipid is between 36% and about 65%, between 40% and about 60%, between about 20% and about 35%, between about 10% and about 20%, about 25%, about 30%, about 35% or about 40%,
xxii) the level of new ω3 fatty acids in the total fatty acid content of the extracted lipid is between 9% and about 33%, between about 10% and about 20%, between about 20% and about 30%, between about 12% and about 25%, about 13%, about 15%, about 17% or about 20%,
xxiii) the ratio of total 06 fatty acids: total ω3 fatty acids in the fatty acid content of the extracted lipid is between about 1.0 and about 3.0, between about 0.1 and about 1, between about 0.1 and about 0.5, less than about 0.50, less than about 0.40, less than about 0.30, less than about 0.20, less than about 0.15, about 1.0, about 0.1 or about 0.2,
xxiv) the ratio of new ω6 fatty acids: new ω3 fatty acids in the fatty acid content of the extracted lipid is between about 1.0 and about 3.0, between about 0.1 and about 1, between about 0.1 and about 0.5, less than about 0.50, less than about 0.40, less than about 0.30, less than about 0.20, less than about 0.15, about 0.1, about 0.2 or about 1.0,
xxv) the fatty acid composition of the lipid is based on an efficiency of conversion of oleic acid to LA by Δ12-desaturase of at least about 60%, at least about 70%, at least about 80%, between about 60% and about 98%, between about 70% and about 95%, or between about 75% and about 90%,
xxvi) the fatty acid composition of the lipid is based on an efficiency of conversion of ALA to SDA by Δ6-desaturase of at least about 30%, at least about 40%, at least about 50%, at least about 60%, at least about 70%, between about 30% and about 70%, between about 35% and about 60%, or between about 50% and about 70%,
xxvii) the fatty acid composition of the lipid is based on an efficiency of conversion of SDA to ETA acid by Δ6-elongase of at least about 60%, at least about 70%, at least about 75%, between about 60% and about 95%, between about 70% and about 88%, or between about 75% and about 85%,
xxviii) the fatty acid composition of the lipid is based on an efficiency of conversion of ETA to EPA by Δ5-desaturase of at least about 60%, at least about 70%, at least about 75%, between about 60% and about 99%, between about 70% and about 99%, or between about 75% and about 98%,
xxix) the fatty acid composition of the lipid is based on an efficiency of conversion of EPA to DPA by Δ5-elongase of at least about 80%, at least about 85%, at least about 90%, between about 50% and about 95%, or between about 85% and about 95%,
xxx) the fatty acid composition of the lipid is based on an efficiency of conversion of DPA to DHA by Δ4-desaturase of at least about 80%, at least about 90%, at least about 93%, between about 50% and about 95%, between about 80% and about 95%, or between about 85% and about 95%,
xxxi) the fatty acid composition of the lipid is based on an efficiency of conversion of oleic acid to DHA of at least about 10%, at least about 15%, at least about 20%, between about 10% and about 50%, between about 10% and about 30%, or between about 10% and about 25%,
xxxii) the fatty acid composition of the lipid is based on an efficiency of conversion of LA to DHA of at least about 15%, at least about 20%, at least about 22%, at least about 25%, between about 15% and about 50%, between about 20% and about 40%, or between about 20% and about 30%,
xxxiii) the fatty acid composition of the lipid is based on an efficiency of conversion of ALA to DHA of at least about 17%, at least about 22%, at least about 24%, between about 17% and about 55%, between about 22% and about 35%, or between about 24% and about 35%,
xxxiv) the total fatty acid in the extracted lipid has less than 1% C20:1,
xxxv) the triacylglycerol (TAG) content of the lipid is at least about 70%, at least about 80%, at least about 90%, at least 95%, between about 70% and about 99%, or between about 90% and about 99%,
xxxvi) the lipid comprises diacylglycerol (DAG),
xxxvii) the lipid comprises less than about 10%, less than about 5%, less than about 1%, or between about 0.001% and about 5%, free (non-esterified) fatty acids and/or phospholipid, or is essentially free thereof,
xxxviii) at least 70%, or at least 80%, of the DHA esterified in the form of TAG is in the sn-1 or sn-3 position of the TAG,
xxxix) the most abundant DHA-containing TAG species in the lipid is DHA/18:3/18:3 (TAG 58:12), and
xl) the lipid comprises tri-DHA TAG (TAG 66:18).

3. The lipid of claim 1 or claim 2 which is in the form of an oil, wherein at least about 90%, or least about 95%, at least about 98%, or between about 95% and about 98%, by weight of the oil is the lipid.

4. The lipid according to any one of claims 1 to 3 which further comprises one or more sterols.

5. The lipid of claim 4 which is in the form of an oil, and which comprises less than about 10 mg of sterols/g of oil, less than about 7 mg of sterols/g of oil, between about 1.5 mg and about 10 mg of sterols/g of oil, or between about 1.5 mg and about 7 mg of sterols/g of oil.

6. The lipid of claim 4 or claim 5 which comprises one or more or all of campesterol/24-methylcholesterol, Δ5-stigmasterol, eburicol, β-sitosterol/24-ethylcholesterol, Δ5-avenasterol/isofucosterol, Δ7-stigmasterol/stigmast-7-en-3β-ol, and Δ7-avenasterol.

7. The lipid according to any one of claims 4 to 6 which comprises less than about 0.5 mg of cholesterol/g of oil, less than about 0.25 mg of cholesterol/g of oil, between about 0 mg and about 0.5 mg of cholesterol/g of oil, or between about 0 mg and about 0.25 mg of cholesterol/g of oil, or which is essentially free of cholesterol.

8. The lipid according to any one of claims 1 to 7, wherein the lipid is an oil, preferably oil from an oilseed.

9. The lipid of claim 8, wherein the lipid is Brassica sp. oil, Gossypium hirsutum oil, Linum usitatissimum oil, Helianthus sp. oil, Carthamus tinctorius oil, Glycine max oil, Zea mays oil, Arabidopsis thaliana oil, Sorghum bicolor oil, Sorghum vulgare oil, Avena sativa oil, Trifolium sp. oil, Elaesis guineenis oil, Nicotiana benthamiana oil, Hordeum vulgare oil, Lupinus angustifolius oil, Oryza sativa oil, Oryza glaberrima oil, Camelina sativa oil, Crambe abyssinica oil, Miscanthus x giganteus oil, or Miscanthus sinensis oil.

10. A process for producing extracted plant lipid, comprising the steps of
i) obtaining a plant part comprising lipid, the lipid comprising fatty acids in an esterified form, the fatty acids comprising oleic acid, palmitic acid, ω6 fatty acids which comprise linoleic acid (LA) and γ-linolenic acid (GLA), ω3 fatty acids which comprise α-linolenic acid (ALA), stearidonic acid (SDA), docosapentaenoic acid (DPA) and docosahexaenoic acid (DHA), and optionally one or more of eicosapentaenoic acid (EPA) and eicosatetraenoic acid (ETA), wherein the level of DHA in the total fatty acid content of extractable lipid in the plant part is about 7% to 20%, and
ii) extracting lipid from the plant part,
wherein the level of DHA in the total fatty acid content of the extracted lipid is about 7% to 20%.

11. The process of claim 10, wherein the extracted lipid has one or more of the features defined in claims 2 to 9.

12. The process of claim 10 or claim 11, wherein the plant part is a seed, preferably an oilseed.

13. The process of claim 12, wherein the seed is Brassica sp., Gossypium hirsutum, Linum usitatissimum, Helianthus sp., Carthamus tinctorius, Glycine max, Zea mays, Arabidopsis thaliana, Sorghum bicolor, Sorghum vulgare, Avena sativa, Trifolium sp., Elaesis guineenis, Nicotiana benthamiana, Hordeum vulgare, Lupinus angustifolius, Oryza sativa, Oryza glaberrima, Camelina sativa, or Crambe abyssinica, preferably a Brassica napus, B juncea or C. sativa seed.

14. The process of claim 12 or claim 13, wherein the seed comprises at least about 18 mg, at least about 22 mg, at least about 26 mg, between about 18 mg and about 100 mg, between about 22 mg and about 70 mg, or between about 24 mg and about 50 mg, of DHA per gram of seed.

15. The process according to any one of claims 10 to 14, wherein the plant part comprises exogenous polynucleotides encoding one of the following sets of enzymes:
i) an ω3-desaturase, a Δ6-desaturase, a Δ5-desaturase, a Δ4-desaturase, a Δ6-elongase and a Δ5-elongase,
ii) a Δ15-desaturase, a Δ6-desaturase, a Δ5-desaturase, a Δ4-desaturase, a Δ6-elongase and a Δ5-elongase,
iii) a Δ12-desaturase, a Δ6-desaturase, a Δ5-desaturase, a Δ4-desaturase, a Δ6-elongase and an Δ5-elongase,
iv) a Δ12-desaturase, a ω3-desaturase or a Δ15-desaturase, a Δ6-desaturase, a Δ5-desaturase, a Δ4-desaturase, a Δ6-elongase and an Δ5-elongase,
v) an ω3-desaturase, a Δ8-desaturase, a Δ5-desaturase, a Δ4-desaturase, a Δ9-elongase and an Δ5-elongase,
vi) a Δ15-desaturase, a Δ8-desaturase, a Δ5-desaturase, a Δ4-desaturase, a Δ9-elongase and a Δ5-elongase,
vii) a Δ12-desaturase, a Δ8-desaturase, a Δ5-desaturase, a Δ4-desaturase, a Δ9-elongase and an Δ5-elongase, or
viii) a Δ12-desaturase, a ω3-desaturase or a Δ15-desaturase, a Δ8-desaturase, a Δ5-desaturase, a Δ4-desaturase, a Δ9-elongase and an Δ5-elongase,
and wherein each polynucleotide is operably linked to one or more promoters that are capable of directing expression of said polynucleotides in a cell of the plant part.

16. The process of claim 15, wherein the plant part has one or more or all of the following features
i) the Δ12-desaturase converts oleic acid to linoleic acid in one or more cells of the plant with an efficiency of at least about 60%, at least about 70%, at least about 80%, between about 60% and about 95%, between about 70% and about 90%, or between about 75% and about 85%,
ii) the ω3-desaturase converts ω6 fatty acids to ω3 fatty acids in one or more cells of the plant with an efficiency of at least about 65%, at least about 75%, at least about 85%, between about 65% and about 95%, between about 75% and about 91%, or between about 80% and about 91%,
iii) the Δ6-desaturase converts ALA to SDA in one or more cells of the plant with an efficiency of at least about 30%, at least about 40%, at least about 50%, at least about 60%, at least about 70%, between about 30% and about 70%, between about 35% and about 60%, or between about 50% and about 70%,
iv) the Δ6-desaturase converts linoleic acid to γ-linolenic acid in one or more cells of the plant with an efficiency of less than about 5%, less than about 2.5%, less than about 1%, between about 0.1% and about 5%, between about 0.5% and about 2.5%, or between about 0.5% and about 1%,
v) the Δ6-elongase converts SDA to ETA in one or more cells of the plant with an efficiency of at least about 60%, at least about 70%, at least about 75%, between about 60% and about 95%, between about 70% and about 80%, or between about 75% and about 80%,
vi) the Δ5-desaturase converts ETA to EPA in one or more cells of the plant with an efficiency of at least about 60%, at least about 70%, at least about 75%, at least about 80%, at least about 90%, between about 60% and about 95%, between about 70% and about 95%, or between about 75% and about 95%,
vii) the Δ5-elongase converts EPA to DPA in one or more cells of the plant with an efficiency of at least about 80%, at least about 85%, at least about 90%, between about 50% and about 90%, or between about 85% and about 95%,
viii) the Δ4-desaturase converts DPA to DHA in one or more cells of the plant with an efficiency of at least about 80%, at least about 90%, at least about 93%, between about 50% and about 95%, between about 80% and about 95%, or between about 85% and about 95%,
ix) the efficiency of conversion of oleic acid to DHA in one or more cells of the plant part is at least about 10%, at least about 15%, at least about 20%, between about 10% and about 50%, between about 10% and about 30%, or between about 10% and about 25%,
x) the efficiency of conversion of LA to DHA in one or more cells of the plant part is at least about 15%, at least about 20%, at least about 22%, at least about 25%, between about 15% and about 50%, between about 20% and about 40%, or between about 20% and about 30%,
xi) the efficiency of conversion of ALA to DHA in one or more cells of the plant part is at least about 17%, at least about 22%, at least about 24%, between about 17% and about 55%, between about 22% and about 35%, or between about 24% and about 35%,
xii) one or more cells of the plant part comprise at least about 15%, at least about 20%, between about 15% and about 30%, or between about 22.5% and about 27.5%, more ω3 fatty acids than corresponding cells lacking the exogenous polynucleotides,
xiii) the Δ6-desaturase preferentially desaturates α-linolenic acid (ALA) relative to linoleic acid (LA),
xiv) the Δ6-elongase also has Δ9-elongase activity,
xv) the Δ12-desaturase also has Δ15-desaturase activity,
xvi) the Δ6-desaturase also has Δ8-desaturase activity,
xvii) the Δ8-desaturase also has Δ6-desaturase activity or does not have Δ6-desaturase activity,
xviii) the Δ15-desaturase also has ω3-desaturase activity on GLA,
xix) the ω3-desaturase also has Δ15-desaturase activity on LA,
xx) the ω3-desaturase desaturates both LA and/or GLA,
xxi) the ω3-desaturase preferentially desaturates GLA relative to LA,
xxii) the level of DHA in the plant part is based on an efficiency of conversion of oleic acid to DHA in the plant part of at least about 10%, at least about 15%, at least about 20%, between about 10% and about 50%, between about 15% and about 30%, or between about 20% and about 25%,
xxiii) the level of DHA in the plant part is based on an efficiency of conversion of LA to DHA in the plant part of at least about 15%, at least about 20%, at least about 22%, between about 15% and about 60%, between about 20% and about 40%, or between about 22% and about 30%,
xxiv) the level of DHA in the plant part is based on an efficiency of conversion of ALA to DHA in the plant part of at least about 17%, at least about 22%, at least about 24%, between about 17% and about 65%, between about 22% and about 35%, or between about 24% and about 35%
xxx) one or more or all of the desaturases have greater activity on an acyl-CoA substrate than a corresponding acyl-PC substrate,
xxxi) the Δ6-desaturase has greater Δ6-desaturase activity on ALA than LA as fatty acid substrate,
xxxii) the Δ6-desaturase has greater Δ6-desaturase activity on ALA-CoA as fatty acid substrate than on ALA joined to the sn-2 position of PC as fatty acid substrate,
xxxiii) the Δ6-desaturase has at least about a 2-fold greater Δ6-desaturase activity, at least 3-fold greater activity, at least 4-fold greater activity, or at least 5-fold greater activity, on ALA as a substrate compared to LA,
xxxiv) the Δ6-desaturase has greater activity on ALA-CoA as fatty acid substrate than on ALA joined to the sn-2 position of PC as fatty acid substrate,
xxxv) the Δ6-desaturase has at least about a 5-fold greater Δ6-desaturase activity or at least 10-fold greater activity, on ALA-CoA as fatty acid substrate than on ALA joined to the sn-2 position of PC as fatty acid substrate,
xxxvi) the desaturase is a front-end desaturase,
xxxvii) the Δ6-desaturase has no detectable Δ5-desaturase activity on ETA.

17. The process of claim 15 or claim 16, wherein the plant part has one or more or all of the following features
i) the Δ12-desaturase comprises amino acids having a sequence as provided in SEQ ID NO:10, a biologically active fragment thereof, or an amino acid sequence which is at least 50% identical to SEQ ID NO:10,
ii) the ω3-desaturase comprises amino acids having a sequence as provided in SEQ ID NO:12, a biologically active fragment thereof, or an amino acid sequence which is at least 50% identical to SEQ ID NO:12,
iii) the Δ6-desaturase comprises amino acids having a sequence as provided in SEQ ID NO:16, a biologically active fragment thereof, or an amino acid sequence which is at least 50% identical to SEQ ID NO:16,
iv) the Δ6-elongase comprises amino acids having a sequence as provided in SEQ ID NO:25, a biologically active fragment thereof such as SEQ ID NO:26, or an amino acid sequence which is at least 50% identical to SEQ ID NO:25 and/or SEQ ID NO:26,
v) the Δ5-desaturase comprises amino acids having a sequence as provided in SEQ ID NO:30, a biologically active fragment thereof, or an amino acid sequence which is at least 50% identical to SEQ ID NO:30,
vi) the Δ5-elongase comprises amino acids having a sequence as provided in SEQ ID NO:37, a biologically active fragment thereof, or an amino acid sequence which is at least 50% identical to SEQ ID NO:37,
vii) the Δ4-desaturase comprises amino acids having a sequence as provided in SEQ ID NO:41, a biologically active fragment thereof, or an amino acid sequence which is at least 50% identical to SEQ ID NO:41.

18. The process according to any one of claims 15 to 17, wherein the plant part further comprises an exogenous polynucleotide encoding a diacylglycerol acyltransferase (DGAT), monoacylglycerol acyltransferase (MGAT), glycerol-3-phosphate acyltransferase (GPAT), 1-acyl-glycerol-3-phosphate acyltransferase (LPAAT) preferably an LPAAT which can use a C22 polyunsaturated fatty acyl-CoA substrate, acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), phosphatidylcholine:diacylglycerol choline phosphotransferase (PDCT), acyl-CoA synthase (ACS), or a combination of two or more thereof.

19. The process according to any one of claim 10 to 18, wherein the plant part further comprises an introduced mutation or an exogenous polynucleotide which down regulates the production and/or activity of an endogenous enzyme in a cell of the plant part selected from FAE1, DGAT, MGAT, GPAT, LPAAT, LPCAT, PLA₂, PLC, PLD, CPT, PDAT, a thioesterase such as FATB, or a Δ12-desaturase, or a combination of two or more thereof.

20. The process according to any one of claims 15 to 19, wherein at least one, or all, of the promoters are seed specific promoters.

21. The process of claim 20, wherein at least one, or all, of the promoters have been obtained from oil biosynthesis or accumulation genes such as oleosin, or from seed storage protein genes such as conlinin.

22. The process according to any one of claim 15 to 21, wherein the promoter(s) directing expression of the exogenous polynucleotides encoding the Δ4-desaturase and the Δ5-elongase initiate expression of the polynucleotides in developing seed of the plant part before, or reach peak expression before, the promoter(s) directing expression of the exogenous polynucleotides encoding the Δ12-desaturase and the ω3-desaturase.

23. The process according to any one of claims 15 to 22, wherein the exogenous polynucleotides are covalently linked in a DNA molecule, preferably a T-DNA molecule, integrated into the genome of cells of the plant part and preferably where the number of such DNA molecules integrated into the genome of the cells of the plant part is not more than one, two or three, or is two or three.

24. The process of claim 23, wherein the plant comprises at least two different, exogenous polynucleotides each encoding a Δ6-desaturase which have the same or different amino acid sequences.

25. The process according to any one of claims 15 to 24, wherein the total oil content of the plant part comprising the exogenous polynucleotides is at least about 40%, or at least about 50%, or at least about 60%, or at least about 70%, or between about 50% and about 80% of the total oil content of a corresponding plant part lacking the exogenous polynucleotides.

26. The process according to any one of claims 10 to 25, wherein the lipid is in the form of an oil, preferably a seedoil from an oilseed, and wherein at least about 90%, or about least 95%, at least about 98%, or between about 95% and about 98%, by weight of the lipid is triacylglycerols.

27. The process according to any one of claims 10 to 26 which further comprises treating the lipid to increase the level of DHA as a percentage of the total fatty acid content.

28. The process of claim 27, wherein the treatment is transesterification.

29. Lipid, or oil comprising the lipid, produced using the process according to any one of claims 10 to 28.

30. A chimeric genetic construct comprising in order a first gene, a second gene, a third gene, a fourth gene, a fifth gene and a sixth gene which are all covalently linked on a single DNA molecule,
wherein the first, second and third genes are joined together as a first gene cluster and the fourth, fifth and sixth genes are joined together as a second gene cluster,
wherein each gene comprises a promoter, a coding region and a transcription terminator and/or polyadenylation region such that each promoter is operably linked to the coding region and transcription terminator and/or polyadenylation region,
wherein each promoter is independently identical or different to the other promoters such that the DNA molecule comprises three, four, five or six different promoters,
wherein one or more or all of the promoters are heterologous with respect to the coding region to which it is operably linked,
wherein the direction of transcription of the first gene is away from the third gene and opposite to the direction of transcription of the third gene,
wherein the direction of transcription of the fourth gene is away from the sixth gene and opposite to the direction of transcription of the sixth gene,
wherein the direction of transcription of the second gene is the same as for the first gene or the third gene,
wherein the direction of transcription of the fifth gene is the same as for the fourth gene or the sixth gene,
wherein the transcription terminator and/or polyadenylation region of the second gene is spaced apart from the promoter of the first or third genes, whichever is closer, by a first spacer region of between about 0.2 and about 3.0 kilobases,
wherein the first gene cluster is spaced apart from the second gene cluster by a second spacer region of between about 1.0 and about 10.0 kilobases, and
wherein the transcription terminator and/or polyadenylation region of the fifth gene is spaced apart from the promoter of the fourth or sixth genes, whichever is closer, by a third spacer region of between about 0.2 and about 3.0 kilobases.

31. The genetic construct of claim 30, wherein the DNA molecule comprises a seventh gene which is spaced apart from the first gene cluster or the second gene cluster, whichever is closer, by a spacer region of between about 1.0 and about 10.0 kilobases.

32. The genetic construct of claim 30 or claim 31, wherein the DNA molecule comprises two or more different transcription terminator and/or polyadenylation regions.

33. The genetic construct according to any one of claims 30 to 32, wherein at least one of the spacer regions comprises a matrix attachment region (MAR).

34. The genetic construct according to any one of claims 30 to 33, wherein the DNA molecule comprises right and left border regions flanking the genes and is a T-DNA molecule.

35. The genetic construct according to any one of claims 30 to 34 which is in an Agrobacterium cell or is integrated into the genome of a plant cell.

36. The genetic construct according to any one of claims 30 to 35, wherein at least one of the genes encodes a fatty acid desaturase or a fatty acid elongase.

37. The genetic construct of claim 36, comprising genes encoding a set of enzymes as defined in claim 15, and/or wherein one or more of the genes encode an enzyme as defined in claim 16 or claim 17.

38. An isolated and/or exogenous polynucleotide comprising:
i) a sequence of nucleotides selected from any one of SEQ ID NOs: 1 to 9, 11, 14, 18, 22, 23, 28, 34, 35, 39 or 45, and/or
ii) a sequence of nucleotides which are at least 95% identical or 99% identical to one or more of the sequences set forth in SEQ ID NOs: 1 to 9, 11, 14, 18, 22, 23, 28, 34, 35, 39 or 45.

39. A vector or genetic construct comprising the polynucleotide according to claim 38 and/or the genetic construct according to any one of claims 30 to 37.

40. The vector or genetic construct of claim 39, wherein the sequence of nucleotides selected from any one of SEQ ID NOs: 11, 14, 18, 22, 23, 28, 34, 35, 39 or 45, or the sequence of nucleotides which is at least 95% identical or 99% identical to one or more of the sequences set forth in SEQ ID NOs: 11, 14, 18, 22, 23, 28, 34, 35, 39 or 45, is operably linked to a promoter.

41. A host cell comprising exogenous polynucleotides encoding one of the following sets of enzymes;
i) an ω3-desaturase, a Δ6-desaturase, a Δ5-desaturase, a Δ4-desaturase, a Δ6-elongase and a Δ5-elongase,
ii) a Δ15-desaturase, a Δ6-desaturase, a Δ5-desaturase, a Δ4-desaturase, a Δ6-elongase and a Δ5-elongase,
iii) a Δ12-desaturase, a Δ6-desaturase, a Δ5-desaturase, a Δ4-desaturase, a Δ6-elongase and an Δ5-elongase,
iv) a Δ12-desaturase, a ω3-desaturase or a Δ15-desaturase, a Δ6-desaturase, a Δ5-desaturase, a Δ4-desaturase, a Δ6-elongase and an Δ5-elongase,
v) an ω3-desaturase, a Δ8-desaturase, a Δ5-desaturase, a Δ4-desaturase, a Δ9-elongase and an Δ5-elongase,
vi) a Δ15-desaturase, a Δ8-desaturase, a Δ5-desaturase, a Δ4-desaturase, a Δ9-elongase and a Δ5-elongase,
vii) a Δ12-desaturase, a Δ8-desaturase, a Δ5-desaturase, a Δ4-desaturase, a Δ9-elongase and an Δ5-elongase, or
viii) a Δ12-desaturase, a ω3-desaturase or a Δ15-desaturase, a Δ8-desaturase, a Δ5-desaturase, a Δ4-desaturase, a Δ9-elongase and an Δ5-elongase,
and wherein each polynucleotide is operably linked to one or more promoters that are capable of directing expression of said polynucleotides in the cell.

42. The cell of claim 41, comprising lipid as defined in any one of claims 1 to 9, or wherein one or more or all of the desaturases or elongases have one or more of the features as defined in claim 16 or claim 17.

43. A host cell comprising
i) a first exogenous polynucleotide encoding a Δ12-desaturase which comprises amino acids having a sequence as provided in SEQ ID NO:10, a biologically active fragment thereof, or an amino acid sequence which is at least 50% identical to SEQ ID NO:10, and
ii) a second exogenous polynucleotide encoding a ω3-desaturase which comprises amino acids having a sequence as provided in SEQ ID NO:12, a biologically active fragment thereof, or an amino acid sequence which is at least 50% identical to SEQ ID NO:12,
wherein each polynucleotide is operably linked to one or more promoters that are capable of directing expression of said polynucleotides in the cell.

44. A host cell comprising one or more of the polynucleotide according to claim 38, the genetic construct according to any one of claims 30 to 37, or the vector or genetic construct of claim 39 or claim 40.

45. The cell according to any one of claim 41 to 44 which is in a plant, in a plant part and/or is a mature plant seed cell.

46. The cell of claim 45, wherein the plant or plant seed is an oilseed plant or an oilseed, respectively.

47. A transgenic non-human organism comprising a cell according to any one of claims 41 to 46.

48. The transgenic non-human organism of claim 47 which is a transgenic plant.

49. An oilseed plant comprising
a) lipid in its seed, the lipid comprising fatty acids in an esterified form, and
b) exogenous polynucleotides encoding one of the following sets of enzymes;
i) a Δ12-desaturase, a fungal ω3-desaturase and/or fungal Δ15-desaturase, a Δ6-desaturase, a Δ5-desaturase, a Δ4-desaturase, a Δ6-elongase and an Δ5-elongase, or
ii) a Δ12-desaturase, a fungal ω3-desaturase and/or fungal Δ15-desaturase, a Δ8-desaturase, a Δ5-desaturase, a Δ4-desaturase, a Δ9-elongase and an Δ5-elongase,
wherein each polynucleotide is operably linked to one or more seed-specific promoters that are capable of directing expression of said polynucleotides in developing seed of the plant, wherein the fatty acids comprise oleic acid, palmitic acid, ω6 fatty acids which comprise linoleic acid (LA) and γ-linolenic acid (GLA), ω3 fatty acids which comprise α-linolenic acid (ALA), stearidonic acid (SDA), docosapentaenoic acid (DPA) and docosahexaenoic acid (DHA), and optionally eicosapentaenoic acid (EPA) and/or eicosatetraenoic acid (ETA), and wherein the level of DHA in the total fatty acid content of the lipid is about 7% to 20%.

50. The plant of claim 49, wherein the plant is a canola, Glycine max, Camelina sativa or Arabidopsis thaliana plant.

51. The plant of claim 49 or claim 50, wherein one or more of the desaturases is capable of using an acyl-CoA substrate.

52. The plant of claim 51, wherein one or more of the Δ6-desaturase, Δ5-desaturase, Δ4-desaturase and Δ8-desaturase, if present, is capable of using an acyl-CoA substrate, preferably each of the i) Δ6-desaturase, Δ5-desaturase and Δ4-desaturase or ii) Δ5-desaturase, Δ4-desaturase and Δ8-desaturase is capable of using an acyl-CoA substrate.

53. The plant according to any one of claims 49 to 52, wherein mature, harvested seed of the plant has a DHA content of at least about 28 mg per gram seed, preferably at least about 32 mg per gram seed, at least about 36 mg per gram seed, at least about 40 mg per gram seed, more preferably at least about 44 mg per gram seed or at least about 48 mg per gram seed.

54. A Brassica napus, B. juncea or Camelina sativa plant which is capable of producing seed comprising DHA, wherein mature, harvested seed of the plant has a DHA content of at least about 28 mg per gram seed, preferably at least about 32 mg per gram seed, at least about 36 mg per gram seed, at least about 40 mg per gram seed, more preferably at least about 44 mg per gram seed or at least about 48 mg per gram seed.

55. A plant cell of a plant according to any one of claims 49 to 54 comprising the exogenous polynucleotides.

56. A plant part, preferably a seed, which has one or more of the following features
i) is from a plant according to any one of claims 48 to 54,
ii) comprises lipid as defined in any one of claims 1 to 9,
iii) can be used in a process according to any one of claims 10 to 28,
iv) comprises a genetic construct according to any one of claims 30 to 37, or
v) comprises a set of exogenous polynucleotides defined in any one of claims 14, 41 or 48 to 52.

57. Mature, harvested Brassica napus, B. juncea or Camelina sativa seed comprising DHA and a moisture content of between about 4% and about 15% by weight, wherein the DHA content of the seed at least about 28 mg per gram seed, preferably at least about 32 mg per gram seed, at least about 36 mg per gram seed, at least about 40 mg per gram seed, more preferably at least about 44 mg per gram seed or at least about 48 mg per gram seed.

58. The cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56 or the seed of claim 57, which can be used to produce extracted lipid comprising one or more or all of the features defined in any one of claims 1 to 9.

59. A method of producing a cell according any one of claims 41 to 45, the method comprising
a) introducing into the cell, preferably a cell which is not capable of synthesising a LC-PUFA, the gene construct according to any one of claims 30 to 37, the isolated and/or exogenous polynucleotide of claim 38, the vector or genetic construct of claim 39 or claim 40, one or more of the combinations of exogenous polynucleotides defined in any one of claims 14, 41 to 43 or 48 to 52,
b) optionally, expressing the genes or polynucleotide(s) in the cell;
c) optionally, analysing the fatty acid composition of the cell, and
d) optionally, selecting a cell which express the genes or polynucleotide(s).

60. The method of claim 59, wherein lipid in the cell has one or more of the features defined in claims 1 to 9.

61. The method of claim 59 or claim 60, wherein the gene construct, the isolated and/or exogenous polynucleotide, the vector, the genetic construct or combinations of exogenous polynucleotides, become stably integrated into the genome of the cell.

62. The method according to any one of claims 59 to 61, wherein the cell is a plant cell, and the method further comprises the step of regenerating a transformed plant from the cell of step a).

63. The method of claim 62, wherein the genes and/or exogenous polynucleotide(s) are expressed transiently in the cell.

64. A cell produced using the method according to any one of claims 59 to 63.

65. A method of producing seed, the method comprising,
a) growing a plant according to any one of claims 48 to 54, or a plant which produces a part as defined in any one of claims 10 to 28, 56 or 57, preferably in a field as part of a population of at least 1000 such plants or in an area of at least 1 hectare planted at a standard planting density,
b) harvesting seed from the plant or plants, and
c) optionally, extracting lipid from the seed, preferably to produce oil with a total DHA yield of at least 60 kg DHA/hectare.

66. The plant, plant cell, plant part or seed of any one of claims 48 to 57, which has one or more of the following features
i) the oil is as defined in any one of claims 2, or 4 to 7,
ii) the plant part or seed is capable of being used in a process according to any one of claims 14, 16 to 19, 21 to 28,
iii) the exogenous polynucleotides are comprised in a genetic construct according to any one of claims 30 to 35,
iv) the exogenous polynucleotides comprise an exogenous polynucleotide of claim 38,
v) the plant cell is a cell according to claim 43, and
vi) the seed was produced according to the method of claim 65.

67. A method of producing one or more fatty acid desaturases and/or fatty acid elongases, or one or more fatty acid desaturases and one or more fatty acid elongases, the method comprising expressing in a cell or cell free expression system the gene construct of claim 36 or claim 37, the isolated and/or exogenous polynucleotide of claim 38, the vector or genetic construct of claim 39 or claim 40, one or more of the combinations of exogenous polynucleotides defined in any one of claims 14, 41 to 43 or 48 to 52.

68. Lipid, or oil, produced by, or obtained from, using the process according to any one of claims 10 to 28, the cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56, the seed of claim 57, or the plant, plant cell, plant part or seed of claim 66.

69. The lipid or oil of claim 29 or claim 68, which is obtained by extraction of oil from an oilseed.

70. The lipid or oil of claim 29, 68 or 69 which is canola oil (Brassica napus, Brassica rapa ssp.), mustard oil (Brassica juncea), other Brassica oil, sunflower oil (Helianthus annus), linseed oil (Linum usitatissimum), soybean oil (Glycine max), safflower oil (Carthamus tinctorius), corn oil (Zea mays), tobacco oil (Nicotiana tabacum), peanut oil (Arachis hypogaea), palm oil, cottonseed oil (Gossypium hirsutum), coconut oil (Cocos nucifera), avocado oil (Persea americana), olive oil (Olea europaea), cashew oil (Anacardium occidentale), macadamia oil (Macadamia intergrifolia), almond oil (Prunus amygdalus) or Arabidopsis seed oil (Arabidopsis thaliana).

71. A fatty acid produced by, or obtained from, using the process accoding to any one of claims 10 to 28, the cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56, the seed of claim 57, or the plant, plant cell, plant part or seed of claim 66.

72. Seedmeal obtained from seed according to any one of claims 56, 57 or 66.

73. A composition comprising one or more of the lipid or oil according to any one of claims 29 or 59 to 51, the fatty acid of claim 62, the genetic construct according to any one of claims 30 to 37, the isolated and/or exogenous polynucleotide of claim 38, the vector or genetic construct of claim 39 or claim 40, the cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56, the seed of claim 57, the plant, plant cell, plant part or seed of claim 66, or the seedmeal of claim 72.

74. Feedstuffs, cosmetics or chemicals comprising one or more of the lipid or oil according to any one of claims 1 to 9, 29 or 59, the fatty acid of claim 62, the gene construct according to any one of claims 30 to 37, the isolated and/or exogenous polynucleotide of claim 38, the vector or genetic construct of claim 39 or claim 40, the cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56, the seed of claim 57, the plant, plant cell, plant part or seed of claim 66, the seedmeal of claim 72, or the composition of claim 73.

75. A method of producing a feedstuff, the method comprising mixing one or more of the lipid or oil according to any one of claims 1 to 9, 29 or 59, the fatty acid of claim 62, the gene construct according to any one of claims 30 to 37, the isolated and/or exogenous polynucleotide of claim 38, the vector or genetic construct of claim 39 or claim 40, the cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56, the seed of claim 57, the plant, plant cell, plant part or seed of claim 66, the seedmeal of claim 72, or the composition of claim 73, with at least one other food ingredient.

76. A method of treating or preventing a condition which would benefit from a PUFA, the method comprising administering to a subject one or more of the lipid or oil according to any one of claims 1 to 9, 29 or 59, the fatty acid of claim 62, the gene construct according to any one of claims 30 to 37, the isolated and/or exogenous polynucleotide of claim 38, the vector or genetic construct of claim 39 or claim 40, the cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56, the seed of claim 57, the plant, plant cell, plant part or seed of claim 66, the seedmeal of claim 72, the composition of claim 73, or the feedstuff of claim 74.

77. The method of claim 76, wherein the condition is cardiac arrhythmia's, angioplasty, inflammation, asthma, psoriasis, osteoporosis, kidney stones, AIDS, multiple sclerosis, rheumatoid arthritis, Crohn's disease, schizophrenia, cancer, foetal alcohol syndrome, attention deficient hyperactivity disorder, cystic fibrosis, phenylketonuria, unipolar depression, aggressive hostility, adrenoleukodystophy, coronary heart disease, hypertension, diabetes, obesity, Alzheimer's disease, chronic obstructive pulmonary disease, ulcerative colitis, restenosis after angioplasty, eczema, high blood pressure, platelet aggregation, gastrointestinal bleeding, endometriosis, premenstrual syndrome, myalgic encephalomyelitis, chronic fatigue after viral infections or an ocular disease.

78. Use of one or more of the lipid or oil according to any one of claims 1 to 9, 29 or 59, the fatty acid of claim 62, the gene construct according to any one of claims 30 to 37, the isolated and/or exogenous polynucleotide of claim 38, the vector or genetic construct of claim 39 or claim 40 the cell according to any one of claims 41 to 46 or 55, the transgenic organism of claim 47 or claim 48, the oilseed plant according to any one or claims 49 to 53, the Brassica napus, B. juncea or Camelina sativa plant of claim 54, the plant part of claim 49 or claim 56, the seed of claim 57, the plant, plant cell, plant part or seed of claim 66, the seedmeal of claim 72, the composition of claim 73, or the feedstuff of claim 74 for the manufacture of a medicament for treating or preventing a condition which would benefit from a PUFA.

79. A method of trading seed, comprising obtaining seed of any one of claims 56, 57 or 66, and trading the obtained seed for pecuniary gain.

80. The method of claim 79, wherein obtaining the seed comprises cultivating the plant according to any one of claims 48 to 54 or 66, and/or harvesting the seed from the plants.

81. The method of claim 80, wherein obtaining the seed further comprises placing the seed in a container and/or storing the seed.

82. The method of claim according to any one of claims 79 to 81, wherein obtaining the seed further comprises transporting the seed to a different location.

83. The method of claim according to any one of claims 79 to 82, wherein the trading is conducted using electronic means such as a computer.

84. A process of producing bins of seed comprising:
a) swathing, windrowing and/or or reaping above-ground parts of plants comprising seed as defined in any one of claims 56, 57 or 66,
b) threshing and/or winnowing the parts of the plants to separate the seed from the remainder of the plant parts, and
c) sifting and/or sorting the seed separated in step b), and loading the sifted and/or sorted seed into bins, thereby producing bins of seed.

85. A process for producing ethyl esters of polyunsaturated fatty acids, the process comprising transesterifying triacylglycerols in extracted plant lipid, wherein the extracted plant lipid comprises fatty acids esterified in the form, the fatty acids comprising oleic acid, palmitic acid, ω6 fatty acids which comprise linoleic acid (LA), ω3 fatty acids which comprise α-linolenic acid (ALA), and docosahexaenoic acid (DHA), and optionally one or more of stearidonic acid (SDA), eicosapentaenoic acid (EPA), docosapentaenoic acid (DPA) and eicosatetraenoic acid (ETA), wherein the level of DHA in the total fatty acid content of the extracted lipid is about 7% to 20%, thereby producing the ethyl esters.
